(19) United States
(12) Patent Application Publication
(10) Pub. No.: US 2026/0015357 A1
(43) Pub. Date: Jan. 15, 2026

(54) MODULATORS OF NLRP3 INFLAMMASOME AND RELATED PRODUCTS AND METHODS

(71) Applicant: Neumora Therapeutics, Inc., Watertown, MA (US)

(72) Inventors: Anne-Marie BEAUSOLEIL, Watertown, MA (US); Ryan HUDSON, Watertown, MA (US)

(21) Appl. No.: 18/880,470

(22) PCT Filed: Jun. 30, 2023
(86) PCT No.: PCT/US2023/026796

§ 371 Date: Dec. 31, 2024

Related U.S. Application Data
(60) Provisional application No. 63/357,916, filed on Jul. 1, 2022.

(51) Int. Cl. C07D 487/04; A61K 31/502; A61K 31/5025; C07D 401/12; C07D 491/048; C07D 491/056; C07D 495/04; C07D 498/04; C07D 513/04

(52) U.S. Cl. CPC C07D 487/04; A61K 31/502; A61K 31/5025; C07D 401/12; C07D 491/048; C07D 491/056; C07D 495/04; C07D 498/04; C07D 513/04

(57) ABSTRACT

Compounds are provided for inhibiting NLRP3 inflammasome generally, or for treating a NLRP3 inflammasome dependent condition more specifically, by contacting the NLRP3 inflammasome or administering to a subject in need thereof, respectively, an effective amount of a compound having structure (I): or a pharmaceutically acceptable salt, isomer, hydrate, solvate or isotope thereof, wherein A, B, X₁, R₁, R₂, R₃, m, n, and p are as defined herein. Pharmaceutical compositions containing such compounds, as well as the compounds themselves, are also provided.

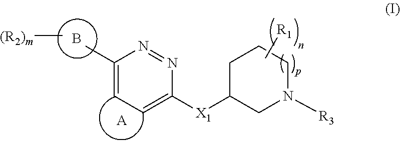

### BACKGROUND

### Technical Field

The invention relates to modulators of NLRP3 inflammasome, to products containing the same, as well as to methods of their use and preparation.

### Description of the Related Art

The NOD-like receptor protein 3 (NLRP3) is a key protein that interacts with, apoptosis-associated speck-like protein (ASC) and procaspase-1 to form the NLRP3 inflammasome. The activation of the NLRP3 inflammasome produces inflammatory mediators, such as interleukin-1β (IL-1β) and interleukin-18 (IL-18), thereby contributing to the activation of the innate immune system. The dysregulation of innate immunity contributes to various diseases.

The innate immune response protects the host from invading microorganisms. The formation of the NLRP3 inflammasome activates caspase-1, which leads to the maturation and secretion of IL-1β and IL-18, cleavage of gasdermin-D and, finally the initiation of cell death via pyroptosis. The system is alerted to the presence of the invading microorganism by cytokine release and is working to resolve the inflammation by eliminating the infected cells.

However, nonmicrobial compounds of either endogenous or exogenous origin are also effective inducers of NLRP3-dependent inflammation, allergic responses, or other forms of inflammation. For example, NLRP3 may be implicated in Parkinson's disease and/or Alzheimer's disease, suggesting that misfolded, oligomerized or aggregated proteins e.g. alpha-synuclein lead to the activation of the NLRP3 inflammasome. Environmental particulates such as inhaled asbestos and silica also activate the NLRP3 inflammasome, and high levels of IL-1β are involved in the development of asbestosis and silicosis, two progressive pulmonary diseases leading to fibrosis.

Emerging studies have revealed the involvement of increased production of IL-1β and IL-18 by the NLRP3 inflammasome can contribute to the onset and progression of various diseases such as neuroinflammation-related disorders, for example, brain infection, acute injury, multiple sclerosis, Alzheimer's disease, and neurodegenerative diseases; cardiovascular diseases, cardiovascular risk reduction, atherosclerosis, type I and type II diabetes and related complications, inflammatory skin diseases, acne, hidradenitis suppurativa, asthma, age-related macular degeneration, or cancer related diseases. Those disorders that are immune or inflammatory in nature are usually difficult to diagnose or treat efficiently.

Accordingly, there is a need in the art for compounds that modulate the NLRP3 inflammasome for the purpose of treating diseases in which modulation of the NLRP3 inflammasome would be beneficial.

The present invention fulfills these needs and provides other advantages as evident from the following description.

### BRIEF SUMMARY

In an embodiment a compound is provided having structure (I):

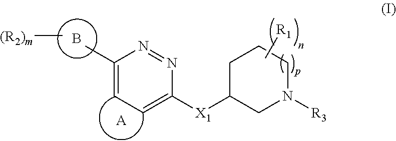

or a pharmaceutically acceptable salt, isomer, hydrate, solvate or isotope thereof, wherein A, B, X₁, R₁, R₂, R₃, m, n, and p are as defined herein.

In another embodiment, pharmaceutical compositions are provided comprising a carrier or excipient and a compound having structure (I), or a pharmaceutically acceptable salt, isomer, hydrate, solvate or isotope thereof.

In a further embodiment, a method is provided for treating a NLRP3 inflammasome-dependent condition by administering to a subject in need thereof an effective amount of a compounds of structure (I), or a pharmaceutically acceptable salt, isomer, hydrate, solvate or isotope thereof, or a pharmaceutical composition comprising the same. In some embodiments, the NLRP3 inflammasome-dependent condition is a neuroinflammation-related disorder or a neurodegenerative disease. In some embodiments, NLRP3 inflammasome activity is modulated such that there is no cardiotoxicity risk at physiologically relevant exposures for NLRP modulatory effect.

### DETAILED DESCRIPTION

### Definitions

As used herein, the following terms have the meaning defined below, unless the context indicates otherwise.

“Modulating” NLRP3 inflammasome means that the compound interacts with NLRP3 in a manner such that it blocks its ATPase function and the downstream formation of the NLRP3 inflammasome complex. In the above context, the compound acts to inhibit, or block, activation of the NLRP3 inflammasome and therefore the compound can also be described as an inhibitor.

NLRP3 refers to NOD-Like Receptor Protein 3. NLRP3 is a protein-coding gene, and an exemplary sequence may be found at https://www.uniprot.org/uniprot/Q96P20. NLRP3 functions as an ATPase that is auto-inhibited under normal conditions. NLRP3, unlike other inflammasomes, requires a two step activation process, which can be triggered by a variety of pathogen-associated molecular patterns (PAMPs) and damage-associated molecular patterns (DAMPs). The first step primes the NLRP3 inflammasome via recognition of PAMPs, DAMPs, or pathological species via pattern recognition receptors (PRRs) (e.g. Toll-like receptors). Priming increases NLRP3, pro-IL1β, and pro-IL18 expression. Activation of NLRP3 is triggered by a second signal, such as ATP, K⁺ efflux, other cellular stress, or pathological species, which leads to ATP hydrolysis and activation of the enzyme. This activation leads to oligomerization of the inflammasome complex, resulting in auto-cleavage and activation of caspase-1. Active caspase-1 then cleaves pro-IL1β and pro-IL18. Active IL1β and IL18 then are released from the cell through a gasdermin D pore, and release of such cytokines triggers a downstream inflammatory response and cell death via pyroptosis.

“Effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

“Alkyl” means a saturated or unsaturated straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of saturated straight chain alkyl groups include, but are not limited to, methyl, ethyl, n-propyl, n-butyl, n-pentyl-, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups.

“Halo” or “halogen” refers to fluorine, chlorine, bromine, and iodine.

“Hydroxyl” refers to —OH.

“Cyano” refers to —CN.

“Oxo” refers to the ═O substituent.

“Haloalkyl” refers to alkyl as defined above with one or more hydrogen atoms replaced with halogen. Examples of haloalkyl groups include, but are not limited to, —CF₃, —CHF₂, and the like.

“Alkoxy” refers to alkyl as defined above joined by way of an oxygen atom (i.e., —O-alkyl). Examples of alkoxy groups include, but are not limited to, methoxy, ethoxy, n-propoxy, n-butoxy, isopropoxy, sec-butoxy, tert-butoxy, and the like.

“Haloalkoxy” refers to haloalkyl as defined above joined by way of an oxygen atom (i.e., —O-haloalkyl). Examples of haloalkoxy groups include, but are not limited to, —OCF₃, and the like.

“Aminyl” refers to —NH₂, —NHalkyl or N(alkyl)₂, wherein alkyl is as defined above. Examples of amino or aminyl include, but are not limited to —NH₂, —NHCH₃, —N(CH₃)₂, and the like.

“Alkylsulfonyl” refers to alkyl as described above joined by way of a sulfonyl (i.e., —S(O)₂-alkyl). Examples of alkylsulfonyl groups include, but are not limited to,

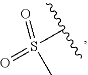 [SMILES: *S(C)(=O)=O]

and the like.

“Carbocyclyl” or “carbocyclic ring” refers to a stable, aromatic or non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is attached to the rest of the molecule by a single bond. Carbocyclyl includes aryl and cycloalkyl groups.

“Cycloalkyl” refers to non-aromatic ring moieties containing 3 or more ring members. In some embodiments, cycloalkyl includes 3 to 8 ring members. In some embodiments, cycloalkyl includes 3 to 5 ring members. For example, cycloalkyl includes cyclopropyl, cyclobutyl, cyclopentyl, cyclohexyl, and the like.

“Aryl” groups are cyclic aromatic hydrocarbons that do not contain heteroatoms. Representative aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl.

“Heterocycle” or “heterocyclyl” or “heterocyclic ring” refers to aromatic and non-aromatic ring moieties containing 3 or more ring members, of which one or more is a heteroatom such as, but not limited to, N, O, S, or P. In some embodiments, heterocyclyl include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

“One or more” when referring to substitution can mean a single substituent, two substituents, three substituents, and the like, as appropriate for the compound structure as understood by a person of skill in the art.

“Isomer” is used herein to encompass all chiral, diastereomeric or racemic forms of a structure (also referred to as a stereoisomer, as opposed to a structural or positional isomer), unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure by weight. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out). All compounds with an asterisk (*) adjacent to a tertiary or quaternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of structure (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine-19 is longest-lived. Thus, an isotope of a compound having the structure of structure (I) includes, but not limited to, compounds of structure (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the invention include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, Phydroxybutyric, salicylic, galactaric, and galacturonic acid.

The compounds of the disclosure (i.e., compounds of structure (I) and embodiments thereof), or their pharmaceutically acceptable salts may contain one or more centers of geometric asymmetry and may thus give rise to enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also included.

Although pharmaceutically unacceptable salts are not generally useful as medicaments, such salts may be useful, for example as intermediates in the synthesis of compounds having the structure of Formula 1, for example in their purification by recrystallization.

As used herein, the phrase “NLRP3 inflammasome dependent condition” means a condition wherein modulating NLRP3 provides a medical benefit to the patient or subject.

In some embodiments, the NLRP3 inflammasome dependent condition is inflammation, an inflammatory disease, an immune disease, cancer, infections including viral infections, central nervous system diseases, metabolic diseases, cardiovascular diseases, respiratory diseases, liver diseases, renal diseases, ocular diseases, skin diseases, psychological diseases or blood diseases.

In one embodiment, the NLRP3 inflammasome dependent condition is neuroinflammation-related disorders or neurodegenerative diseases.

In one embodiment, the invention provides a method for inhibiting NLRP3 inflammasome with an effective amount of a pharmaceutical composition as described herein.

In another embodiment, the invention provides a method of treating a NLRP3 inflammasome dependent condition by administering to a subject in need thereof an effective amount of a pharmaceutical composition as described herein. In certain embodiments, the NLRP3 inflammasome dependent condition is a neuroinflammation-related disorder(s) or a neurodegenerative disease(s).

As used herein the phrase “inflammation” refers to inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity. Examples of inflammation that may be treated or prevented include inflammatory responses occurring in connection with, or as a result of:
(a) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(b) a joint condition such as osteoarthritis, systemic juvenile idiopathic arthritis, adult-onset Still's disease, relapsing polychondritis, rheumatoid arthritis, juvenile chronic arthritis, crystal induced arthropathy (e.g. pseudo-gout, gout), or a seronegative spondyloarthropathy (e.g. ankylosing spondylitis, psoriatic arthritis or Reiter's disease);
(c) a muscular condition such as polymyositis or myasthenia gravis;
(d) a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(e) a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
(f) a vascular condition such as atherosclerosis, Behcet's disease, vasculitides, or Wegener's granulomatosis;
(g) an immune condition, e.g. autoimmune condition, such as systemic lupus erythematosus (SLE), Sjogren's syndrome, systemic sclerosis, Hashimoto's thyroiditis, type 1 diabetes, idiopathic thrombocytopenia purpura, or Graves disease;
(h) an ocular condition such as uveitis, allergic conjunctivitis, or vernal conjunctivitis;
(i) a nervous condition such as multiple sclerosis or encephalomyelitis;
(j) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, Epstein Barr virus, viral encephalitis/aseptic meningitis, or pelvic inflammatory disease;
(k) a renal condition such as mesangial proliferative glomerulonephritis, nephrotic syndrome, nephritis, glomerular nephritis, acute renal failure, uremia, or nephritic syndrome;
(l) a lymphatic condition such as Castleman's disease;
(m) a condition of, or involving, the immune system, such as hyper IgE syndrome, lepromatous leprosy, familial hemophagocytic lymphohistiocytosis, or graft versus host disease;
(n) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;
(o) a cancer, including those cancers listed herein below;
(p) a burn, wound, trauma, hemorrhage or stroke;
(q) radiation exposure; and/or
(r) obesity; and/or
(s) pain such as inflammatory hyperalgesia.

As used herein the phrase “an inflammatory disease” means for example, inflammation occurring as a result of an inflammatory disorder, e.g. an autoinflammatory disease, such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anemia with B-cell immunodeficiency, periodic fevers and developmental delay (SIFD).

As used herein the phrase “an immune disease” means for example, autoimmune diseases, such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjogren's syndrome, systemic sclerosis a systemic connective tissue disorder, Takayasu's arteritis, temporal arteritis, warm autoimmune hemolytic anemia, Wegener's granulomatosis, alopecia universalis, Beliefs disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, giant cell arteritis, vitiligo or vulvodynia.

As used herein the phrase “cancer” means for example, lung cancer, renal cell carcinoma, non-small cell lung carcinoma (NSCLC), Langerhans cell histiocytosis (LCH), myeloproliferative neoplasm (MPN), pancreatic cancer, gastric cancer, myelodysplastic syndrome (MDS), leukemia including acute lymphocytic leukemia (ALL) and acute myeloid leukemia (AML), promyelocytic leukemia (APML, or APL), adrenal cancer, anal cancer, basal and squamous cell skin cancer, bile duct cancer, bladder cancer, bone cancer, brain and spinal cord tumors, breast cancer, cervical cancer, chronic lymphocytic leukemia (CLL), chronic myeloid leukemia (CML), chronic myelomonocytic leukemia (CMML), colorectal cancer, endometrial cancer, esophagus cancer, Ewing family of tumors, eye cancer, gallbladder cancer, gastrointestinal carcinoid tumors, gastrointestinal stromal tumor (GIST), gestational trophoblastic disease, glioma, Hodgkin lymphoma, Kaposi sarcoma, kidney cancer, laryngeal and hypopharyngeal cancer, liver cancer, lung carcinoid tumor, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumors, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumor.

As used herein the phrase “infections including viral infections” means for example, viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordetella pertussis, Burkholderia pseudomallei, Corynebacterium diphtheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from Schistosoma, roundworms, tapeworms or flukes), and prion infections.

As used herein the phrase “central nervous system diseases” means for example, Parkinson's disease, Alzheimer's disease, Frontotemporal dementia, dementia, motor neuron disease, Huntington's disease, cerebral malaria, brain injury from pneumococcal meningitis, intracranial aneurysms, traumatic brain injury, multiple sclerosis, and amyotrophic lateral sclerosis.

As used herein the phrase “neuroinflammation-related diseases” means for example, multiple sclerosis, brain infection, acute injury, neurodegenerative disease, Parkinson's disease or Alzheimer's disease.

As used herein the phrase “neurodegenerative disease” means for example, Alzheimer's disease, Parkinson's disease, multiple sclerosis, or amyotrophic lateral sclerosis.

In one embodiment, neurodegenerative diseases are characterized by deep involvement of cell mediating neuroinflammatory processes.

As used herein the phrase “metabolic diseases” means for example, type 2 diabetes (T2D), atherosclerosis, obesity, gout, and pseudo-gout.

As used herein the phrase “cardiovascular diseases” means for example, hypertension, ischemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, cardiovascular risk reduction (CvRR), and pericarditis including Dressler's syndrome.

As used herein the phrase “respiratory diseases” means for example, chronic obstructive pulmonary disorder (COPD), asthma such as allergic asthma and steroid-resistant asthma, asbestosis, silicosis, nanoparticle induced inflammation, cystic fibrosis, and idiopathic pulmonary fibrosis.

As used herein the phrase “liver diseases” means for example, non-alcoholic fatty liver disease (NAFLD) and nonalcoholic steatohepatitis (NASH) including advanced fibrosis stages F3 and F4, alcoholic fatty liver disease (AFLD), and alcoholic steatohepatitis (ASH).

As used herein the phrase “renal diseases” means for example, acute kidney disease, hyperoxaluria, chronic kidney disease, oxalate nephropathy, nephrocalcinosis, glomerulonephritis, and diabetic nephropathy;

As used herein the phrase “ocular diseases” means for example, diseases of the ocular epithelium, age-related macular degeneration (AMD) (dry and wet), uveitis, corneal infection, diabetic retinopathy, optic nerve damage, dry eye, and glaucoma.

As used herein the phrase “skin diseases” means for example, dermatitis such as contact dermatitis and atopic dermatitis, contact hypersensitivity, sunburn, skin lesions, hidradenitis suppurativa (HS), other cyst-causing skin diseases, and acne conglobate.

As used herein the phrase “psychological diseases” means for example, depression, and psychological stress.

As used herein the phrase “blood diseases” means for example, sickle cell disease.

As used herein, the term “administration” refers to providing a compound, or a pharmaceutical composition comprising the compound as described herein. The compound or composition can be administered by another person to the subject or it can be self-administered by the subject. Non-limiting examples of routes of administration are oral, parenteral (e.g., intravenous), or topical.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human). A subject to be treated according to the methods described herein may be one who has been diagnosed with a NLRP3 inflammasome dependent condition, such as inflammation, an inflammatory disease, an immune disease, cancer, infections including viral infections; central nervous system diseases, metabolic diseases, cardiovascular diseases, respiratory diseases, liver diseases, renal diseases, ocular diseases, skin diseases, psychological diseases or blood diseases.

Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. The term “patient” may be used interchangeably with the term “subject.” A subject may refer to an adult or pediatric subject.

### Compounds

As detailed above, the present disclosure provides compounds showing significant activity as NLRP3 inflammasome antagonists (i.e., as NLRP3 inflammasome inhibitors). Accordingly, in some embodiments a compound is provided having structure (A):

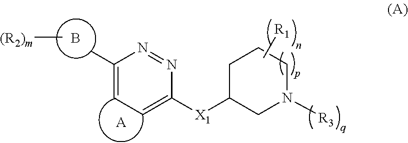

or a pharmaceutically acceptable salt, isomer, hydrate, solvate or isotope thereof, wherein:
ring A forms a fused 5-membered carbocyclic or heterocyclic ring optionally substituted by one or more R₄;
ring B is piperidinyl or an aryl ring;
X₁ is CRₐR_{b}, NR₄, or O;
Rₐ and R_{b} are each independently H or alkyl;
R₁ is halo;
R₂ is halo, OH, CN, alkyl, haloalkyl, alkoxy, haloalkoxy, or cycloalkyl;
R₃ is H, cycloalkyl, or alkyl optionally substituted with one or more R_{3′};
R_{3′} is OH, halo, alkoxy, aminyl, alkylsulfonyl, cycloalkyl, aryl, or heterocyclyl;
each R₄ is independently H, halo, or alkyl, or when attached to a carbon atom, two R₄ may join together to form oxo;
m is 0-5;
n is 0-4;
p is 0-2; and
q is 1 or 2.

In some embodiments a compound is provided having structure (I):

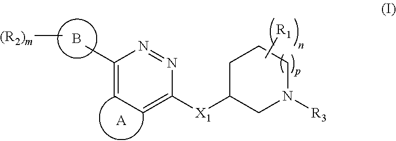

or a pharmaceutically acceptable salt, isomer, hydrate, solvate or isotope thereof, wherein:
ring A forms a fused 5-membered carbocyclic or heterocyclic ring optionally substituted by one or more R₄;
ring B is an aryl ring;
X₁ is CRₐR_{b}, NR₄, or O;
Rₐ and R_{b} are each independently H or alkyl;
R₁ is halo;
R₂ is halo, OH, CN, alkyl, haloalkyl, alkoxy, haloalkoxy, or cycloalkyl;
R₃ is H, cycloalkyl, or alkyl optionally substituted with one or more R_{3′};
R_{3′} is OH, halo, or cycloalkyl;
each R₄ is independently H or alkyl, or when attached to a carbon atom, two R₄ may join together to form oxo;
m is 0-4;
n is 0-4; and
p is 0-2.

In other embodiments, a compound is provided having structure (I):

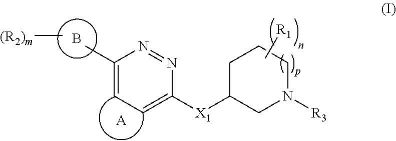

or a pharmaceutically acceptable salt, isomer, hydrate, solvate or isotope thereof, wherein:
ring A forms a fused 5-membered carbocyclic or heterocyclic ring optionally substituted by one or more R₄;
ring B is an aryl ring;
X₁ is CRₐR_{b}, NR₄, or O;
Rₐ and R_{b} are each independently H or alkyl;
R₁ is halo;
R₂ is halo, OH, alkyl, or haloalkyl;
R₃ is alkyl;
each R₄ is independently H or alkyl, or when attached to a carbon atom, two R₄ may join together to form oxo;
m is 0-2;
n is 0-2; and
p is 1.

In other embodiments, a compound is provided having structure (la):

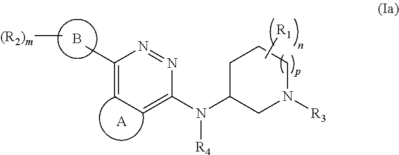

or a pharmaceutically acceptable salt, isomer, hydrate, solvate or isotope thereof, wherein A, B, R₁, R₂, R₃, R₄, m, n, and p are as defined herein.

In additional embodiments, a compound is provided having structure (Ib):

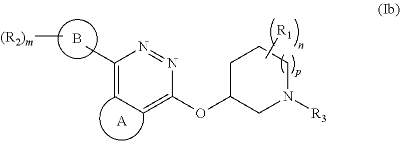

or a pharmaceutically acceptable salt, isomer, hydrate, solvate or isotope thereof, wherein A, B, R₁, R₂, R₃, m, n, and p are as defined herein.

In further embodiments, a compound is provided having structure (Ic):

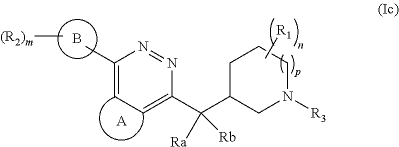

or a pharmaceutically acceptable salt, isomer, hydrate, solvate or isotope thereof, wherein A, B, R₁, R₂, R₃, m, n, and p are as defined herein.

In some embodiments, a compound is provided wherein

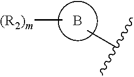

has one of the following structures:

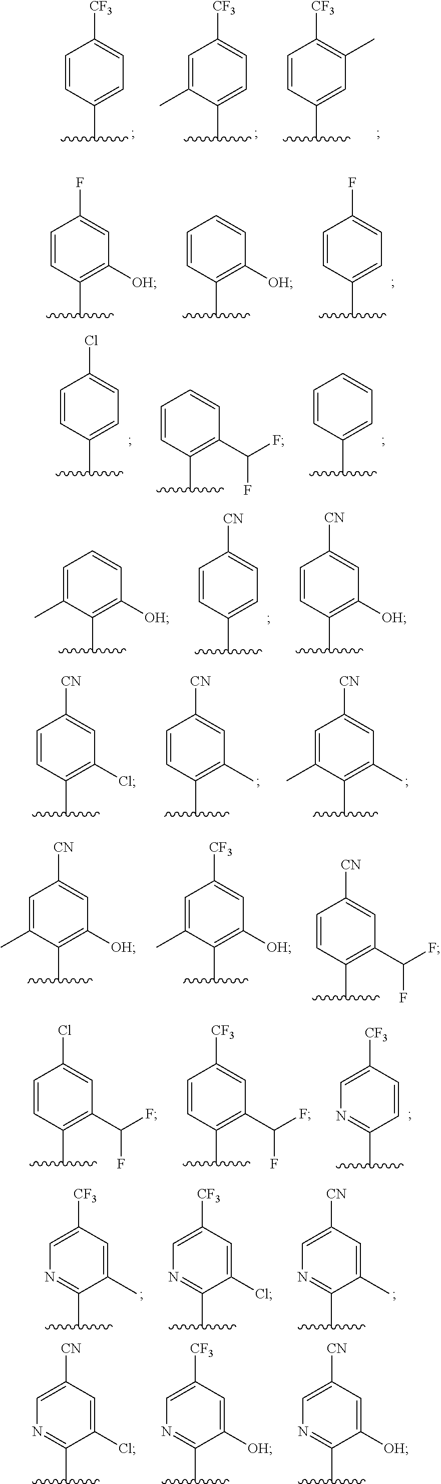

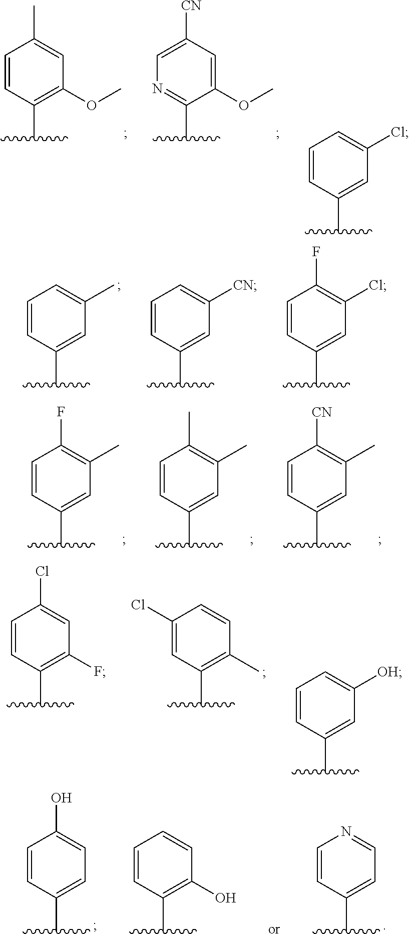

In some embodiments, a compound is provided wherein

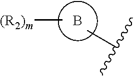

has one of the following structures:

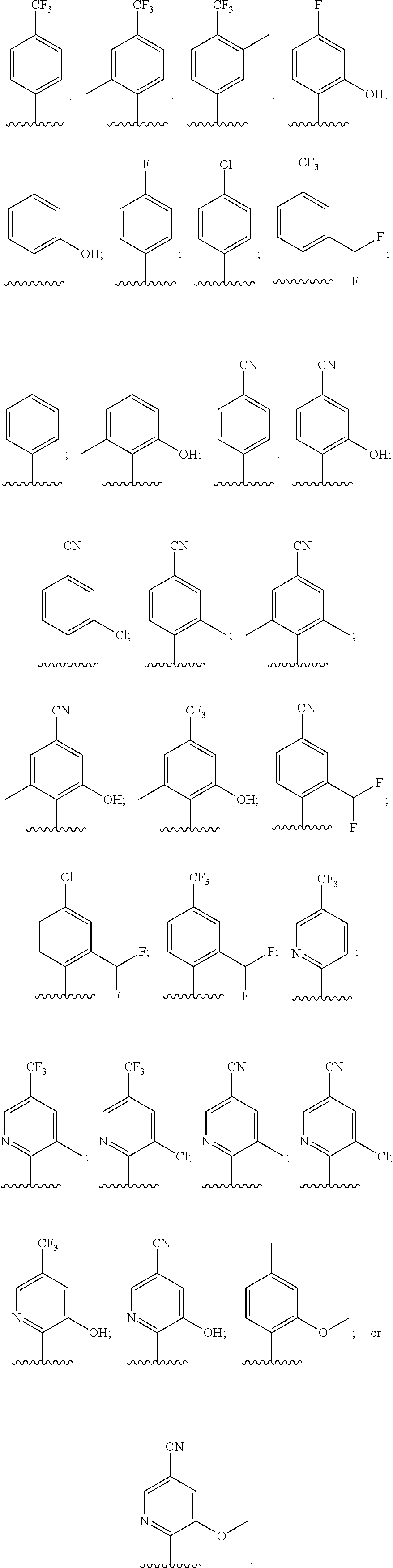

In some embodiments, a compound is provided wherein

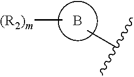

has one of the following structures:

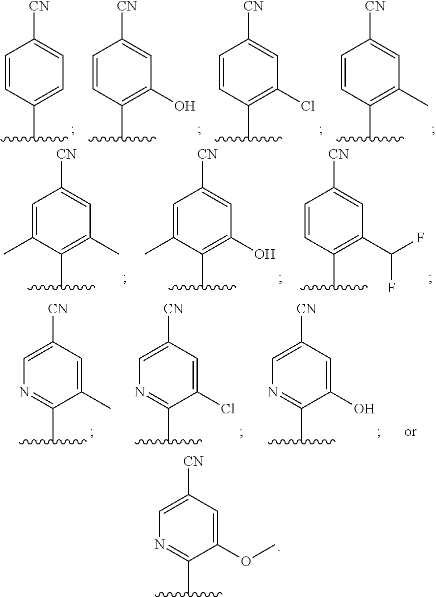

In some embodiments, a compound is provided wherein

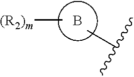

has one of the following structures:

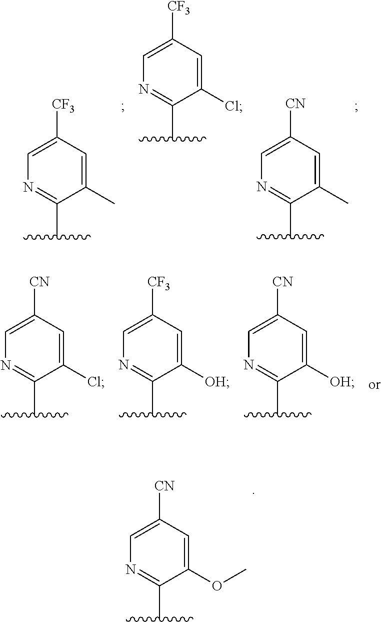

In some embodiments, a compound is provided wherein B is piperidinyl.

In some embodiments, a compound is provided having structure (I′):

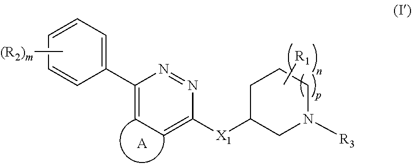

or a pharmaceutically acceptable salt, isomer, hydrate, solvate or isotope thereof, wherein:
ring A forms a fused 5-membered carbocyclic or heterocyclic ring optionally substituted by one or more R₄;
X₁ is CRₐR_{b}, NR₄, or O;
Rₐ and R_{b} are each independently H or alkyl;
R₁ is halo;
R₂ is halo, OH, CN, alkyl, haloalkyl, alkoxy, haloalkoxy, or cycloalkyl;
R₃ is H, cycloalkyl, or alkyl optionally substituted with one or more R_{3′};
R_{3′} is OH, halo, or cycloalkyl;
each R₄ is independently H or alkyl, or when attached to a carbon atom, two R₄ may join together to form oxo;
m is 0-4;
n is 0-4; and
p is 0-2.

In other embodiments, a compound is provided having structure (I′):

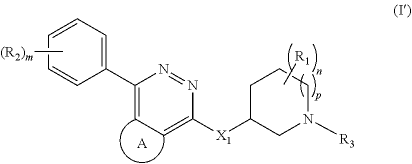

or a pharmaceutically acceptable salt, isomer, hydrate, solvate or isotope thereof, wherein:
ring A forms a fused 5-membered carbocyclic or heterocyclic ring optionally substituted by one or more R₄;
X₁ is CRₐR_{b}, NR₄, or O;
Rₐ and R_{b} are each independently H or alkyl;
R₁ is halo;
R₂ is halo, OH, alkyl, or haloalkyl;
R₃ is alkyl;
each R₄ is independently H or alkyl, or when attached to a carbon atom, two R₄ may join together to form oxo;
m is 0-2;
n is 0-2; and
p is 1.

In additional embodiments, a compound is provided having structure (I′a):

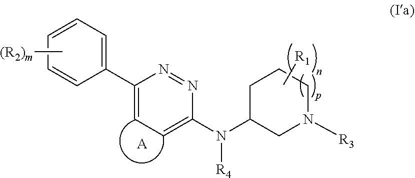

or a pharmaceutically acceptable salt, isomer, hydrate, solvate or isotope thereof, wherein A, R₁, R₂, R₃, R₄, m, n, and p are as defined herein.

In further embodiments, a compound is provided having structure (I′b):

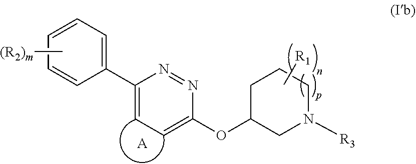

or a pharmaceutically acceptable salt, isomer, hydrate, solvate or isotope thereof, wherein A, R₁, R₂, R₃, R₄, m, n, and p are as defined herein.

In yet other embodiments, a compound is provided having structure (I′c):

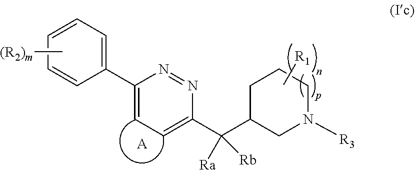

or a pharmaceutically acceptable salt, isomer, hydrate, solvate or isotope thereof, wherein A, B, R₁, R₂, R₃, Rₐ, R_{b}, m, n, and p are as defined herein.

In some embodiments, a compound is provided having structure (I″):

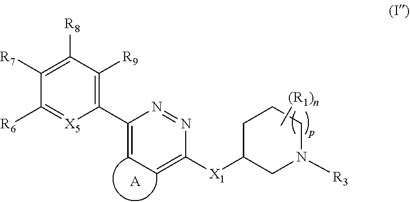

or a pharmaceutically acceptable salt, isomer, hydrate, solvate or isotope thereof, wherein:
ring A forms a fused 5-membered carbocyclic or heterocyclic ring optionally substituted by one or more R₄;
X₁ is CRₐR_{b}, NR₄, or O;
X₅ is CR₅ or N;
Rₐ and R_{b} are each independently H or alkyl;
R₁ is halo;
R₃ is H, cycloalkyl, or alkyl optionally substituted with one or more R_{3′};
R_{3′} is OH, halo, or cycloalkyl;
each R₄ is independently H or alkyl, or when attached to a carbon atom, two R₄ may join together to form oxo;
R₅ is H, halo, OH, CN, alkyl, haloalkyl, alkoxy, haloalkoxy, or cycloalkyl;
R₆ is H, halo, OH, CN, alkyl, haloalkyl, alkoxy, haloalkoxy, or cycloalkyl;
R₇ is H, halo, OH, CN, alkyl, haloalkyl, alkoxy, haloalkoxy, or cycloalkyl;
R₈ is H, halo, OH, CN, alkyl, haloalkyl, alkoxy, haloalkoxy, or cycloalkyl;
R₉ is H, halo, OH, CN, alkyl, haloalkyl, alkoxy, haloalkoxy, or cycloalkyl;
n is 0-4; and
p is 0-2.

In some embodiments, a compound is provided having structure (I″):

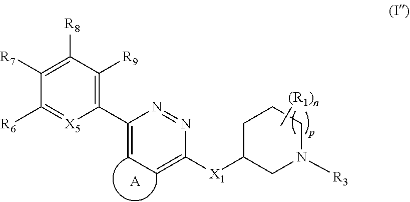

or a pharmaceutically acceptable salt, isomer, hydrate, solvate or isotope thereof, wherein:
ring A forms a fused 5-membered heterocyclic ring comprising nitrogen and optionally substituted by one or more R₄; and
R₇ is CN.

In some embodiments, a compound is provided having structure (I″a):

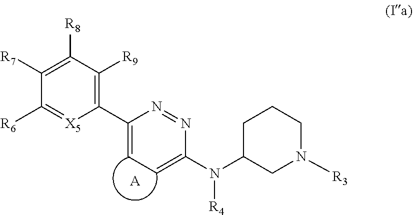

or a pharmaceutically acceptable salt, isomer, hydrate, solvate or isotope thereof, wherein:
ring A forms a fused 5-membered heterocyclic ring optionally substituted by one or more R₄;
X₅ is CR₅ or N;
R₃ is H, cycloalkyl, or alkyl optionally substituted with one or more R_{3′};
R_{3′} is OH, halo, or cycloalkyl;
each R₄ is independently H or alkyl, or when attached to a carbon atom, two R₄ may join together to form oxo;
R₅ is H, halo, OH, CN, alkyl, haloalkyl, alkoxy, haloalkoxy, or cycloalkyl;
R₆ is H, halo, OH, CN, alkyl, haloalkyl, alkoxy, haloalkoxy, or cycloalkyl;
R₇ is H, halo, OH, CN, alkyl, haloalkyl, alkoxy, haloalkoxy, or cycloalkyl;
R₈ is H, halo, OH, CN, alkyl, haloalkyl, alkoxy, haloalkoxy, or cycloalkyl; and
R₉ is H, halo, OH, CN, alkyl, haloalkyl, alkoxy, haloalkoxy, or cycloalkyl.

In some embodiments, a compound is provided having structure (I″a):

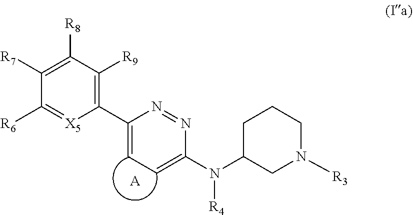

or a pharmaceutically acceptable salt, isomer, hydrate, solvate or isotope thereof, wherein:
   ring A forms a fused 5-membered heterocyclic ring comprising nitrogen and optionally substituted by one or more R₄;
X₅ is CR₅;
R₇ is CN.

In some embodiments, a compound is provided having structure (I″a):

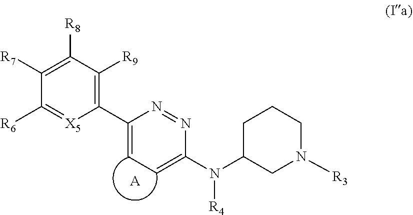

or a pharmaceutically acceptable salt, isomer, hydrate, solvate or isotope thereof, wherein:
   ring A forms a fused 5-membered heterocyclic ring comprising nitrogen and optionally substituted by one or more R₄;
X₅ is CR₅; and
R₅ is H, halo, CN, alkyl, haloalkyl, alkoxy, haloalkoxy, or cycloalkyl;
R₇ is CN; and
R₉ is H, halo, CN, alkyl, haloalkyl, alkoxy, haloalkoxy, or cycloalkyl.

In some embodiments, a compound is provided having structure (I″a):

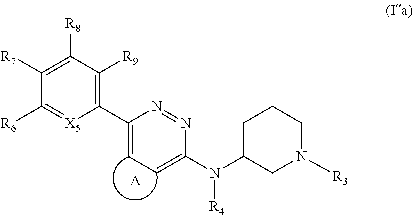

or a pharmaceutically acceptable salt, isomer, hydrate, solvate or isotope thereof, wherein:
X₅ is N; and
R₇ is CN or haloalkyl.

In some embodiments, a compound is provided having structure (I″a):

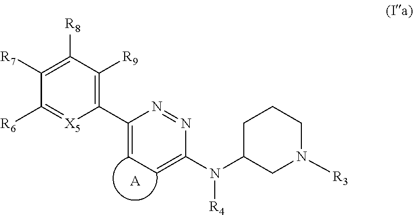

or a pharmaceutically acceptable salt, isomer, hydrate, solvate or isotope thereof, wherein:
X₅ is N;
R₅ is H, halo, CN, alkyl, haloalkyl, alkoxy, haloalkoxy, or cycloalkyl;
R₇ is CN or haloalkyl; and
R₉ is H, halo, CN, alkyl, haloalkyl, alkoxy, haloalkoxy, or cycloalkyl.

In some embodiments, a compound is provided wherein ring A forms a fused 5-membered carbocyclic ring. In other embodiments, a compound is provided wherein ring A forms a fused 5-membered heterocyclic ring. In certain embodiments, a compound is provided wherein ring A forms a fused 5-membered ring having one of the following structures:

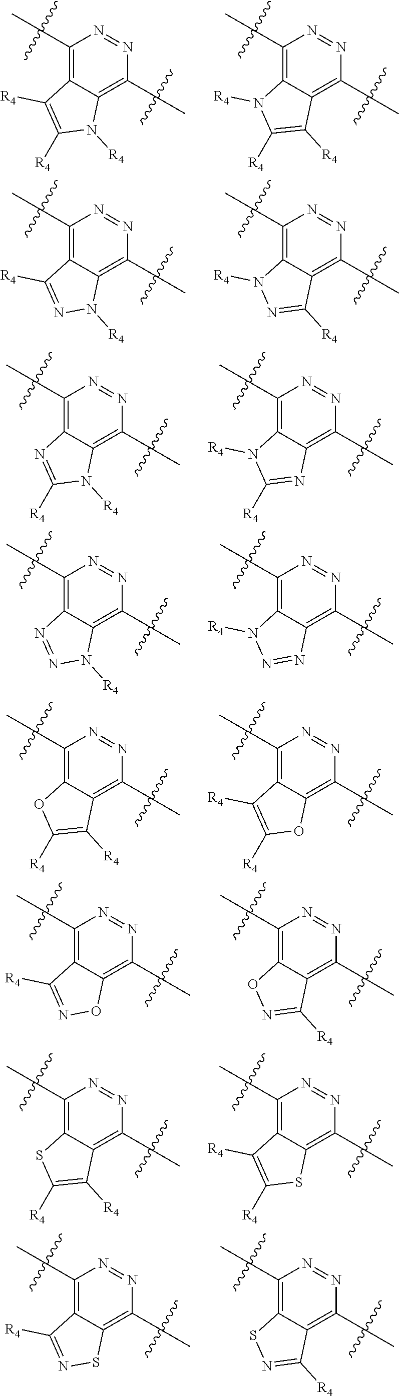

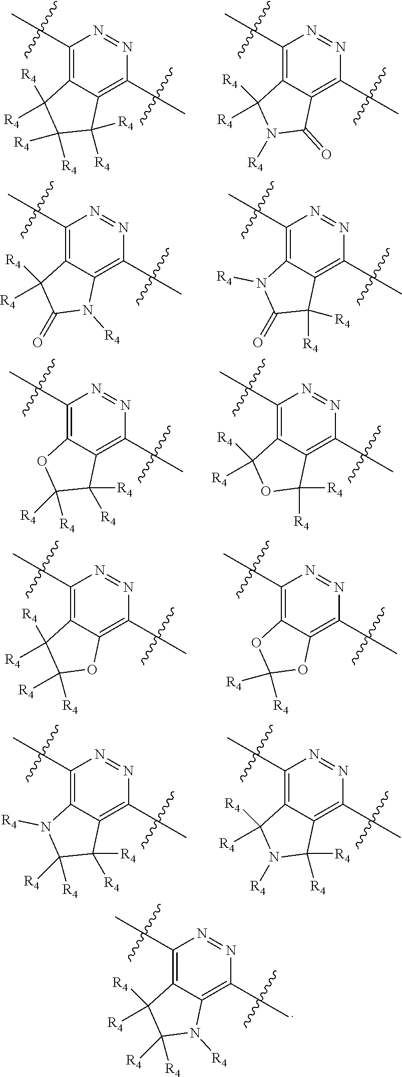

In yet other embodiments, a compound is provided wherein ring A forms a fused 5-membered ring having one of the following structures:

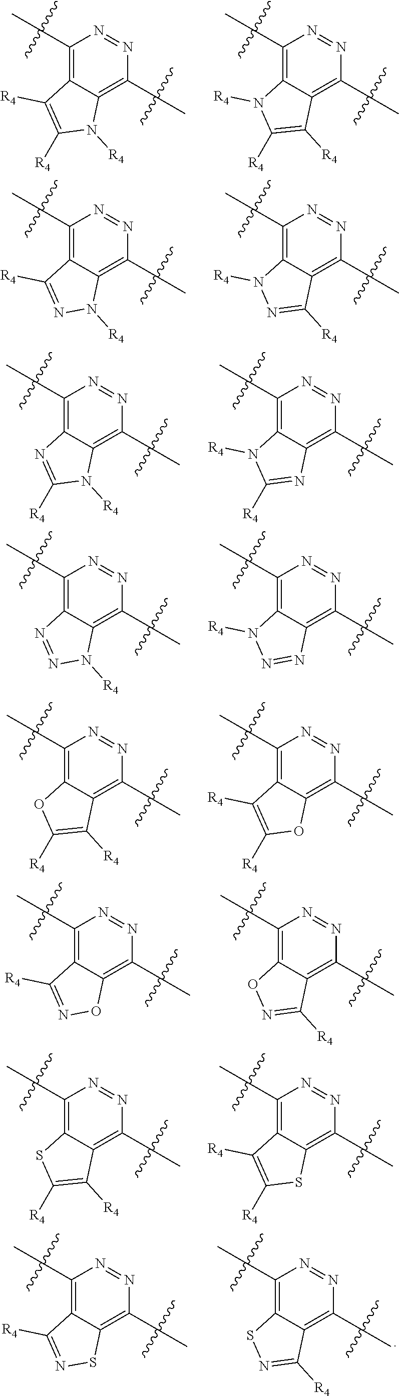

In further embodiments, a compound is provided wherein ring A forms a fused 5-membered ring having one of the following structures:

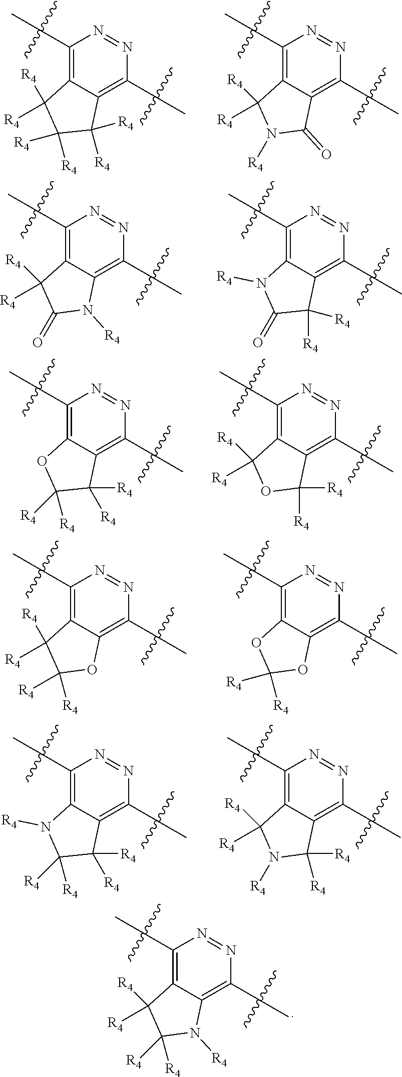

In some embodiments, a compound is provided wherein ring A forms a fused 5-membered ring having one of the following structures:

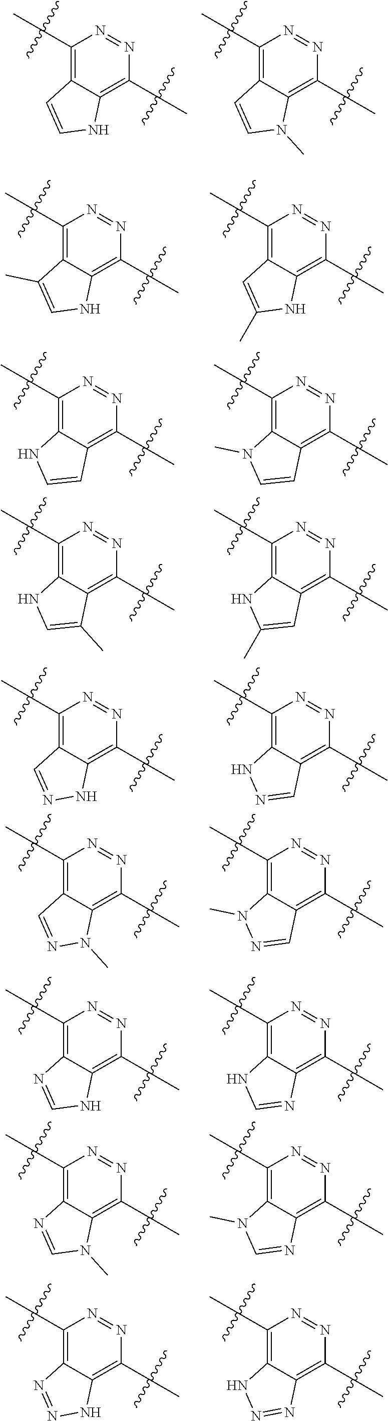

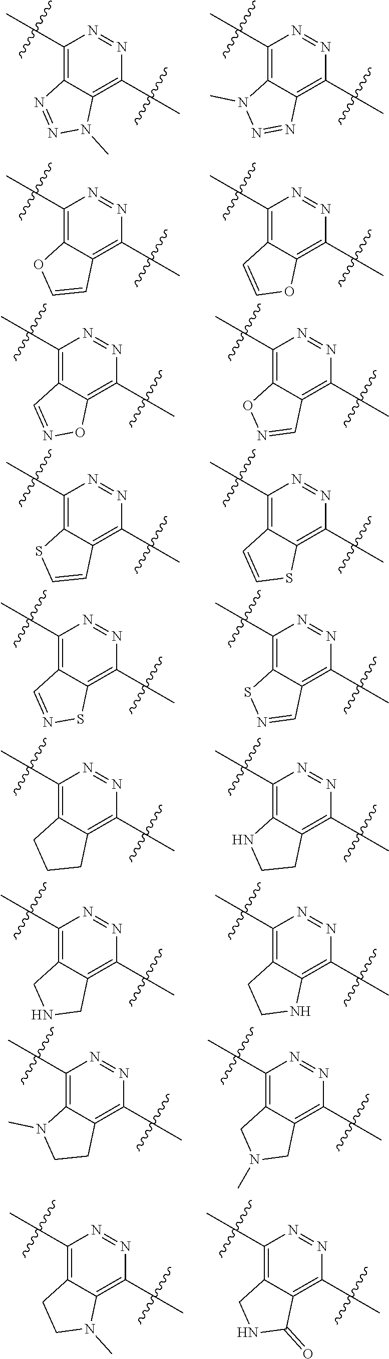

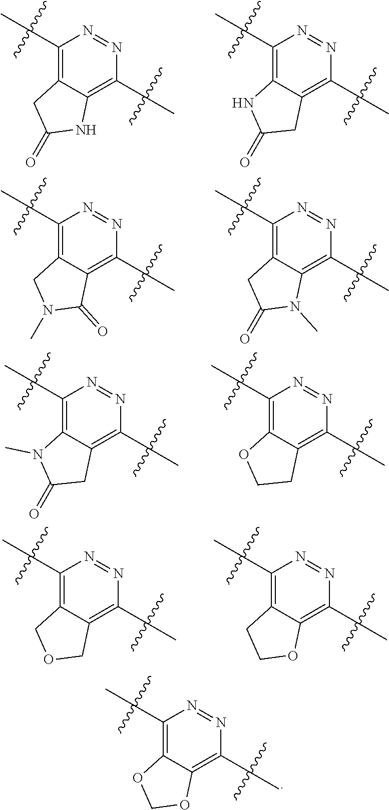

In other embodiments, a compound is provided wherein ring A forms a fused 5-membered ring having one of the following structures:

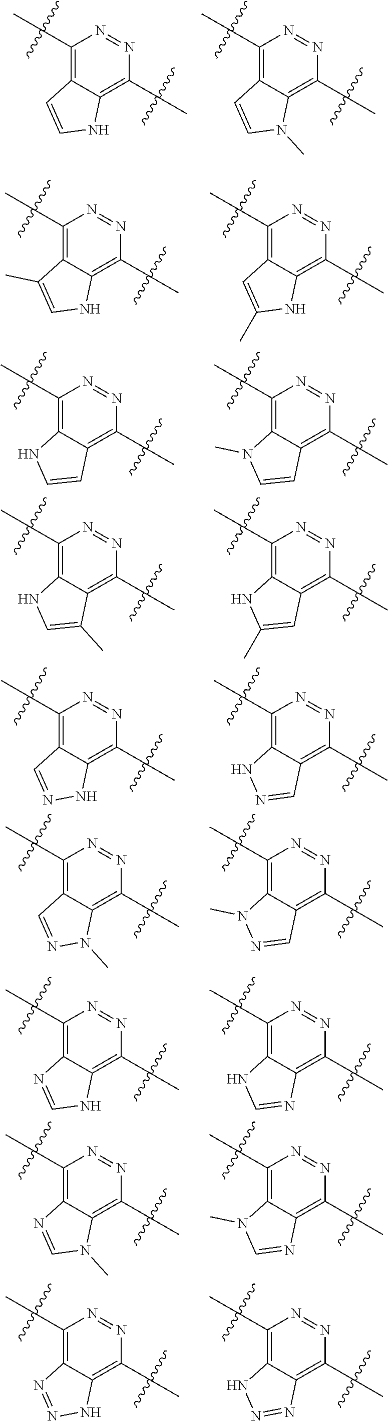

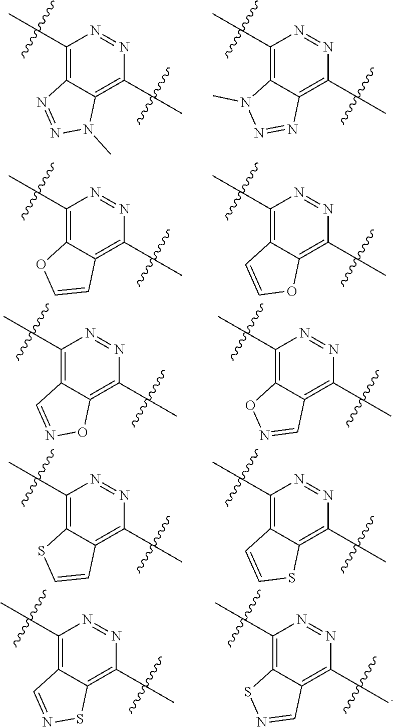

In yet other embodiments, a compound is provided wherein ring A forms a fused 5-membered ring having one of the following structures:

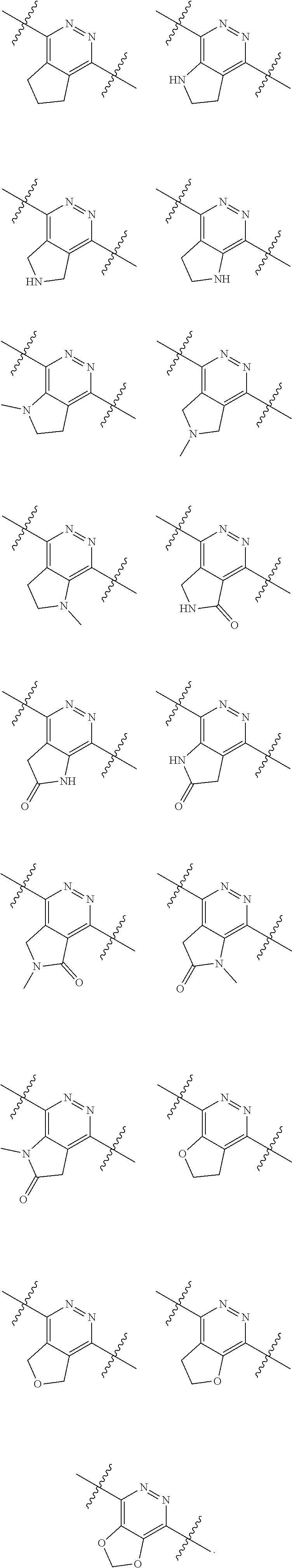

In yet other embodiments, a compound is provided having structure (I′):

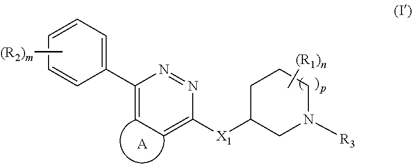

or a pharmaceutically acceptable salt, isomer, hydrate, solvate or isotope thereof, wherein ring A forms a fused 5-membered carbocyclic ring optionally substituted by one or more R₄ and wherein A, X₁, R₁, R₂, R₃, m, n, and p are as defined herein. In some embodiments, a compound is provided wherein ring A forms a fused 5-membered non-aromatic carbocyclic ring.

In some embodiments, a compound is provided wherein ring A forms a fused 5-membered carbocyclic ring having the following structure:

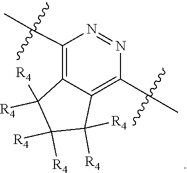

In certain embodiments, a compound is provided wherein ring A forms a fused 5-membered carbocyclic ring having the following structure:

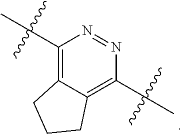 [SMILES: *c1nnc(*)c2c1CCC2]

In some embodiments, a compound is provided wherein ring A forms a fused 5-membered carbocyclic ring having the following structure:

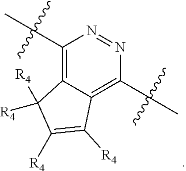

In certain embodiments, a compound is provided wherein ring A forms a fused 5-membered carbocyclic ring having the following structure

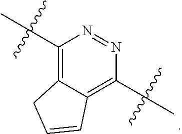 [SMILES: *c1nnc(*)c2c1C=CC2]

In some embodiments, a compound is provided wherein ring A forms a fused 5-membered carbocyclic ring having the following structure:

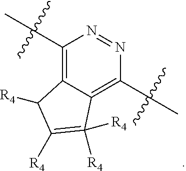

In certain embodiments, a compound is provided wherein ring A forms a fused 5-membered carbocyclic ring having the following structure:

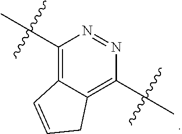 [SMILES: *c1nnc(*)c2c1C=CC2]

In some embodiments, a compound is provided having structure (I′):

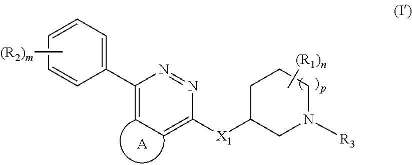

or a pharmaceutically acceptable salt, isomer, hydrate, solvate or isotope thereof, wherein ring A forms a fused 5-membered heterocyclic ring and wherein A, X₁, R₁, R₂, R₃, m, n, and p are as defined herein. In certain embodiments, a compound is provided wherein ring A forms a fused 5-membered heterocyclic ring comprising 1-3 nitrogen atoms optionally substituted by one or more R₄. In certain embodiments, a compound is provided wherein ring A forms a fused 5-membered heterocyclic ring optionally substituted by one or more R₄, wherein the heteroatoms consist of 1-3 nitrogen atoms. In some embodiments, a compound is provided wherein ring A forms a fused 5-membered heterocyclic ring having one of the following structures:

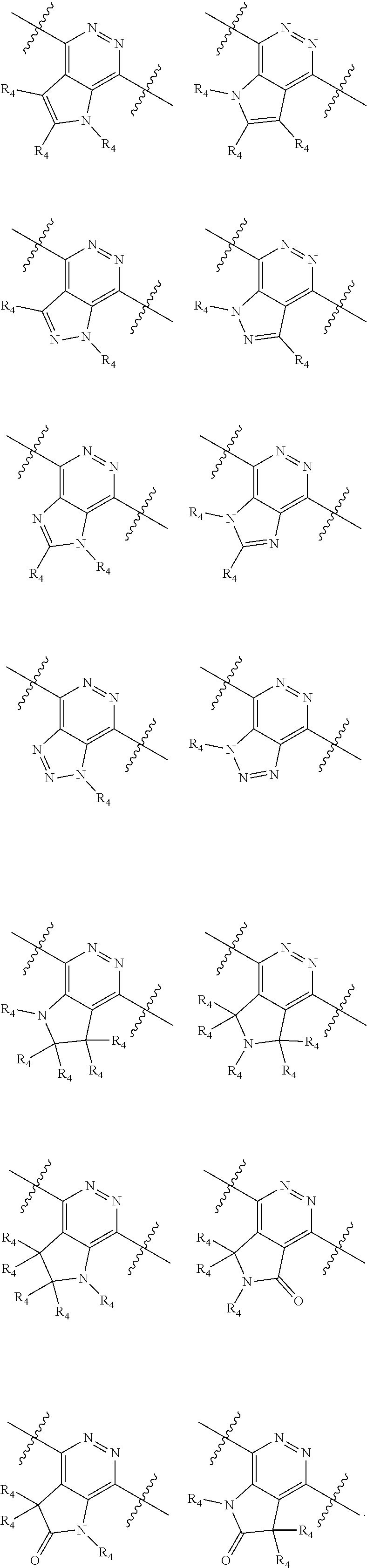

In other embodiments, a compound is provided wherein ring A forms a fused 5-membered heterocyclic ring having one of the following structures:

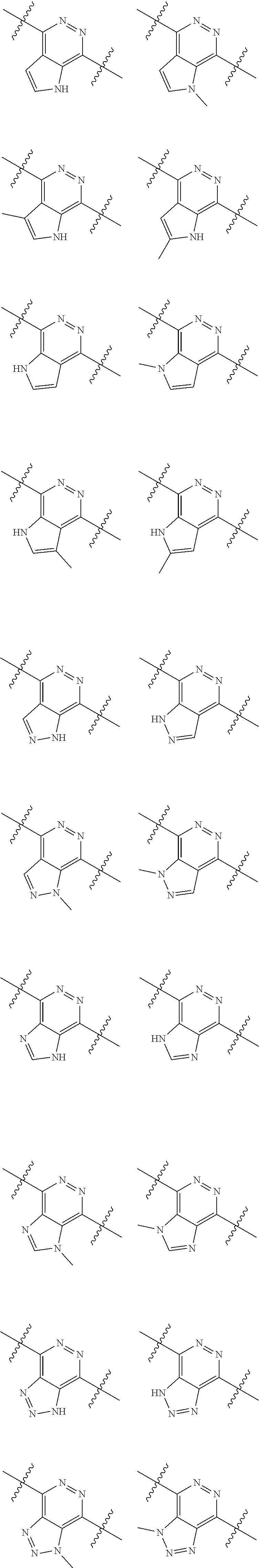

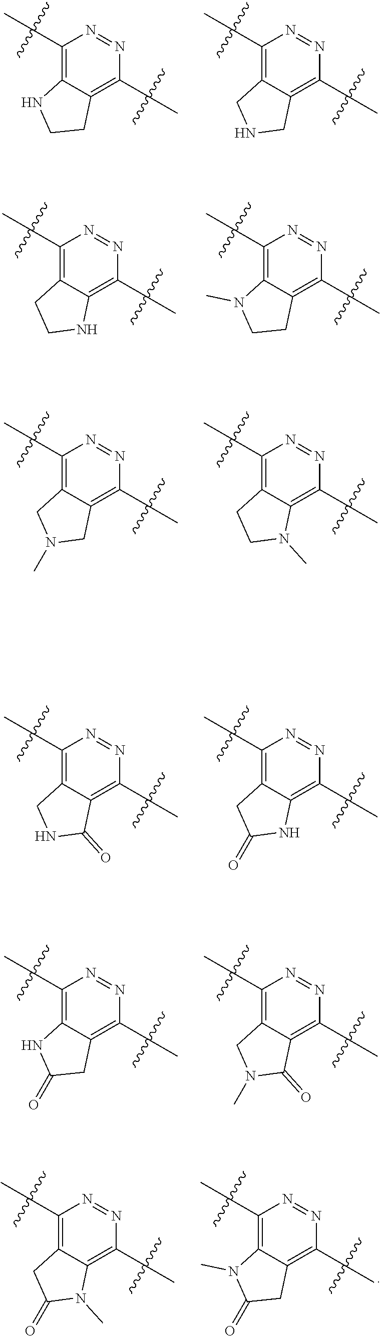

In yet other embodiments, a compound is provided wherein ring A forms a fused 5-membered heterocyclic ring having one of the following structures:

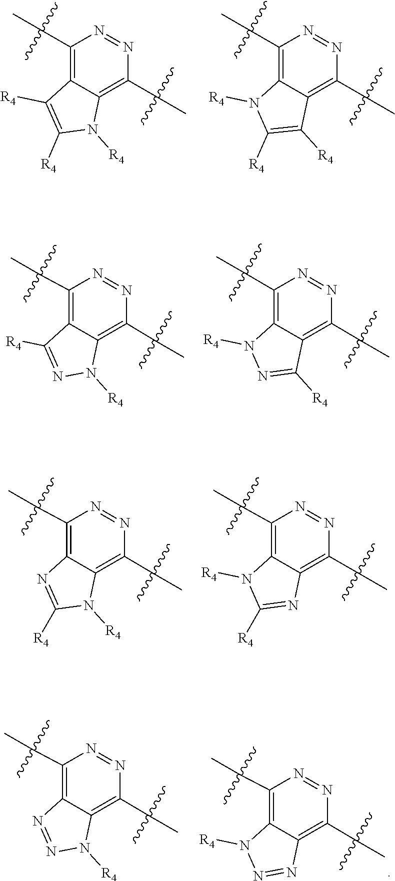

In additional embodiments, a compound is provided wherein ring A forms a fused 5-membered heterocyclic ring having one of the following structures:

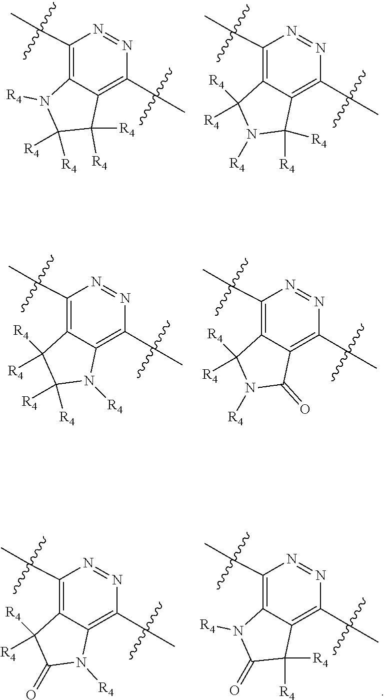

In further embodiments, a compound is provided wherein ring A forms a fused 5-membered heterocyclic ring having one of the following structures:

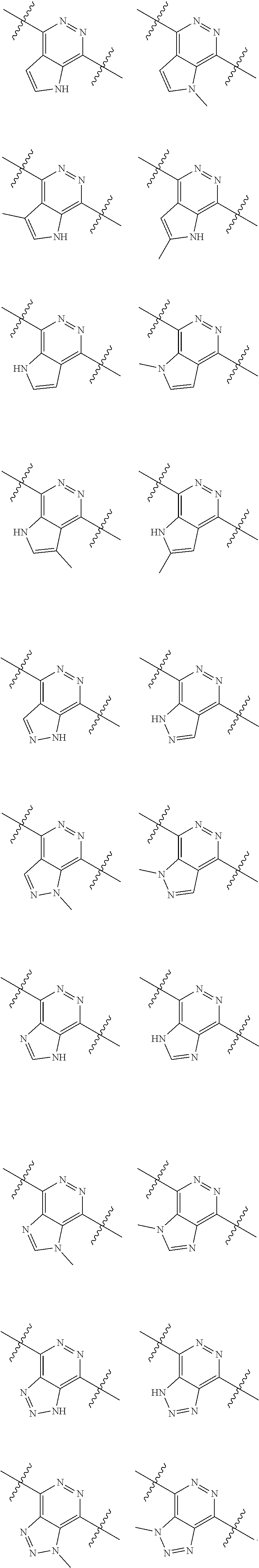

In some embodiments, a compound is provided wherein ring A forms a fused 5-membered heterocyclic ring having one of the following structures:

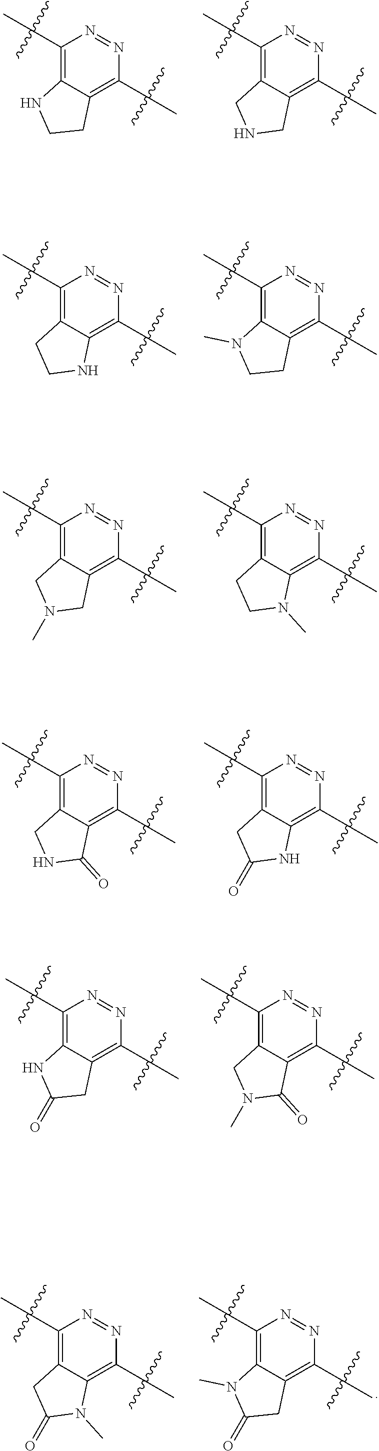

In some embodiments, a compound is provided wherein ring A forms a fused 5-membered heterocyclic ring having one of the following structures:

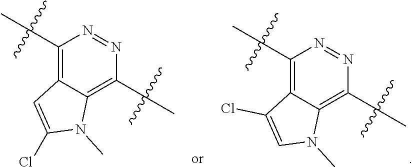 [SMILES: *c1nnc(*)c2c1cc(Cl)n2C ; *c1nnc(*)c2c1c(Cl)cn2C]

In some embodiments, a compound is provided wherein ring A forms a fused 5-membered heterocyclic ring having one of the following structures:

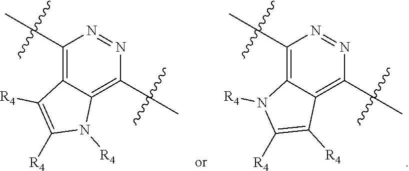

In some embodiments, a compound is provided having structure (I′):

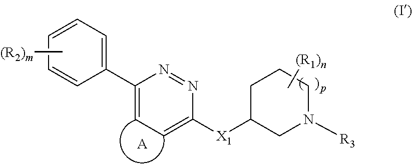

or a pharmaceutically acceptable salt, isomer, hydrate, solvate or isotope thereof, wherein ring A forms a fused 5-membered heterocyclic ring comprising at least one oxygen atom optionally substituted by one or more R₄ wherein A, X₁, R₁, R₂, R₃, m, n, and p are as defined herein. In certain embodiments, a compound is provided wherein ring A forms a fused 5-membered heterocyclic ring optionally substituted by one or more R₄, wherein the heteroatoms comprise oxygen and nitrogen atoms. In certain embodiments, a compound is provided wherein ring A forms a fused 5-membered heterocyclic ring optionally substituted by one or more R₄, wherein the heteroatoms consist of oxygen and nitrogen atoms. In certain embodiments, a compound is provided wherein ring A forms a fused 5-membered heterocyclic ring optionally substituted by one or more R₄, wherein the heteroatoms consist of 1 nitrogen atom and 1-2 oxygen atoms. In certain embodiments, a compound is provided wherein ring A forms a fused 5-membered heterocyclic ring optionally substituted by one or more R₄, wherein the heteroatoms consist of 1-2 oxygen atoms. In some embodiments, a compound is provided wherein ring A forms a fused 5-membered heterocyclic ring having one of the following structures:

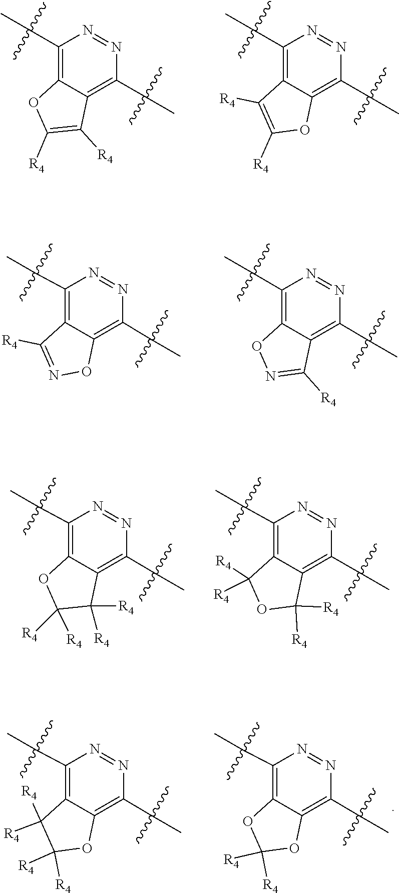

In other embodiments, a compound is provided wherein ring A forms a fused 5-membered heterocyclic ring having one of the following structures:

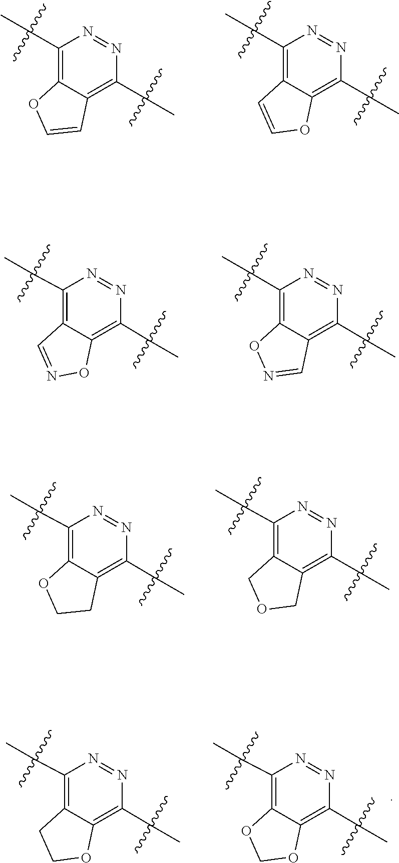

In yet other embodiments, a compound is provided wherein ring A forms a fused 5-membered heterocyclic ring having one of the following structures:

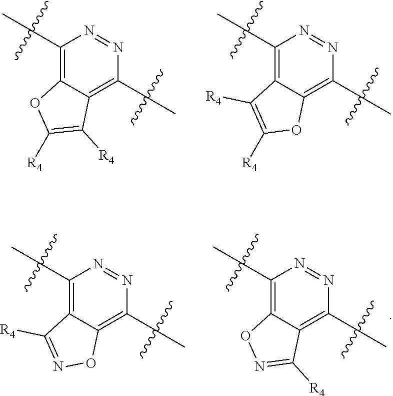

In additional embodiments, a compound is provided wherein ring A forms a fused 5-membered heterocyclic ring having one of the following structures:

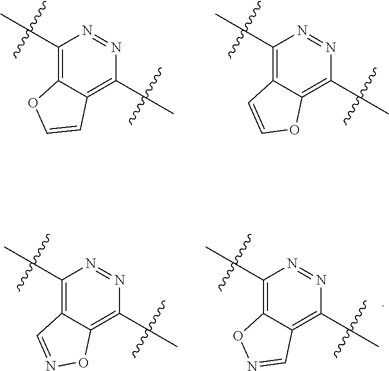 [SMILES: *c1nnc(*)c2occc12 ; *c1nnc(*)c2occc12 ; *c1nnc(*)c2oncc12 ; *c1nnc(*)c2oncc12]

In yet other embodiments, a compound is provided wherein ring A forms a fused 5-membered heterocyclic ring having one of the following structures:

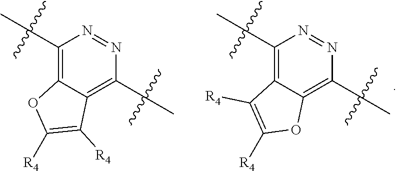

In additional embodiments, a compound is provided wherein ring A forms a fused 5-membered heterocyclic ring having one of the following structures:

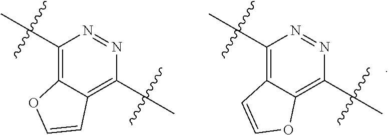 [SMILES: *c1nnc(*)c2occc12 ; *c1nnc(*)c2occc12]

In yet other embodiments, a compound is provided wherein ring A forms a fused 5-membered heterocyclic ring having one of the following structures:

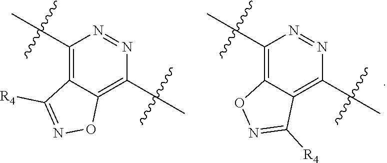

In additional embodiments, a compound is provided wherein ring A forms a fused 5-membered heterocyclic ring having one of the following structures:

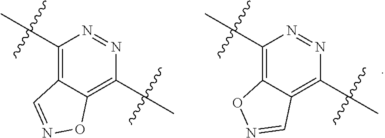 [SMILES: *c1nnc(*)c2oncc12 ; *c1nnc(*)c2oncc12]

In further embodiments, a compound is provided wherein ring A forms a fused 5-membered heterocyclic ring having one of the following structures:

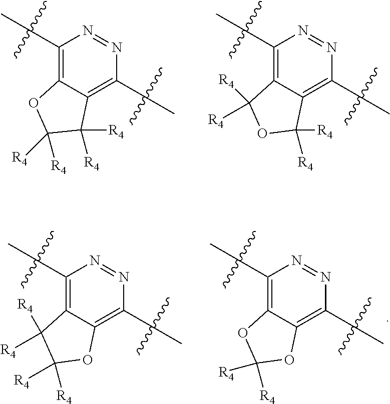

In some embodiments, a compound is provided wherein ring A forms a fused 5-membered heterocyclic ring having one of the following structures:

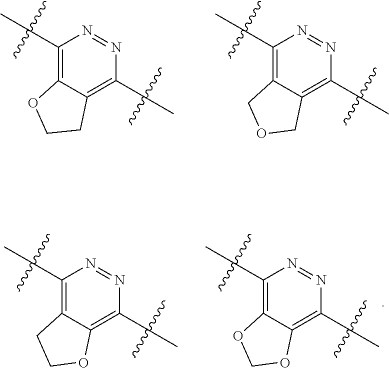 [SMILES: *c1nnc(*)c2c1CCO2 ; *c1nnc(*)c2c1COC2 ; *c1nnc(*)c2c1CCO2 ; *c1nnc(*)c2c1OCO2]

In some embodiments, a compound is provided having structure (I′):

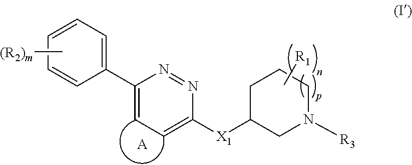

or a pharmaceutically acceptable salt, isomer, hydrate, solvate or isotope thereof, wherein ring A forms a fused 5-membered heterocyclic ring comprising at least one sulfur atom optionally substituted by one or more R₄ wherein A, X₁, R₁, R₂, R₃, m, n, and p are as defined herein. In certain embodiments, a compound is provided wherein ring A forms a fused 5-membered heterocyclic ring optionally substituted by one or more R₄, wherein the heteroatoms comprise sulfur and nitrogen atoms. In certain embodiments, a compound is provided wherein ring A forms a fused 5-membered heterocyclic ring optionally substituted by one or more R₄, wherein the heteroatoms consist of sulfur and nitrogen atoms. In certain embodiments, a compound is provided wherein ring A forms a fused 5-membered heterocyclic ring optionally substituted by one or more R₄, wherein the heteroatoms consist of 1 nitrogen atom and 1 sulfur atoms. In certain embodiments, a compound is provided wherein ring A forms a fused 5-membered heterocyclic ring optionally substituted by one or more R₄, wherein the heteroatoms consist of 1 sulfur atom. In some embodiments, a compound is provided wherein ring A forms a fused 5-membered heterocyclic ring having one of the following structures:

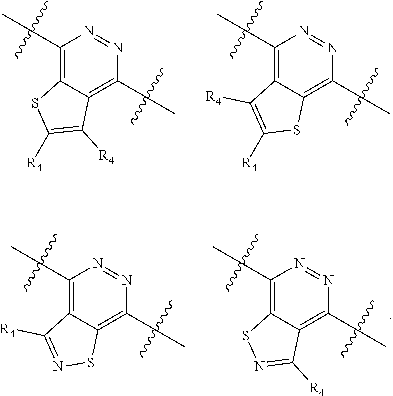

In other embodiments, a compound is provided wherein ring A forms a fused 5-membered heterocyclic ring having one of the following structures:

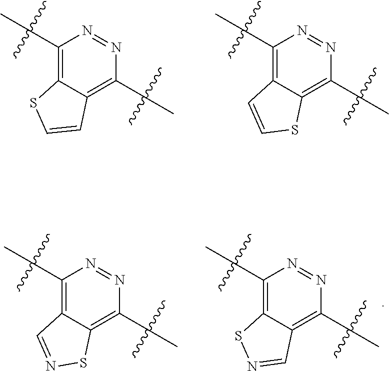 [SMILES: *c1nnc(*)c2sccc12 ; *c1nnc(*)c2sccc12 ; *c1nnc(*)c2sncc12 ; *c1nnc(*)c2sncc12]

In yet other embodiments, a compound is provided wherein ring A forms a fused 5-membered heterocyclic ring having one of the following structures:

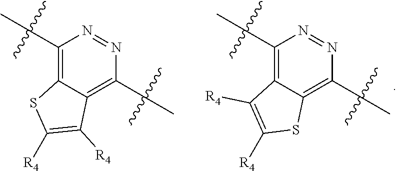

In additional embodiments, a compound is provided wherein ring A forms a fused 5-membered heterocyclic ring having one of the following structures:

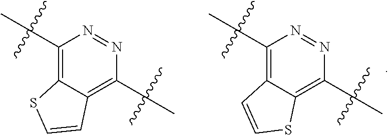 [SMILES: *c1nnc(*)c2sccc12 ; *c1nnc(*)c2sccc12]

In yet other embodiments, a compound is provided wherein ring A forms a fused 5-membered heterocyclic ring having one of the following structures:

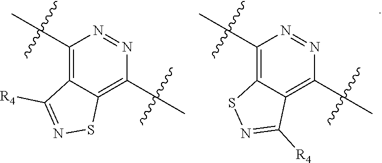

In additional embodiments, a compound is provided wherein ring A forms a fused 5-membered heterocyclic ring having one of the following structures:

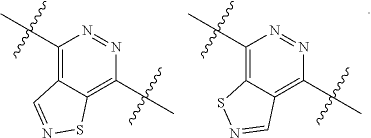 [SMILES: *c1nnc(*)c2sncc12 ; *c1nnc(*)c2sncc12]

In some embodiments, a compound is provided having structure (II):

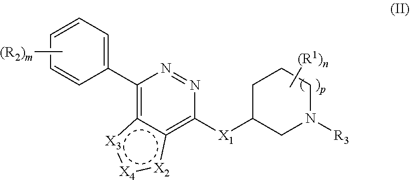

or a pharmaceutically acceptable salt, isomer, hydrate, solvate or isotope thereof, wherein:
[symbol] indicates an aromatic or non-aromatic ring system; and
X₂, X₃, and X₄ are each independently ═N—, —NR₄—, —O—, or —S—, —C(R₄)₂—, or ═CR₄—.
X₁ is CRₐR_{b}, NR₄, or O;
Rₐ and R_{b} are each independently H or alkyl;
R₁ is halo;
R₂ is halo, OH, CN, alkyl, haloalkyl, alkoxy, haloalkoxy, or cycloalkyl;
R₃ is H, cycloalkyl, or alkyl optionally substituted with one or more R3′;
R3′ is OH, halo, or cycloalkyl;
each R₄ is independently H or alkyl, or when attached to a carbon atom, two R₄ may join together to form oxo;
m is 0-4;
n is 0-4; and
p is 0-2.

In other embodiments, a compound is provided having structure (II):

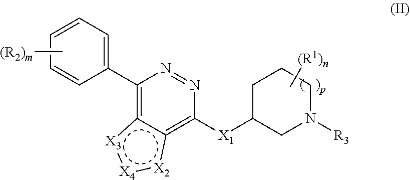

or a pharmaceutically acceptable salt, isomer, hydrate, solvate or isotope thereof, wherein:
[symbol] indicates an aromatic or non-aromatic ring system; and
X₂, X₃, and X₄ are each independently ═N—, —NR₄—, —O—, or —S—, —C(R₄)₂—, or ═CR₄—.
X₁ is CRₐR_{b}, NR₄, or O;
Rₐ and R_{b} are each independently H or alkyl;
R₁ is halo;
R₂ is halo, OH, alkyl, or haloalkyl;
R₃ is alkyl;
each R₄ is independently H or alkyl, or when attached to a carbon atom, two R₄ may join together to form oxo;
m is 0-2;
n is 0-2; and
p is 1.

In additional embodiments, a compound is provided having structure II):

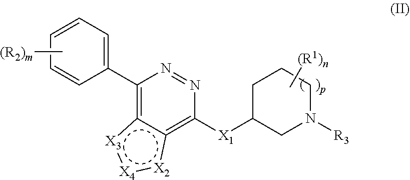

or a pharmaceutically acceptable salt, isomer, hydrate, solvate or isotope thereof, wherein X₁, R₁, R₂, R₃, R₄, m, n, and p are as defined herein, and wherein:
[symbol] indicates an non-aromatic ring system; and
X₂, X₃, and X₄ are each independently —C(R₄)₂—.

In further embodiments, a compound is provided having structure (II):

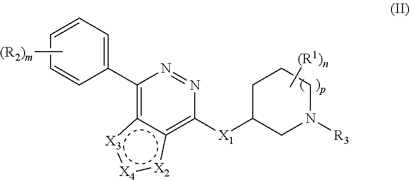

or a pharmaceutically acceptable salt, isomer, hydrate, solvate or isotope thereof, wherein X₁, R₁, R₂, R₃, R₄, m, n, and p are as defined herein, and wherein:
[symbol] indicates an non-aromatic ring system; and
X₂, X₃, and X₄ are each independently —O— or —C(R₄)₂—.

In yet other embodiments, a compound is provided having structure (II):

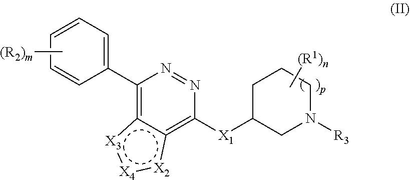

or a pharmaceutically acceptable salt, isomer, hydrate, solvate or isotope thereof, wherein X₁, R₁, R₂, R₃, R₄, m, n, and p are as defined herein, and wherein:
[symbol] indicates an non-aromatic ring system; and
X₂, X₃, and X₄ are each independently —NR₄— or —C(R₄)₂—.

In some embodiments, a compound is provided having structure (II):

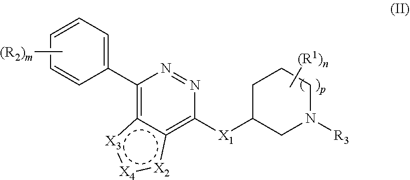

or a pharmaceutically acceptable salt, isomer, hydrate, solvate or isotope thereof, wherein X₁, R₁, R₂, R₃, R₄, m, n, and p are as defined herein, and wherein:
[symbol] indicates an aromatic ring system; and
X₂, X₃, and X₄ are each independently ═N—, —NR₄— or ═CR₄—, wherein at least one of X₂ or X₃ is —NR₄—.

In other embodiments, a compound is provided having structure (II):

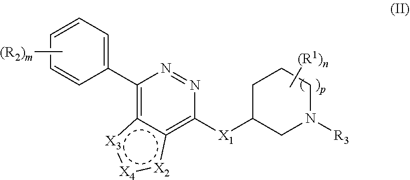

or a pharmaceutically acceptable salt, isomer, hydrate, solvate or isotope thereof, wherein X₁, R₁, R₂, R₃, R₄, m, n, and p are as defined herein, and wherein:
[symbol] indicates an aromatic ring system; and
X₂, X₃, and X₄ are each independently —O—, ═CR₄—, or ═N—, wherein at least one of X₂ or X₃ is —O—.

In additional embodiments, a compound is provided having structure (II):

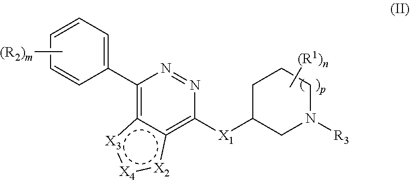

or a pharmaceutically acceptable salt, isomer, hydrate, solvate or isotope thereof, wherein X₁, R₁, R₂, R₃, R₄, m, n, and p are as defined herein, and wherein:
[symbol] indicates an aromatic ring system; and
X₂, X₃, and X₄ are each independently —S—, ═CR₄—, or ═N—, wherein at least one of X₂ or X₃ is —S—.

In some embodiments, a compound is provided having structure (IIa):

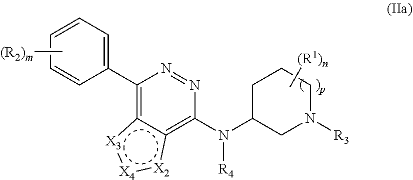

or a pharmaceutically acceptable salt, isomer, hydrate, solvate or isotope thereof, wherein X₂, X₃, X₄, R₁, R₂, R₃, R₄, m, n, and p are as defined herein.

In other embodiments, a compound is provided having structure (IIb):

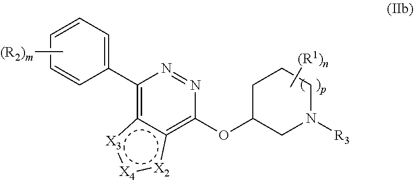

or a pharmaceutically acceptable salt, isomer, hydrate, solvate or isotope thereof, wherein X₂, X₃, X₄, R₁, R₂, R₃, R₄, m, n, and p are as defined herein.

In additional embodiments, a compound is provided having structure (IIc):

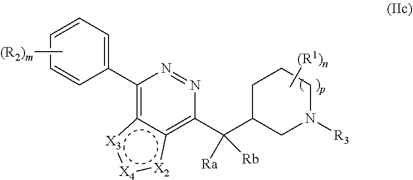

or a pharmaceutically acceptable salt, isomer, hydrate, solvate or isotope thereof, wherein X₂, X₃, X₄, R₁, R₂, R₃, R₄, Rₐ, R_{b}, m, n, and p are as defined herein.

In some embodiments, a compound is provided having structure (IIIa-i):

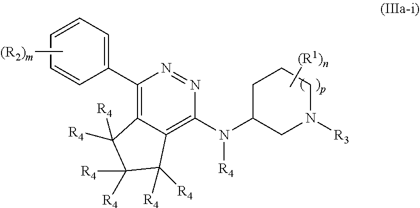

or a pharmaceutically acceptable salt, isomer, hydrate, solvate or isotope thereof, wherein R₁, R₂, R₃, R₄, m, n, and p are as defined herein.

In other embodiments, a compound is provided having structure (IIIa-ii):

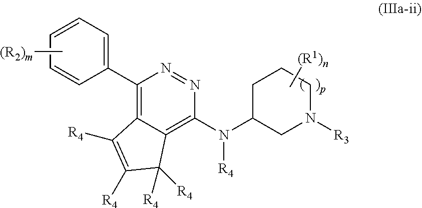

or a pharmaceutically acceptable salt, isomer, hydrate, solvate or isotope thereof, wherein R₁, R₂, R₃, R₄, m, n, and p are as defined herein.

In additional embodiments, a compound is provided having structure (IIIa-iii):

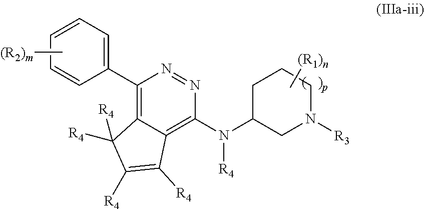

or a pharmaceutically acceptable salt, isomer, hydrate, solvate or isotope thereof, wherein R₁, R₂, R₃, R₄, m, n, and p are as defined herein.

In further embodiments, a compound is provided having structure (IIIb-i):

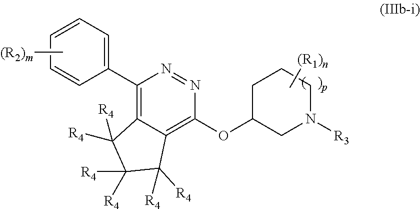

or a pharmaceutically acceptable salt, isomer, hydrate, solvate or isotope thereof, wherein R₁, R₂, R₃, R₄, m, n, and p are as defined herein.

In some embodiments, a compound is provided having structure (IIIb-ii):

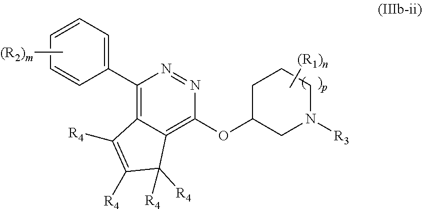

or a pharmaceutically acceptable salt, isomer, hydrate, solvate or isotope thereof, wherein R₁, R₂, R₃, R₄, m, n, and p are as defined herein.

In other embodiments, a compound is provided having structure (IIIb-iii):

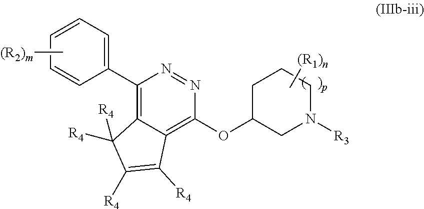

or a pharmaceutically acceptable salt, isomer, hydrate, solvate or isotope thereof, wherein R₁, R₂, R₃, R₄, m, n, and p are as defined herein.

In additional embodiments, a compound is provided having structure (IIIc-i):

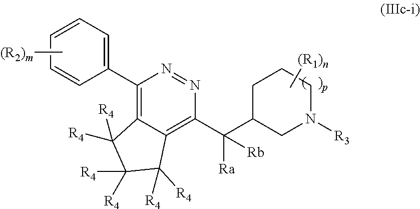

or a pharmaceutically acceptable salt, isomer, hydrate, solvate or isotope thereof, wherein R₁, R₂, R₃, R₄, Rₐ, R_{b}, m, n, and p are as defined herein.

In further embodiments, a compound is provided having structure (IIIc-ii):

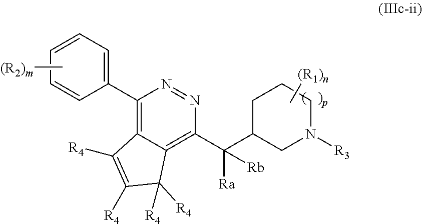

or a pharmaceutically acceptable salt, isomer, hydrate, solvate or isotope thereof, wherein R₁, R₂, R₃, R₄, Rₐ, R_{b}, m, n, and p are as defined herein.

In some embodiments, a compound is provided having structure (IIIc-iii):

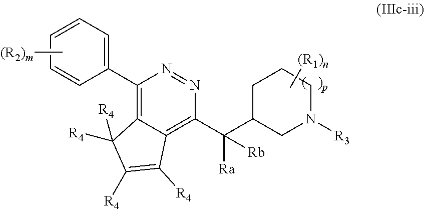

or a pharmaceutically acceptable salt, isomer, hydrate, solvate or isotope thereof, wherein R₁, R₂, R₃, R₄, Rₐ, R_{b}, m, n, and p are as defined herein.

In other embodiments, a compound is provided having structure (IVa-i):

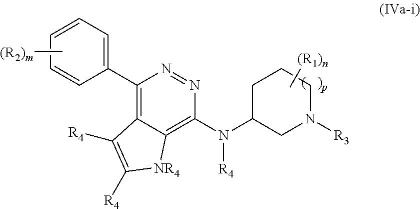

or a pharmaceutically acceptable salt, isomer, hydrate, solvate or isotope thereof, wherein R₁, R₂, R₃, R₄, m, n, and p are as defined herein.

In other embodiments, a compound is provided having structure (IVa-i):

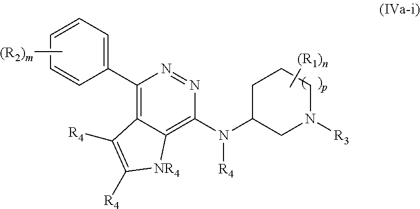

or a pharmaceutically acceptable salt, isomer, hydrate, solvate or isotope thereof, wherein:
R₁ is halo;
R₂ is halo, CN, alkyl, haloalkyl, alkoxy, haloalkoxy, or cycloalkyl;
R₃ is alkylsulfonyl, cycloalkyl, aryl, heterocyclyl, or alkyl optionally substituted with one or more R_{3′};
R_{3′} is OH, halo, alkoxy, aminyl, or cycloalkyl;
each R₄ is independently H, halo, or alkyl when attached to a carbon atom and H or alkyl when attached to a nitrogen atom;
m is 0-4;
n is 0-4; and
p is 0-2.

In additional embodiments, a compound is provided having structure (IVa-ii):

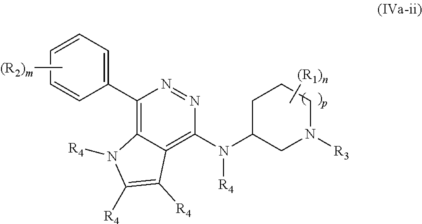

or a pharmaceutically acceptable salt, isomer, hydrate, solvate or isotope thereof, wherein R₁, R₂, R₃, R₄, m, n, and p are as defined herein.

In further embodiments, a compound is provided having structure (IVa-iv):

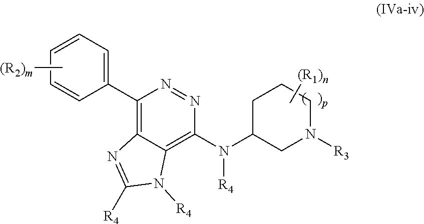

or a pharmaceutically acceptable salt, isomer, hydrate, solvate or isotope thereof, wherein R₁, R₂, R₃, R₄, m, n, and p are as defined herein.

In some embodiments, a compound is provided having structure (IVa-v):

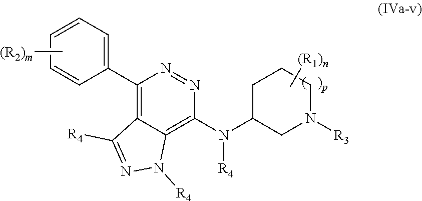

or a pharmaceutically acceptable salt, isomer, hydrate, solvate or isotope thereof, wherein R₁, R₂, R₃, R₄, m, n, and p are as defined herein.

In other embodiments, a compound is provided having structure (IVa-vi):

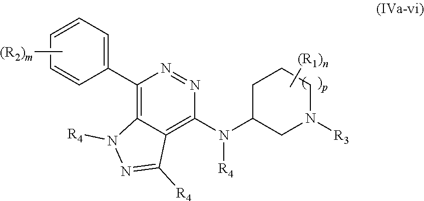

or a pharmaceutically acceptable salt, isomer, hydrate, solvate or isotope thereof, wherein R₁, R₂, R₃, R₄, m, n, and p are as defined herein.

In additional embodiments, a compound is provided having structure (IVa-vii):

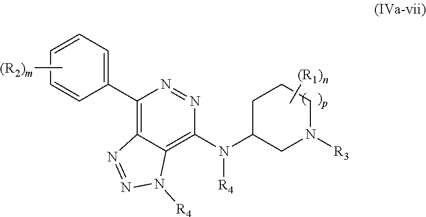

or a pharmaceutically acceptable salt, isomer, hydrate, solvate or isotope thereof, wherein R₁, R₂, R₃, R₄, m, n, and p are as defined herein.

In further embodiments, a compound is provided having structure (IVa-ix):

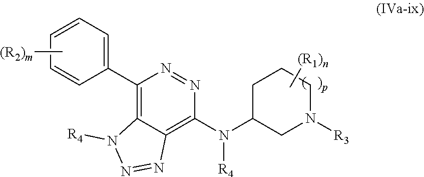

or a pharmaceutically acceptable salt, isomer, hydrate, solvate or isotope thereof, wherein R₁, R₂, R₃, R₄, m, n, and p are as defined herein.

In some embodiments, a compound is provided having structure (IVb-i):

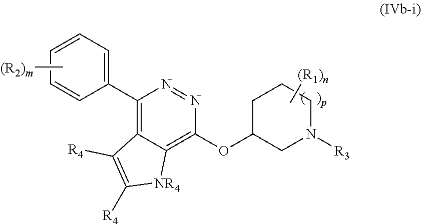

or a pharmaceutically acceptable salt, isomer, hydrate, solvate or isotope thereof, wherein R₁, R₂, R₃, R₄, m, n, and p are as defined herein.

In other embodiments, a compound is provided having structure (IVb-ii):

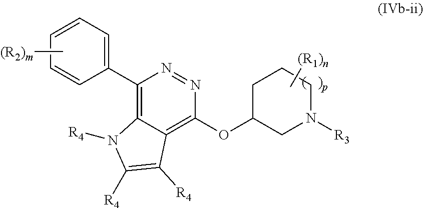

or a pharmaceutically acceptable salt, isomer, hydrate, solvate or isotope thereof, wherein R₁, R₂, R₃, R₄, m, n, and p are as defined herein.

In additional embodiments, a compound is provided having structure (IVb-iv):

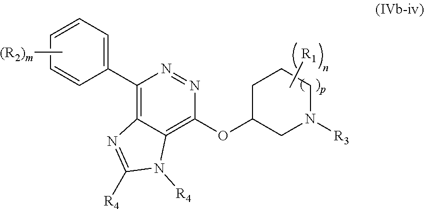

or a pharmaceutically acceptable salt, isomer, hydrate, solvate or isotope thereof, wherein R₁, R₂, R₃, R₄, m, n, and p are as defined herein.

In further embodiments, a compound is provided having structure (IVb-v):

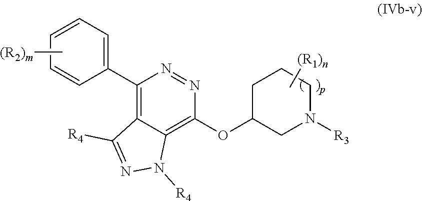

or a pharmaceutically acceptable salt, isomer, hydrate, solvate or isotope thereof, wherein R₁, R₂, R₃, R₄, m, n, and p are as defined herein.

In some embodiments, a compound is provided having structure (IVb-vi):

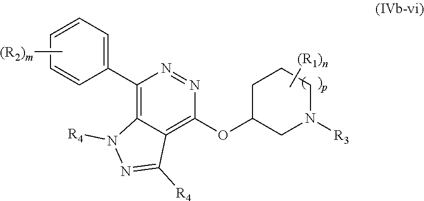

or a pharmaceutically acceptable salt, isomer, hydrate, solvate or isotope thereof, wherein R₁, R₂, R₃, R₄, m, n, and p are as defined herein.

In other embodiments, a compound is provided having structure (IVb-vii):

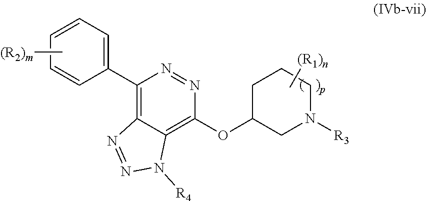

or a pharmaceutically acceptable salt, isomer, hydrate, solvate or isotope thereof, wherein R₁, R₂, R₃, R₄, m, n, and p are as defined herein.

In additional embodiments, a compound is provided having structure (IVb-ix):

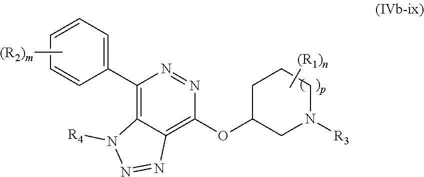

or a pharmaceutically acceptable salt, isomer, hydrate, solvate or isotope thereof, wherein R₁, R₂, R₃, R₄, m, n, and p are as defined herein.

In further embodiments, a compound is provided having structure (IVc-i):

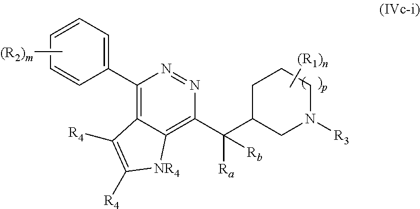

or a pharmaceutically acceptable salt, isomer, hydrate, solvate or isotope thereof, wherein R₁, R₂, R₃, R₄, Rₐ, R_{b}, m, n, and p are as defined herein.

In some embodiments, a compound is provided having structure (IVc-ii):

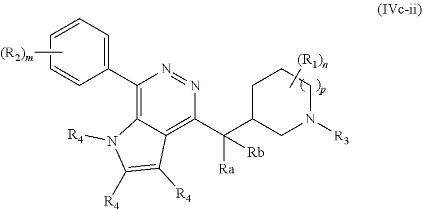

or a pharmaceutically acceptable salt, isomer, hydrate, solvate or isotope thereof, wherein R₁, R₂, R₃, R₄, Rₐ, R_{b}, m, n, and p are as defined herein.

In other embodiments, a compound is provided having structure (IVc-iv):

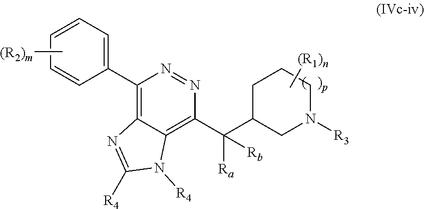

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (IVc-v):

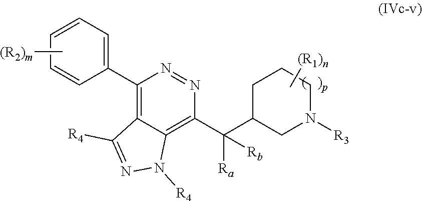

or a pharmaceutically acceptable salt, isomer, hydrate, solvate or isotope thereof, wherein R₁, R₂, R₃, R₄, Rₐ, R_{b}, m, n, and p are as defined herein.

In further embodiments, a compound is provided having structure (IVc-vi):

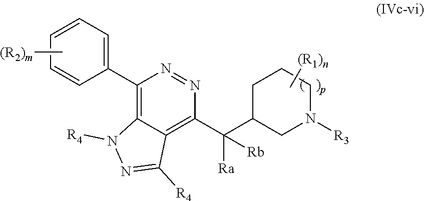

or a pharmaceutically acceptable salt, isomer, hydrate, solvate or isotope thereof, wherein R₁, R₂, R₃, R₄, Rₐ, R_{b}, m, n, and p are as defined herein.

In some embodiments, a compound is provided having structure (IVc-vii):

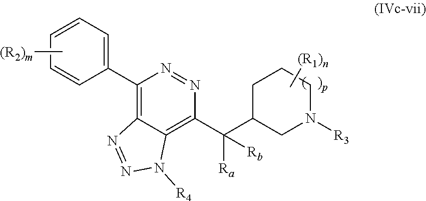

or a pharmaceutically acceptable salt, isomer, hydrate, solvate or isotope thereof, wherein R₁, R₂, R₃, R₄, Rₐ, R_{b}, m, n, and p are as defined herein.

In other embodiments, a compound is provided having structure (IVc-ix):

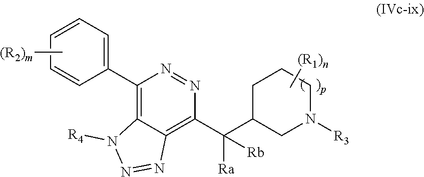

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (Va-i):

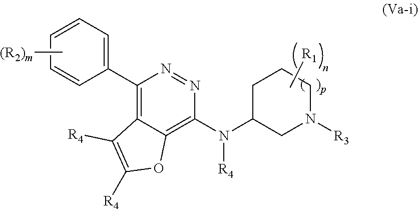

or a pharmaceutically acceptable salt, isomer, hydrate, solvate or isotope thereof, wherein R₁, R₂, R₃, R₄, m, n, and p are as defined herein.

In additional embodiments, a compound is provided having structure (Va-i):

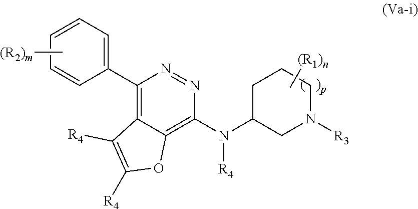

or a pharmaceutically acceptable salt, isomer, hydrate, solvate or isotope thereof, wherein:
R₁ is halo;
R₂ is halo, CN, alkyl, haloalkyl, or cycloalkyl;
R₃ is alkylsulfonyl, cycloalkyl, aryl, heterocyclyl, or alkyl optionally substituted with one or more R_{3′};
R_{3′} is OH, halo, alkoxy, aminyl, or cycloalkyl;
each R₄ is independently H, halo, or alkyl when attached to a carbon atom and H or alkyl when attached to a nitrogen atom;
m is 0-4;
n is 0-4; and
p is 0-2.

In further embodiments, a compound is provided having structure (Va-ii):

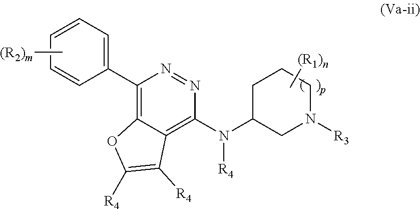

or a pharmaceutically acceptable salt, isomer, hydrate, solvate or isotope thereof, wherein R₁, R₂, R₃, R₄, m, n, and p are as defined herein.

In some embodiments, a compound is provided having structure (Va-iii):

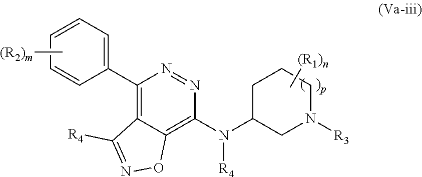

or a pharmaceutically acceptable salt, isomer, hydrate, solvate or isotope thereof, wherein R₁, R₂, R₃, R₄, m, n, and p are as defined herein.

In other embodiments, a compound is provided having structure (Va-iv):

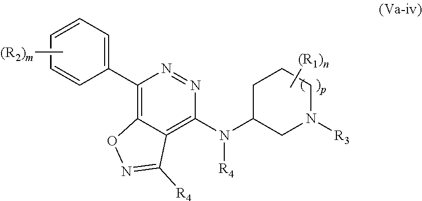

or a pharmaceutically acceptable salt, isomer, hydrate, solvate or isotope thereof, wherein R₁, R₂, R₃, R₄, m, n, and p are as defined herein.

In additional embodiments, a compound is provided having structure (Vb-i):

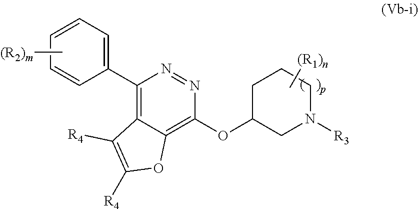

or a pharmaceutically acceptable salt, isomer, hydrate, solvate or isotope thereof, wherein R₁, R₂, R₃, R₄, m, n, and p are as defined herein.

In further embodiments, a compound is provided having structure (Vb-ii):

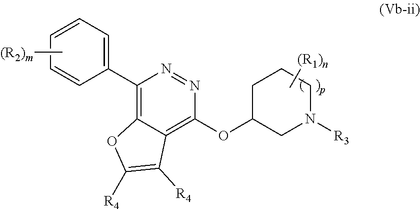

or a pharmaceutically acceptable salt, isomer, hydrate, solvate or isotope thereof, wherein R₁, R₂, R₃, R₄, m, n, and p are as defined herein.

In some embodiments, a compound is provided having structure (Vb-iii):

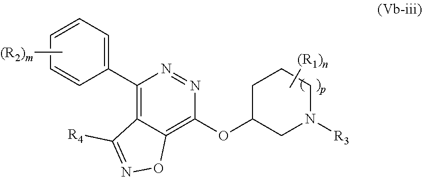

or a pharmaceutically acceptable salt, isomer, hydrate, solvate or isotope thereof, wherein R₁, R₂, R₃, R₄, m, n, and p are as defined herein.

In other embodiments, a compound is provided having structure (Vb-iv):

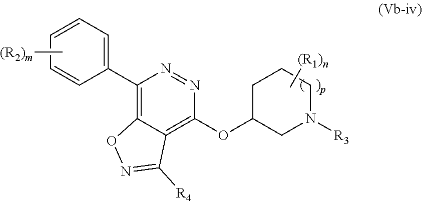

or a pharmaceutically acceptable salt, isomer, hydrate, solvate or isotope thereof, wherein R₁, R₂, R₃, R₄, m, n, and p are as defined herein.

In additional embodiments, a compound is provided having structure (Vc-i):

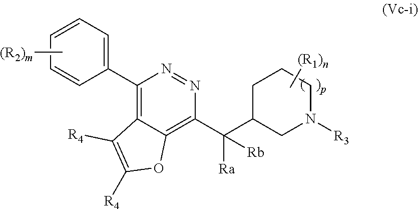

or a pharmaceutically acceptable salt, isomer, hydrate, solvate or isotope thereof, wherein R₁, R₂, R₃, R₄, Rₐ, R_{b}, m, n, and p are as defined herein.

In further embodiments, a compound is provided having structure (Vc-ii):

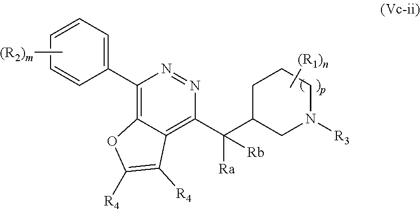

or a pharmaceutically acceptable salt, isomer, hydrate, solvate or isotope thereof, wherein R₁, R₂, R₃, R₄, Rₐ, R_{b}, m, n, and p are as defined herein.

In some embodiments, a compound is provided having structure (Vc-iii):

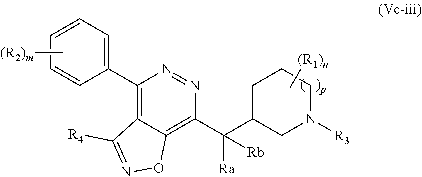

or a pharmaceutically acceptable salt, isomer, hydrate, solvate or isotope thereof, wherein R₁, R₂, R₃, R₄, Rₐ, R_{b}, m, n, and p are as defined herein.

In other embodiments, a compound is provided having structure (Vc-iv):

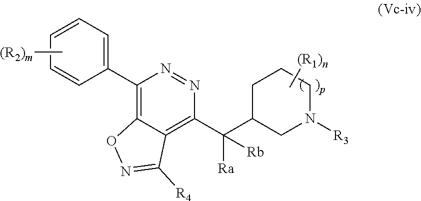

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (VIa-i):

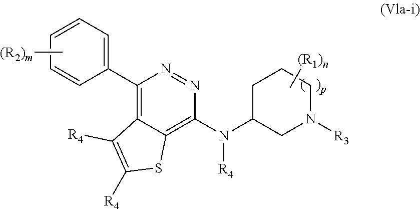

or a pharmaceutically acceptable salt, isomer, hydrate, solvate or isotope thereof, wherein R₁, R₂, R₃, R₄, m, n, and p are as defined herein.

In additional embodiments, a compound is provided having structure (VIa-i):

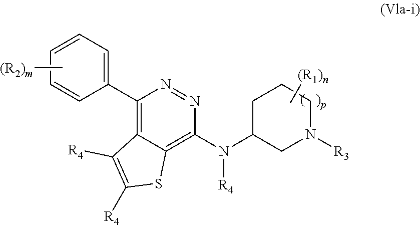

or a pharmaceutically acceptable salt, isomer, hydrate, solvate or isotope thereof, wherein:
   R₁ is halo;
   R₂ is halo, CN, alkyl, haloalkyl, alkoxy, haloalkoxy, or cycloalkyl;
   R₃ is alkylsulfonyl, cycloalkyl, aryl, heterocyclyl, or alkyl optionally substituted with one or more R_{3′};
   R_{3′} is OH, halo, alkoxy, aminyl, or cycloalkyl;
   each R₄ is independently H, halo, or alkyl when attached to a carbon atom and H or alkyl when attached to a nitrogen atom;
   m is 0-4;
   n is 0-4; and
   p is 0-2,
   wherein R₃ is not ethyl.

In further embodiments, a compound is provided having structure (VIa-ii):

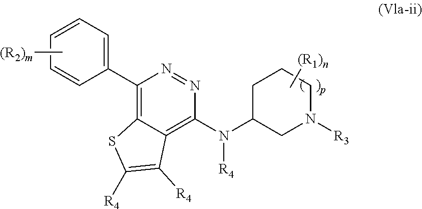

or a pharmaceutically acceptable salt, isomer, hydrate, solvate or isotope thereof, wherein R₁, R₂, R₃, R₄, m, n, and p are as defined herein.

In some embodiments, a compound is provided having structure (VIa-iii):

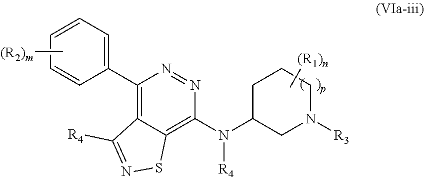

or a pharmaceutically acceptable salt, isomer, hydrate, solvate or isotope thereof, wherein R₁, R₂, R₃, R₄, m, n, and p are as defined herein.

In other embodiments, a compound is provided having structure (VIa-iv):

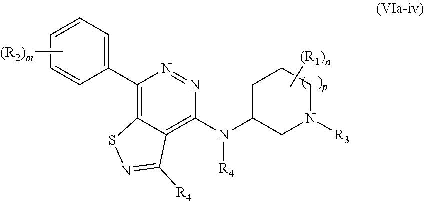

or a pharmaceutically acceptable salt, isomer, hydrate, solvate or isotope thereof, wherein R₁, R₂, R₃, R₄, m, n, and p are as defined herein.

In additional embodiments, a compound is provided having structure (VIb-i):

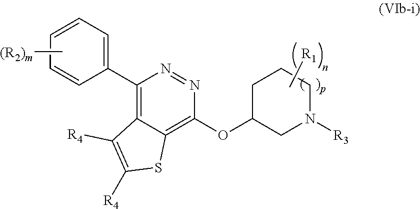

or a pharmaceutically acceptable salt, isomer, hydrate, solvate or isotope thereof, wherein R₁, R₂, R₃, R₄, m, n, and p are as defined herein.

In further embodiments, a compound is provided having structure (VIb-ii):

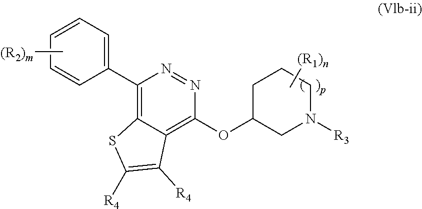

or a pharmaceutically acceptable salt, isomer, hydrate, solvate or isotope thereof, wherein R₁, R₂, R₃, R₄, m, n, and p are as defined herein.

In some embodiments, a compound is provided having structure (VIb-iii):

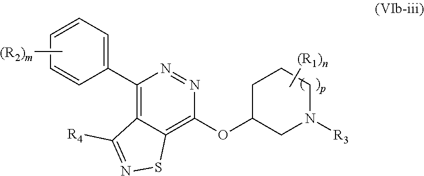

or a pharmaceutically acceptable salt, isomer, hydrate, solvate or isotope thereof, wherein R₁, R₂, R₃, R₄, m, n, and p are as defined herein.

In other embodiments, a compound is provided having structure (VIb-iv):

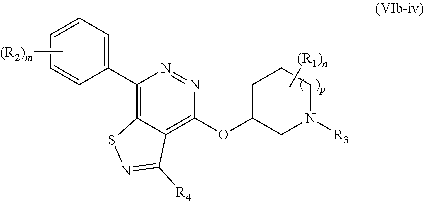

or a pharmaceutically acceptable salt, isomer, hydrate, solvate or isotope thereof, wherein R₁, R₂, R₃, R₄, m, n, and p are as defined herein.

In additional embodiments, a compound is provided having structure (VIc-i):

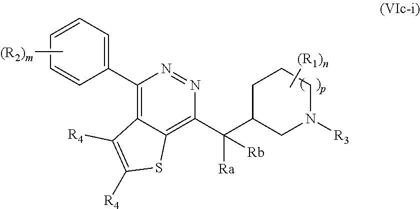

or a pharmaceutically acceptable salt, isomer, hydrate, solvate or isotope thereof, wherein R₁, R₂, R₃, R₄, Rₐ, R_{b}, m, n, and p are as defined herein.

In further embodiments, a compound is provided having structure (VIc-ii):

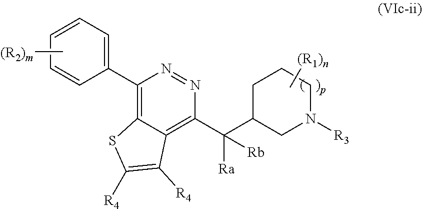

or a pharmaceutically acceptable salt, isomer, hydrate, solvate or isotope thereof, wherein R₁, R₂, R₃, R₄, Rₐ, R_{b}, m, n, and p are as defined herein.

In some embodiments, a compound is provided having structure (VIc-iii):

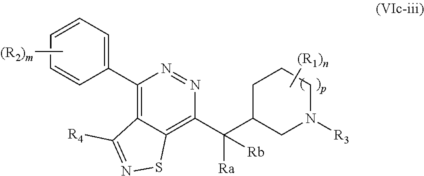

or a pharmaceutically acceptable salt, isomer, hydrate, solvate or isotope thereof, wherein R₁, R₂, R₃, R₄, Rₐ, R_{b}, m, n, and p are as defined herein.

In other embodiments, a compound is provided having structure (VIc-iv):

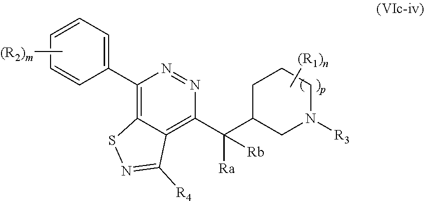

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (VIIa-i):

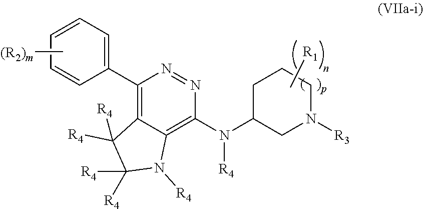

or a pharmaceutically acceptable salt, isomer, hydrate, solvate or isotope thereof, wherein R₁, R₂, R₃, R₄, m, n, and p are as defined herein.

In further embodiments, a compound is provided having structure (VIIa-ii):

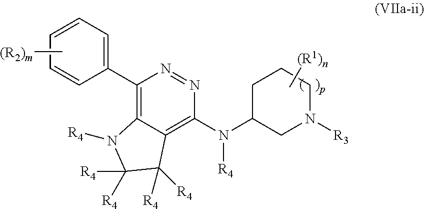

or a pharmaceutically acceptable salt, isomer, hydrate, solvate or isotope thereof, wherein R₁, R₂, R₃, R₄, m, n, and p are as defined herein.

In some embodiments, a compound is provided having structure (VIIa-iii):

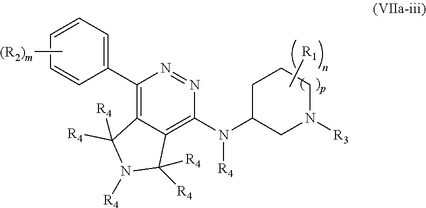

or a pharmaceutically acceptable salt, isomer, hydrate, solvate or isotope thereof, wherein R₁, R₂, R₃, R₄, m, n, and p are as defined herein.

In other embodiments, a compound is provided having structure (VIIb-i):

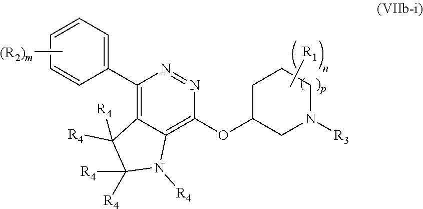

or a pharmaceutically acceptable salt, isomer, hydrate, solvate or isotope thereof, wherein R₁, R₂, R₃, R₄, m, n, and p are as defined herein.

In additional embodiments, a compound is provided having structure (VIIb-ii):

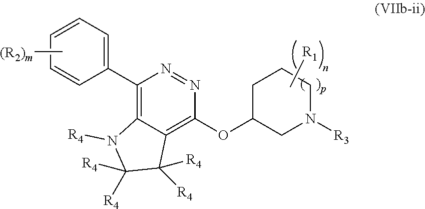

or a pharmaceutically acceptable salt, isomer, hydrate, solvate or isotope thereof, wherein R₁, R₂, R₃, R₄, m, n, and p are as defined herein.

In further embodiments, a compound is provided having structure (VIIb-iii):

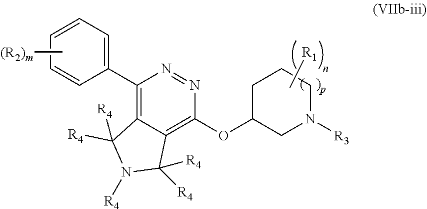

or a pharmaceutically acceptable salt, isomer, hydrate, solvate or isotope thereof, wherein R₁, R₂, R₃, R₄, m, n, and p are as defined herein.

In some embodiments, a compound is provided having structure (VIIc-i):

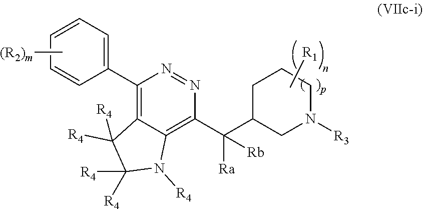

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (VIIc-ii):

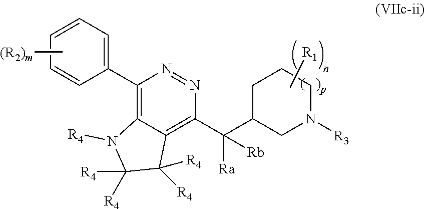

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (VIIc-iii):

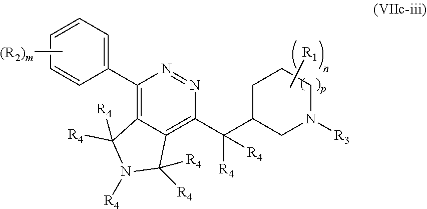

or a pharmaceutically acceptable salt, isomer, hydrate, solvate or isotope thereof, wherein R₁, R₂, R₃, R₄, Rₐ, R_{b}, m, n, and p are as defined herein.

In further embodiments, a compound is provided having structure (Villa-i):

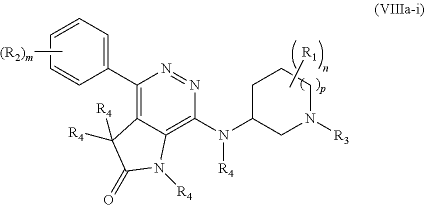

or a pharmaceutically acceptable salt, isomer, hydrate, solvate or isotope thereof, wherein R₁, R₂, R₃, R₄, m, n, and p are as defined herein.

In some embodiments, a compound is provided having structure (VIIIa-ii):

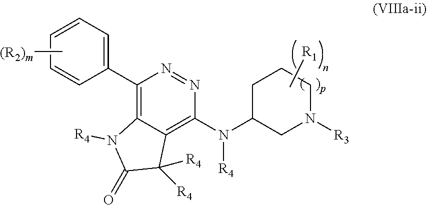

or a pharmaceutically acceptable salt, isomer, hydrate, solvate or isotope thereof, wherein R₁, R₂, R₃, R₄, m, n, and p are as defined herein.

In other embodiments, a compound is provided having structure (VIIIa-iii):

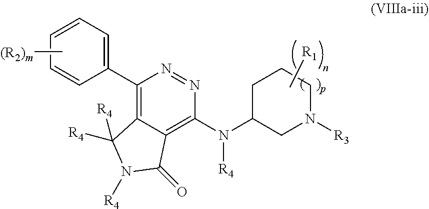

or a pharmaceutically acceptable salt, isomer, hydrate, solvate or isotope thereof, wherein R₁, R₂, R₃, R₄, m, n, and p are as defined herein.

In additional embodiments, a compound is provided having structure (VIIIa-iv):

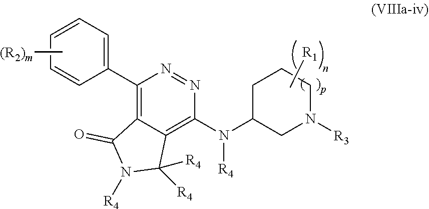

or a pharmaceutically acceptable salt, isomer, hydrate, solvate or isotope thereof, wherein R₁, R₂, R₃, R₄, m, n, and p are as defined herein.

In further embodiments, a compound is provided having structure (VIIIb-i):

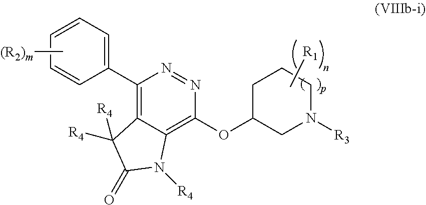

or a pharmaceutically acceptable salt, isomer, hydrate, solvate or isotope thereof, wherein R₁, R₂, R₃, R₄, m, n, and p are as defined herein.

In some embodiments, a compound is provided having structure (VIIIb-ii):

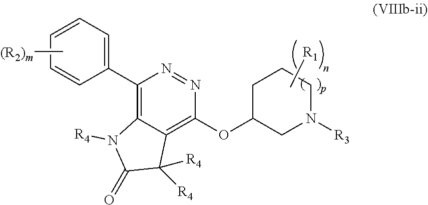

or a pharmaceutically acceptable salt, isomer, hydrate, solvate or isotope thereof, wherein R₁, R₂, R₃, R₄, m, n, and p are as defined herein.

In other embodiments, a compound is provided having structure (VIIIb-iii):

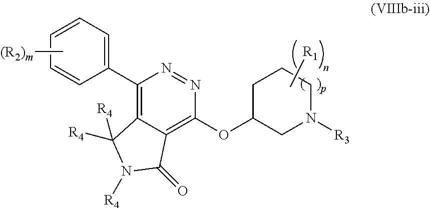

or a pharmaceutically acceptable salt, isomer, hydrate, solvate or isotope thereof.

In additional embodiments, a compound is provided having structure (VIIIb-iv):

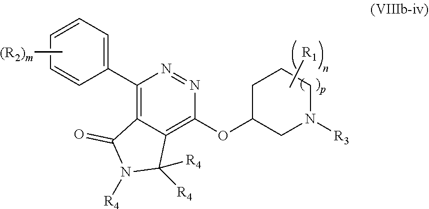

or a pharmaceutically acceptable salt, isomer, hydrate, solvate or isotope thereof, wherein R₁, R₂, R₃, R₄, m, n, and p are as defined herein.

In further embodiments, a compound is provided having structure (VIIIc-i):

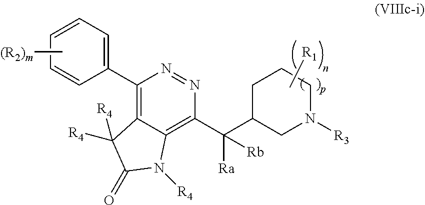

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (VIIIc-ii):

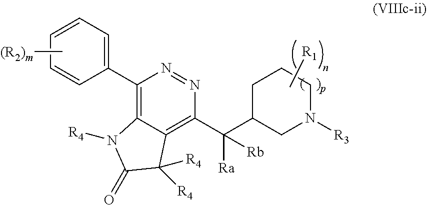

or a pharmaceutically acceptable salt, isomer, hydrate, solvate or isotope thereof, wherein R₁, R₂, R₃, R₄, Rₐ, R_{b}, m, n, and p are as defined herein.

In other embodiments, a compound is provided having structure (VIIIc-iii):

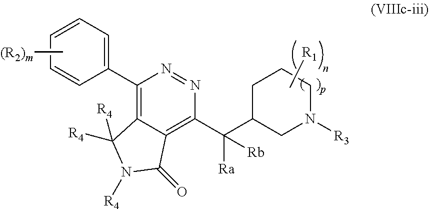

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (VIIIc-iv):

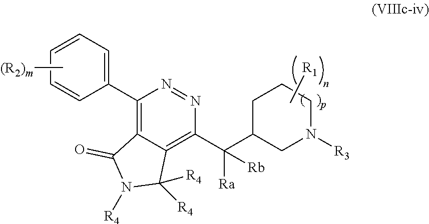

or a pharmaceutically acceptable salt, isomer, hydrate, solvate or isotope thereof, wherein R₁, R₂, R₃, R₄, Rₐ, R_{b}, m, n, and p are as defined herein.

In further embodiments, a compound is provided having structure (IXa-i):

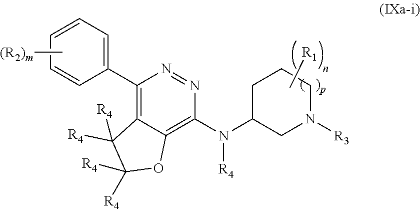

or a pharmaceutically acceptable salt, isomer, hydrate, solvate or isotope thereof, wherein R₁, R₂, R₃, R₄, m, n, and p are as defined herein.

In some embodiments, a compound is provided having structure (IXa-ii):

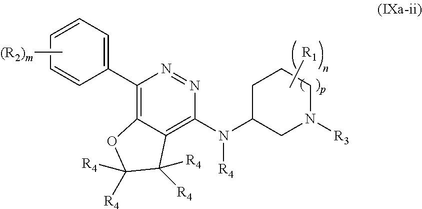

or a pharmaceutically acceptable salt, isomer, hydrate, solvate or isotope thereof, wherein R₁, R₂, R₃, R₄, m, n, and p are as defined herein.

In other embodiments, a compound is provided having structure (IXa-iii):

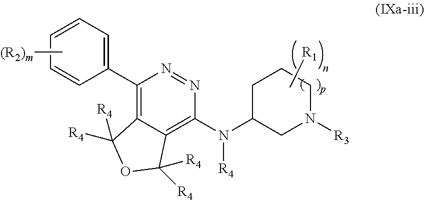

or a pharmaceutically acceptable salt, isomer, hydrate, solvate or isotope thereof, wherein R₁, R₂, R₃, R₄, m, n, and p are as defined herein.

In additional embodiments, a compound is provided having structure (IXa-iv):

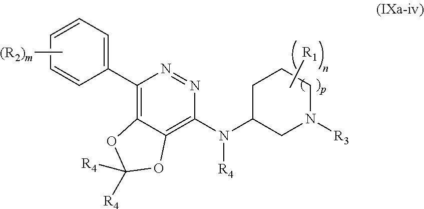

or a pharmaceutically acceptable salt, isomer, hydrate, solvate or isotope thereof, wherein R₁, R₂, R₃, R₄, m, n, and p are as defined herein.

In further embodiments, a compound is provided having structure (IXb-i):

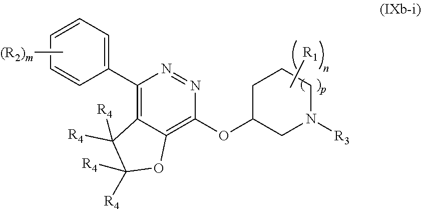

or a pharmaceutically acceptable salt, isomer, hydrate, solvate or isotope thereof, wherein R₁, R₂, R₃, R₄, m, n, and p are as defined herein.

In some embodiments, a compound is provided having structure (IXb-ii):

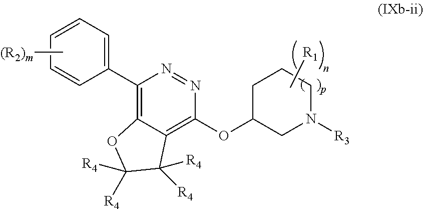

or a pharmaceutically acceptable salt, isomer, hydrate, solvate or isotope thereof, wherein R₁, R₂, R₃, R₄, m, n, and p are as defined herein.

In additional embodiments, a compound is provided having structure (IXb-iii):

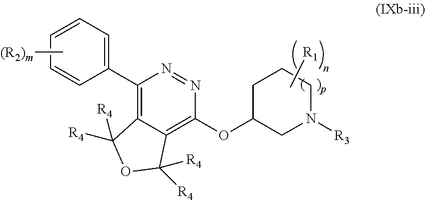

or a pharmaceutically acceptable salt, isomer, hydrate, solvate or isotope thereof, wherein R₁, R₂, R₃, R₄, m, n, and p are as defined herein.

In additional embodiments, a compound is provided having structure (IXb-iv):

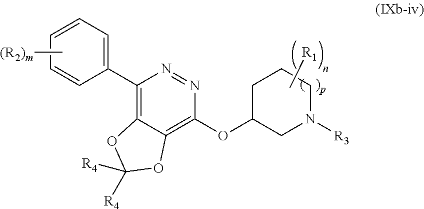

or a pharmaceutically acceptable salt, isomer, hydrate, solvate or isotope hereof, wherein R₁, R₂, R₃, R₄, m, n, and p are as defined herein.

In further embodiments, a compound is provided having structure (IXc-i):

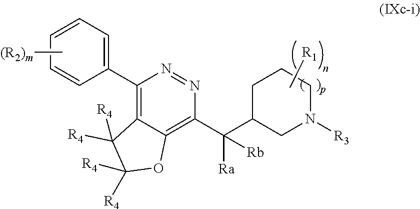

or a pharmaceutically acceptable salt, isomer, hydrate, solvate or isotope thereof, wherein R₁, R₂, R₃, R₄, Rₐ, R_{b}, m, n, and p are as defined herein.

In some embodiments, a compound is provided having structure (IXc-ii):

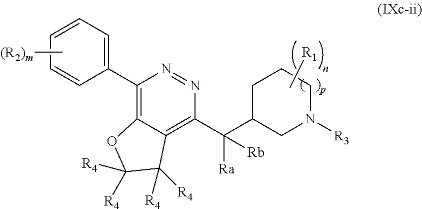

or a pharmaceutically acceptable salt, isomer, hydrate, solvate or isotope thereof, wherein R₁, R₂, R₃, R₄, Rₐ, R_{b}, m, n, and p are as defined herein.

In other embodiments, a compound is provided having structure (IXc-iii):

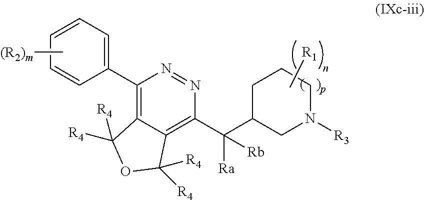

or a pharmaceutically acceptable salt, isomer, hydrate, solvate or isotope thereof, wherein R₁, R₂, R₃, R₄, Rₐ, R_{b}, m, n, and p are as defined herein.

In additional embodiments, a compound is provided having structure (IXc-iv):

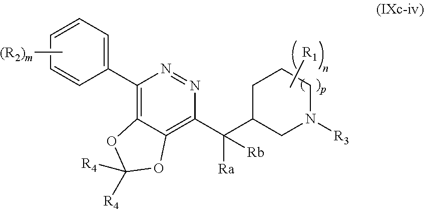

or a pharmaceutically acceptable salt, isomer, hydrate, solvate or isotope thereof, wherein R₁, R₂, R₃, R₄, Rₐ, R_{b}, m, n, and p are as defined herein.

In more specific embodiments, each of the structures listed herein, as appropriate, include the following more specific embodiments.

In some embodiments, a compound is provided wherein

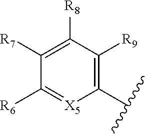

has one of the following structures:

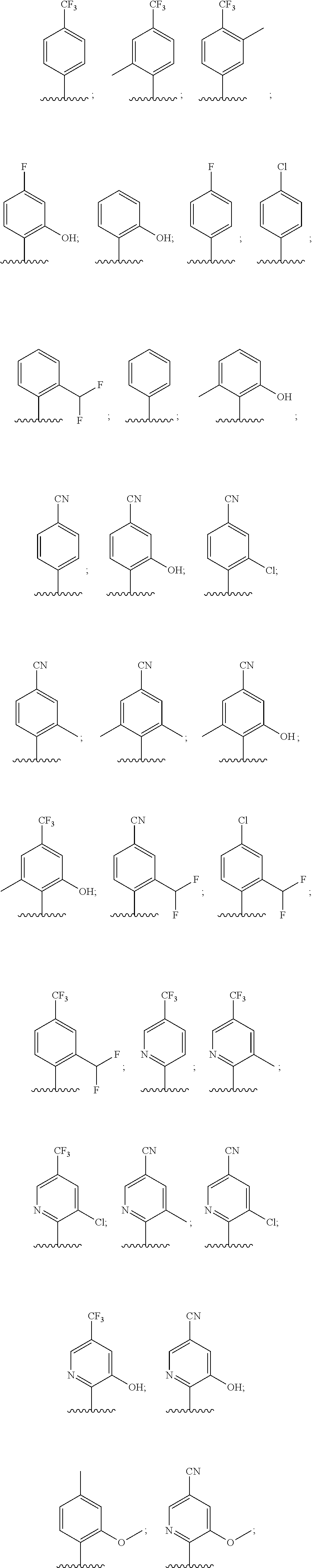

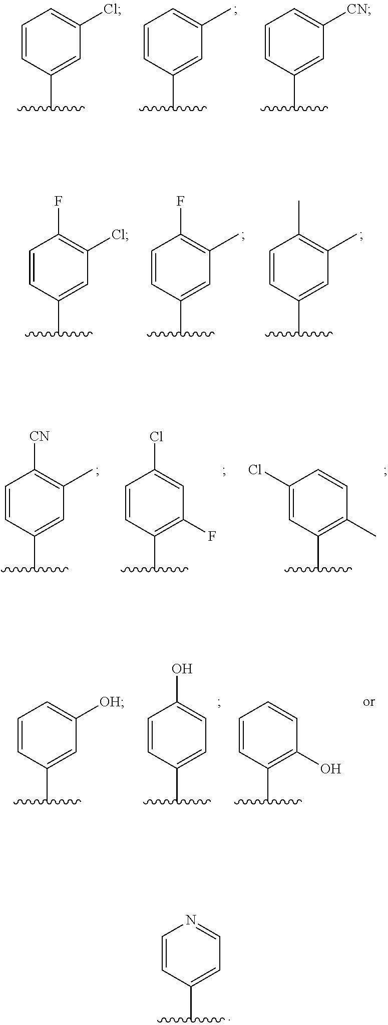

In some embodiments, a compound is provided wherein

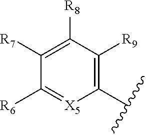

has one of the following structures:

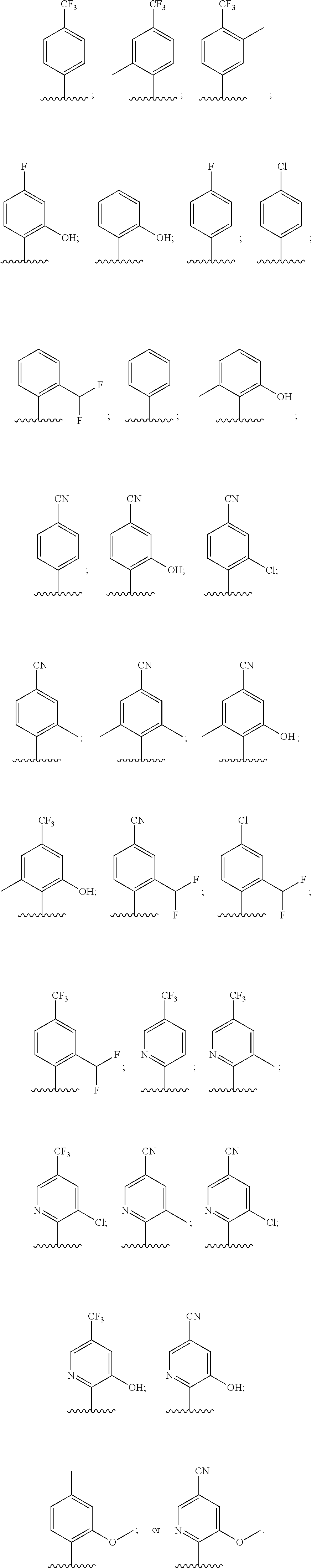

In some embodiments, a compound is provided wherein

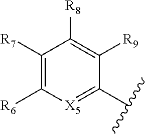

has one of the following structures:

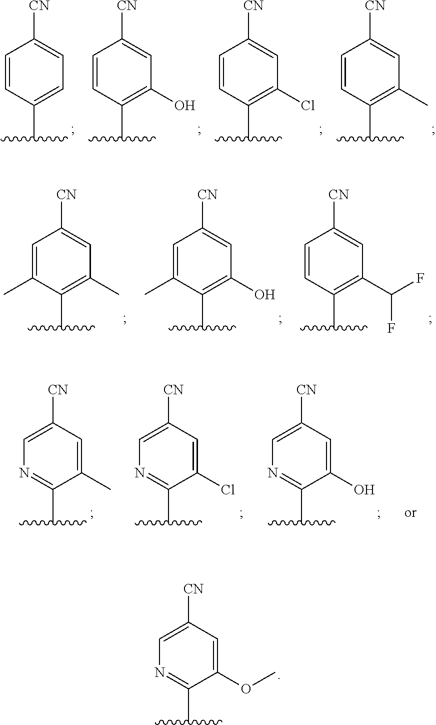

In some embodiments, a compound is provided wherein

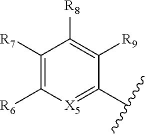

has one of the following structures:

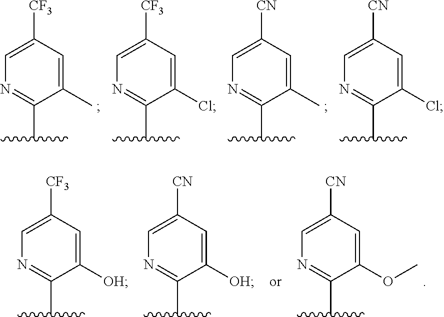

In some embodiments, a compound is provided wherein X is CRₐR_{b}. In other embodiments, a compound is provided wherein Rₐ and R_{b} are both H. In additional embodiments, a compound is provided wherein one of Rₐ and R_{b} is H and the other is alkyl. In further embodiments, a compound is provided wherein Rₐ and R_{b} are both alkyl. In further embodiments, a compound is provided wherein Rₐ and R_{b} are both C₁-C₆ alkyl. In specific embodiments, a compound is provided wherein the alkyl is methyl. In some embodiments, a compound is provided wherein the alkyl is ethyl. In other embodiments, a compound is provided wherein the alkyl is propyl. In yet other embodiments, a compound is provided wherein the alkyl is isopropyl. In additional embodiments, a compound is provided wherein the alkyl is n-butyl. In further embodiments, a compound is provided wherein the alkyl is sec-butyl. In some embodiments, a compound is provided wherein the alkyl is iso-butyl. In other embodiments, a compound is provided wherein the alkyl is tert-butyl.

In some embodiments, a compound is provided wherein X is NR₄. In certain embodiments, a compound is provided wherein R₄ is H. In other embodiments, a compound is provided wherein R₄ is alkyl. In further embodiments, a compound is provided wherein R₄ is C₁-C₆ alkyl. In yet other embodiments, a compound is provided wherein R₄ is methyl. In some embodiments, a compound is provided wherein R₄ is ethyl. In other embodiments, a compound is provided wherein R₄ is propyl. In yet other embodiments, a compound is provided wherein R₄ is isopropyl. In additional embodiments, a compound is provided wherein R₄ is n-butyl. In further embodiments, a compound is provided wherein R₄ is sec-butyl. In some embodiments, a compound is provided wherein R₄ is iso-butyl. In other embodiments, a compound is provided wherein R₄ is tert-butyl.

In some embodiments, a compound is provided wherein X is O.

In some embodiments, a compound is provided wherein R₁ is F. In other embodiments, a compound is provided, wherein R₁ is Cl. In additional embodiments, a compound is provided wherein R₁ is Br. In further embodiments, a compound is provided wherein R₁ is I.

In some embodiments, a compound is provided wherein R₂ is halo. In certain embodiments, a compound is provided wherein R₂ is F. In other embodiments, a compound is provided wherein R₂ is Cl. In additional embodiments, a compound is provided wherein R₂ is Br. In further embodiments, a compound is provided wherein R₂ is I. In some embodiments, a compound is provided wherein R₂ is OH. In other embodiments, a compound is provided wherein R₂ is CN. In specific embodiments, a compound is provided wherein R₂ is alkyl. In further embodiments, a compound is provided wherein R₂ is C₁-C₆ alkyl. In yet other embodiments, a compound is provided wherein R₂ is methyl. In some embodiments, a compound is provided wherein R₂ is ethyl. In other embodiments, a compound is provided wherein R₂ is propyl. In yet other embodiments, a compound is provided wherein R₂ is isopropyl. In additional embodiments, a compound is provided wherein R₂ is n-butyl. In further embodiments, a compound is provided wherein R₂ is sec-butyl. In some embodiments, a compound is provided wherein R₂ is iso-butyl. In other embodiments, a compound is provided wherein R₂ is tert-butyl. In some embodiments, a compound is provided wherein R₂ is haloalkyl. In specific embodiments, a compound is provided wherein R₂ is C₁-C₆ haloalkyl. In certain embodiments, a compound is provided wherein R₂ is CF₃. In other embodiments, a compound is provided wherein R₂ is CHF₂. In some embodiments, a compound is provided wherein R₂ is alkoxy. In specific embodiments, a compound is provided wherein R₂ is C₁-C₆ alkoxy. In certain embodiments, a compound is provided wherein R₂ is OCH₃. In some embodiments, a compound is provided wherein R₂ is haloalkoxy. In specific embodiments, a compound is provided wherein R₂ is C₁-C₆ haloalkoxy. In other embodiments, a compound is provided wherein R₂ is OCF₃. In some embodiments, a compound is provided wherein R₂ is cycloalkyl. In specific embodiments, a compound is provided wherein R₂ is C₃-C₈ cycloalkyl. In other embodiments, a compound is provided wherein R₂ is cyclopropyl. In yet other embodiments, a compound is provided wherein R₂ is cyclobutyl. In additional embodiments, a compound is provided wherein R₂ is cyclopentyl.

In some embodiments, a compound is provided wherein R₃ is H. In other embodiments, a compound is provided wherein R₃ is not H. In certain embodiments, a compound is provided wherein R₃ is alkyl. In specific embodiments, a compound is provided wherein R₃ is C₁-C₆ alkyl. In yet other embodiments, a compound is provided wherein R₃ is methyl. In some embodiments, a compound is provided wherein R₃ is ethyl. In other embodiments, a compound is provided wherein R₃ is propyl. In yet other embodiments, a compound is provided wherein R₃ is isopropyl. In additional embodiments, a compound is provided wherein R₃ is n-butyl. In further embodiments, a compound is provided wherein R₃ is sec-butyl. In some embodiments, a compound is provided wherein R₃ is iso-butyl. In other embodiments, a compound is provided wherein R₃ is tert-butyl.

In some embodiments, a compound is provided wherein R₃ is alkyl substituted with one or more F. In certain embodiments, a compound is provided wherein R₃ is —CH₂CH₂F. In other embodiments, a compound is provided wherein R₃ is —CH₂CHF₂. In yet other embodiments, a compound is provided wherein R₃ is —CH₂CF₃.

In some embodiments, a compound is provided wherein R₃ is alkyl substituted with one or more cycloalkyl. In certain embodiments, a compound is provided wherein R₃ is

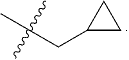 [SMILES: *CC1CC1]

In other embodiments, a compound is provided wherein R₃ is alkyl substituted with one or more OH. In certain embodiments, a compound is provided wherein R₃ is

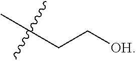 [SMILES: *CCO]

In certain embodiments, a compound is provided wherein R₃ is

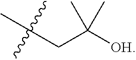 [SMILES: *CC(C)(C)O]

In some embodiments, a compound is provided wherein R₃ is alkyl substituted with one or more alkoxy. In certain embodiments, a compound is provided wherein R₃ is

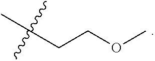 [SMILES: *CCOC]

In some embodiments, a compound is provided wherein R₃ is alkyl substituted with one or more aminyl. In certain embodiments, a compound is provided wherein R₃ is

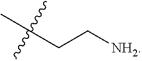 [SMILES: *CCN]

In certain embodiments, a compound is provided wherein R₃ is

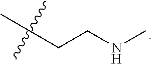 [SMILES: *CCNC]

In certain embodiments, a compound is provided wherein R₃ is

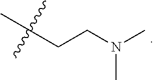 [SMILES: *CCN(C)C]

In some embodiments, a compound is provided wherein R₃ is alkylsulfonyl. In certain embodiments, a compound is provided wherein R₃ is

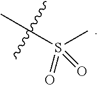 [SMILES: *S(C)(=O)=O]

In some embodiments, a compound is provided wherein R₃ is cycloalkyl. In specific embodiments, a compound is provided wherein R₃ is C₃-C₈ cycloalkyl. In other embodiments, a compound is provided wherein R₃ is cyclopropyl. In yet other embodiments, a compound is provided wherein R₃ is cyclobutyl. In additional embodiments, a compound is provided wherein R₃ is cyclopentyl.

In some embodiments, a compound is provided wherein R₃ is aryl. In specific embodiments, a compound is provided wherein R₃ is phenyl.

In some embodiments, a compound is provided wherein R₃ is heterocyclyl. In specific embodiments, a compound is provided wherein R₃ is saturated heterocyclyl. In certain embodiments, a compound is provided wherein R₃ is

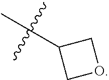 [SMILES: *C1COC1]

In some embodiments, a compound is provided wherein R₃ is heteroaryl. In specific embodiments, a compound is provided wherein R₃ is piperidinyl.

In some embodiments, a compound is provided wherein

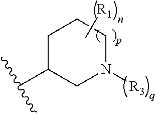

has the following structure:

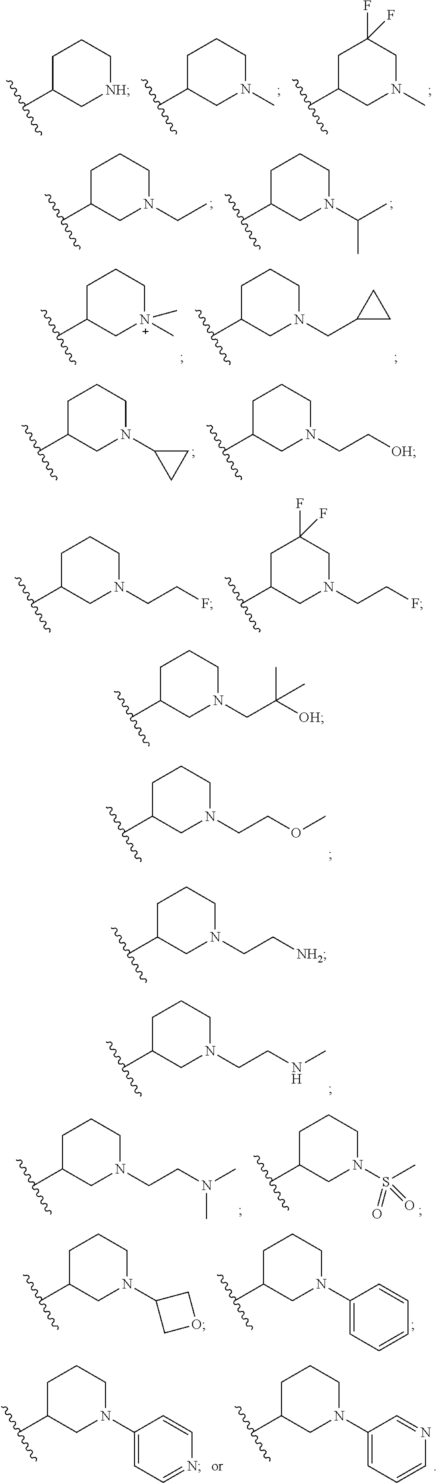

In some embodiments, a compound is provided wherein an R₄ is H. In other embodiments, a compound is provided wherein an R₄ is alkyl. In certain embodiments, a compound is provided wherein an R₄ is C₁-C₆ alkyl. In specific embodiments, a compound is provided wherein an R₄ is methyl. In additional embodiments, a compound is provided wherein an R₄ is ethyl.

In some embodiments, a compound is provided wherein each R₄ is H or methyl. In other embodiments, a compound is provided wherein each R₄ is H or ethyl. In some embodiments, a compound is provided wherein when attached to a carbon atom, two R₄ join together to form oxo.

In some embodiments, a compound is provided wherein X₅ is CR₅. In other embodiments, a compound is provided wherein X₅ is N.

In some embodiments, a compound is provided wherein R₅ is H. In other embodiments, a compound is provided wherein R₅ is halo. In additional embodiments, a compound is provided wherein R₅ is OH. In further embodiments, a compound is provided wherein R₅ is CN. In yet other embodiments, a compound is provided wherein R₅ is alkyl. In some embodiments, a compound is provided wherein R₅ is haloalkyl. In other embodiments, a compound is provided wherein R₅ is alkoxy. In additional embodiments, a compound is provided wherein R₅ is haloalkoxy. In further embodiments, a compound is provided wherein R₅ is cycloalkyl. In yet other embodiments, a compound is provided wherein R₅ is H or alkyl.

In some embodiments, a compound is provided wherein R₆ is H. In other embodiments, a compound is provided wherein R₆ is halo. In additional embodiments, a compound is provided wherein R₆ is OH. In further embodiments, a compound is provided wherein R₆ is CN. In yet other embodiments, a compound is provided wherein R₆ is alkyl. In some embodiments, a compound is provided wherein R₆ is haloalkyl. In other embodiments, a compound is provided wherein R₆ is alkoxy. In additional embodiments, a compound is provided wherein R₆ is haloalkoxy. In further embodiments, a compound is provided wherein R₆ is cycloalkyl. In yet other embodiments, a compound is provided wherein R₆ is H or halo. In yet other embodiments, a compound is provided wherein R₆ is H or Cl.

In some embodiments, a compound is provided wherein R₇ is H. In other embodiments, a compound is provided wherein R₇ is halo. In additional embodiments, a compound is provided wherein R₇ is OH. In further embodiments, a compound is provided wherein R₇ is CN. In yet other embodiments, a compound is provided wherein R₇ is alkyl. In some embodiments, a compound is provided wherein R₇ is haloalkyl. In other embodiments, a compound is provided wherein R₇ is alkoxy. In additional embodiments, a compound is provided wherein R₇ is haloalkoxy. In further embodiments, a compound is provided wherein R₇ is cycloalkyl. In specific embodiments, a compound is provided wherein R₇ is CN or haloalkyl. In specific embodiments, a compound is provided wherein R₇ is CN or CF₃. In yet other embodiments, a compound is provided wherein R₇ is H, halo, OH, CN, alkyl, or haloalkyl. In yet other embodiments, a compound is provided wherein R₇ is H, F, Cl, OH, CN, methyl, or CF₃.

In some embodiments, a compound is provided wherein R₈ is H. In other embodiments, a compound is provided wherein R₈ is halo. In additional embodiments, a compound is provided wherein R₈ is OH. In further embodiments, a compound is provided wherein R₈ is CN. In yet other embodiments, a compound is provided wherein R₈ is alkyl. In some embodiments, a compound is provided wherein R₈ is haloalkyl. In other embodiments, a compound is provided wherein R₈ is alkoxy. In additional embodiments, a compound is provided wherein R₈ is haloalkoxy. In further embodiments, a compound is provided wherein R₈ is cycloalkyl. In yet other embodiments, a compound is provided wherein R₈ is H, alkyl, halo, or OH. In additional embodiments, a compound is provided wherein R₈ is H or alkyl. In further embodiments, a compound is provided wherein R₈ is H or methyl. In other embodiments, a compound is provided wherein R₈ is H, methyl, F, Cl, or OH. In other embodiments, a compound is provided wherein R₈ is H, methyl, Cl, or OH.

In some embodiments, a compound is provided wherein R₉ is H. In other embodiments, a compound is provided wherein R₉ is halo. In additional embodiments, a compound is provided wherein R₉ is OH. In further embodiments, a compound is provided wherein R₉ is CN. In yet other embodiments, a compound is provided wherein R₉ is alkyl. In some embodiments, a compound is provided wherein R₉ is haloalkyl. In other embodiments, a compound is provided wherein R₉ is alkoxy. In additional embodiments, a compound is provided wherein R₉ is haloalkoxy. In further embodiments, a compound is provided wherein R₉ is cycloalkyl. In yet other embodiments, a compound is provided wherein R₉ is H, OH, halo, haloalkyl, alkyl, or alkoxy. In yet other embodiments, a compound is provided wherein R₉ is H, OH, Cl, CHF₂, methyl, or methoxy. In yet other embodiments, a compound is provided wherein R₉ is H, OH, F, Cl, CHF₂, CF₃, methyl, or methoxy.

In other embodiments, a compound is provided wherein R₁₀ is halo. In additional embodiments, a compound is provided wherein R₁₀ is OH. In further embodiments, a compound is provided wherein R₁₀ is CN. In yet other embodiments, a compound is provided wherein R₁₀ is alkyl. In some embodiments, a compound is provided wherein R₁₀ is haloalkyl. In other embodiments, a compound is provided wherein R₁₀ is alkoxy. In additional embodiments, a compound is provided wherein R₁₀ is haloalkoxy. In further embodiments, a compound is provided wherein R₁₀ is aminyl. In some embodiments, a compound is provided wherein R₁₀ is alkylsulfonyl. In other embodiments, a compound is provided wherein R₁₀ is carbocyclyl. In yet other embodiments, a compound is provided wherein R₁₀ is cycloalkyl. In additional embodiments, a compound is provided wherein R₁₀ is aryl. In further embodiments, a compound is provided wherein R₁₀ is heterocyclyl. In some embodiments, a compound is provided wherein R₁₀ is saturated heterocyclyl. In other embodiments, a compound is provided wherein R₁₀ is heteroaryl. In certain embodiments, a compound is provided wherein R₁₀ is N(CH₃)₂.

In some embodiments, a compound is provided wherein m is 0. In other embodiments, a compound is provided wherein m is 1. In additional embodiments, a compound is provided wherein m is 2. In further embodiments, a compound is provided wherein m is 3. In yet other embodiments, a compound is provided wherein m is 4. In further embodiments, a compound is provided wherein m is 5.

In some embodiments, a compound is provided wherein n is 0. In other embodiments, a compound is provided wherein n is 1. In additional embodiments, a compound is provided wherein n is 2. In further embodiments, a compound is provided wherein n is 3. In yet other embodiments, a compound is provided wherein n is 4.

In some embodiments, a compound is provided wherein p is 0. In other embodiments, a compound is provided wherein p is 1. In yet other embodiments, a compound is provided wherein p is 2.

In other embodiments, a compound is provided wherein q is 1. In yet other embodiments, a compound is provided wherein q is 2.

In other embodiments, a compound is provided wherein y is 1. In additional embodiments, a compound is provided wherein y is 2. In further embodiments, a compound is provided wherein y is 3. In yet other embodiments, a compound is provided wherein y is 4. In some embodiments, a compound is provided wherein y is 5. In yet other embodiments, a compound is provided wherein y is 6.

In some embodiments, a compound is provided wherein p is 1, n is 0, and m is 0. In other embodiments, a compound is provided wherein p is 1, n is 0, and m is 1. In additional embodiments, a compound is provided wherein p is 1, n is 0, and m is 2. In further embodiments, a compound is provided wherein p is 1, n is 2, and m is 1.

In one embodiment, a compound is selected from one of the compounds listed in Table 1, or a pharmaceutically acceptable salt, isomer, hydrate, solvate or isotope thereof.

TABLE 1
Representative compounds having Structure (I)

| Cpd | |
|---|---|
| No. | Structure |
| 1 | 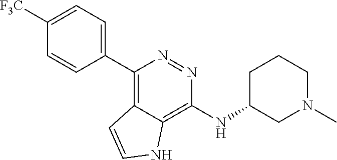 [SMILES: CN1CCC[C@@H](Nc2nnc(-c3ccc(C(F)(F)F)cc3)c3cc[nH]c23)C1] |
| 2 | 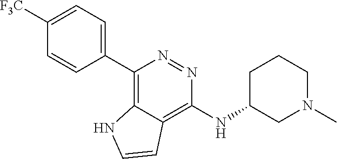 [SMILES: CN1CCC[C@@H](Nc2nnc(-c3ccc(C(F)(F)F)cc3)c3[nH]ccc23)C1] |
| 3 | 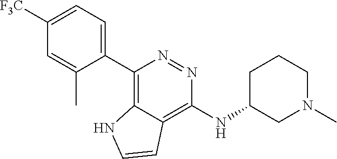 [SMILES: Cc1cc(C(F)(F)F)ccc1-c1nnc(N[C@@H]2CCCN(C)C2)c2cc[nH]c12] |
| 4 | 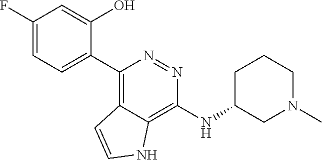 [SMILES: CN1CCC[C@@H](Nc2nnc(-c3ccc(F)cc3O)c3cc[nH]c23)C1] |
| 5 | 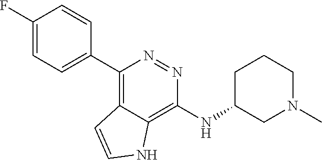 [SMILES: CN1CCC[C@@H](Nc2nnc(-c3ccc(F)cc3)c3cc[nH]c23)C1] |
| 6 | 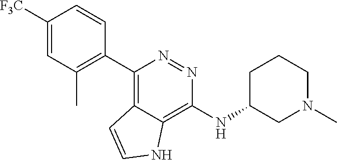 [SMILES: Cc1cc(C(F)(F)F)ccc1-c1nnc(N[C@@H]2CCCN(C)C2)c2[nH]ccc12] |
| 7 | 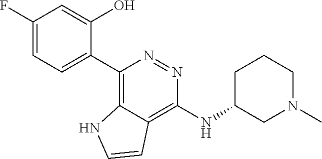 [SMILES: CN1CCC[C@@H](Nc2nnc(-c3ccc(F)cc3O)c3[nH]ccc23)C1] |
| 8 | 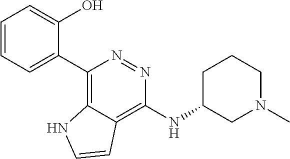 [SMILES: CN1CCC[C@@H](Nc2nnc(-c3ccccc3O)c3[nH]ccc23)C1] |
| 9 | 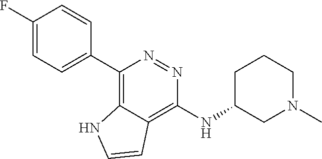 [SMILES: CN1CCC[C@@H](Nc2nnc(-c3ccc(F)cc3)c3[nH]ccc23)C1] |
| 10 | 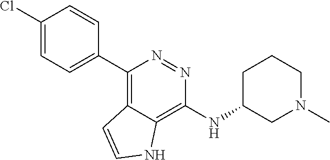 [SMILES: CN1CCC[C@@H](Nc2nnc(-c3ccc(Cl)cc3)c3cc[nH]c23)C1] |
| 11 | 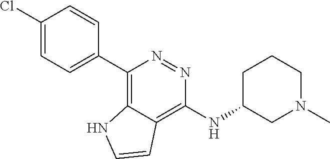 [SMILES: CN1CCC[C@@H](Nc2nnc(-c3ccc(Cl)cc3)c3[nH]ccc23)C1] |
| 12 | 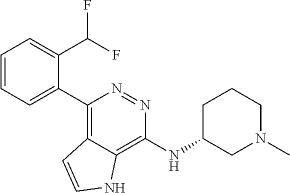 [SMILES: CN1CCC[C@@H](Nc2nnc(-c3ccccc3C(F)F)c3cc[nH]c23)C1] |
| 13 | 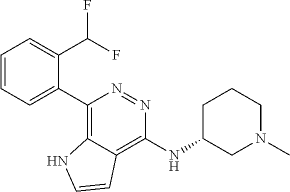 [SMILES: CN1CCC[C@@H](Nc2nnc(-c3ccccc3C(F)F)c3[nH]ccc23)C1] |
| 14 | 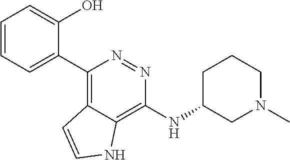 [SMILES: CN1CCC[C@@H](Nc2nnc(-c3ccccc3O)c3cc[nH]c23)C1] |
| 15 | 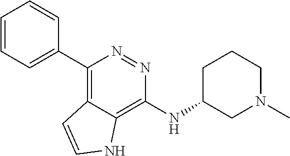 [SMILES: CN1CCC[C@@H](Nc2nnc(-c3ccccc3)c3cc[nH]c23)C1] |
| 16 | 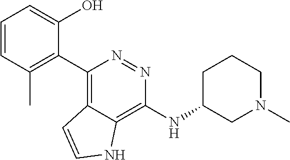 [SMILES: Cc1cccc(O)c1-c1nnc(N[C@@H]2CCCN(C)C2)c2[nH]ccc12] |
| 17 | 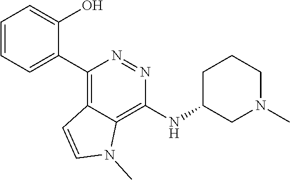 [SMILES: CN1CCC[C@@H](Nc2nnc(-c3ccccc3O)c3ccn(C)c23)C1] |
| 18 | 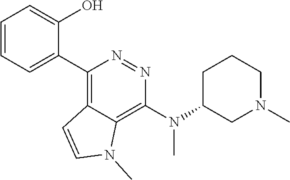 [SMILES: CN1CCC[C@@H](N(C)c2nnc(-c3ccccc3O)c3ccn(C)c23)C1] |
| 19 | 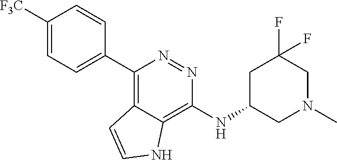 [SMILES: CN1C[C@H](Nc2nnc(-c3ccc(C(F)(F)F)cc3)c3cc[nH]c23)CC(F)(F)C1] |
| 20 | 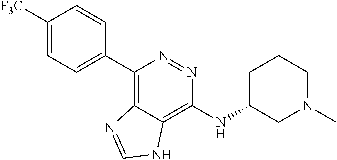 [SMILES: CN1CCC[C@@H](Nc2nnc(-c3ccc(C(F)(F)F)cc3)c3nc[nH]c23)C1] |
| 21 | 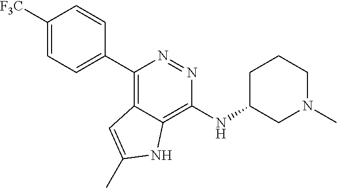 [SMILES: Cc1cc2c(-c3ccc(C(F)(F)F)cc3)nnc(N[C@@H]3CCCN(C)C3)c2[nH]1] |
| 22 | 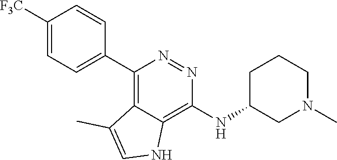 [SMILES: Cc1c[nH]c2c(N[C@@H]3CCCN(C)C3)nnc(-c3ccc(C(F)(F)F)cc3)c12] |
| 23 | 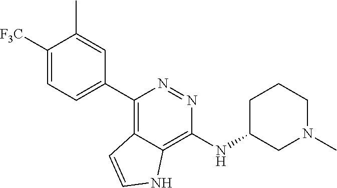 [SMILES: Cc1cc(-c2nnc(N[C@@H]3CCCN(C)C3)c3[nH]ccc23)ccc1C(F)(F)F] |
| 24 | 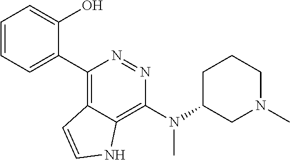 [SMILES: CN1CCC[C@@H](N(C)c2nnc(-c3ccccc3O)c3cc[nH]c23)C1] |
| 25 | 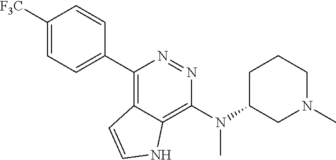 [SMILES: CN1CCC[C@@H](N(C)c2nnc(-c3ccc(C(F)(F)F)cc3)c3cc[nH]c23)C1] |
| 26 | 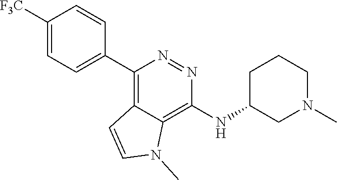 [SMILES: CN1CCC[C@@H](Nc2nnc(-c3ccc(C(F)(F)F)cc3)c3ccn(C)c23)C1] |
| 27 | 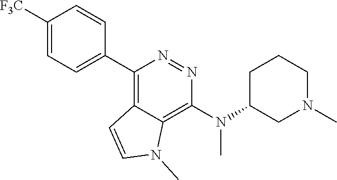 [SMILES: CN1CCC[C@@H](N(C)c2nnc(-c3ccc(C(F)(F)F)cc3)c3ccn(C)c23)C1] |
| 28 | 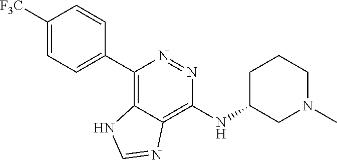 [SMILES: CN1CCC[C@@H](Nc2nnc(-c3ccc(C(F)(F)F)cc3)c3[nH]cnc23)C1] |
| 29 | 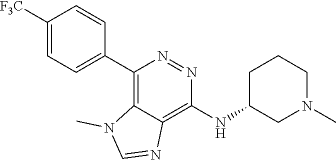 [SMILES: CN1CCC[C@@H](Nc2nnc(-c3ccc(C(F)(F)F)cc3)c3c2ncn3C)C1] |
| 30 | 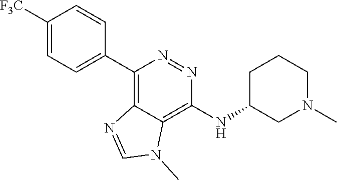 [SMILES: CN1CCC[C@@H](Nc2nnc(-c3ccc(C(F)(F)F)cc3)c3ncn(C)c23)C1] |
| 31 | 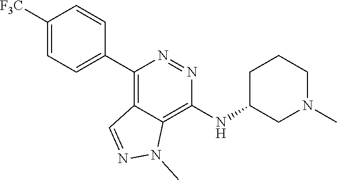 [SMILES: CN1CCC[C@@H](Nc2nnc(-c3ccc(C(F)(F)F)cc3)c3cnn(C)c23)C1] |
| 32 | 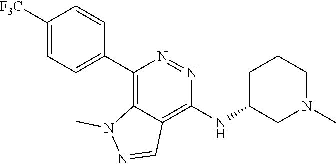 [SMILES: CN1CCC[C@@H](Nc2nnc(-c3ccc(C(F)(F)F)cc3)c3c2cnn3C)C1] |
| 33 | 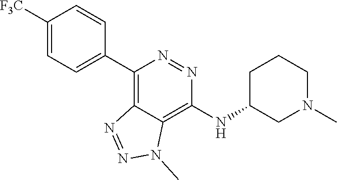 [SMILES: CN1CCC[C@@H](Nc2nnc(-c3ccc(C(F)(F)F)cc3)c3nnn(C)c23)C1] |
| 34 | 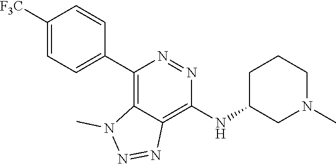 [SMILES: CN1CCC[C@@H](Nc2nnc(-c3ccc(C(F)(F)F)cc3)c3c2nnn3C)C1] |
| 35 | 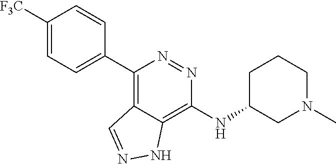 [SMILES: CN1CCC[C@@H](Nc2nnc(-c3ccc(C(F)(F)F)cc3)c3cn[nH]c23)C1] |
| 36 | 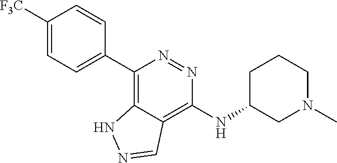 [SMILES: CN1CCC[C@@H](Nc2nnc(-c3ccc(C(F)(F)F)cc3)c3[nH]ncc23)C1] |
| 37 | 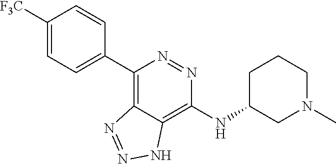 [SMILES: CN1CCC[C@@H](Nc2nnc(-c3ccc(C(F)(F)F)cc3)c3nn[nH]c23)C1] |
| 38 | 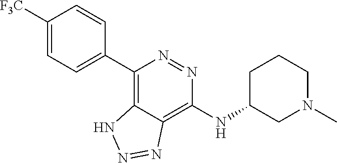 [SMILES: CN1CCC[C@@H](Nc2nnc(-c3ccc(C(F)(F)F)cc3)c3[nH]nnc23)C1] |
| 39 | 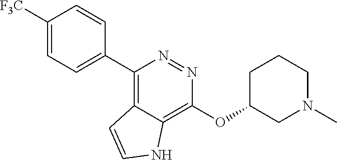 [SMILES: CN1CCC[C@@H](Oc2nnc(-c3ccc(C(F)(F)F)cc3)c3cc[nH]c23)C1] |
| 40 | 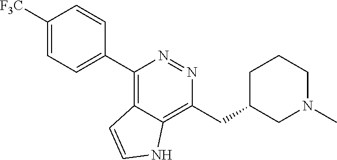 [SMILES: CN1CCC[C@@H](Cc2nnc(-c3ccc(C(F)(F)F)cc3)c3cc[nH]c23)C1] |
| 41 | 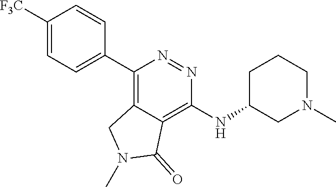 [SMILES: CN1CCC[C@@H](Nc2nnc(-c3ccc(C(F)(F)F)cc3)c3c2C(=O)N(C)C3)C1] |
| 42 | 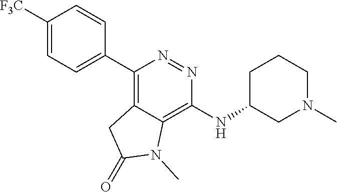 [SMILES: CN1CCC[C@@H](Nc2nnc(-c3ccc(C(F)(F)F)cc3)c3c2N(C)C(=O)C3)C1] |
| 43 | 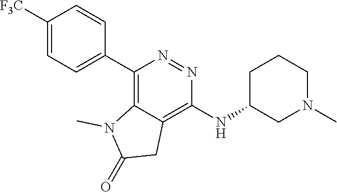 [SMILES: CN1CCC[C@@H](Nc2nnc(-c3ccc(C(F)(F)F)cc3)c3c2CC(=O)N3C)C1] |
| 44 | 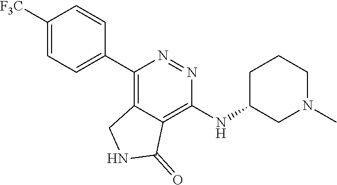 [SMILES: CN1CCC[C@@H](Nc2nnc(-c3ccc(C(F)(F)F)cc3)c3c2C(=O)NC3)C1] |
| 45 | 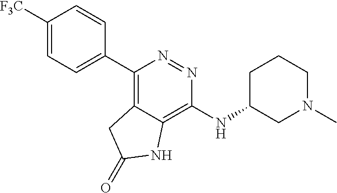 [SMILES: CN1CCC[C@@H](Nc2nnc(-c3ccc(C(F)(F)F)cc3)c3c2NC(=O)C3)C1] |
| 46 | 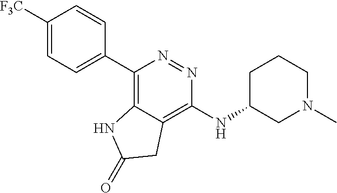 [SMILES: CN1CCC[C@@H](Nc2nnc(-c3ccc(C(F)(F)F)cc3)c3c2CC(=O)N3)C1] |
| 47 | 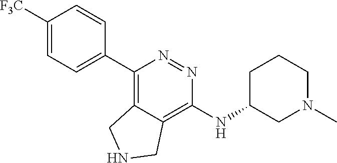 [SMILES: CN1CCC[C@@H](Nc2nnc(-c3ccc(C(F)(F)F)cc3)c3c2CNC3)C1] |
| 48 | 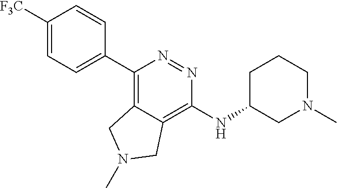 [SMILES: CN1Cc2c(N[C@@H]3CCCN(C)C3)nnc(-c3ccc(C(F)(F)F)cc3)c2C1] |
| 49 | 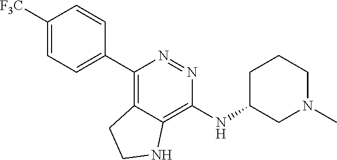 [SMILES: CN1CCC[C@@H](Nc2nnc(-c3ccc(C(F)(F)F)cc3)c3c2NCC3)C1] |
| 50 | 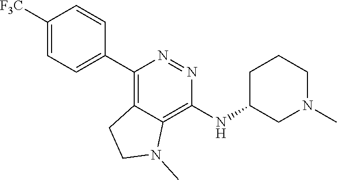 [SMILES: CN1CCC[C@@H](Nc2nnc(-c3ccc(C(F)(F)F)cc3)c3c2N(C)CC3)C1] |
| 51 | 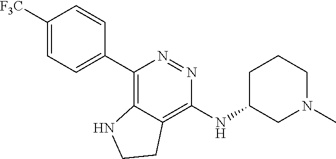 [SMILES: CN1CCC[C@@H](Nc2nnc(-c3ccc(C(F)(F)F)cc3)c3c2CCN3)C1] |
| 52 | 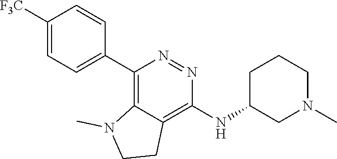 [SMILES: CN1CCC[C@@H](Nc2nnc(-c3ccc(C(F)(F)F)cc3)c3c2CCN3C)C1] |
| 53 | 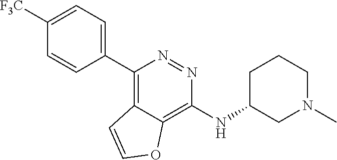 [SMILES: CN1CCC[C@@H](Nc2nnc(-c3ccc(C(F)(F)F)cc3)c3ccoc23)C1] |
| 54 | 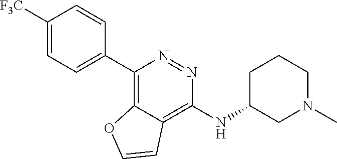 [SMILES: CN1CCC[C@@H](Nc2nnc(-c3ccc(C(F)(F)F)cc3)c3occc23)C1] |
| 55 | 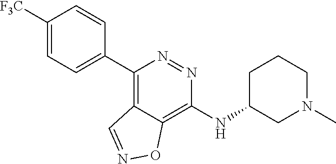 [SMILES: CN1CCC[C@@H](Nc2nnc(-c3ccc(C(F)(F)F)cc3)c3cnoc23)C1] |
| 56 | 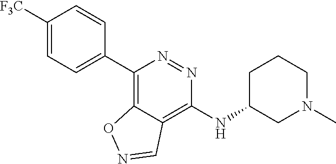 [SMILES: CN1CCC[C@@H](Nc2nnc(-c3ccc(C(F)(F)F)cc3)c3oncc23)C1] |
| 57 | 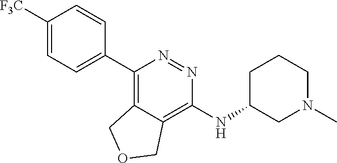 [SMILES: CN1CCC[C@@H](Nc2nnc(-c3ccc(C(F)(F)F)cc3)c3c2COC3)C1] |
| 58 | 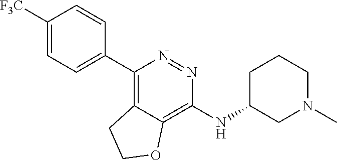 [SMILES: CN1CCC[C@@H](Nc2nnc(-c3ccc(C(F)(F)F)cc3)c3c2OCC3)C1] |
| 59 | 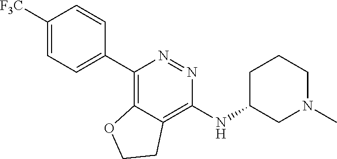 [SMILES: CN1CCC[C@@H](Nc2nnc(-c3ccc(C(F)(F)F)cc3)c3c2CCO3)C1] |
| 60 | 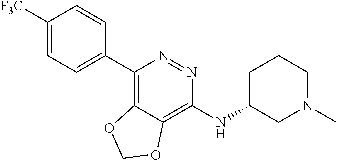 [SMILES: CN1CCC[C@@H](Nc2nnc(-c3ccc(C(F)(F)F)cc3)c3c2OCO3)C1] |
| 61 | 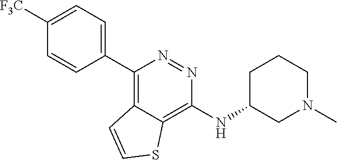 [SMILES: CN1CCC[C@@H](Nc2nnc(-c3ccc(C(F)(F)F)cc3)c3ccsc23)C1] |
| 62 | 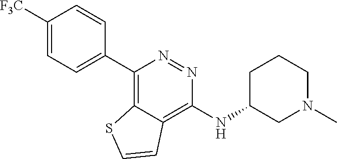 [SMILES: CN1CCC[C@@H](Nc2nnc(-c3ccc(C(F)(F)F)cc3)c3sccc23)C1] |
| 63 | 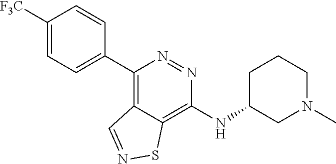 [SMILES: CN1CCC[C@@H](Nc2nnc(-c3ccc(C(F)(F)F)cc3)c3cnsc23)C1] |
| 64 | 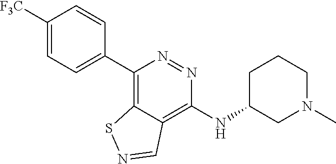 [SMILES: CN1CCC[C@@H](Nc2nnc(-c3ccc(C(F)(F)F)cc3)c3sncc23)C1] |
| 65 | 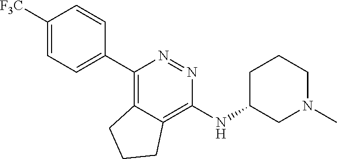 [SMILES: CN1CCC[C@@H](Nc2nnc(-c3ccc(C(F)(F)F)cc3)c3c2CCC3)C1] |
| 66 | 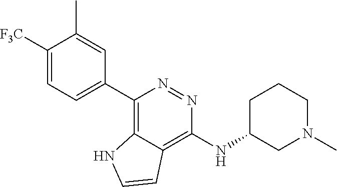 [SMILES: Cc1cc(-c2nnc(N[C@@H]3CCCN(C)C3)c3cc[nH]c23)ccc1C(F)(F)F] |
| 67 | 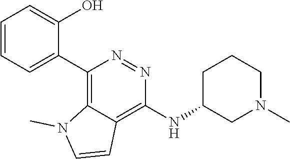 [SMILES: CN1CCC[C@@H](Nc2nnc(-c3ccccc3O)c3c2ccn3C)C1] |
| 68 | 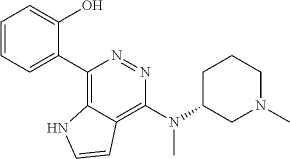 [SMILES: CN1CCC[C@@H](N(C)c2nnc(-c3ccccc3O)c3[nH]ccc23)C1] |
| 69 | 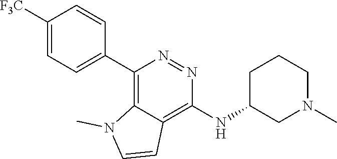 [SMILES: CN1CCC[C@@H](Nc2nnc(-c3ccc(C(F)(F)F)cc3)c3c2ccn3C)C1] |
| 70 | 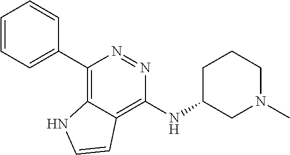 [SMILES: CN1CCC[C@@H](Nc2nnc(-c3ccccc3)c3[nH]ccc23)C1] |
| 71 | 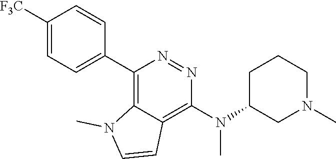 [SMILES: CN1CCC[C@@H](N(C)c2nnc(-c3ccc(C(F)(F)F)cc3)c3c2ccn3C)C1] |
| 72 | 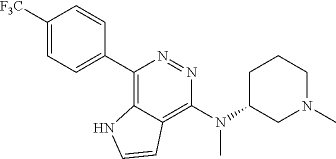 [SMILES: CN1CCC[C@@H](N(C)c2nnc(-c3ccc(C(F)(F)F)cc3)c3[nH]ccc23)C1] |
| 73 | 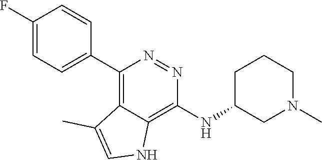 [SMILES: Cc1c[nH]c2c(N[C@@H]3CCCN(C)C3)nnc(-c3ccc(F)cc3)c12] |
| 74 | 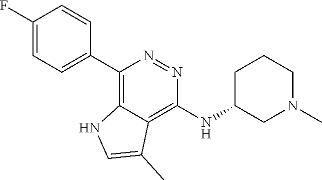 [SMILES: Cc1c[nH]c2c(-c3ccc(F)cc3)nnc(N[C@@H]3CCCN(C)C3)c12] |
| 75 | 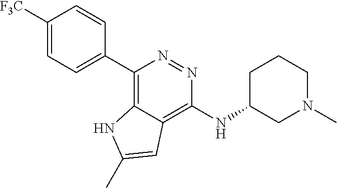 [SMILES: Cc1cc2c(N[C@@H]3CCCN(C)C3)nnc(-c3ccc(C(F)(F)F)cc3)c2[nH]1] |
| 76 | 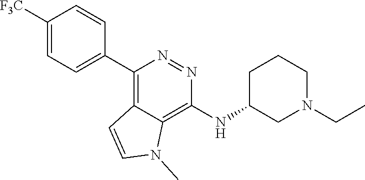 [SMILES: CCN1CCC[C@@H](Nc2nnc(-c3ccc(C(F)(F)F)cc3)c3ccn(C)c23)C1] |
| 77 | 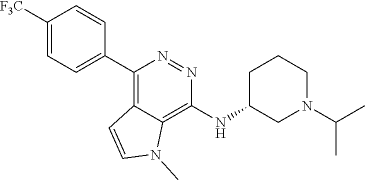 [SMILES: CC(C)N1CCC[C@@H](Nc2nnc(-c3ccc(C(F)(F)F)cc3)c3ccn(C)c23)C1] |
| 78 | 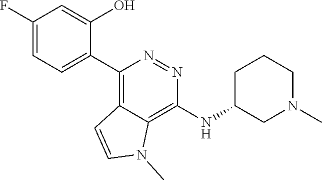 [SMILES: CN1CCC[C@@H](Nc2nnc(-c3ccc(F)cc3O)c3ccn(C)c23)C1] |
| 79 | 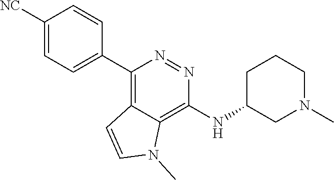 [SMILES: CN1CCC[C@@H](Nc2nnc(-c3ccc(C#N)cc3)c3ccn(C)c23)C1] |
| 80 | 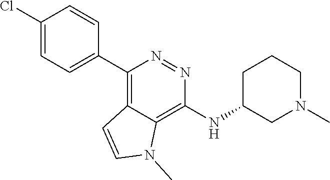 [SMILES: CN1CCC[C@@H](Nc2nnc(-c3ccc(Cl)cc3)c3ccn(C)c23)C1] |
| 81 | 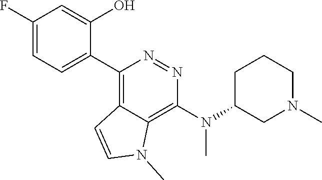 [SMILES: CN1CCC[C@@H](N(C)c2nnc(-c3ccc(F)cc3O)c3ccn(C)c23)C1] |
| 82 | 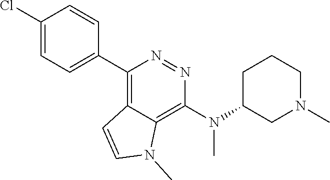 [SMILES: CN1CCC[C@@H](N(C)c2nnc(-c3ccc(Cl)cc3)c3ccn(C)c23)C1] |
| 83 | 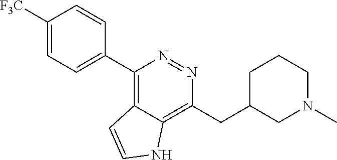 [SMILES: CN1CCCC(Cc2nnc(-c3ccc(C(F)(F)F)cc3)c3cc[nH]c23)C1] |
| 84 | 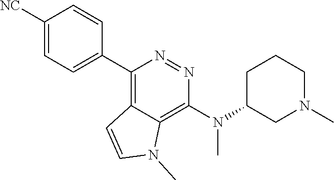 [SMILES: CN1CCC[C@@H](N(C)c2nnc(-c3ccc(C#N)cc3)c3ccn(C)c23)C1] |
| 85 | 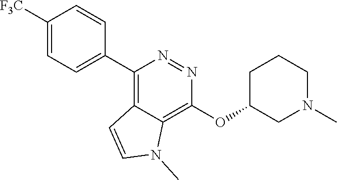 [SMILES: CN1CCC[C@@H](Oc2nnc(-c3ccc(C(F)(F)F)cc3)c3ccn(C)c23)C1] |
| 86 | 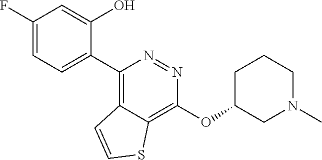 [SMILES: CN1CCC[C@@H](Oc2nnc(-c3ccc(F)cc3O)c3ccsc23)C1] |
| 87 | 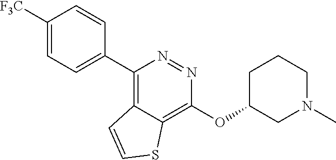 [SMILES: CN1CCC[C@@H](Oc2nnc(-c3ccc(C(F)(F)F)cc3)c3ccsc23)C1] |
| 88 | 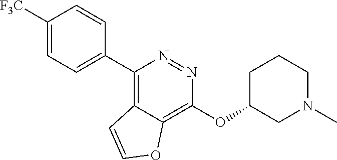 [SMILES: CN1CCC[C@@H](Oc2nnc(-c3ccc(C(F)(F)F)cc3)c3ccoc23)C1] |
| 89 | 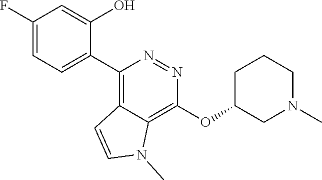 [SMILES: CN1CCC[C@@H](Oc2nnc(-c3ccc(F)cc3O)c3ccn(C)c23)C1] |
| 90 | 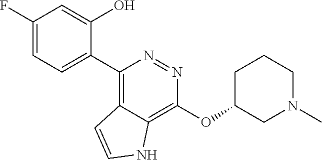 [SMILES: CN1CCC[C@@H](Oc2nnc(-c3ccc(F)cc3O)c3cc[nH]c23)C1] |
| 91 | 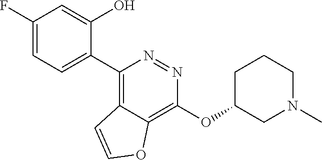 [SMILES: CN1CCC[C@@H](Oc2nnc(-c3ccc(F)cc3O)c3ccoc23)C1] |
| 92 | 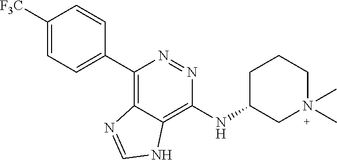 |
| 93 | 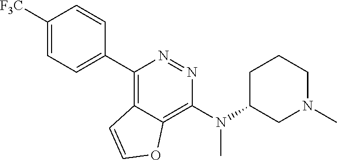 [SMILES: CN1CCC[C@@H](N(C)c2nnc(-c3ccc(C(F)(F)F)cc3)c3ccoc23)C1] |
| 94 | 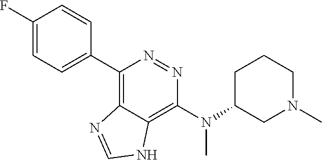 [SMILES: CN1CCC[C@@H](N(C)c2nnc(-c3ccc(F)cc3)c3nc[nH]c23)C1] |
| 95 | 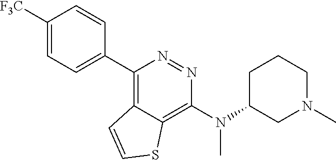 [SMILES: CN1CCC[C@@H](N(C)c2nnc(-c3ccc(C(F)(F)F)cc3)c3ccsc23)C1] |
| 96 | 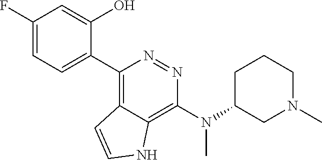 [SMILES: CN1CCC[C@@H](N(C)c2nnc(-c3ccc(F)cc3O)c3cc[nH]c23)C1] |
| 97 | 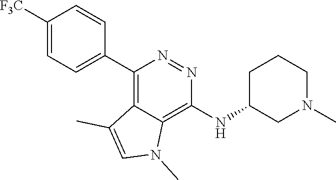 [SMILES: Cc1cn(C)c2c(N[C@@H]3CCCN(C)C3)nnc(-c3ccc(C(F)(F)F)cc3)c12] |
| 98 | 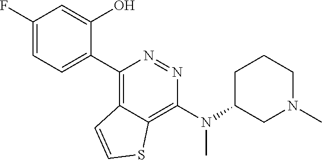 [SMILES: CN1CCC[C@@H](N(C)c2nnc(-c3ccc(F)cc3O)c3ccsc23)C1] |
| 99 | 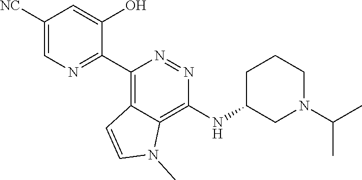 [SMILES: CC(C)N1CCC[C@@H](Nc2nnc(-c3ncc(C#N)cc3O)c3ccn(C)c23)C1] |
| 100 | 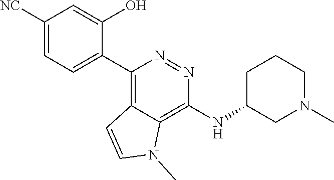 [SMILES: CN1CCC[C@@H](Nc2nnc(-c3ccc(C#N)cc3O)c3ccn(C)c23)C1] |
| 101 | 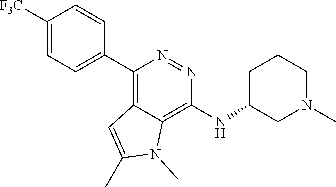 [SMILES: Cc1cc2c(-c3ccc(C(F)(F)F)cc3)nnc(N[C@@H]3CCCN(C)C3)c2n1C] |
| 102 | 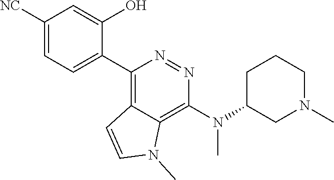 [SMILES: CN1CCC[C@@H](N(C)c2nnc(-c3ccc(C#N)cc3O)c3ccn(C)c23)C1] |
| 103 | 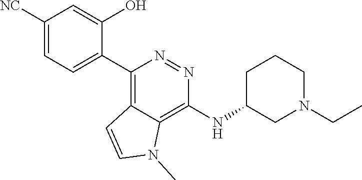 [SMILES: CCN1CCC[C@@H](Nc2nnc(-c3ccc(C#N)cc3O)c3ccn(C)c23)C1] |
| 104 | 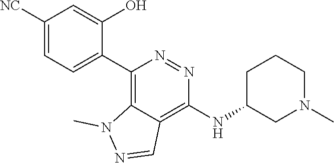 [SMILES: CN1CCC[C@@H](Nc2nnc(-c3ccc(C#N)cc3O)c3c2cnn3C)C1] |
| 105 | 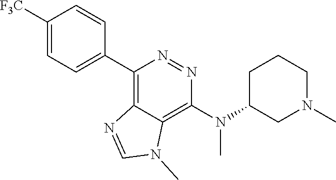 [SMILES: CN1CCC[C@@H](N(C)c2nnc(-c3ccc(C(F)(F)F)cc3)c3ncn(C)c23)C1] |
| 106 | 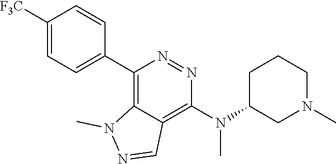 [SMILES: CN1CCC[C@@H](N(C)c2nnc(-c3ccc(C(F)(F)F)cc3)c3c2cnn3C)C1] |
| 107 | 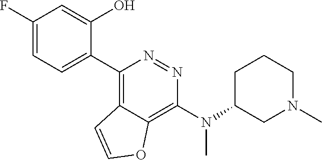 [SMILES: CN1CCC[C@@H](N(C)c2nnc(-c3ccc(F)cc3O)c3ccoc23)C1] |
| 108 | 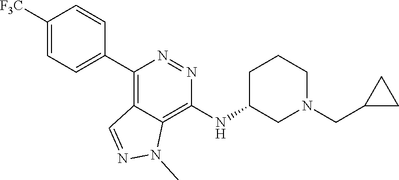 [SMILES: Cn1ncc2c(-c3ccc(C(F)(F)F)cc3)nnc(N[C@@H]3CCCN(CC4CC4)C3)c21] |
| 109 | 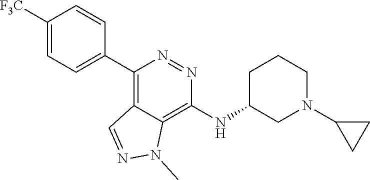 [SMILES: Cn1ncc2c(-c3ccc(C(F)(F)F)cc3)nnc(N[C@@H]3CCCN(C4CC4)C3)c21] |
| 110 | 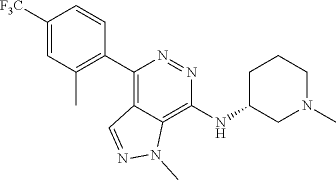 [SMILES: Cc1cc(C(F)(F)F)ccc1-c1nnc(N[C@@H]2CCCN(C)C2)c2c1cnn2C] |
| 111 | 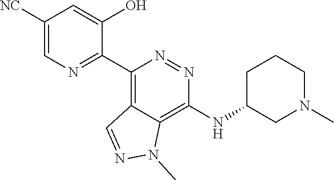 [SMILES: CN1CCC[C@@H](Nc2nnc(-c3ncc(C#N)cc3O)c3cnn(C)c23)C1] |
| 112 | 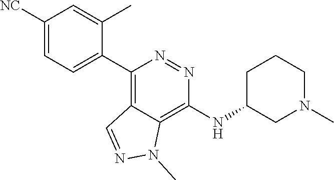 [SMILES: Cc1cc(C#N)ccc1-c1nnc(N[C@@H]2CCCN(C)C2)c2c1cnn2C] |
| 113 | 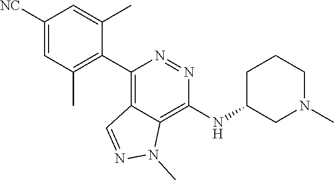 [SMILES: Cc1cc(C#N)cc(C)c1-c1nnc(N[C@@H]2CCCN(C)C2)c2c1cnn2C] |
| 114 | 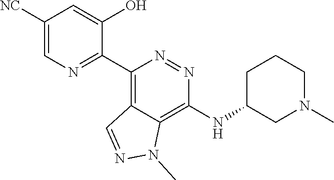 [SMILES: CN1CCC[C@@H](Nc2nnc(-c3ncc(C#N)cc3O)c3cnn(C)c23)C1] |
| 115 | 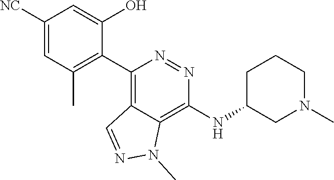 [SMILES: Cc1cc(C#N)cc(O)c1-c1nnc(N[C@@H]2CCCN(C)C2)c2c1cnn2C] |
| 116 | 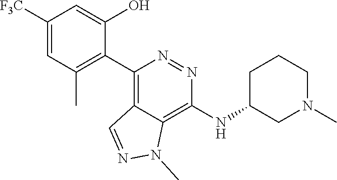 [SMILES: Cc1cc(C(F)(F)F)cc(O)c1-c1nnc(N[C@@H]2CCCN(C)C2)c2c1cnn2C] |
| 117 | 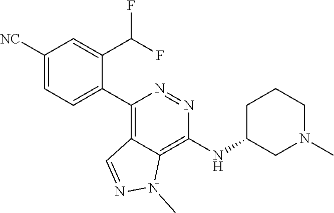 [SMILES: CN1CCC[C@@H](Nc2nnc(-c3ccc(C#N)cc3C(F)F)c3cnn(C)c23)C1] |
| 118 | 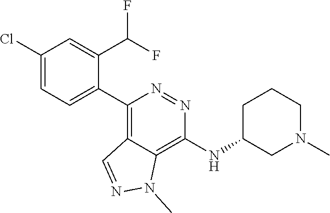 [SMILES: CN1CCC[C@@H](Nc2nnc(-c3ccc(Cl)cc3C(F)F)c3cnn(C)c23)C1] |
| 119 | 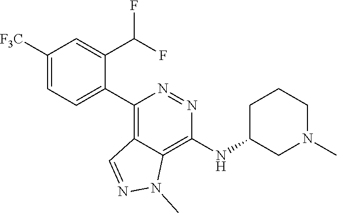 [SMILES: CN1CCC[C@@H](Nc2nnc(-c3ccc(C(F)(F)F)cc3C(F)F)c3cnn(C)c23)C1] |
| 120 | 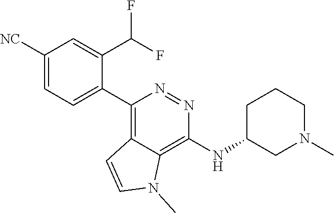 [SMILES: CN1CCC[C@@H](Nc2nnc(-c3ccc(C#N)cc3C(F)F)c3ccn(C)c23)C1] |
| 121 | 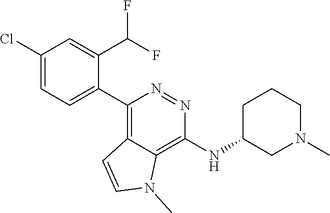 [SMILES: CN1CCC[C@@H](Nc2nnc(-c3ccc(Cl)cc3C(F)F)c3ccn(C)c23)C1] |
| 122 | 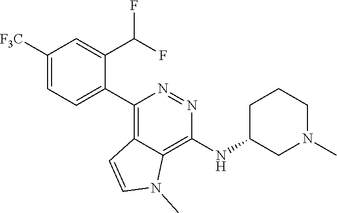 [SMILES: CN1CCC[C@@H](Nc2nnc(-c3ccc(C(F)(F)F)cc3C(F)F)c3ccn(C)c23)C1] |
| 123 | 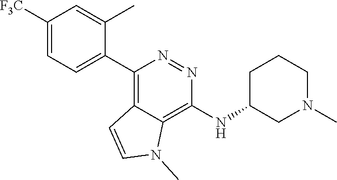 [SMILES: Cc1cc(C(F)(F)F)ccc1-c1nnc(N[C@@H]2CCCN(C)C2)c2c1ccn2C] |
| 124 | 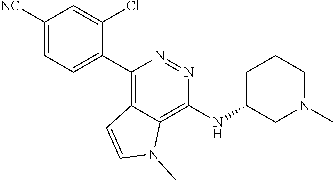 [SMILES: CN1CCC[C@@H](Nc2nnc(-c3ccc(C#N)cc3Cl)c3ccn(C)c23)C1] |
| 125 | 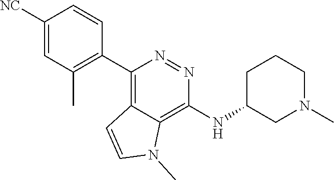 [SMILES: Cc1cc(C#N)ccc1-c1nnc(N[C@@H]2CCCN(C)C2)c2c1ccn2C] |
| 126 | 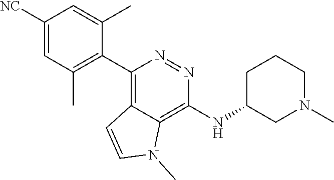 [SMILES: Cc1cc(C#N)cc(C)c1-c1nnc(N[C@@H]2CCCN(C)C2)c2c1ccn2C] |
| 127 | 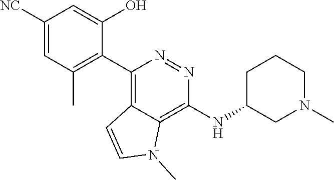 [SMILES: Cc1cc(C#N)cc(O)c1-c1nnc(N[C@@H]2CCCN(C)C2)c2c1ccn2C] |
| 128 | 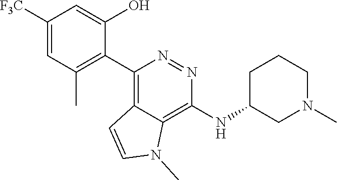 [SMILES: Cc1cc(C(F)(F)F)cc(O)c1-c1nnc(N[C@@H]2CCCN(C)C2)c2c1ccn2C] |
| 129 | 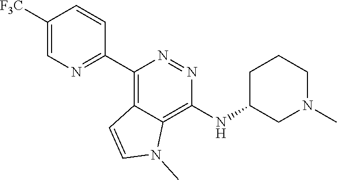 [SMILES: CN1CCC[C@@H](Nc2nnc(-c3ccc(C(F)(F)F)cn3)c3ccn(C)c23)C1] |
| 130 | 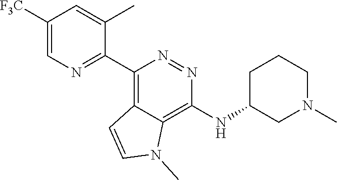 [SMILES: Cc1cc(C(F)(F)F)cnc1-c1nnc(N[C@@H]2CCCN(C)C2)c2c1ccn2C] |
| 131 | 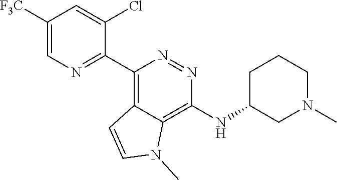 [SMILES: CN1CCC[C@@H](Nc2nnc(-c3ncc(C(F)(F)F)cc3Cl)c3ccn(C)c23)C1] |
| 132 | 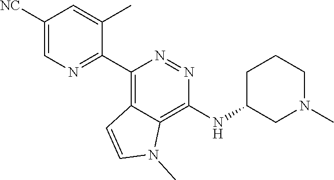 [SMILES: Cc1cc(C#N)cnc1-c1nnc(N[C@@H]2CCCN(C)C2)c2c1ccn2C] |
| 133 | 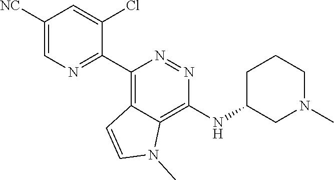 [SMILES: CN1CCC[C@@H](Nc2nnc(-c3ncc(C#N)cc3Cl)c3ccn(C)c23)C1] |
| 134 | 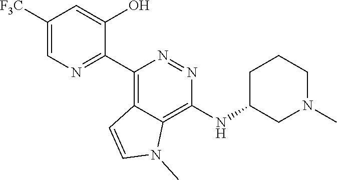 [SMILES: CN1CCC[C@@H](Nc2nnc(-c3ncc(C(F)(F)F)cc3O)c3ccn(C)c23)C1] |
| 135 | 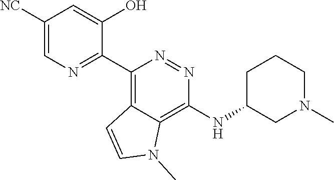 [SMILES: CN1CCC[C@@H](Nc2nnc(-c3ncc(C#N)cc3O)c3ccn(C)c23)C1] |
| 136 | 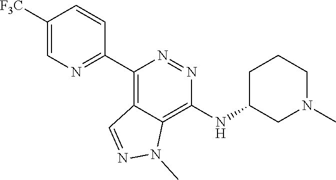 [SMILES: CN1CCC[C@@H](Nc2nnc(-c3ccc(C(F)(F)F)cn3)c3cnn(C)c23)C1] |
| 137 | 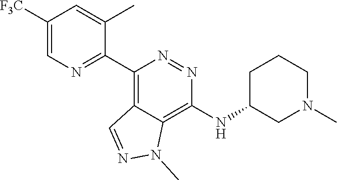 [SMILES: Cc1cc(C(F)(F)F)cnc1-c1nnc(N[C@@H]2CCCN(C)C2)c2c1cnn2C] |
| 138 | 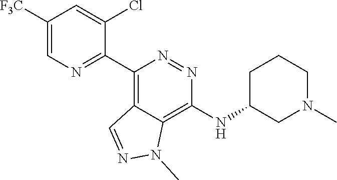 [SMILES: CN1CCC[C@@H](Nc2nnc(-c3ncc(C(F)(F)F)cc3Cl)c3cnn(C)c23)C1] |
| 139 | 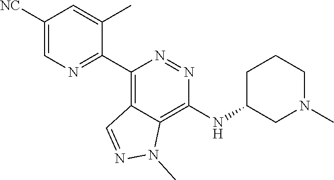 [SMILES: Cc1cc(C#N)cnc1-c1nnc(N[C@@H]2CCCN(C)C2)c2c1cnn2C] |
| 140 | 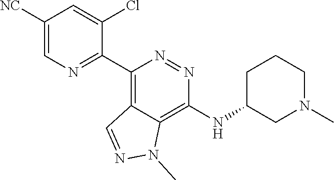 [SMILES: CN1CCC[C@@H](Nc2nnc(-c3ncc(C#N)cc3Cl)c3cnn(C)c23)C1] |
| 141 | 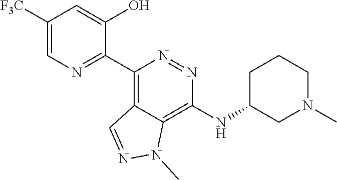 [SMILES: CN1CCC[C@@H](Nc2nnc(-c3ncc(C(F)(F)F)cc3O)c3cnn(C)c23)C1] |
| 142 | 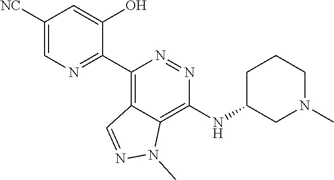 [SMILES: CN1CCC[C@@H](Nc2nnc(-c3ncc(C#N)cc3O)c3cnn(C)c23)C1] |
| 143 | 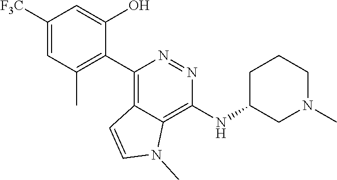 [SMILES: Cc1cc(C(F)(F)F)cc(O)c1-c1nnc(N[C@@H]2CCCN(C)C2)c2c1ccn2C] |
| 144 | 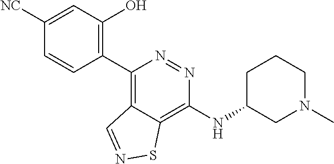 [SMILES: CN1CCC[C@@H](Nc2nnc(-c3ccc(C#N)cc3O)c3cnsc23)C1] |
| 145 | 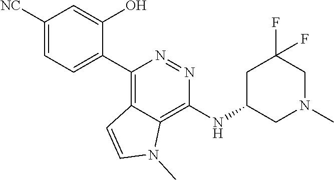 [SMILES: CN1C[C@H](Nc2nnc(-c3ccc(C#N)cc3O)c3ccn(C)c23)CC(F)(F)C1] |
| 146 | 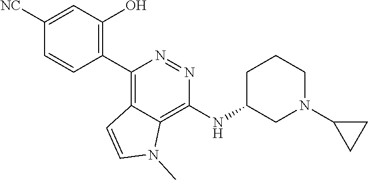 [SMILES: Cn1ccc2c(-c3ccc(C#N)cc3O)nnc(N[C@@H]3CCCN(C4CC4)C3)c21] |
| 147 | 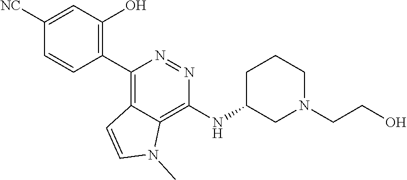 [SMILES: Cn1ccc2c(-c3ccc(C#N)cc3O)nnc(N[C@@H]3CCCN(CCO)C3)c21] |
| 148 | 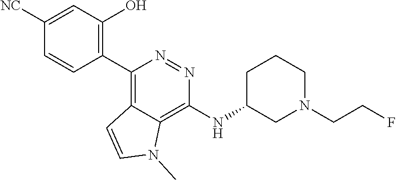 [SMILES: Cn1ccc2c(-c3ccc(C#N)cc3O)nnc(N[C@@H]3CCCN(CCF)C3)c21] |
| 149 | 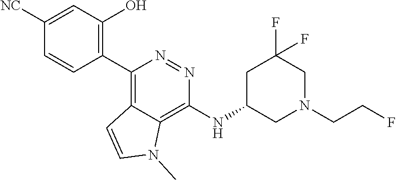 [SMILES: Cn1ccc2c(-c3ccc(C#N)cc3O)nnc(N[C@H]3CN(CCF)CC(F)(F)C3)c21] |
| 150 | 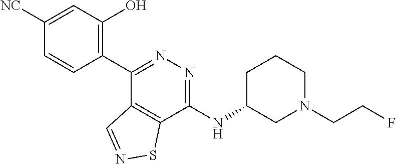 [SMILES: N#Cc1ccc(-c2nnc(N[C@@H]3CCCN(CCF)C3)c3sncc23)c(O)c1] |
| 151 | 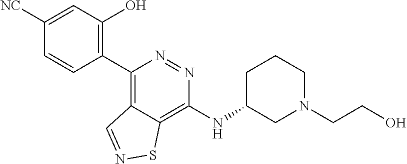 [SMILES: N#Cc1ccc(-c2nnc(N[C@@H]3CCCN(CCO)C3)c3sncc23)c(O)c1] |
| 152 | 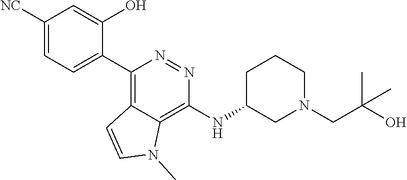 [SMILES: Cn1ccc2c(-c3ccc(C#N)cc3O)nnc(N[C@@H]3CCCN(CC(C)(C)O)C3)c21] |
| 153 | 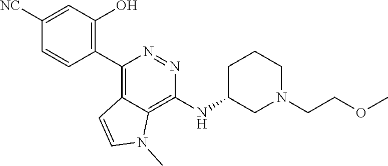 [SMILES: COCCN1CCC[C@@H](Nc2nnc(-c3ccc(C#N)cc3O)c3ccn(C)c23)C1] |
| 154 | 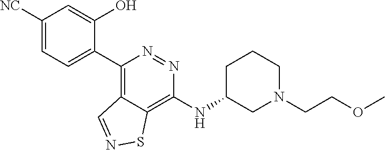 [SMILES: COCCN1CCC[C@@H](Nc2nnc(-c3ccc(C#N)cc3O)c3cnsc23)C1] |
| 155 | 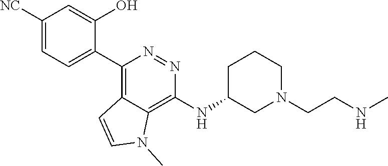 [SMILES: CNCCN1CCC[C@@H](Nc2nnc(-c3ccc(C#N)cc3O)c3ccn(C)c23)C1] |
| 156 | 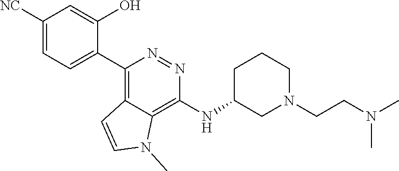 [SMILES: CN(C)CCN1CCC[C@@H](Nc2nnc(-c3ccc(C#N)cc3O)c3ccn(C)c23)C1] |
| 157 | 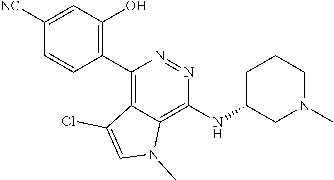 [SMILES: CN1CCC[C@@H](Nc2nnc(-c3ccc(C#N)cc3O)c3c(Cl)cn(C)c23)C1] |
| 158 | 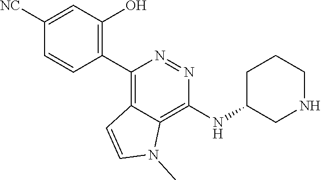 [SMILES: Cn1ccc2c(-c3ccc(C#N)cc3O)nnc(N[C@@H]3CCCNC3)c21] |
| 159 | 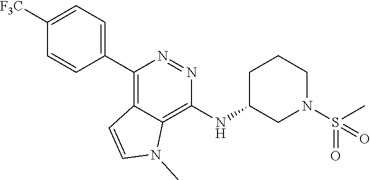 [SMILES: Cn1ccc2c(-c3ccc(C(F)(F)F)cc3)nnc(N[C@@H]3CCCN(S(C)(=O)=O)C3)c21] |
| 160 | 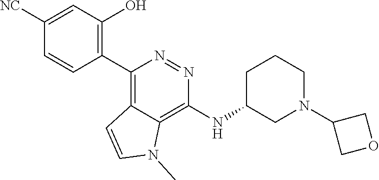 [SMILES: Cn1ccc2c(-c3ccc(C#N)cc3O)nnc(N[C@@H]3CCCN(C4COC4)C3)c21] |
| 161 | 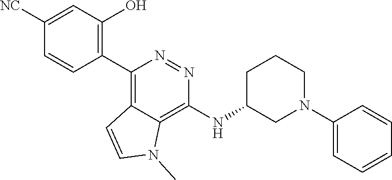 [SMILES: Cn1ccc2c(-c3ccc(C#N)cc3O)nnc(N[C@@H]3CCCN(c4ccccc4)C3)c21] |
| 162 | 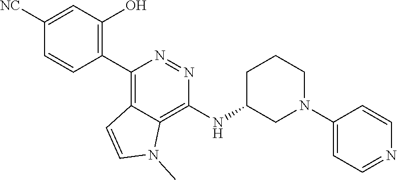 [SMILES: Cn1ccc2c(-c3ccc(C#N)cc3O)nnc(N[C@@H]3CCCN(c4ccncc4)C3)c21] |
| 163 | 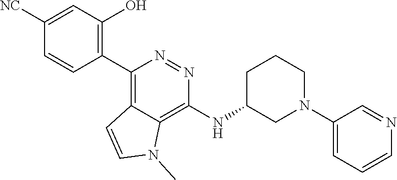 [SMILES: Cn1ccc2c(-c3ccc(C#N)cc3O)nnc(N[C@@H]3CCCN(c4cccnc4)C3)c21] |
| 164 | 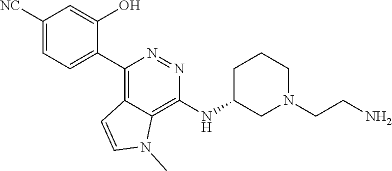 [SMILES: Cn1ccc2c(-c3ccc(C#N)cc3O)nnc(N[C@@H]3CCCN(CCN)C3)c21] |
| 165 | 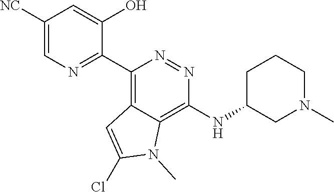 [SMILES: CN1CCC[C@@H](Nc2nnc(-c3ncc(C#N)cc3O)c3cc(Cl)n(C)c23)C1] |
| 166 | 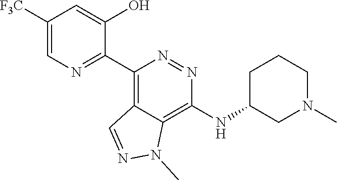 [SMILES: CN1CCC[C@@H](Nc2nnc(-c3ncc(C(F)(F)F)cc3O)c3cnn(C)c23)C1] |
| 167 | 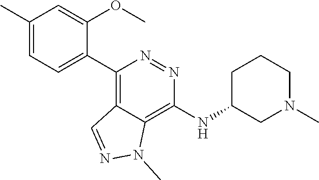 |
| 168 | 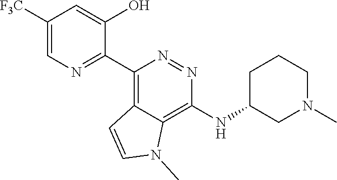 [SMILES: CN1CCC[C@@H](Nc2nnc(-c3ncc(C(F)(F)F)cc3O)c3ccn(C)c23)C1] |
| 169 | 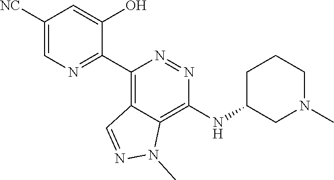 [SMILES: CN1CCC[C@@H](Nc2nnc(-c3ncc(C#N)cc3O)c3cnn(C)c23)C1] |
| 170 | 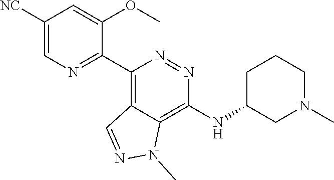 [SMILES: COc1cc(C#N)cnc1-c1nnc(N[C@@H]2CCCN(C)C2)c2c1cnn2C] |

### Pharmaceutical Compositions

In certain embodiments, the invention provides a pharmaceutical composition comprising a compound of structure (I), or a pharmaceutically acceptable salt, isomer, hydrate, solvate or isotope thereof, together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxy ethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxymethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, isotope or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for administration to a pediatric subject (e.g., solution, syrup, suspension, elixir, powder for reconstitution as suspension or solution, dispersible/effervescent tablet, chewable tablet, lollipop, freezer pops, troches, oral thin strips, orally disintegrating tablet, orally disintegrating strip, and sprinkle oral powder or granules); or in any other formulation described herein. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In some embodiments, the pharmaceutical composition comprising a compound of structure (I) or a pharmaceutically acceptable salt, isomer, hydrate, solvate or isotope thereof, with at least one pharmaceutically acceptable carrier, diluent, or excipient further comprises a second therapeutic agent.

As used herein, the term “pharmaceutically acceptable carrier” refers to any ingredient other than the disclosed compounds, or a pharmaceutically acceptable isomer, racemate, hydrate, solvate, isotope or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal, or parenteral, including intravenous, subcutaneous and/or intramuscular. In one embodiment, the route of administration is oral. In another embodiment, the route of administration is topical.

Dosage forms can be administered once a day, or more than once a day, such as twice or thrice daily. Alternatively, dosage forms can be administered less frequently than daily, such as every other day, or weekly, if found to be advisable by a prescribing physician or drug's prescribing information. Dosing regimens include, for example, dose titration to the extent necessary or useful for the indication to be treated, thus allowing the patient's body to adapt to the treatment, to minimize or avoid unwanted side effects associated with the treatment, and/or to maximize the therapeutic effect of the present compounds. Other dosage forms include delayed or controlled-release forms. Suitable dosage regimens and/or forms include those set out, for example, in the latest edition of the Physicians' Desk Reference, incorporated herein by reference.

In one embodiment, the invention provides an oral pharmaceutical composition comprising structure (I) or a pharmaceutically acceptable salt, isomer, hydrate, solvate or isotope thereof, together with at least one pharmaceutically acceptable oral carrier, diluent, or excipient. In another embodiment, the invention provides a topical pharmaceutical composition comprising a compound of structure (I) or a pharmaceutically acceptable salt, isomer, hydrate, solvate or isotope thereof, together with at least one pharmaceutically acceptable topical carrier, diluent, or excipient.

In another embodiment, there are provided methods of making a composition of a compound described herein including formulating a compound of the invention with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation. In some embodiments, the composition is formulated into a pediatric dosage form suitable for treating a pediatric subject.

In certain embodiments, the invention provides a compound having structure (I) or a pharmaceutically acceptable salt, isomer, hydrate, solvate or isotope thereof. Such compounds can be synthesized using standard synthetic techniques known to those skilled in the art. For example, compounds of the present invention can be synthesized using appropriately modified synthetic procedures set forth in the following Examples and Reaction Schemes.

To this end, the reactions, processes, and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

All reagents, for which the synthesis is not described in the experimental part, are either commercially available, or are known compounds or may be formed from known compounds by known methods by a person skilled in the art. The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC using methods as described.

Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention, which is sufficiently acidic, an ammonium salt. A salt of this type can either be transformed into its free base or free acid form, respectively, by various methods known to a person skilled in the art, or be used as salts in subsequent biological assays. It is to be understood that the specific form of a compound of the present invention as isolated and as described herein is not necessarily the only form in which said compound can be applied to a biological assay in order to quantify the specific biological activity.

Chemical names were generated using the ChemDraw naming software (Version 17.0.0.206) by PerkinElmer Informatics, Inc. In some cases, generally accepted names of commercially available reagents were used in place of names generated by the naming software.

### Methods of Treatment

In some embodiments, the invention provides a method for treating an NLRP3 inflammasome dependent condition, wherein “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

In some embodiments, the invention provides a method for treating an NLRP3 inflammasome dependent condition in a subject, wherein “subject” refers to an animal (e.g., a mammal, such as a human). A subject to be treated according to the methods described herein may be one who has been diagnosed with a NLRP3 inflammasome dependent condition, such as inflammation, an inflammatory disease, an immune disease, cancer, infections including viral infections; central nervous system diseases, metabolic diseases, cardiovascular diseases, respiratory diseases, liver diseases, renal diseases, ocular diseases, skin diseases, psychological diseases or blood diseases.

Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. The term “patient” may be used interchangeably with the term “subject.” A subject may refer to an adult or pediatric subject.

The route of administration can be any route which effectively transports the active compound of the invention to the appropriate or desired site of action, such as oral, nasal, pulmonary, buccal, subdermal, intradermal, transdermal, or parenteral, including intravenous, subcutaneous and/or intramuscular. In one embodiment, the route of administration is oral. In another embodiment, the route of administration is topical.

Dosage forms can be administered once a day, or more than once a day, such as twice or thrice daily. Alternatively, dosage forms can be administered less frequently than daily, such as every other day, or weekly, if found to be advisable by a prescribing physician or drug's prescribing information. Dosing regimens include, for example, dose titration to the extent necessary or useful for the indication to be treated, thus allowing the patient's body to adapt to the treatment, to minimize or avoid unwanted side effects associated with the treatment, and/or to maximize the therapeutic effect of the present compounds. Other dosage forms include delayed or controlled-release forms. Suitable dosage regimens and/or forms include those set out, for example, in the latest edition of the Physicians' Desk Reference, incorporated herein by reference.

In one embodiment, the invention provides an oral pharmaceutical composition comprising a compound of a structure as described herein, or a pharmaceutically acceptable salt, isomer, hydrate, solvate or isotope thereof, together with at least one pharmaceutically acceptable oral carrier, diluent, or excipient. In another embodiment, the invention provides a topical pharmaceutical composition comprising a compound of a structure as described herein, or a pharmaceutically acceptable salt, isomer, hydrate, solvate or isotope thereof, together with at least one pharmaceutically acceptable topical carrier, diluent, or excipient. In another embodiment, the invention provides an parenteral pharmaceutical composition comprising a compound of a structure as described herein, or a pharmaceutically acceptable salt, isomer, hydrate, solvate or isotope thereof, together with at least one pharmaceutically acceptable topical carrier, diluent, or excipient.

In some embodiments, the invention provides a method for treating an NLRP3 inflammasome dependent condition, wherein modulating NLRP3 provides a medical benefit to the patient or subject.

In some embodiments, the NLRP3 inflammasome dependent condition is inflammation, an inflammatory disease, an immune disease, cancer, infections including viral infections; central nervous system diseases, metabolic diseases, cardiovascular diseases, respiratory diseases, liver diseases, renal diseases, ocular diseases, skin diseases, psychological diseases or blood diseases.

In one embodiment, the NLRP3 inflammasome dependent condition is neuroinflammation-related disorders or neurodegenerative diseases.

In one embodiment, the invention provides a method for inhibiting NLRP3 inflammasome with an effective amount of a pharmaceutical composition as described herein.

In another embodiment, the invention provides a method of treating a NLRP3 inflammasome dependent condition by administering to a subject in need thereof an effective amount of a pharmaceutical composition as described herein. In certain embodiments, the NLRP3 inflammasome dependent condition is a neuroinflammation-related disorder(s) or a neurodegenerative disease(s).

In another embodiment, the invention provides a method of treating a NLRP3 inflammasome dependent condition including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity. Examples of inflammation that may be treated or prevented include inflammatory responses occurring in connection with, or as a result of:
(a) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(b) a joint condition such as osteoarthritis, systemic juvenile idiopathic arthritis, adult-onset Still's disease, relapsing polychondritis, rheumatoid arthritis, juvenile chronic arthritis, crystal induced arthropathy (e.g. pseudo-gout, gout), or a seronegative spondyloarthropathy (e.g. ankylosing spondylitis, psoriatic arthritis or Reiter's disease);
(c) a muscular condition such as polymyositis or myasthenia gravis;
(d) a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(e) a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
(f) a vascular condition such as atherosclerosis, Behcet's disease, vasculitides, or Wegener's granulomatosis;
(g) an immune condition, e.g. autoimmune condition, such as systemic lupus erythematosus (SLE), Sjogren's syndrome, systemic sclerosis, Hashimoto's thyroiditis, type I diabetes, idiopathic thrombocytopenia purpura, or Graves disease;
(h) an ocular condition such as uveitis, allergic conjunctivitis, or vernal conjunctivitis;
(i) a nervous system condition such as multiple sclerosis or encephalomyelitis;
(j) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, epstein-barr virus, viral encephalitis/aseptic meningitis, or pelvic inflammatory disease;
(k) a renal condition such as mesangial proliferative glomerulonephritis, nephrotic syndrome, nephritis, glomerular nephritis, acute renal failure, uremia, or nephritic syndrome;
(l) a lymphatic condition such as Castleman's disease;
(m) a condition of, or involving, the immune system, such as hyper IgE syndrome, lepromatous leprosy, familial hemophagocytic lymphohistiocytosis, or graft versus host disease;
(n) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;
(o) a cancer, including those cancers listed herein below;
(p) a burn, wound, trauma, haemorrhage or stroke;
(q) radiation exposure; and/or
(r) obesity;
(s) pain such as inflammatory hyperalgesia; and/or
(t) neurodegenerative disorders, such as Alzheimer's disease, Parkinson's disease, multiple sclerosis, or amyotrophic lateral sclerosis.

In another embodiment, the invention provides a method of treating a NLRP3 inflammasome dependent condition such as an inflammatory disease. For example, inflammation occurring as a result of an inflammatory disorder, e.g. an autoinflammatory disease, such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

In another embodiment, the invention provides a method of treating a NLRP3 inflammasome dependent condition such as an immune disease. For example, auto-immune diseases, such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjogren's syndrome, systemic sclerosis a systemic connective tissue disorder, Takayasu's arteritis, temporal arteritis, warm autoimmune hemolytic anemia, Wegener's granulomatosis, alopecia universalis, Beliefs disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, giant cell arteritis, vitiligo or vulvodynia.

In another embodiment, the invention provides a method of treating a NLRP3 inflammasome dependent condition such as cancer. For example, lung cancer, renal cell carcinoma, non-small cell lung carcinoma (NSCLC), Langerhans cell histiocytosis (LCH), myeloproliferative neoplasm (MPN), pancreatic cancer, gastric cancer, myelodysplastic syndrome (MDS), leukaemia including acute lymphocytic leukaemia (ALL) and acute myeloid leukaemia (AML), promyelocytic leukemia (APML, or APL), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour.

In another embodiment, the invention provides a method of treating a NLRP3 inflammasome dependent condition such as an infection, including viral infections. For example, viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxyiruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes), and prion infections.

In another embodiment, the invention provides a method of treating a NLRP3 inflammasome dependent condition such as a central nervous system disease. For example, Parkinson's disease, Alzheimer's disease, Frontotemporal dementia, dementia, motor neuron disease, Huntington's disease, cerebral malaria, brain injury from pneumococcal meningitis, intracranial aneurysms, traumatic brain injury, multiple sclerosis, and amyotrophic lateral sclerosis.

In another embodiment, the invention provides a method of treating a NLRP3 inflammasome dependent condition such as a neuroinflammation-related disease. For example, multiple sclerosis, brain infection, acute injury, neurodegenerative disease, Parkinson's disease or Alzheimer's disease.

In another embodiment, the invention provides a method of treating a NLRP3 inflammasome dependent condition such as a neurodegenerative disease. For example, Alzheimer's disease, Parkinson's disease, multiple sclerosis, or amyotrophic lateral sclerosis.

In one embodiment, neurodegenerative diseases are characterized by deep involvement of cell mediating neuroinflammatory processes.

In another embodiment, the invention provides a method of treating a NLRP3 inflammasome dependent condition such as a metabolic disease. For example, type 2 diabetes (T2D), atherosclerosis, obesity, gout, and pseudo-gout.

In another embodiment, the invention provides a method of treating a NLRP3 inflammasome dependent condition such as a cardiovascular disease. For example, hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, cardiovascular risk reduction (CvRR), and pericarditis including Dressler's syndrome.

In another embodiment, the invention provides a method of treating a NLRP3 inflammasome dependent condition such as a respiratory disease. For example, chronic obstructive pulmonary disorder (COPD), asthma such as allergic asthma and steroid-resistant asthma, asbestosis, silicosis, nanoparticle induced inflammation, cystic fibrosis, and idiopathic pulmonary fibrosis.

In another embodiment, the invention provides a method of treating a NLRP3 inflammasome dependent condition such as a liver disease. For example, non-alcoholic fatty liver disease (NAFLD) and nonalcoholic steatohepatitis (NASH) including advanced fibrosis stages F3 and F4, alcoholic fatty liver disease (AFLD), and alcoholic steatohepatitis (ASH).

In another embodiment, the invention provides a method of treating a NLRP3 inflammasome dependent condition such as a renal disease. For example, acute kidney disease, hyperoxaluria, chronic kidney disease, oxalate nephropathy, nephrocalcinosis, glomerulonephritis, and diabetic nephropathy; In another embodiment, the invention provides a method of treating a NLRP3 inflammasome dependent condition such as an ocular disease. For example, diseases of the ocular epithelium, age-related macular degeneration (AMD) (dry and wet), uveitis, corneal infection, diabetic retinopathy, optic nerve damage, dry eye, and glaucoma.

In another embodiment, the invention provides a method of treating a NLRP3 inflammasome dependent condition such as a skin disease. For example, dermatitis such as contact dermatitis and atopic dermatitis, contact hypersensitivity, sunburn, skin lesions, hidradenitis suppurativa (HS), other cyst-causing skin diseases, and acne conglobate.

In another embodiment, the invention provides a method of treating a NLRP3 inflammasome dependent condition such as a psychological disease. For example, depression, and psychological stress.

In another embodiment, the invention provides a method of treating a NLRP3 inflammasome dependent condition such as a blood disease. For example, sickle cell disease.

### EXAMPLES

### General Methods

Proton nuclear magnetic resonance (¹H NMR, 400 MHz) spectra were obtained in solution of deuterochloroform (CDCl₃), deuteromethanol (CD₃OD) or dimethyl sulfoxide-D₆ (DMSO-D₆). High-performance liquid chromatography (HPLC) retention times, purities, and mass spectra (LCMS) were obtained using Shimadzu LCMS 2010 (Shim-pack XR-ODS 3.0*30 mm 2.2 μm) operating in ES (+) ionization mode. Flow rate: 0.8 mL/min, acquire time: 3 min, wavelength: UV220, oven, temperature: 50° C.

The following additional abbreviations are used: acetic acid (AcOH), ammonia (NH₃), 2,2′-bis(diphenylphosphino)-1,1′-binaphthyl (BINAP), n-butyllithium (n-BuLi), cesium carbonate (Cs₂CO₃), degree Celsius (° C.), dichloromethane (DCM), (2-dicyclohexylphosphino-2′,4′,6′-triisopropyl-1,1′-biphenyl)[2-(2′-amino-1,1′-biphenyl)]palladium(II) methanesulfonate (XPhosPdG3), N,N-diisopropylethylamine (DIEA), dimethylformamide (DMF), ethanol (EtOH), ethyl acetate (EtOAc), formaldehyde (HCHO), gram (g), hour (h), hydrazine hydrate (NH₂NH₂·H₂O), hydrochloric acid (HCl), iodomethane (CH₃I), manganese dioxide (MnO₂), methanol (MeOH), N-methyl-2-pyrrolidone (NMP), milligram (mg), milliliter (mL), millimole (mmol), minute (min), molar (M), normal (N), palladium II acetate (Pd(OAc)₂), percent (%), petroleum ether (PE), phosphoryl chloride (POCl₃), potassium carbonate (K₂CO₃), potential of hydrogen (pH), preparative high-performance liquid chromatography (prep-HPLC), silver carbonate (Ag₂CO₃), sodium bicarbonate (NaHCO₃), sodium cyanoborohydride (NaBH₃CN), sodium hydride (NaH), sodium sulfate (Na₂SO₄, tetrahydrofuran (THF), tetramethylethylenediamine (TMEDA), triethylamine (Et₃N), trimethylsilyldiazomethane (TMSCHN₂), 2-(trimethylsilyl)ethoxymethyl chloride (SEMCl), water (H₂O).

### Example 1

### General Synthetic Route A

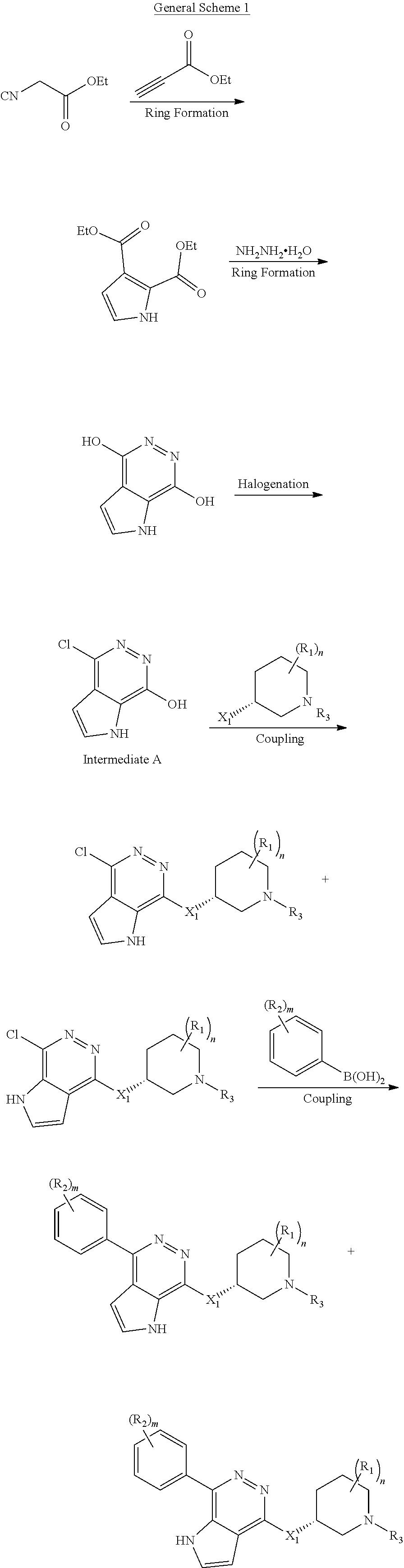

### Synthesis of (R)—N-(1-methylpiperidin-3-yl)-4-(4-(trifluoromethyl)phenyl)-1H-pyrrolo[2,3-d]pyridazin-7-amine (Compound 1) and (R)—N-(1-methylpiperidin-3-yl)-7-(4-(trifluoromethyl)phenyl)-1H-pyrrolo[2,3-d]pyridazin-4-amine (Compound 2)

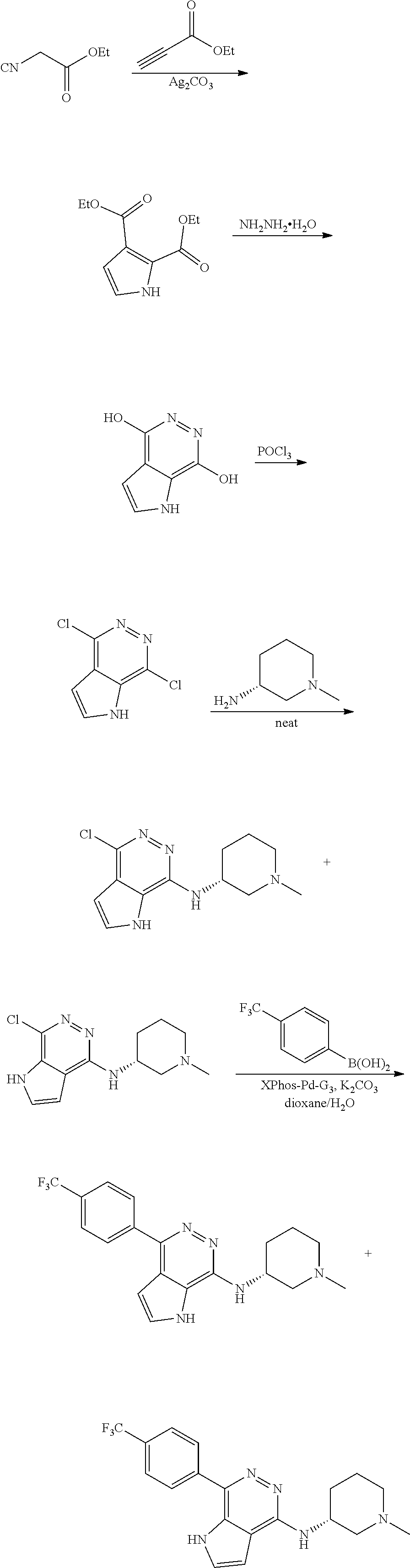

### Synthesis of diethyl 1H-pyrrole-2,3-dicarboxylate

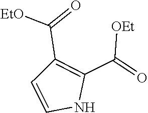 [SMILES: CCOC(=O)c1cc[nH]c1C(=O)OCC]

To a mixture of ethyl 2-isocyanoacetate (13.84 g, 122.3 mmol) and Ag₂CO₃ (3.37 g, 12.2 mmol) in dioxane (450 mL) was slowly added ethyl prop-2-ynoate (18.0 g, 183 mmol). The resulting mixture was stirred at 80° C. for 2 h. The mixture was then concentrated under reduced pressure to afford the crude residue. The crude residue was purified by silica gel column chromatography (20% ethyl acetate in petroleum ether) to afford desired product diethyl 1H-pyrrole-2,3-dicarboxylate (22.06 g, 85% yield) as a yellow oil.

### Synthesis of 1H-pyrrolo[2,3-d]pyridazine-4,7-diol

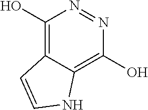 [SMILES: Oc1nnc(O)c2[nH]ccc12]

A solution of diethyl 1H-pyrrole-2,3-dicarboxylate (11.0 g, 52.1 mmol) in NH₂NH₂·H₂O (61.8 g, 1.21 mmol) was stirred at 120° C. for 16 h under nitrogen atmosphere. The resulting mixture was then concentrated under reduced pressure to afford the crude residue. The crude residue was triturated in EtOH (20 mL), filtered and dried to afford desired product 1H-pyrrolo[2,3-d]pyridazine-4,7-diol (8.98 g) as a yellow solid. The desired product was used in the next step without further purification.

### Synthesis of 4,7-dichloro-1H-pyrrolo[2,3-d]pyridazine

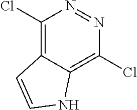 [SMILES: Clc1nnc(Cl)c2[nH]ccc12]

A mixture of 1H-pyrrolo[2,3-d]pyridazine-4,7-diol (1.53 g, 10.1 mmol) in POCl₃ (15 mL) was stirred at 110° C. for 6 h under nitrogen atmosphere. The resulting mixture was poured into water (100 mL) and a saturated NaHCO₃ aqueous solution was added to adjust pH to 7. The mixture was then extracted with EtOAc (3×70 mL). The organic layers were combined, dried over anhydrous Na₂SO₄, filtered and concentrated under reduced pressure to afford the crude residue. The crude residue was purified by silica gel column chromatography (DCM:MeOH 1:0 to 5:1) to afford desired product 4,7-dichloro-1H-pyrrolo[2,3-d]pyridazine (978 mg, 51% yield) as a yellow solid.

### Synthesis of (R)-4-chloro-N-(1-methylpiperidin-3-yl)-1H-pyrrolo[2,3-d]pyridazin-7-amine and (R)-7-chloro-N-(1-methylpiperidin-3-yl)-1H-pyrrolo[2,3-d]pyridazin-4-amine

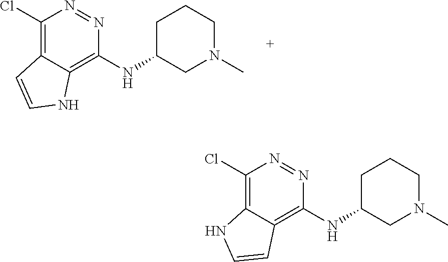 [SMILES: CN1CCC[C@@H](Nc2nnc(Cl)c3cc[nH]c23)C1 ; CN1CCC[C@@H](Nc2nnc(Cl)c3[nH]ccc23)C1]

A mixture of 4,7-dichloro-1H-pyrrolo[2,3-d]pyridazine (300 mg, 1.60 mmol) and (3R)-1-methylpiperidin-3-amine (364 mg, 3.19 mmol) was degassed and purged with nitrogen for 10 min. The mixture was stirred at 140° C. for 16 h under nitrogen atmosphere. The resulting crude residue was purified by silica gel column chromatography (24% MeOH in DCM with 1% Et₃N) to afford a mixture of desired products (R)-4-chloro-N-(1-methylpiperidin-3-yl)-1H-pyrrolo[2,3-d]pyridazin-7-amine and (R)-7-chloro-N-(1-methylpiperidin-3-yl)-1H-pyrrolo[2,3-d]pyridazin-4-amine (150 mg, 13% yield) as a yellow solid.

### Synthesis of (R)—N-(1-methylpiperidin-3-yl)-4-(4-(trifluoromethyl)phenyl)-1H-pyrrolo[2,3-d]pyridazin-7-amine (Compound 1) and (R)—N-(1-methylpiperidin-3-yl)-7-(4-(trifluoromethyl)phenyl)-1H-pyrrolo[2,3-d]pyridazin-4-amine (Compound 2)

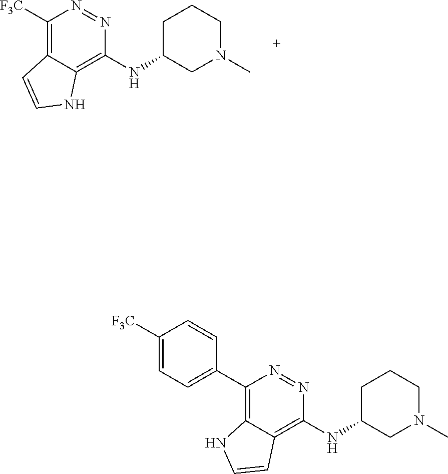 [SMILES: CN1CCC[C@@H](Nc2nnc(C(F)(F)F)c3cc[nH]c23)C1 ; CN1CCC[C@@H](Nc2nnc(-c3ccc(C(F)(F)F)cc3)c3[nH]ccc23)C1]

To a mixture of (4-(trifluoromethyl)phenyl)boronic acid (429 mg, 2.26 mmol) in 1,4-dioxane (3 mL) and H₂O (0.6 mL) were added a mixture of (R)-4-chloro-N-(1-methylpiperidin-3-yl)-1H-pyrrolo[2,3-d]pyridazin-7-amine and (R)-7-chloro-N-(1-methylpiperidin-3-yl)-1H-pyrrolo[2,3-d]pyridazin-4-amine (400 mg, 1.51 mmol), K₂CO₃ (1.47 mg, 4.52 mmol) and XPhos-Pd-G3 (191 mg, 0.226 mmol). The resulting mixture was stirred at 100° C. for 16 h under nitrogen atmosphere. The mixture was then concentrated under reduced pressure to afford the crude residue. The crude residue was purified by silica gel column chromatography (DCM:MeOH 5:1 with 1% Et₃N) to afford a mixture of desired products (R)—N-(1-methylpiperidin-3-yl)-4-(4-(trifluoromethyl)phenyl)-1H-pyrrolo[2,3-d]pyridazin-7-amine and (R)—N-(1-methylpiperidin-3-yl)-7-(4-(trifluoromethyl)phenyl)-1H-pyrrolo[2,3-d]pyridazin-4-amine (232 mg). The mixture of desired products was repurified by prep-HPLC (0.05% NH₃·H₂O as additive) to afford desired products (R)—N-(1-methylpiperidin-3-yl)-7-(4-(trifluoromethyl)phenyl)-1H-pyrrolo[2,3-d]pyridazin-4-amine (Compound 2, 28 mg, 5% yield, peak 1) as an off-white solid and (R)—N-(1-methylpiperidin-3-yl)-4-(4-(trifluoromethyl)phenyl)-1H-pyrrolo[2,3-d]pyridazin-7-amine (Compound 1, 5.5 mg, 1% yield, peak 2) as an off-white solid.

### Example 2

### General Synthetic Route B

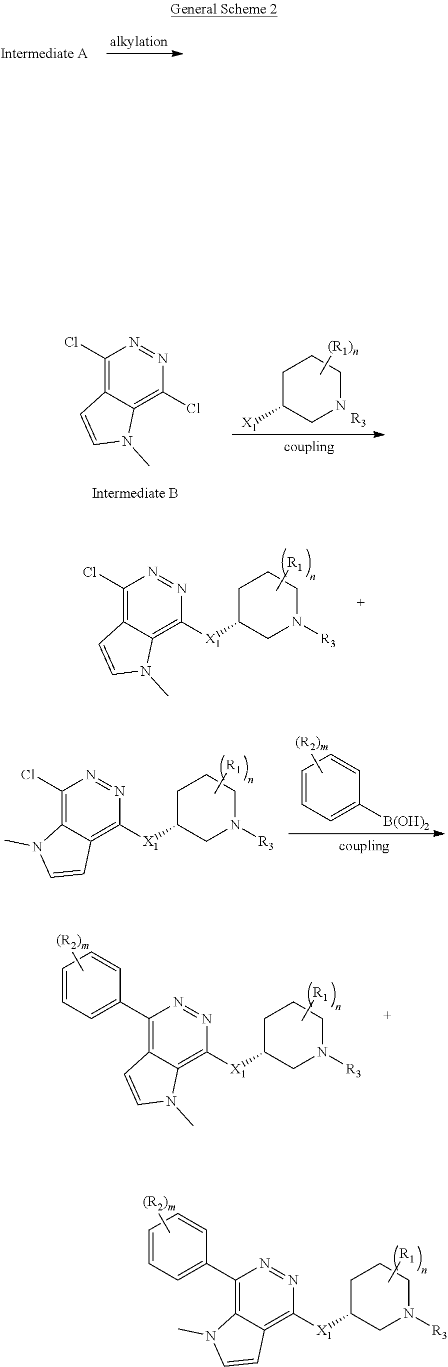

Synthesis of (R)-2-(1-methyl-7-((1-methylpiperidin-3-yl)amino)-1H-pyrrolo[2,3-d]pyridazin-4-yl)phenol and (Compound 17) (R)-2-(1-methyl-4-((1-methylpiperidin-3-yl)amino)-1H-pyrrolo[2,3-d]pyridazin-7-yl)phenol (Compound 67)

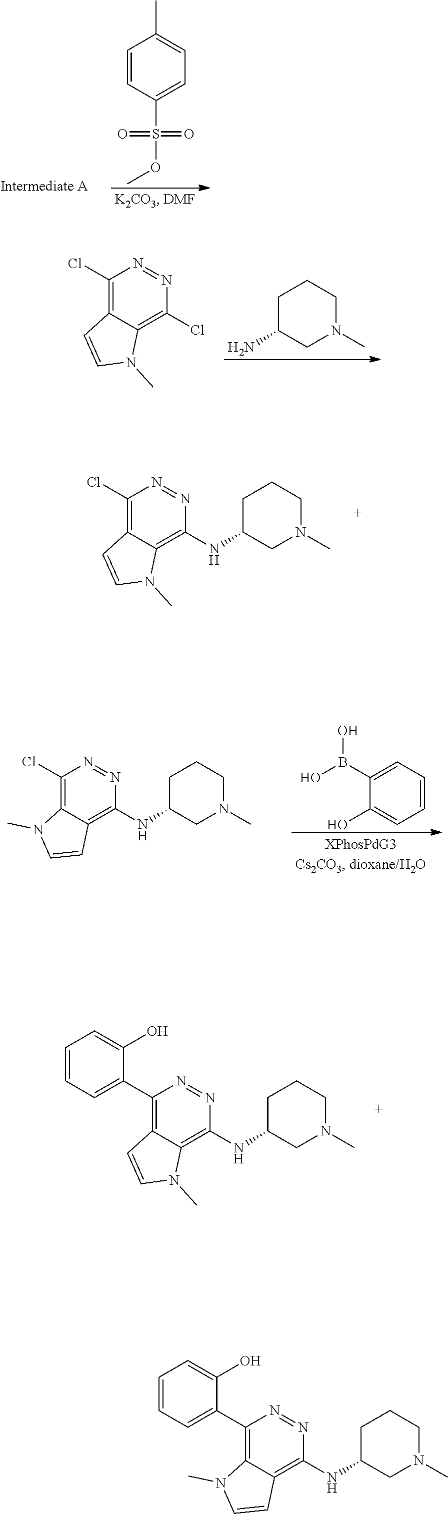

### Synthesis of 4,7-dichloro-1-methyl-1H-pyrrolo[2,3-d]pyridazine

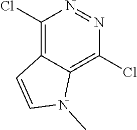

To a solution of Intermediate A (2 g, 11 mmol) in DMF (8 mL) were added methyl 4-methylbenzenesulfonate (5.94 g, 31.9 mmol) and K₂CO₃ (8.82 g, 63.8 mmol). The resulting mixture was stirred at room temperature for 2 h under nitrogen atmosphere. The mixture was then diluted with H₂O (40 mL) and extracted with DCM (3×30 mL). The organic layers were combined, dried over anhydrous Na₂SO₄, filtered and concentrated under reduced pressure to afford the crude residue. The crude residue was purified by silica gel column chromatography (PE:EtOAc, 10:1 to 1:1) to afford desired product 4,7-dichloro-1-methyl-1H-pyrrolo[2,3-d]pyridazine (690 mg, 31%) as an off-white solid.

### Synthesis of (R)-4-chloro-1-methyl-N-(1-methylpiperidin-3-yl)-1H-pyrrolo[2,3-d]pyridazin-7-amine and (R)-7-chloro-1-methyl-N-(1-methylpiperidin-3-yl)-1H-pyrrolo[2,3-d]pyridazin-4-amine

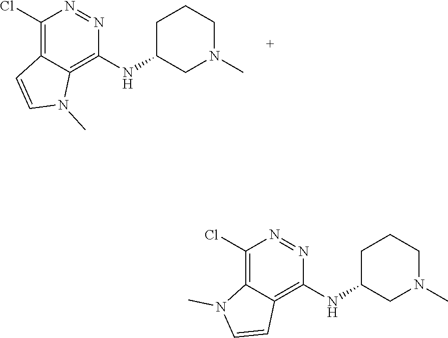

A mixture of 4,7-dichloro-1-methyl-1H-pyrrolo[2,3-d]pyridazine (200 mg, 0.990 mmol) and (3R)-1-methylpiperidin-3-amine (227 mg, 1.98 mmol) was stirred at 140° C. for 16 h under nitrogen atmosphere. The resulting crude residue was purified by silica gel column chromatography (DCM:MeOH, 10:1 to 5:1) to afford a mixture of desired products (R)-4-chloro-1-methyl-N-(1-methylpiperidin-3-yl)-1H-pyrrolo[2,3-d]pyridazin-7-amine and (R)-7-chloro-1-methyl-N-(1-methylpiperidin-3-yl)-1H-pyrrolo[2,3-d]pyridazin-4-amine (70 mg) as a colorless oil.

### Synthesis of (R)-2-(1-methyl-7-((1-methylpiperidin-3-yl)amino)-1H-pyrrolo[2,3-d]pyridazin-4-yl)phenol (Compound 17) and (R)-2-(1-methyl-4-((1-methylpiperidin-3-yl)amino)-1H-pyrrolo[2,3-d]pyridazin-7-yl)phenol (Compound 67)

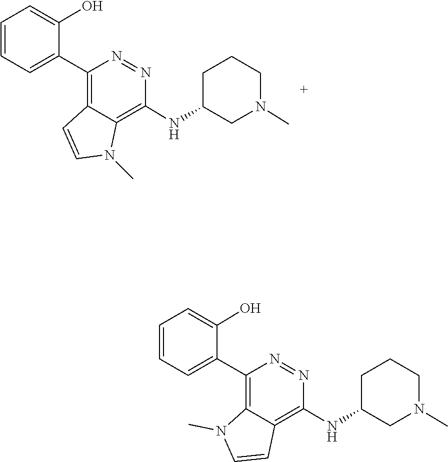 [SMILES: CN1CCC[C@@H](Nc2nnc(-c3ccccc3O)c3ccn(C)c23)C1 ; CN1CCC[C@@H](Nc2nnc(-c3ccccc3O)c3c2ccn3C)C1]

To a solution of a mixture of (R)-4-chloro-1-methyl-N-(1-methylpiperidin-3-yl)-1H-pyrrolo[2,3-d]pyridazin-7-amine and (R)-7-chloro-1-methyl-N-(1-methylpiperidin-3-yl)-1H-pyrrolo[2,3-d]pyridazin-4-amine (70 mg, 0.3 mmol) in 1,4-dioxane (2 mL) and H₂O (0.5 mL) were added 2-(4,4,5,5-tetramethyl-1,3,2-dioxaborolan-2-yl)phenol (95 mg, 0.43 mmol), Cs₂CO₃ (350 mg, 1.07 mmol) and XPhosPdG3 (31 mg, 0.036 mmol). The resulting mixture was stirred at 100° C. for 5 h under nitrogen atmosphere. The mixture was then diluted with H₂O (30 mL) and extracted with DCM (3×20 mL). The organic layers were combined, dried over anhydrous Na₂SO₄, filtered and concentrated under reduced pressure to afford the crude residue. The crude residue was purified by prep-HPLC (0.05% NH₃·H₂O as additive) to afford desired products (R)-2-(1-methyl-7-((1-methylpiperidin-3-yl)amino)-1H-pyrrolo[2,3-d]pyridazin-4-yl)phenol (Compound 17, 4.5 mg, 4%, peak 1) and (R)-2-(1-methyl-4-((1-methylpiperidin-3-yl)amino)-1H-pyrrolo[2,3-d]pyridazin-7-yl)phenol (Compound 67, 17.4 mg, 14%, peak 2) as white solids.

### Example 3

### General Synthetic Route C

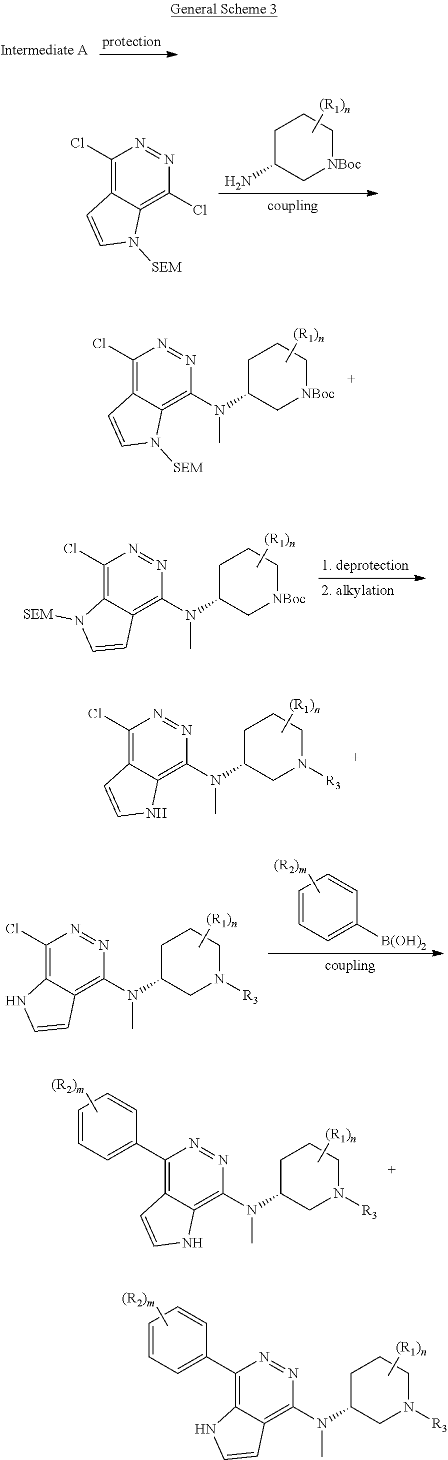

Synthesis of (R)-2-(7-(methyl(1-methylpiperidin-3-yl)amino)-1H-pyrrolo[2,3-d]pyridazin-4-yl)phenol (Compound 24) and (R)-2-(4-(methyl(1-methylpiperidin-3-yl)amino)-1H-pyrrolo[2,3-d]pyridazin-7-yl)phenol (Compound 68)

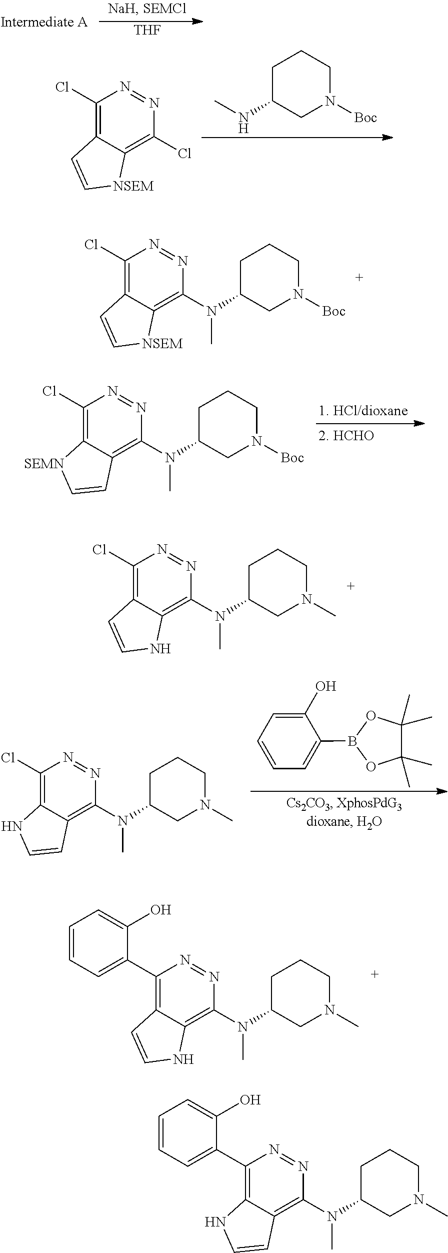

### Synthesis of 4,7-dichloro-1-((2-(trimethylsilyl)ethoxy)methyl)-1H-pyrrolo[2,3-d]pyridazine

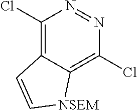 [SMILES: C[Si](C)(C)CCOCn1ccc2c(Cl)nnc(Cl)c21]

To a solution of Intermediate A (500 mg, 2.66 mmol) in THF (5 mL) was added 60% NaH in mineral oil (159 mg, 3.99 mmol) at 0° C. The mixture was stirred at 0° C. for 30 min under nitrogen atmosphere. SEM-CI (665 mg, 3.99 mmol) was then added and the resulting mixture was stirred at room temperature for an additional 14 h. The mixture was diluted with H₂O (60 mL) and extracted with DCM (3×50 mL). The organic layers were combined, dried over anhydrous Na₂SO₄, filtered and concentrated under reduced pressure to afford the crude residue. The crude residue was purified by silica gel column chromatography (DCM:MeOH, 1:0 to 20:1) to afford desired product 4,7-dichloro-1-((2-(trimethylsilyl)ethoxy)methyl)-1H-pyrrolo[2,3-d]pyridazine (1.10 g, 93%) as a yellow solid.

### Synthesis of tert-butyl (R)-3-((4-chloro-1-((2-(trimethylsilyl)ethoxy)methyl)-1H-pyrrolo[2,3-d]pyridazin-7-yl)(methyl)amino)piperidine-1-carboxylate and tert-butyl (R)-3-((7-chloro-1-((2-(trimethylsilyl)ethoxy)methyl)-1H-pyrrolo[2,3-d]pyridazin-4-yl)(methyl)amino)piperidine-1-carboxylate

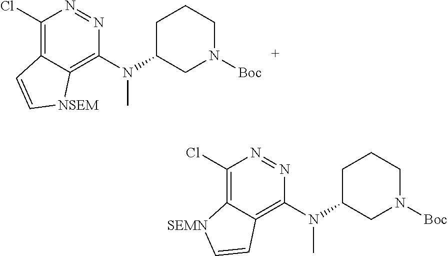 [SMILES: CN(c1nnc(Cl)c2ccn(COCC[Si](C)(C)C)c12)[C@@H]1CCCN(C(=O)OC(C)(C)C)C1 ; CN(c1nnc(Cl)c2c1ccn2COCC[Si](C)(C)C)[C@@H]1CCCN(C(=O)OC(C)(C)C)C1]

A mixture of 4,7-dichloro-1-((2-(trimethylsilyl)ethoxy)methyl)-1H-pyrrolo[2,3-d]pyridazine (948 mg, 2.98 mmol) and tert-butyl (3R)-3-(methylamino)piperidine-1-carboxylate (1.28 g, 5.96 mmol) was stirred at 140° C. for 16 h under nitrogen atmosphere. The resulting crude residue was purified by silica gel column chromatography (DCM:MeOH, 1:0 to 5:1) to afford a mixture of desired products tert-butyl (R)-3-((4-chloro-1-((2-(trimethylsilyl)ethoxy)methyl)-1H-pyrrolo[2,3-d]pyridazin-7-yl)(methyl)amino)piperidine-1-carboxylate and tert-butyl (R)-3-((7-chloro-1-((2-(trimethylsilyl)ethoxy)methyl)-1H-pyrrolo[2,3-d]pyridazin-4-yl)(methyl)amino)piperidine-1-carboxylate (447 mg) as a yellow solid.

### Synthesis of (R)-4-chloro-N-methyl-N-(piperidin-3-yl)-1H-pyrrolo[2,3-d]pyridazin-7-amine and (R)-7-chloro-N-methyl-N-(piperidin-3-yl)-1H-pyrrolo[2,3-d]pyridazin-4-amine

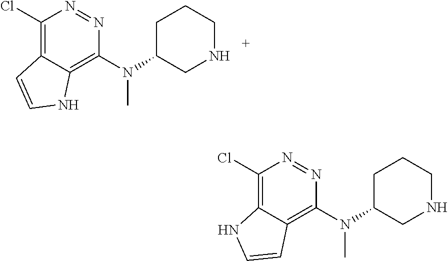 [SMILES: CN(c1nnc(Cl)c2cc[nH]c12)[C@@H]1CCCNC1 ; CN(c1nnc(Cl)c2[nH]ccc12)[C@@H]1CCCNC1]

A solution of a mixture of tert-butyl (R)-3-((4-chloro-1-((2-(trimethylsilyl)ethoxy)methyl)-1H-pyrrolo[2,3-d]pyridazin-7-yl)(methyl)amino)piperidine-1-carboxylate and tert-butyl (R)-3-((7-chloro-1-((2-(trimethylsilyl)ethoxy)methyl)-1H-pyrrolo[2,3-d]pyridazin-4-yl)(methyl)amino)piperidine-1-carboxylate (447 mg, 0.901 mmol) in 4M HCl in 1,4-dioxane (5 mL) was stirred at room temperature for 2 h. The resulting mixture was then concentrated under reduced pressure to afford a mixture of crude products (R)-4-chloro-N-methyl-N-(piperidin-3-yl)-1H-pyrrolo[2,3-d]pyridazin-7-amine and (R)-7-chloro-N-methyl-N-(piperidin-3-yl)-1H-pyrrolo[2,3-d]pyridazin-4-amine (447 mg) as a yellow solid. The mixture of crude products was used in the next step without further purification.

### Synthesis of (R)-4-chloro-N-methyl-N-(1-methylpiperidin-3-yl)-1H-pyrrolo[2,3-d]pyridazin-7-amine and (R)-7-chloro-N-methyl-N-(1-methylpiperidin-3-yl)-1H-pyrrolo[2,3-d]pyridazin-4-amine

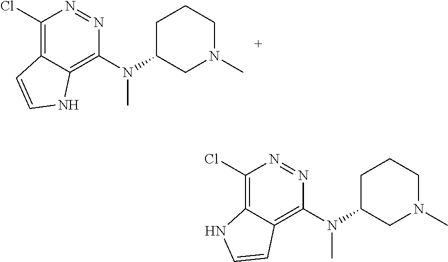 [SMILES: CN1CCC[C@@H](N(C)c2nnc(Cl)c3cc[nH]c23)C1 ; CN1CCC[C@@H](N(C)c2nnc(Cl)c3[nH]ccc23)C1]

To a solution of a mixture of (R)-4-chloro-N-methyl-N-(piperidin-3-yl)-1H-pyrrolo[2,3-d]pyridazin-7-amine and (R)-7-chloro-N-methyl-N-(piperidin-3-yl)-1H-pyrrolo[2,3-d]pyridazin-4-amine (447 mg, 1.48 mmol) in THF (10 mL) and H₂O (1 mL) were added 37% HCHO in H₂O (144 mg, 1.77 mmol), NaBH₃CN (139 mg, 2.22 mmol) and AcOH (8.88 mg, 0.148 mmol). The resulting mixture was stirred at 50° C. for 2 h. The mixture was then poured into H₂O (70 mL) and a saturated NaHCO₃ aqueous solution was added to adjust the pH to ˜8. The mixture was then extracted with EtOAc (3×50 mL). The organic layers were combined, dried over anhydrous Na₂SO₄, filtered and concentrated under reduced pressure to afford the crude residue. The crude residue was purified by silica gel column chromatography (DCM:MeOH, 1:0 to 5:1) to afford a mixture of desired products (R)-4-chloro-N-methyl-N-(1-methylpiperidin-3-yl)-1H-pyrrolo[2,3-d]pyridazin-7-amine and (R)-7-chloro-N-methyl-N-(1-methylpiperidin-3-yl)-1H-pyrrolo[2,3-d]pyridazin-4-amine (128 mg) as a yellow solid.

### Synthesis of (R)-2-(7-(methyl(1-methylpiperidin-3-yl)amino)-1H-pyrrolo[2,3-d]pyridazin-4-yl)phenol (Compound 24) and (R)-2-(4-(methyl(1-methylpiperidin-3-yl)amino)-1H-pyrrolo[2,3-d]pyridazin-7-yl)phenol (Compound 68)

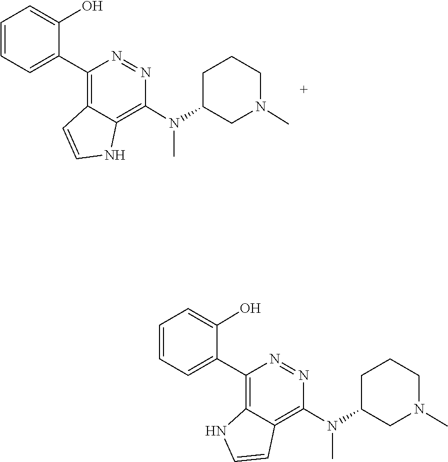 [SMILES: CN1CCC[C@@H](N(C)c2nnc(-c3ccccc3O)c3cc[nH]c23)C1 ; CN1CCC[C@@H](N(C)c2nnc(-c3ccccc3O)c3[nH]ccc23)C1]

To a solution of a mixture of (R)-4-chloro-N-methyl-N-(1-methylpiperidin-3-yl)-1H-pyrrolo[2,3-d]pyridazin-7-amine and (R)-7-chloro-N-methyl-N-(1-methylpiperidin-3-yl)-1H-pyrrolo[2,3-d]pyridazin-4-amine (80 mg, 0.29 mmol) in 1,4-dioxane (4 mL) and H₂O (1 mL) were added 2-(4,4,5,5-tetramethyl-1,3,2-dioxaborolan-2-yl)phenol (69 mg, 0.31 mmol), Cs₂CO₃ (280 mg, 0.858 mmol) and XPhosPdG₃ (24 mg, 0.028 mmol). The resulting mixture was stirred at 100° C. for 4 h under nitrogen atmosphere. The mixture was then diluted with H₂O (30 mL) and extracted with DCM (3×20 mL). The organic layers were combined, dried over anhydrous Na₂SO₄, filtered and concentrated under reduced pressure to afford the crude residue. The crude residue was purified by prep-HPLC (0.05% NH₃·H₂O as additive) to afford desired products (R)-2-(4-(methyl(1-methylpiperidin-3-yl)amino)-1H-pyrrolo[2,3-d]pyridazin-7-yl)phenol (Compound 68, 16.9 mg, 16%, peak 1) and (R)-2-(7-(methyl(1-methylpiperidin-3-yl)amino)-1H-pyrrolo[2,3-d]pyridazin-4-yl)phenol (Compound 24, 5 mg, 5%, peak 2) as off-white solids.

### Example 4

### General Synthetic Route D

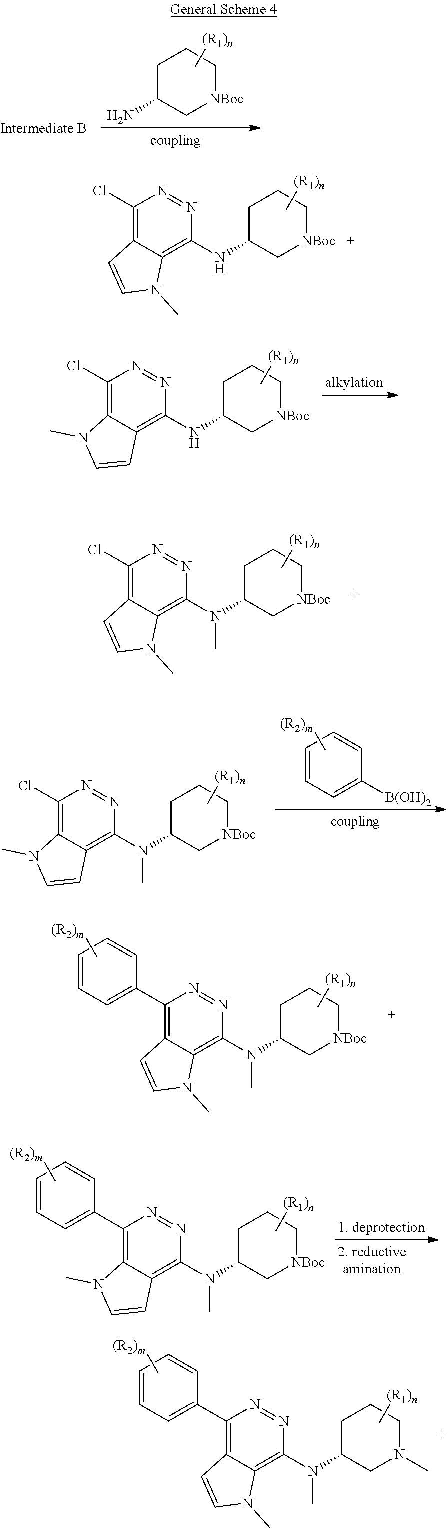

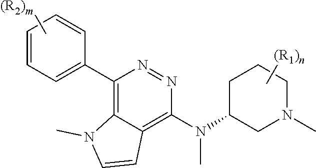

### Synthesis of (R)—N,1-dimethyl-N-(1-methylpiperidin-3-yl)-4-(4-(trifluoromethyl)phenyl)-1H-pyrrolo[2,3-d]pyridazin-7-amine (Compound 27)

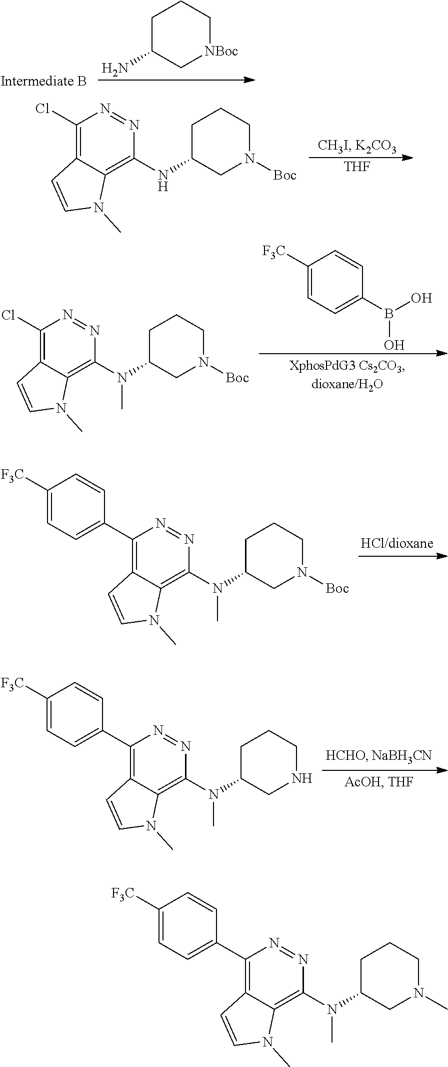

### Synthesis of tert-butyl (R)-3-((4-chloro-1-methyl-1H-pyrrolo[2,3-d]pyridazin-7-yl)amino)piperidine-1-carboxylate

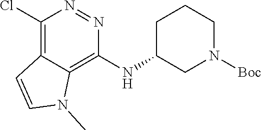 [SMILES: Cn1ccc2c(Cl)nnc(N[C@@H]3CCCN(C(=O)OC(C)(C)C)C3)c21]

To a solution of Intermediate B (2 g, 9.9 mmol) and (R)-tert-butyl 3-aminopiperidine-1-carboxylate (1.98 g, 9.9 mmol) in toluene (25 mL) were added Cs₂CO₃ (9.68 g, 29.7 mmol), Pd(OAc)₂ (111 mg, 0.50 mmol) and BINAP (617 mg, 0.99 mmol). The resulting mixture was stirred at 110° C. for 5 h under nitrogen atmosphere. The mixture was then poured into H₂O (40 mL) and extracted with EtOAc (3×40 mL). The organic layers were combined, washed with brine (10 mL), dried over anhydrous Na₂SO₄, filtered and concentrated under reduced pressure to afford a crude residue. The crude residue was purified by silica gel column chromatography (80% EtOAc in PE) to afford desired product tert-butyl (R)-3-((4-chloro-1-methyl-1H-pyrrolo[2,3-d]pyridazin-7-yl)amino)piperidine-1-carboxylate (1.37 g, 37.8%) as a yellow solid.

### Synthesis of tert-butyl (R)-3-((4-chloro-1-methyl-1H-pyrrolo[2,3-d]pyridazin-7-yl)(methyl)amino)piperidine-1-carboxylate

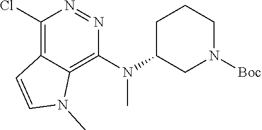 [SMILES: CN(c1nnc(Cl)c2ccn(C)c12)[C@@H]1CCCN(C(=O)OC(C)(C)C)C1]

To a solution of tert-butyl (R)-3-((4-chloro-1-methyl-1H-pyrrolo[2,3-d]pyridazin-7-yl)amino)piperidine-1-carboxylate (500 mg, 1.37 mmol) in THF (6 mL) was added 60% NaH in mineral oil (82 mg, 2.1 mmol). The mixture was stirred at room temperature for 30 min under nitrogen atmosphere. CH₃I (233 mg, 1.64 mmol) was then added, and the resulting mixture was stirred at room temperature for an additional 14 h. The mixture was then poured into H₂O (40 mL) and extracted with EtOAc (3×40 mL). The organic layers were combined, washed with brine (10 mL), dried over anhydrous Na₂SO₄, filtered and concentrated under reduced pressure to afford a crude residue. The crude residue was purified by silica gel column chromatography (60% EtOAc in PE) to afford desired product tert-butyl (R)-3-((4-chloro-1-methyl-1H-pyrrolo[2,3-d]pyridazin-7-yl)(methyl)amino)piperidine-1-carboxylate (350 mg, 67%) as a yellow solid.

### Synthesis of tert-butyl (R)-3-(methyl(1-methyl-4-(4-(trifluoromethyl)phenyl)-1H-pyrrolo[2,3-d]pyridazin-7-yl)amino)piperidine-1-carboxylate

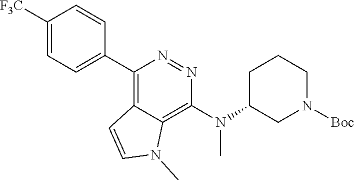 [SMILES: CN(c1nnc(-c2ccc(C(F)(F)F)cc2)c2ccn(C)c12)[C@@H]1CCCN(C(=O)OC(C)(C)C)C1]

To a solution of tert-butyl (R)-3-((4-chloro-1-methyl-1H-pyrrolo[2,3-d]pyridazin-7-yl)(methyl)amino)piperidine-1-carboxylate (150 mg, 0.395 mmol) in 1,4-dioxane (3 mL) and H₂O (0.3 mL) were added (4-(trifluoromethyl)phenyl)boronic acid (90 mg, 0.47 mmol), Cs₂CO₃ (386 mg, 1.18 mmol) and XPhosPdG3 (33 mg, 0.04 mmol). The resulting mixture was stirred at 100° C. for 14 h under nitrogen atmosphere. The mixture was then poured into H₂O (20 mL) and extracted with EtOAc (3×20 mL). The organic layers were combined, washed with brine (20 mL), dried over anhydrous Na₂SO₄, filtered and concentrated under reduced pressure to afford a crude residue. The crude residue was purified by silica gel column chromatography (50% EtOAc in PE) to afford desired product tert-butyl (R)-3-(methyl(1-methyl-4-(4-(trifluoromethyl)phenyl)-1H-pyrrolo[2,3-d]pyridazin-7-yl)amino)piperidine-1-carboxylate (170 mg, 88%) as a yellow solid.

### Synthesis of (R)—N,1-dimethyl-N-(piperidin-3-yl)-4-(4-(trifluoromethyl)phenyl)-1H-pyrrolo[2,3-d]pyridazin-7-amine

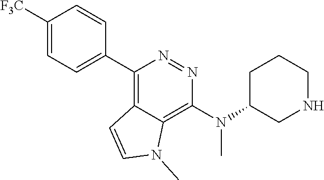 [SMILES: CN(c1nnc(-c2ccc(C(F)(F)F)cc2)c2ccn(C)c12)[C@@H]1CCCNC1]

To a solution of tert-butyl (R)-3-(methyl(1-methyl-4-(4-(trifluoromethyl)phenyl)-1H-pyrrolo[2,3-d]pyridazin-7-yl)amino)piperidine-1-carboxylate (170 mg, 0.347 mmol) in DCM (1 mL) was added 4M HCl in 1,4-dioxane (5 mL). The resulting mixture was stirred at room temperature for 1 h. The mixture was then concentrated under reduced pressure to afford desired crude product (R)—N,1-dimethyl-N-(piperidin-3-yl)-4-(4-(trifluoromethyl)phenyl)-1H-pyrrolo[2,3-d]pyridazin-7-amine (230 mg) as a yellow solid. The crude product was used in the next step without further purification.

### Synthesis of (R)—N,1-dimethyl-N-(1-methylpiperidin-3-yl)-4-(4-(trifluoromethyl)phenyl)-1H-pyrrolo[2,3-d]pyridazin-7-amine (Compound 27)

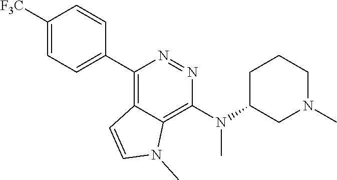 [SMILES: CN1CCC[C@@H](N(C)c2nnc(-c3ccc(C(F)(F)F)cc3)c3ccn(C)c23)C1]

To a solution of (R)—N,1-dimethyl-N-(piperidin-3-yl)-4-(4-(trifluoromethyl)phenyl)-1H-pyrrolo[2,3-d]pyridazin-7-amine (160 mg, 0.346 mmol) in THF (2 mL) were added 37% HCHO in H₂O (34 mg, 0.42 mmol), NaBH₃CN (33 mg, 0.52 mmol) and AcOH (48 mg, 0.35 mmol). The resulting mixture was stirred at 50° C. for 2 h. The mixture was then concentrated under reduced pressure to afford a crude residue. The crude residue was purified by prep-HPLC (0.05% NH3·H2O as additive) to afford desired product (R)—N,1-dimethyl-N-(1-methylpiperidin-3-yl)-4-(4-(trifluoromethyl)phenyl)-1H-pyrrolo[2,3-d]pyridazin-7-amine (Compound 27, 13 mg, 9%,) as a white solid.

### Example 5

### General Synthetic Route E

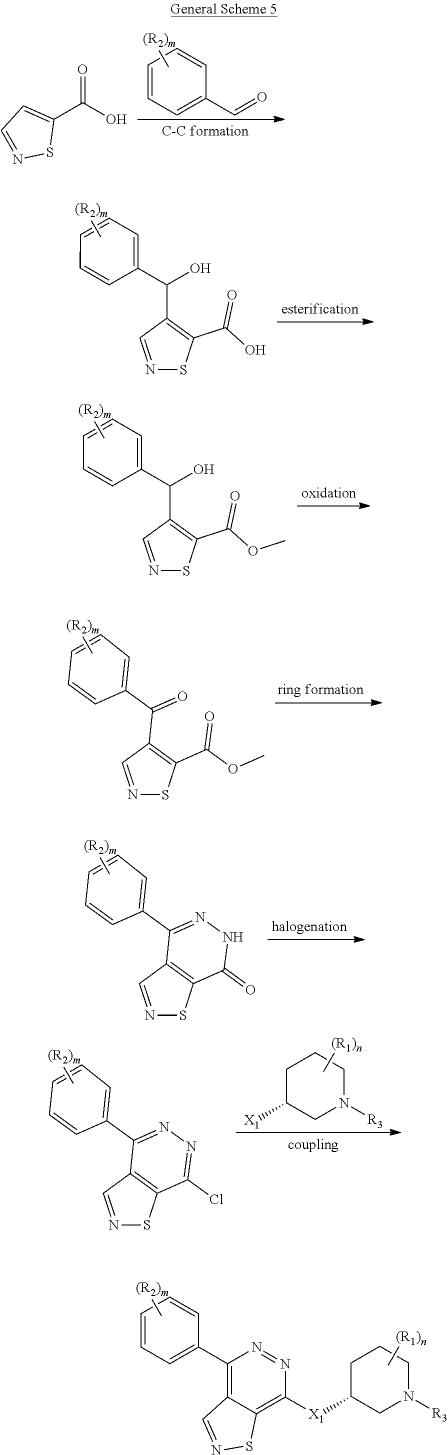

### Synthesis of (R)—N-(1-methylpiperidin-3-yl)-4-(4-(trifluoromethyl)phenyl)isothiazolo[4,5-d]pyridazin-7-amine (Compound 63)

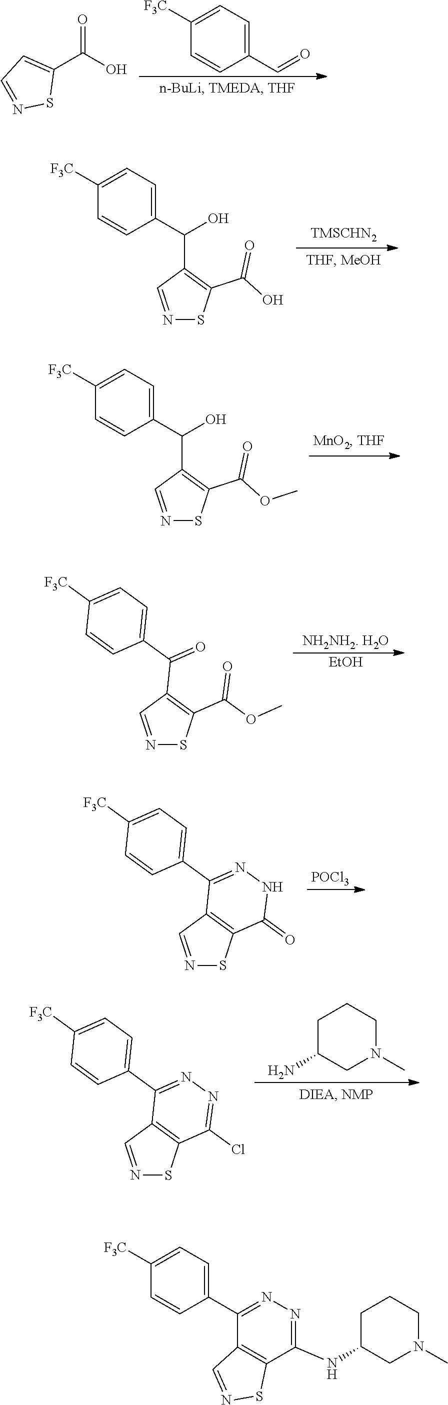

### Synthesis of 4-(hydroxy(4-(trifluoromethyl)phenyl)methyl)isothiazole-5-carboxylic acid

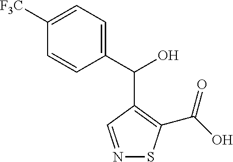 [SMILES: O=C(O)c1sncc1C(O)c1ccc(C(F)(F)F)cc1]

To a solution of isothiazole-5-carboxylic acid (5 g, 38.7 mmol) and TMEDA (11.2 g, 96.8 mmol) in THF (50 mL) was added 2.5 M n-BuLi in hexanes (38.7 mL, 96.8 mmol) at −78° C. The mixture was stirred at −78° C. for 1 h under nitrogen atmosphere. A solution of 4-(trifluoromethyl)benzaldehyde (14.2 g, 81.3 mmol) in THF (15 mL) was then added, and the resulting mixture was stirred at room temperature for an additional 3 h. The mixture was acidified with 1 N aqueous HCl to adjust the pH to ˜4, then poured into H₂O (50 mL) and extracted with EtOAc (3×100 mL). The organic layers were combined, washed with brine (200 mL), dried over anhydrous Na₂SO₄, filtered and concentrated under reduced pressure to afford a crude residue. The crude residue was purified by silica gel column chromatography (25% EtOAc in PE) to afford desired product 4-(hydroxy(4-(trifluoromethyl)phenyl)methyl)isothiazole-5-carboxylic acid (1.77 g, 15%) as a yellow solid.

### Synthesis of methyl 4-(hydroxy(4-(trifluoromethyl)phenyl)methyl)isothiazole-5-carboxylate

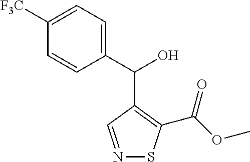 [SMILES: COC(=O)c1sncc1C(O)c1ccc(C(F)(F)F)cc1]

To a solution of 4-(hydroxy(4-(trifluoromethyl)phenyl)methyl)isothiazole-5-carboxylic acid (570 mg, 1.88 mmol) in MeOH (5 mL) and THF (5 mL) was added 2M diazomethyl(trimethyl)silane in hexanes (1.88 mL, 3.76 mmol). The resulting mixture was stirred at room temperature for 14 h under nitrogen atmosphere. The mixture was then acidified with AcOH to adjust the pH to ˜5, then poured into H₂O (10 mL) and extracted with EtOAc (2×10 mL). The organic layers were combined, washed with brine (10 mL), dried over anhydrous Na₂SO₄, filtered and concentrated under reduced pressure to afford a crude residue. The crude residue was purified by silica gel column chromatography (25% EtOAc in PE) to afford desired product methyl 4-(hydroxy(4-(trifluoromethyl)phenyl)methyl)isothiazole-5-carboxylate (280 mg, 47%) as a yellow oil.

### Synthesis of methyl 4-(4-(trifluoromethyl)benzoyl)isothiazole-5-carboxylate

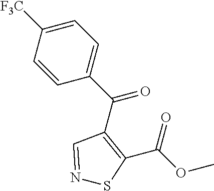 [SMILES: COC(=O)c1sncc1C(=O)c1ccc(C(F)(F)F)cc1]

To a solution of methyl 4-(hydroxy(4-(trifluoromethyl)phenyl)methyl)isothiazole-5-carboxylate (280 mg, 0.883 mmol) in THF (3 mL) was added MnO₂ (767 mg, 8.83 mmol). The resulting mixture was stirred at 40° C. for 14 h and was then concentrated under reduced pressure to afford a crude residue. The crude residue was purified by silica gel column chromatography (25% EtOAc in PE) to afford desired product methyl 4-(4-(trifluoromethyl)benzoyl)isothiazole-5-carboxylate (380 mg, >100%) as a white solid.

### Synthesis of 4-(4-(trifluoromethyl)phenyl)isothiazolo[4,5-d]pyridazin-7(6H)-one

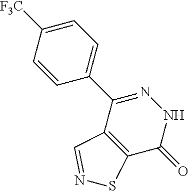 [SMILES: O=c1[nH]nc(-c2ccc(C(F)(F)F)cc2)c2cnsc12]

To a solution of methyl 4-(4-(trifluoromethyl)benzoyl)isothiazole-5-carboxylate (380 mg, 1.21 mmol) in EtOH (5 mL) was added 85% NH₂NH₂·H₂O (710 mg, 12.1 mmol). The resulting mixture was stirred at 80° C. for 12 h. The mixture was then filtered, and the filter cake was washed with EtOH (2 mL) and dried under vacuum to afford desired product 4-(4-(trifluoromethyl)phenyl)isothiazolo[4,5-d]pyridazin-7(6H)-one (230 mg, 64%) as a white solid.

### Synthesis of 7-chloro-4-(4-(trifluoromethyl)phenyl)isothiazolo[4,5-d]pyridazine

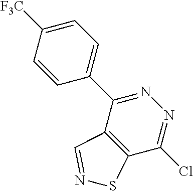 [SMILES: FC(F)(F)c1ccc(-c2nnc(Cl)c3sncc23)cc1]

A solution of 4-(4-(trifluoromethyl)phenyl)isothiazolo[4,5-d]pyridazin-7(6H)-one (230 mg, 0.774 mmol) in POCl₃ (4.4 mL) was stirred at 120° C. for 6 h under nitrogen atmosphere. The resulting mixture was basified with saturated NaHCO₃ aqueous solution to adjust the pH to ˜8, then poured into H₂O (10 mL) and extracted with EtOAc (2×10 mL). The organic layers were combined, washed with brine (10 mL), dried over anhydrous Na₂SO₄, filtered and concentrated under reduced pressure to afford desired crude product 7-chloro-4-(4-(trifluoromethyl)phenyl)isothiazolo[4,5-d]pyridazine (239 mg) as a yellow solid. The crude product was used in the next step without further purification.

### Synthesis of (R)—N-(1-methylpiperidin-3-yl)-4-(4-(trifluoromethyl)phenyl)isothiazolo[4,5-d]pyridazin-7-amine (Compound 63)

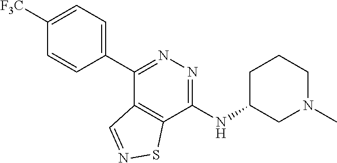 [SMILES: CN1CCC[C@@H](Nc2nnc(-c3ccc(C(F)(F)F)cc3)c3cnsc23)C1]

To a solution of 7-chloro-4-(4-(trifluoromethyl)phenyl)isothiazolo[4,5-d]pyridazine (239 mg, 0.757 mmol) in NMP (3 mL) were added (3R)-1-methylpiperidin-3-amine (104 mg, 0.908 mmol) and DIEA (0.4 mL, 2.27 mmol). The resulting mixture was stirred at 130° C. for 12 h under nitrogen atmosphere. The mixture was then poured into H₂O (10 mL) and extracted with EtOAc (2×10 mL). The organic layers were combined, washed with brine (10 mL), dried over anhydrous Na₂SO₄, filtered and concentrated under reduced pressure to afford a crude residue. The crude residue was purified by prep-HPLC (0.05% NH₃H₂O as additive) to afford (R)—N-(1-methylpiperidin-3-yl)-4-(4-(trifluoromethyl)phenyl)isothiazolo[4,5-d]pyridazin-7-amine (Compound 63, 17 mg, 6%) as an off-white solid.

### Example 6

### Synthesis of Representative Compounds

The Compounds listed in Table 2 below were prepared by procedures similar to the ones described in the representative schemes found in the Examples with appropriate variations in reactants, quantities of reagents, protections and deprotections, solvents and reaction conditions. The characterization data of the compounds are also summarized herein in Table 2.

TABLE 2

| | General | |
|---|---|---|
| Cpd | Synthetic | |
| No. | Route | Characterization |
| 1 | A | ¹H NMR (400 MHz, DMSO-d₆): δ |
| | | 11.83 (brs, 1H), 8.07 (d, J = 8.0 |
| | | Hz, 2H), 7.87 (d, J = 8.4 Hz, 2H), |
| | | 7.44 (s, 1H), 6.89 (d, J = 2.8 Hz, |
| | | 1H), 6.70 (d, J = 8.0 Hz, 1H), 4.32- |
| | | 4.42 (m, 1H), 3.02-3.08 (m, 1H), |
| | | 2.66-2.72 (m, 1H), 2.20 (s, 3H), |
| | | 1.83-2.03 (m, 3H), 1.69-1.80 (m, |
| | | 1H), 1.54-1.67 (m, 1H), 1.31-1.43 |
| | | (m, 1H). |
| | | LCMS[M + H]+ = 376.2 |
| 2 | A | ¹H NMR (400 MHz, DMSO-d₆): δ |
| | | 11.84 (brs, 1H), 8.17 (d, J = 8.0 |
| | | Hz, 2H), 7.86 (d, J = 8.4 Hz, 2H), |
| | | 7.65 (s, 1H), 6.75 (d, J = 2.8 Hz, |
| | | 1H), 6.60 (d, J = 7.6 Hz, 1H), 4.36- |
| | | 4.48 (m, 1H), 2.87-2.96 (m, 1H), |
| | | 2.56-2.65 (m, 1H), 2.04-2.25 (m, |
| | | 5H), 1.85-1.96 (m, 1H), 1.75-1.81 |
| | | (m, 1H), 1.55-1.65 (m, 1H), 1.39- |
| | | 1.50 (m, 1H). |
| | | LCMS[M + H]+ = 376.2 |
| 3 | A | ¹H NMR (400 MHz, DMSO-d₆): |
| | | δ11.42-11.68 (m, 1H), 8.22 (s, |
| | | 2H), 7.77 (s, 1H), 7.68 (br d, J = |
| | | 7.6 Hz, 1H), 7.57 (d, J = 7.6 Hz, |
| | | 1H), 7.37 (s, 1H), 6.80-6.90 (m, |
| | | 1H), 6.66-6.75 (m, 1H), 4.35-4.39 |
| | | (m, 1H), 3.09-3.15 (m, 1H), 2.73- |
| | | 2.77 (m, 1H), 2.23-2.28 (m, 6H), |
| | | 1.94-2.04 (m, 3H), 1.74-1.80 (m, |
| | | 1H), 1.59-1.66 (m, 1H), 1.36-1.44 |
| | | (m, 1H). |
| | | LCMS[M + H]+ = 390.2 |
| 4 | A | ¹H NMR (400 MHz, DMSO-d₆): δ |
| | | 12.06-12.36 (m, 1H), 8.12-8.22 |
| | | (m, 1H), 7.78 (d, J = 2.0 Hz, 1H), |
| | | 7.10-7.21 (m, 1H), 6.79-6.84 (m, |
| | | 1H), 6.68-6.78 (m, 2H), 4.29-4.37 |
| | | (m, 1H), 2.84-2.89 (m, 1H), 2.57- |
| | | 2.59 (m, 1H), 2.18-2.25(m, 5H), |
| | | 1.87-1.94 (m, 1H), 1.76-1.82 (m, |
| | | 1H), 1.56-1.64 (m, 1H), 1.40-1.51 |
| | | (m, 1H). |
| | | LCMS[M + H]+ = 342.1 |
| 5 | A | ¹H NMR (400 MHz, DMSO-d₆): δ |
| | | 11.78 (s, 1H), 7.94-8.00 (m, 2H), |
| | | 7.61 (br s, 1H), 7.33 (t, J = 8.8 Hz, |
| | | 2H), 6.66-6.70 (m, 1H), 6.47 (d, J = |
| | | 7.6 Hz, 1H), 4.34-4.42 (m, 1H), |
| | | 2.88-2.94 (m, 1H), 2.52-2.53 (m, |
| | | 1H), 2.21 (s, 3H), 2.01-2.16 (m, |
| | | 2H), 1.86-1.93 (m, 1H), 1.74-1.81 |
| | | (m, 1H), 1.56-1.63 (m, 1H), 1.38- |
| | | 1.48 (m, 1H). |
| | | LCMS[M + H]+ = 326.2 |
| 6 | A | ¹H NMR (400 MHz, DMSO-d₆): δ |
| | | 11.17-12.58 (m, 1H), 8.21 (s, 2H), |
| | | 7.73 (s, 1H), 7.64-7.68 (m, 1H), |
| | | 7.55-7.60 (m, 2H), 6.62-6.75 (m, |
| | | 1H), , 6.22 (d, J = 2.8 Hz, |
| | | 1H), 4.40-4.50 (m, 1H), 2.95-3.06 |
| | | (m, 1H), 2.55-2.61 (m, 1H), 2.31 |
| | | (s, 3H), 2.28 (s, 3H), 2.19-2.26 (m, |
| | | 2H), 21.91-1.95 (m, 1H), 1.75- |
| | | 1.87 (m, 1H), 1.62-1.67 (m, 1H), |
| | | 1.46-1.50 (m, 1H). |
| | | LCMS[M + H]+ = 390.1 |
| 7 | A | ¹H NMR (400 MHz, DMSO-d₆): δ |
| | | 8.20 (s, 1.5H), 7.96 (dd, J = 8.8, |
| | | 6.8 Hz, 1H), 7.60 (d, J = 3.2 Hz, |
| | | 1H), 6.96-7.04 (m, 2H), 7.67-7.19 |
| | | (m, 2H), 4.33 (br d, J = 7.6 Hz, |
| | | 1H), 3.11-3.16 (m, 1H), 2.79-2.82 |
| | | (m, 1H), 2.31 (s, 3H), 1.95-2.18 |
| | | (m, 3H), 1.72-1.80 (m, 1H), 1.60- |
| | | 1.70 (m, 1H), 1.35-1.48 (m, 1H). |
| | | LCMS[M + H]+ = 342.1 |
| 8 | A | ¹H NMR (400 MHz, CD3OD): δ |
| | | 8.39 (brs, 1H), 7.51-7.60 (m, 2H), |
| | | 7.27-7.33 (m, 1H), 6.88-6.98 (m, |
| | | 3H), 4.28-4.38 (m, 1H), 3.39-3.55 |
| | | (m, 1H), 3.07-3.18 (m, 1H), 2.69- |
| | | 2.82 (m, 2H), 2.64 (s, 3H), |
| | | 1.95-−2.10 (m, 2H), 1.75-1.88 |
| | | (m, 1H), 1.60-1.79 (m, 1H). |
| | | LCMS[M + H]+ = 324.0 |
| 9 | A | ¹H NMR (400 MHz, DMSO-d₆): δ |
| | | 11.74 (brs, 1H), 7.88 (s, 2H), 7.30- |
| | | 7.44 (m, 3H), 6.85 (s, 1H), 6.57 (d, |
| | | J = 7.2 Hz, 1H), 4.27-4.39 (m, 1H), |
| | | 3.00-3.07 (m, 1H), 2.62-2.70 (m, |
| | | 1H), 2.19 (s, 3H), 1.82-1.98 (m, |
| | | 3H), 1.70-1.78 (m, 1H), 1.53-1.63 |
| | | (m, 1H), 1.29-1.41 (m, 1H). |
| | | LCMS[M + H]+ = 326.2 |
| 10 | A | ¹H NMR (400 MHz, CD₃OD): δ |
| | | 8.47 (brs, 1H), 7.90 (d, J = 8.4 Hz, |
| | | 2H), 7.69 (d, J = 2.8 Hz, 1H), 7.61 |
| | | (d, J = 8.4 Hz, 2H), 6.86 (d, J = 2.8 |
| | | Hz, 1H), 4.52-4.58 (m, 1H), 3.63- |
| | | 3.75 (m, 1H), 3.28-3.32 (m, 1H), |
| | | 3.03-3.21(m, 2H), 2.86(s, 3H), |
| | | 2.14-2.23(m, 2H), 1.93-2.00 (m, |
| | | 1H), 1.82-1.91 (m, 1H). |
| | | LCMS[M + H]+ = 342.0 |
| 11 | A | ¹H NMR (400 MHz, CD₃OD): δ |
| | | 8.51 (s, 1H), 7.8-7.86 (m, 2H), |
| | | 7.59-7.65 (m, 2H), 7.57 (d, J = 2.8 |
| | | Hz, 1H), 6.99 (d, J = 2.8 Hz, 1H), |
| | | 4.43-4.52 (m, 1H), 3.57-3.66 (m, |
| | | 1H), 3.23-3.31 (m, 1H), 2.86-2.99 |
| | | (m, 2H), 2.79 (s, 3H), 2.08-2.22 |
| | | (m, 2H), 1.90-1.98 (m, 1H), 1.77- |
| | | 1.86 (m, 1H). |
| | | LCMS[M + H]+ = 342.2 |
| 12 | A | ¹H NMR (400 MHz, CD₃OD): δ |
| | | 8.51 (s, 1H), 7.85-7.90 (m, 1H), |
| | | 7.67-7.73 (m, 2H), 7.59-7.66 (m, |
| | | 2H), 6.91 (t, J = 55.6 Hz, 1H), 6.45 |
| | | (d, J = 3.2 Hz, 1H), 4.52-4.60 (m, |
| | | 1H), 3.66-3.86 (m, 1H), 3.36-3.54 |
| | | (m, 1H), 2.91-3.17 (m, 2H), 2.85 |
| | | (s, 3H), 2.14-2.26 (m, 2H), 1.94- |
| | | 2.03 (m, 1H), 1.79-1.89 (m, 1H). |
| | | LCMS[M + H]+ = 358.3 |
| 13 | A | ¹H NMR (400 MHz, CD₃OD): δ |
| | | 8.44 (s, 1H), 7.87-7.92 (m, 1H), |
| | | 7.70-7.77 (m, 2H), 7.60-7.66 (m, |
| | | 1H), 7.55 (t, J = 2.8 Hz, 1H), 6.74- |
| | | 7.04 (m, 2H), 4.51-4.58 (m, 1H), |
| | | 3.66-3.75 (m, 1H), 3.34-3.41(m, |
| | | 1H), 2.94-3.07 (m, 2H), 2.84(s, |
| | | 3H), 2.13-2.26 (m, 2H), 1.95-2.03 |
| | | (m, 1H), 1.80-1.90 (m, 1H). |
| | | LCMS[M + H]+ = 358.3 |
| 14 | A | ¹H NMR (400 MHz, DMSO-d₆): δ |
| | | 15.30 (brs, 1H), 12.12 (brs, 1H), |
| | | 8.12 (d, J = 7.6 Hz, 1H), 7.76 (d, J = |
| | | 2.8 Hz, 1H), 7.20-7.30 (m, 1H), |
| | | 7.11 (d, J = 2.8 Hz, 1H), 6.91-6.99 |
| | | (m, 2H), 6.77 (brd, J = 8.0 Hz, 1H), |
| | | 4.32-4.40 (m, 1H), 2.83-2.93 (m, |
| | | 1H), 2.53-2.63 (m, 1H), 2.09-2.24 |
| | | (m, 5H), 1.87-1.95 (m, 1H), 1.74- |
| | | 1.82 (m, 1H), 1.55-1.65 (m, 1H), |
| | | 1.41-1.52 (m, 1H). |
| | | LCMS[M + H]+ = 324.3 |
| 15 | A | ¹H NMR (400 MHz, DMSO-d₆) δ |
| | | 11.84 (brs, 1H), 7.86-8.00 (m, |
| | | 2H), 7.60 (d, J = 3.2 Hz, 1H), 7.47- |
| | | 7.55 (m, 2H), 7.38-7.46 (m, 1H), |
| | | 6.69 (d, J = 2.8 Hz, 1H), 6.47-6.54 |
| | | (m, 1H), 4.33-4.47 (m, 1H), 2.86- |
| | | 3.00 (m, 1H), 2.54-2.56 (m, 1H), |
| | | 2.21 (s, 3H), 2.14-2.18 (m, 1H), |
| | | 2.00-2.10 (m, 1H), 1.86-1.96 (m, |
| | | 1H), 1.73-1.83 (m, 1H), 1.54-1.66 |
| | | (m, 1H), 1.37-1.50 (m, 1H). |
| | | LCMS[M + H]+ = 308.0 |
| 16 | A | LCMS[M + H]+ = 337.2 (calc) |
| 17 | B | ¹H NMR (400 MHz, DMSO-d₆): δ |
| | | 14.77 (brs, 1H), 8.06 (dd, J = 8.0, |
| | | 1.2 Hz, 1H), 7.64 (d, J = 3.2 Hz, |
| | | 1H), 7.24-7.30 (m, 1H), 7.02 (d, J = |
| | | 3.2, 1H), 6.93-6.98 (m, 2H), |
| | | 5.98 (d, J = 7.6 Hz, 1H), 4.28-4.35 |
| | | (m, 1H), 4.20 (s, 3H), 2.87-2.94 |
| | | (m, 1H), 2.61-2.65 (m, 1H), 2.23 |
| | | (s, 3H), 2.13-2.17 (m, 1H), 1.88- |
| | | 1.93 (m, 1H), 1.73-1.78 (m, 1H), |
| | | 1.54-1.62 (m, 2H), 1.31-1.37 (m, 1H). |
| | | LCMS[M + H]+ = 338.3 |
| 18 | D | ¹H NMR (DMSO-d₆, 400 MHz): δ |
| | | 8.21 (brs, 1H), 8.09 (dd, J = 8.4 |
| | | Hz, 1.6 Hz, 1H), 7.79 (d, J = 3.2 |
| | | Hz, 1H), 7.31-7.37 (m, 1H), 7.11 |
| | | (d, J = 3.2 Hz, 2H), 6.96-7.03 (m, |
| | | 2H), 4.14 (s, 3H), 2.91 (s, 3H), |
| | | 2.83-2.88 (m, 1H), 2.66-2.72 (m, |
| | | 1H), 2.13-2.19 (m, 4H), 1.84-1.93 |
| | | (m, 2H), 1.66-1.75 (m, 1H), 1.49- |
| | | 1.60 (m, 2H). |
| | | LCMS[M + H]+ = 352.2 |
| 19 | A | ¹H NMR (400 MHz, DMSO-d₆): δ |
| | | 11.90 (brs, 1H), 8.12-8.20 (m, |
| | | 3H), 7.87 (d, J = 8.4 Hz, 2H), 7.67 |
| | | (d, J = 2.8 Hz, 1H), 6.67-6.79 (m, |
| | | 2H), 4.51-4.69 (m, 1H), 3.00-3.04 |
| | | (m, 1H), 2.81-2.91 (m, 1H), 2.56- |
| | | 2.69 (m, 2H), 2.22-2.36 (m, 4H), |
| | | 1.95-2.10 (m, 1H). |
| | | LCMS[M + H]+ = 412.0 |
| 20 | A | ¹H NMR (DMSO-d₆): δ 8.74 (d, J = |
| | | 8.0 Hz, 2H), 8.43 (s, 1H), 7.87 (d, J = |
| | | 8.4 Hz, 2H), 6.91 (brs, 1H), 4.42- |
| | | 4.53 (m, 1H), 2.79-2.87 (m, 1H), |
| | | 2.43-2.48 (m, 1H), 2.16-2.25 (m, |
| | | 5H), 1.74-1.87 (m, 2H), 1.51-1.63 |
| | | (m, 2H). |
| | | LCMS[M + H]+ = 377.3 |
| 21 | A | ¹H NMR (DMSO-d₆, 400 MHz): δ |
| | | 11.53-12.34 (m, 1H), 8.20 (s, 1H), |
| | | 8.15 (d, J = 8.0 Hz, 2H), 7.84 (d, J = |
| | | 8.4 Hz, 2H), 6.52-6.62 (m, 1H), |
| | | 6.48 (s, 1H), 4.41-4.49 (m, 1H), |
| | | 2.93-3.00 (m, 1H), 2.54-2.61 (m, |
| | | 1H), 2.48 (s, 3H), 2.27-2.33 (m, |
| | | 4H), 2.18-2.26 (m, 1H), 1.78-1.91 |
| | | (m, 2H), 1.58-1.66 (m, 1H), 1.44- |
| | | 1.53 (m, 1H). |
| | | LCMS[M + H]+ = 390.0 |
| 22 | A | LCMS[M + H]+ = 389.2 (calc) |
| 23 | A | ¹H NMR (400 MHz, DMSO-d₆): δ |
| | | 11.82 (brs, 1H), 8.01 (s, 1H), 7.94 |
| | | (d, J = 8.0 Hz, 1H), 7.79 (d, J = 8.0 |
| | | Hz, 1H), 7.64 (d, J = 2.8 Hz, 1H), |
| | | 6.76 (d, J = 3.2 Hz, 1H), 6.59 (d, J = |
| | | 7.6 Hz, 1H), 4.36-4.46 (m, 1H), |
| | | 2.86-2.95 (m, 1H), 2.54-2.56 (m, |
| | | 4H), 2.21 (s, 3H), 2.01-2.18 (m, |
| | | 2H), 1.87-1.95 (m, 1H), 1.74-1.82 |
| | | (m, 1H), 1.55-1.66 (m, 1H), 1.41- |
| | | 1.50 (m, 1H). |
| | | LCMS[M + H]+ = 390.1 |
| 24 | C | ¹H NMR (400 MHz, DMSO-d₆): δ |
| | | 14.87 (brs, 1H), 8.11 (d, J = 7.6 |
| | | Hz, 1H), 7.74 (d, J = 3.2 Hz, 1H), |
| | | 7.24-7.34 (m, 1H), 7.13 (d, J = 3.2 |
| | | Hz, 1H), 6.95-7.02 (m, 2H), 4.30- |
| | | 4.45 (m, 1H), 3.18 (s, 3H), 2.80- |
| | | 2.89 (m, 1H), 2.70-2.77 (m, 1H), |
| | | 2.18 (s, 3H), 2.09 (t, J = 10.4 Hz, |
| | | 1H), 1.61-1.87 (m, 5H). |
| | | LCMS[M + H]+ = 338.3 |
| 25 | C | ¹H NMR (400 MHz, CDCl₃): δ |
| | | 8.69 (brs, 1H), 8.17 (d, J = 8.0 Hz, 2H), |
| | | 7.78 (d, J = 8.4 Hz, 2H), 7.47-7.52 |
| | | (m, 1H), 6.78 (d, J = 2.8 Hz, 1H), |
| | | 4.45-4.53 (m, 1H), 3.90-3.92 (m, |
| | | 1H), 3.25-3.40 (m, 1H), 3.17 (s, |
| | | 3H), 2.98-3.06 (m, 1H), 2.79 (s, |
| | | 3H), 2.58-2.73 (m, 1H), 1.95-2.04 |
| | | (m, 1H), 1.85-1.92 (m, 3H). |
| | | LCMS[M + H]+ = 390.3 |
| 26 | B | ¹H NMR (400 MHz, DMSO-d₆) |
| | | δ 8.12 (d, J = 7.2 Hz, 2H), 7.86 (d, |
| | | J = 7.2 Hz, 2H), 7.42-7.62 (m, |
| | | 1H), 6.55-6.77 (m, 1H), 5.85 (brd, |
| | | J = 5.2 Hz, 1H), 4.30-4.48 (m, 1H), |
| | | 4.15 (s, 3H), 2.77-3.03 (m, 1H), |
| | | 2.51-2.56 (m, 1H), 1.85-2.25 (m, |
| | | 6H), 1.66-1.77 (m, 1H), 1.35-1.62 |
| | | (m, 2H). |
| | | LCMS[M + H]+ = 390.0 |
| 27 | D | ¹H NMR (400 MHz, DMSO-d₆): δ |
| | | 8.20 (d, J= 8.0 Hz, 2H), 7.91 (d, J = |
| | | 7.6 Hz, 2H), 7.66-7.75 (m, 1H), |
| | | 6.72-6.88 (m, 1H), 4.12 (s, 3H), |
| | | 3.48-3.68 (m, 1H), 2.91 (s, 3H), |
| | | 2.79-2.86 (m, 1H), 2.61-2.69 (m, |
| | | 1H), 2.01-2.18 (m, 4H), 1.75-1.86 |
| | | (m, 2H), 1.63-1.73 (m, 1H), 1.41- |
| | | 1.56 (m, 2H). |
| | | LCMS[M + H]+ = 404.2 |
| 28 | B | LCMS[M + H]+ = 376.2 (calc) |
| 29 | B | ¹H NMR (400 MHz, DMSO-d₆): δ |
| | | 8.36 (s, 1H), 7.85-7.89 (m, |
| | | 4H), 6.62 ((brd, J = 8.4 Hz, 1H)), |
| | | 4.38-4.47 (m, 1H), |
| | | 3.53 (s, 3H), 2.80-2.86 (m, 1H), |
| | | 2.51-2.53 (m, |
| | | 1H), 2.20 (s, 3H), 2.07-2.14 (m, |
| | | 2H), 1.76-1.82 (m, 1H), 1.66-1.72 |
| | | (m, 1H), 1.53-1.60 (m, 2H). |
| | | LCMS[M + H]+ = 391.3 |
| 30 | B | ¹H NMR (400 MHz, DMSO-d₆): δ |
| | | 8.81 (d, J = 8.4 Hz, 2H), 8.43 (s, |
| | | 1H), 7.87 (d, J = 8.0 Hz, 2H), |
| | | 6.06 (brd, J = 7.6 Hz, 1H), 4.35- |
| | | 4.48 (m, 1H), 4.16 (s, 3H), 2.92- |
| | | 2.99 (m, 1H), 2.55-2.61 (m, |
| | | 1H), 2.23 (s, 3H), 2.05-2.15 (m, |
| | | 2H), 1.89-1.96 (m, 1H), 1.73-1.81 |
| | | (m, 1H), 1.51-1.63 (m, 2H). |
| | | LCMS[M + H]+ = 391.3 |
| 31 | B | ¹H NMR (400 MHz, DMSO-d₆): δ |
| | | 8.40 (s, 1H), 8.23 (d, J = 8.0 Hz, |
| | | 2H), 8.19 (s, 1H), 7.89 (d, J = 8.4 |
| | | Hz, 2H), 6.36 (d, J = 7.2 Hz, 1H), |
| | | 4.37-4.45 (m, 4H), 2.95-3.03 (m, |
| | | 1H), 2.57-2.69 (m, 1H), 2.26 (s, |
| | | 3H), 2.08-2.21 (m, 2H), 1.90-1.99 |
| | | (m, 1H), 1.74-1.81 (m, 1H), 1.54- |
| | | 1.65 (m, 2H). |
| | | LCMS[M + H]+ = 391.0 |
| 32 | B | ¹H NMR (400 MHz, DMSO-d6): δ |
| | | 8.42 (s, 1H), 7.84-7.91 (m, 4H), |
| | | 7.39 (brd, J = 7.6 Hz, 1H), 4.36- |
| | | 4.45 (m, 1H), 3.68 (s, 3H), 2.97- |
| | | 3.09 (m, 1H), 2.62-2.70 (m, 1H), |
| | | 2.20 (s, 3H), 1.87-2.00 (m, 3H), |
| | | 1.70-1.78(m, 1H), 1.53-1.66 (m, |
| | | 1H), 1.32-1.43 (m, 1H). |
| | | LCMS[M + H]+ = 391.0 |
| 33 | | LCMS[M + H]+ = 391.2 (calc) |
| 34 | | LCMS[M + H]+ = 391.2 (calc) |
| 35 | | LCMS[M + H]+ = 376.2 (calc) |
| 36 | | LCMS[M + H]+ = 376.2 (calc) |
| 37 | | LCMS[M + H]+ = 377.2 (calc) |
| 38 | | LCMS[M + H]+ = 377.2 (calc) |
| 39 | A | ¹H NMR (400 MHz, DMSO-d₆): |
| | | 12.24-12.77 (m, 1H), 8.22 (d, J = |
| | | 8.0 Hz, 2H), 7.91 (d, J = 8.0 Hz, |
| | | 2H), 7.70 (d, J = 2.8 Hz, 1H), 6.84 |
| | | (d, J = 2.8 Hz, 1H), 5.48-5.56 (m, |
| | | 1H), 3.01-3.05 (m, 1H), 2.56-2.59 |
| | | (m, 1H), 2.28-2.35 (m, 1H), 2.22 |
| | | (s, 3H), 2.09-2.15 (m, 2H), 1.83- |
| | | 1.92 (m, 1H), 1.57-1.67 (m, 2H). |
| | | LCMS[M + H]+ = 377.3 |
| 40 | | LCMS[M + H]+ = 374.2 (calc) |
| 41 | | LCMS[M + H]+ = 405.2 (calc) |
| 42 | | LCMS[M + H]+ = 405.2 (calc) |
| 43 | | LCMS[M + H]+ = 405.2 (calc) |
| 44 | | LCMS[M + H]+ = 391.2 (calc) |
| 45 | | LCMS[M + H]+ = 391.2 (calc) |
| 46 | | LCMS[M + H]+ = 391.2 (calc) |
| 47 | | LCMS[M + H]+ = 377.2 (calc) |
| 48 | | LCMS[M + H]+ = 391.2 (calc) |
| 49 | | LCMS[M + H]+ = 377.2 (calc) |
| 50 | | LCMS[M + H]+ = 391.2 (calc) |
| 51 | | LCMS[M + H]+ = 377.2 (calc) |
| 52 | | LCMS[M + H]+ = 391.2 (calc) |
| 53 | | ¹H NMR (400 MHz, DMSO-d₆): δ |
| | | 8.40 (d, J = 2.0 Hz, 1H), 8.19 (d, J = |
| | | 8.0 Hz, 2H), 7.89 (d, J = 8.4 Hz, |
| | | 2H), 7.37 (d, J = 2.0 Hz, 1H), 7.26 |
| | | (br d, J = 8.0 Hz, 1H), 4.35-4.42 |
| | | (m, 1H), 2.96-3.01 (m, 1H), 2.60- |
| | | 2.68 (m, 1H), 2.20 (s, 3H), 1.91- |
| | | 1.99 (m, 3H), 1.70-1.76 (m, 1H), |
| | | 1.57-1.63 (m, 1H), 1.43-1.50 (m, 1H). |
| | | LCMS[M + H]+ = 377.2 |
| 54 | | ¹H NMR (400 MHz, DMSO-d₆): δ |
| | | 8.43 (d, J = 8.4 Hz, 2H), 8.25 (d, J = |
| | | 2.8 Hz, 1H), 7.91 (d, J = 8.4 Hz, |
| | | 2H), 7.35 (d, J = 2.0 Hz, 1H), 7.31 |
| | | (brd, J = 7.6 Hz, 1H), 4.32-4.42 |
| | | (m, 1H), 2.99-3.06 (m, 1H), 2.63- |
| | | 2.70 (m, 1H), 2.20 (s, 3H), 1.86- |
| | | 2.07 (m, 3H), 1.71-1.82 (m, 1H), |
| | | 1.53-1.63 (m, 1H), 1.31-1.43 (m, 1H). |
| | | LCMS[M + H]+ = 377.0 |
| 55 | | LCMS[M + H]+ = 377.2 (calc) |
| 56 | | LCMS[M + H]+ = 377.2 (calc) |
| 57 | | LCMS[M + H]+ = 378.2 (calc) |
| 58 | | ¹H NMR (400 MHz, DMSO-d₆): δ |
| | | 8.04 (d, J = 8.4 Hz, 2H), 7.83 (d, J = |
| | | 8.4 Hz, 2H), 6.16-6.33 (m, 1H), |
| | | 4.73 (t, J = 8.8 Hz, 2H), 4.13-4.35 |
| | | (m, 1H), 3.49 (t, J = 9.2 Hz, 2H), |
| | | 2.81-2.89 (m, 1H), 2.53-2.62 (m, |
| | | 1H), 2.18 (s, 3H), 1.89-2.04 (m, |
| | | 2H), 1.75-1.84 (m, 1H), 1.62-1.72 |
| | | (m 1H), 1.35-1.59 (m, 2H). |
| | | LCMS[M + H]+ = 379.0 |
| 59 | | ¹H NMR (400 MHz, DMSO-d₆) δ |
| | | 8.27 (d, J = 8.0 Hz, 2H), 7.81 (d, J = |
| | | 8.4 Hz, 2H), 6.44 (d, J = 7.6 Hz, |
| | | 1H), 4.76 (t, J = 9.2 Hz, 2H), 4.20- |
| | | 4.34 (m, 1H), 3.08 (t, J = 9.2 Hz, |
| | | 2H), 2.93-3.01 (m, 1H), 2.60-2.69 |
| | | (m, 1H), 2.19 (s, 3H), 1.78-2.00 |
| | | (m, 3H), 1.63-1.76 (m, 1H), 1.49- |
| | | 1.62 (m, 1H), 1.26-1.40 (m, 1H). |
| | | LCMS[M + H]+ = 379.0 |
| 60 | | LCMS[M + H]+ = 380.2 (calc) |
| 61 | | ¹H NMR (400 MHz, DMSO-d₆): δ |
| | | 8.18 (d, J = 5.2 Hz, 1H), 8.03 (d, J = |
| | | 8.4 Hz, 2H), 7.90 (d, J = 8.4 Hz, |
| | | 2H), 7.62 (d, J = 5.2 Hz, 1H), 7.14 |
| | | (d, J = 8.0 Hz, 1H), 4.38-4.48 (m, |
| | | 1H), 3.02-3.08 (m, 1H), 2.72-2.75 |
| | | (m, 1H), 2.21 (s, 3H), 1.89-2.02 |
| | | (m, 3H), 1.70-1.78 (m, 1H), 1.58- |
| | | 1.65 (m, 1H), 1.41-1.48 (m, 1H). |
| | | LCMS[M + H]+ = 393.2 |
| 62 | | ¹H NMR (400 MHz, DMSO-d₆): δ |
| | | 8.18 (d, J = 8.4 Hz, 2H), 8.15 (d, J = |
| | | 5.6 Hz, 1H), 7.99 (d, J = 5.2 Hz, |
| | | 1H), 7.93 (d, J = 8.4 Hz, 2H), 7.20 |
| | | (brd, J = 8.0 Hz, 1H), 4.36-4.46 |
| | | (m, 1H), 3.04-3.09 (m, 1H), 2.66- |
| | | 2.72 (m, 1H), 2.20 (s, 3H), 1.97- |
| | | 2.03 (m, 1H), 1.88-1.95 (m, 2H), |
| | | 1.72-1.78 (m, 1H), 1.54-1.66 (m, |
| | | 1H), 1.34-1.45 (m, 1H). |
| | | LCMS[M + H]+ = 393.2 |
| 63 | E | ¹H NMR (400 MHz, DMSO-d₆): |
| | | δ 9.30 (s, 1H), 8.13 (d, J = 8.0 Hz, |
| | | 1H), 7.89-7.94 (m, 3H), 4.36-4.46 |
| | | (m, 1H), 3.00-3.14 (m, 1H), 2.65- |
| | | 2.75 (m, 1H), 2.21 (s, 3H), 1.85- |
| | | 2.10 (m, 3H), 1.70-1.80 (m, 1H), |
| | | 1.51-1.68 (m, 1H), 1.32-1.49 (m, 1H). |
| | | LCMS[M + H]+ = 394.2 |
| 64 | E | ¹H NMR (400 MHz, DMSO-d₆): δ |
| | | 9.60 (s, 1H), 8.19 (d, J = 8.0 Hz, |
| | | 2H), 7.34-7.99 (m, 3H), 4.42-4.54 |
| | | (m, 1H), 3.02-3.11 (m, 1H), 2.65- |
| | | 2.74 (m, 1H), 2.22 (s, 3H), 1.92- |
| | | 2.07 (m, 3H), 1.74-1.82 (m, 1H), |
| | | 1.55-1.68 (m, 1H), 1.37-1.48 (m, 1H). |
| | | LCMS[M + H]+ = 394.1 |
| 65 | | ¹H NMR (400 MHz, DMSO-d₆): δ |
| | | 7.95 (d, J = 8.0 Hz, 2H), 7.82 (d, J = |
| | | 8.4 Hz, 2H), 6.16 (d, J = 8.0 Hz, |
| | | 1H), 4.13-4.40 (m, 1H), 3.01-3.07 |
| | | (m, 2H), 2.93-2.98 (m, 1H), 2.70- |
| | | 2.77 (m, 2H), 2.64-2.68 (m, 1H), |
| | | 2.18 (s, 3H), 2.02-2.08 (m, 2H), |
| | | 1.82-1.94 (m, 3H), 1.50-1.74 (m, |
| | | 2H), 1.31-1.41 (m, 1H). |
| | | LCMS[M + H]+ = 377.4 |
| 66 | A | ¹H NMR (400 MHz, DMSO-d₆): δ |
| | | 11.83 (brs, 1H), 7.85-7.92 (m, |
| | | 2H), 7.80 ((d, J = 8.4 Hz, 1H), 7.45 |
| | | (d, J = 3.2 Hz, 1H), 6.88 (d, J = 2.8 |
| | | Hz, 1H), 6.69 (d, J = 8.0 Hz, 1H), |
| | | 4.31-4.41 (m, 1H), 3.02-3.08 (m, |
| | | 1H), 2.67-2.71 (m, 1H), 2.55-2.57 |
| | | (m, 3H), 2.19 (s, 3H), 1.83-2.01 |
| | | (m, 3H), 1.70-1.78 (m, 1H), 1.53- |
| | | 1.66 (m, 1H), 1.31-1.42 (m, 1H). |
| | | LCMS[M + H]+ = 390.0 |
| 67 | B | ¹H NMR (400 MHz, DMSO-d₆): δ |
| | | 9.62 (brs, 1H), 7.27-7.32 (m, 2H), |
| | | 7.20-7.29 (m, 1H), 6.91-6.98 (m, |
| | | 2H), 6.76 (d, J = 2.8 Hz, 1H), 6.43 |
| | | (d, J = 8.0 Hz, 1H), 4.26-4.36 (m, |
| | | 1H), 3.39 (s, 3H), 3.01-3.07 (m, |
| | | 1H), 2.55-2.59 (m, 1H), 2.20 (s, |
| | | 3H), 1.83-1.97 (m, 3H), 1.71- |
| | | 1.77 (m, 1H), 1.55-1.64 (m, 1H), |
| | | 1.32-1.41 (m, 1H). |
| | | LCMS[M + H]+ = 338.3 |
| 68 | C | ¹H NMR (400 MHz, DMSO-d₆): δ |
| | | 13.07 (brs, 1H), 8.01-8.06 (m, |
| | | 1H), 7.59 (d, J = 2.8 Hz, 1H), 7.21- |
| | | 7.36 (m, 1H), 6.89-7.03 (m, 2H), |
| | | 6.73 (d, J = 3.2 Hz, 1H), 4.52-4.70 |
| | | (m, 1H), 3.19 (s, 3H), 2.87-2.93 |
| | | (m, 1H), 2.70-2.79 (m, 1H), 2.22 |
| | | (s, 3H), 2.13 (t, J = 10.4 Hz, 1H), |
| | | 1.62-1.86 (m, 5H). |
| | | LCMS[M + H]+ = 338.3 |
| 69 | B | 1H NMR (400 MHz, DMSO-d₆): δ |
| | | 7.86 (d, J = 8.4 Hz, 2H), 7.78 (d, J = |
| | | 8.4 Hz, 2H), 7.40 (d, J = 3.2 Hz, |
| | | 1H), 6.86 (d, J = 3.2 Hz, 1H), 6.63 |
| | | (d, J = 8.0 Hz, 1H), 4.29-4.38 (m, |
| | | 1H), 3.39 (s, 3H), 3.01-3.06 (m, |
| | | 1H), 2.66-2.71 (m, 1H), 2.19 (s, |
| | | 3H), 1.85-1.98 (m, 3H), 1.70-1.76 |
| | | (m, 1H), 1.55-1.64 (m, 1H), 1.32- |
| | | 1.42 (m, 1H). |
| | | LCMS[M + H]+ = 390.0 |
| 70 | A | ¹H NMR (400 MHz, DMSO-d₆) δ |
| | | 11.71 (brs, 1H), 7.78-7.91 (m, |
| | | 2H), 7.50-7.58 (m, 2H), 7.43-7.49 |
| | | (m, 1H), 7.40 (d, J = 2.8 Hz, 1H), |
| | | 6.86 (d, J = 3.2 Hz, 1H), 6.55 (d, J = |
| | | 7.6 Hz, 1H), 4.22-4.46 (m, 1H), |
| | | 2.99-3.11 (m, 1H), 2.63-2.69 (m, |
| | | 1H), 2.20 (s, 3H), 1.94-2.02 (m, |
| | | 1H), 1.81-1.93 (m, 2H), 1.69-1.79 |
| | | (m, 1H), 1.54-1.67 (m, 1H), 1.30- |
| | | 1.39 (m, 1H). |
| | | LCMS[M + H]+ = 308.3 |
| 71 | D | ¹H NMR (400 MHz, DMSO-d₆): δ |
| | | 7.76-7.84 (m, 4H), 7.50 (d, J = 2.4 |
| | | Hz, 1H), 6.69 (d, J = 2.4 Hz, 1H), |
| | | 4.56-4.69 (m, 1H), 3.37 (s, 3H, |
| | | overlapped with water peak |
| | | single), 3.18 (s, 3H), 2.87-2.93 (m, |
| | | 1H), 2.70-2.77 (m, 1H), 2.21 (s, |
| | | 3H), 2.10-2.17 (m, 1H), 1.59-1.85 |
| | | (m, 5H). |
| | | LCMS[M + H]+ = 404.2 |
| 72 | C | ¹H NMR (400 MHz, DMSO-d₆): δ |
| | | 11.94 (brs, 1H), 8.07 (d, J = 8.0 |
| | | Hz, 2H), 7.90 (d, J = 8.4 Hz, 2H), |
| | | 7.53 (d, J = 2.8 Hz, 1H), 6.71-6.77 |
| | | (m, 1H), 4.58-4.76 (m, 1H), 3.22 |
| | | (s, 3H), 2.87-2.92 (m, 1H), 2.74- |
| | | 2.78 (m, 1H), 2.22 (s, 3H), 2.14 (t, |
| | | J = 10.8 Hz, 1H), 1.76-1.85 (m, |
| | | 2H), 1.74-1.79 (m, 1H), 1.60-1.70 |
| | | (m, 2H). |
| | | LCMS[M + H]+ = 390.4 |
| 73 | A | ¹H NMR (400 MHz, DMSO-d₆): δ |
| | | 8.20 (s, 2H), 7.54-7.63 (m, 2H), |
| | | 7.27-7.33 (m, 3H), 6.47-6.58 (m, |
| | | 1H), 4.33-4.37 (m, 1H), 2.98-3.04 |
| | | (m, 1H), 2.58-2.66 (m, 1H), 2.14- |
| | | 2.20 (m, 5H), 1.85-1.90(m, 4H), |
| | | 1.76-1.82 (m, 1H), 1.57-1.64 (m, |
| | | 1H), 1.40-1.48 (m, 1H). |
| | | LCMS[M + H]+ = 340.1 |
| 74 | A | ¹H (400 MHz, DMSO-d₆): δ |
| | | 11.40-11.60 (m, 1H), 8.20 (s, |
| | | 1.5H), 7.83-7.89 (m, 2H), 7.31- |
| | | 7.40 (m, 2H), 7.21 (s, 1H), 5.51- |
| | | 5.58 (m, 1H), 4.35-4.42 (m, 1H), |
| | | 2.75-2.81 (m, 1H), 2.58-2.62 (m, |
| | | 1H), 2.45-2.59 (m, 5H), 2.32-2.35 |
| | | (m, 1H), 2.21-2.27 (m, 4H), 1.72- |
| | | 1.77 (m, 1H), 1.58-1.62 (m, 1H). |
| | | LCMS[M + H]+ = 340.1 |
| 75 | A | ¹H NMR (400 MHz, DMSO-d₆): δ |
| | | 11.55 (brs, 1H), 8.22 (s, 0.6H), |
| | | 8.06 (d, J = 8.0 Hz, 2H), 7.88 (d, J = |
| | | 8.0 Hz, 2H), 6.56 (s, 1H), 6.51 |
| | | (brd, J = 8.0 Hz, 1H), 4.26-4.37 |
| | | (m, 1H), 3.03-3.08 (m, 1H), 2.67- |
| | | 2.72 (m, 1H), 2.43 (s, 3H), 2.22 |
| | | (s, 3H), 1.87-1.98 (m, 3H), 1.71- |
| | | 1.77 (m, 1H), 1.55-1.64 (m, 1H), |
| | | 1.33-1.42 (m, 1H). |
| | | LCMS[M + H]+ = 390.1 |
| 76 | B | ¹H NMR (400 MHz, DMSO-d₆): δ |
| | | 8.12 (d, J = 8.0 Hz, 2H), 7.86 (d, J = |
| | | 8.0 Hz, 2H), 7.53 (d, J = 2.0 Hz, |
| | | 1H), 6.66 (d, J = 2.4 Hz, 1H), 5.90 |
| | | (brd, J = 7.6 Hz, 1H), 4.33-4.43 |
| | | (m, 1H), 4.16 (s, 3H), 2.92- |
| | | 3.004 (m, 1H), 2.53-2.61 (m, 1H), |
| | | 2.34-2.41 (m, 2H), 2.13-2.28 (m, |
| | | 2H), 1.84-1.94 (m, 1H), 1.71-1.79 |
| | | (m, 1H), 1.50-1.65 (m, 2H), 1.03 |
| | | (t, J = 7.2 Hz, 3H). |
| | | LCMS[M + H]+ = 404.2 |
| 77 | B | ¹H NMR (400 MHz, DMSO-d₆): δ |
| | | 8.12 (d, J = 8.0 Hz, 2H), 7.86 (d, J = |
| | | 8.0 Hz, 2H), 7.52-7.56 (m, |
| | | 1H), 6.64-6.67 (m, 1H), 5.94 (brd, |
| | | J = 7.6 Hz, 1H), 4.33-4.50 (m, 1H), |
| | | 4.17 (s, 3H), 2.71-2.94 (m, 2H), |
| | | 2.54-2.60 (m, 1H), 2.36-2.46 (m, |
| | | 2H), 1.47-1.86 (m, 4H), 0.88-1.05 |
| | | (m, 6H). |
| | | LCMS[M + H]+ = 418.2 |
| 78 | B | ¹H NMR (400 MHz, DMSO-d₆): δ |
| | | 8.18 (brs, 1H), 8.08-8.14 (m, 1 H), |
| | | 7.66 (d, J = 3.2 Hz, 1H), 7.05 (d, J = |
| | | 3.2 Hz, 1H), 6.71-6.78 (m, 2H), |
| | | 6.02 (brd, J = 7.6 Hz, 1H), 4.26- |
| | | 4.33 (m, 1H), 4.19 (s, 3H), 2.86- |
| | | 3.00 (m, 1H), 2.53-2.63 (m, 1H, |
| | | overlap with DMSO singal), 2.12- |
| | | 2.27 (m, 5H), 1.85-1.95 (m, 1H), |
| | | 1.72-1.81 (m, 1H), 1.52-1.64 (m, 2H). |
| | | LCMS[M + H]+ = 356.0 |
| 79 | B | ¹H NMR (400 MHz, DMSO-d₆: δ |
| | | 8.07-8.15 (m, 2H), 7.92-8.01 (m, |
| | | 2H), 7.51-7.57 (m, 1H), 6.63-6.68 |
| | | (m, 1H), 5.90 (brd, J = 8.0 Hz, 1H), |
| | | 4.32-4.44 (m, 1H), 4.15 (s, 3H), |
| | | 2.85-3.04 (m, 1H), 2.56 (m, 1H), |
| | | 2.20 (s, 3H), 2.01-2.16 (m, 2H), |
| | | 1.83-1.96 (m, 1H), 1.66-1.79 (m, |
| | | 1H), 1.46-1.64 (m, 2H). |
| | | LCMS[M + H]+ = 347.2 |
| 80 | B | ¹H NMR (400 MHz, DMSO-d₆): δ |
| | | 7.92 (d, J = 8.4 Hz, 2H), 7.56 (d, J = |
| | | 8.8 Hz, 2H), 7.50 (d, J = 2.8 Hz, |
| | | 1H), 6.61 (d, J = 2.8 Hz, 1H), 5.79 |
| | | (brd, J = 7.2 Hz, 1H), 4.28-4.42 |
| | | (m, 1H), 4.14 (s, 3H), 2.86-2.99 |
| | | (m, 1H), 2.51-2.59 (m, 1H), 2.21 |
| | | (s, 3H), 2.03-2.18 (m, 2H), 1.85- |
| | | 1.96 (m, 1H), 1.69-1.79 (m, 1H), |
| | | 1.43-1.64 (m, 2H). |
| | | LCMS[M + H]+ = 356.2 |
| 81 | D | ¹H NMR (400 MHz, DMSO-d₆): δ |
| | | 15.32 (brs, 1H), 8.11-8.20 (m, |
| | | 1H), 7.81 (d, J = 3.2 Hz, 1H), 7.14 |
| | | (d, J = 3.2 Hz, 1H), 6.73-6.87 (m, |
| | | 2H), 4.14 (s, 3H), 3.49-3.59 (m, |
| | | 1H), 2.89 (s, 3H), 2.74-2.83 (m, |
| | | 1H), 2.59-2.67 (m, 1H), 2.01-2.18 |
| | | (m, 4H), 1.74-1.91 (m, 2H), 1.63- |
| | | 1.73 (m, 1H), 1.41-1.59 (m, 2H). |
| | | LCMS[M + H]+ = 370.0 |
| 82 | D | ¹H NMR (400 MHz, DMSO-d₆): δ |
| | | 7.99 (d, J = 8.4 Hz, 2H), 7.67 (d, J = |
| | | 3.2 Hz, 1H), 7.61 (d, J = 8.4 Hz, |
| | | 2H), 6.76 (d, J = 2.8 Hz, 1H), 4.11 |
| | | (s, 3H), 3.51-3.59 (m, 1H), 2.88 (s, |
| | | 3H), 2.78-2.85 (m, 1H), 2.61-2.67 |
| | | (m, 1H), 2.01-2.14 (m, 4H), 1.74- |
| | | 1.90 (m, 2H), 1.62-1.71 (m, 1H), |
| | | 1.43-1.57 (m, 2H). |
| | | LCMS[M + H]+ = 370.0 |
| 83 | A | ¹H NMR (DMSO-d₆, 400 MHz): δ |
| | | 8.28 (d, J = 8.0 Hz, 2H), 7.93 (d, J = |
| | | 8.4 Hz, 2H), 7.86 (d, J = 3.2 Hz, |
| | | 1H), 6.90 (d, J = 2.8 Hz, 1H), 3.03- |
| | | 3.16 (m, 2H), 2.61-2.72 (m, 2H), |
| | | 2.07-2.23 (m, 4H), 1.70-1.91 (m, |
| | | 2H), 1.56-1.67 (m, 2H), 1.34-1.47 |
| | | (m, 1H), 0.98-1.08 (m, 1H). |
| | | LCMS[M + H]+ = 375.0 |
| 84 | D | ¹H NMR (400 MHz, DMSO-d₆): δ |
| | | 8.17 (d, J = 8.0 Hz, 2H), 8.01 (d, J = |
| | | 8.0 Hz, 2H), 7.72 (s, 1H), 6.80 (s, |
| | | 1H), 4.11 (s, 3H), 3.54-3.57 (m, |
| | | 1H), 2.90 (s, 3H), 2.75-2.85 (m, |
| | | 1H), 2.63-2.66 (m, 1H), 2.05-2.18 |
| | | (m, 4H), 1.76-1.93 (m, 2H), 1.66- |
| | | 1.69 (m, 1H), 1.47-1.52 (m, 2H). |
| | | LCMS[M + H]+ = 361.2 |
| 85 | B | ¹H NMR (400 MHz, DMSO-d₆): |
| | | δ 8.16 (d, J = 7.2 Hz, 2H), 7.90 (d, |
| | | J = 7.6 Hz, 2H), 7.61-7.75 (m, |
| | | 1H), 6.60-6.80 (m, 1H), 5.43-5.66 |
| | | (m, 1H), 4.10 (s, 3H), 2.88- |
| | | 2.99 (m, 1H), 2.33-2.48 (m, 2H), |
| | | 1.96-2.31 (m, 5H), 1.76-1.88 (m, |
| | | 1H), 1.50-1.68 (m, 2H). |
| | | LCMS[M + H]+ = 391.2 |
| 86 | | ¹H NMR (400 MHz, DMSO-d₆): δ |
| | | 10.83 (brs, 1H), 8.21 (d, J = 5.6 |
| | | Hz, 1H), 7.47-7.59 (m, 1H), 7.40 |
| | | (d, J = 5.6 Hz, 1H), 6.74-6.93 (m, |
| | | 2H), 5.41-5.55 (m, 1H), 2.89-3.06 |
| | | (m, 1H), 2.54-2.59 (m, 1H), 2.29- |
| | | 2.38 (m, 1H), 2.06-2.26 (m, 5H), |
| | | 1.74-1.86 (m, 1H), 1.51-1.66 (m, 2H). |
| | | LCMS[M + H]+ = 359.9 |
| 87 | | ¹H NMR (400 MHz, DMSO-d₆): δ |
| | | 8.34 (d, J = 5.2 Hz, 1H), 8.08 (d, J = |
| | | 8.4 Hz, 2H), 7.95 (d, J = 8.4 Hz, |
| | | 2H), 7.72 (d, J = 5.2 Hz, 1H), 5.44- |
| | | 5.60 (m, 1H), 2.95-3.04 (m, 1 H), |
| | | 2.52-2.56 (m, 1H), 2.30-2.37 (m, |
| | | 1H), 2.03-2.25 (m, 5H), 1.73-1.88 |
| | | (m, 1H), 1.55-1.66 (m, 2H). |
| | | LCMS[M + H]+ = 394.2 |
| 88 | | ¹H NMR (400 MHz, DMSO-d₆): δ |
| | | 8.47 (s, 1H), 8.23 (d, J = 8.0 Hz, |
| | | 2H), 7.94 (d, J = 8.4 Hz, 2H), 7.45 |
| | | (d, J = 1.6 Hz, 1H), 5.48-5.56 (m, |
| | | 1H), 2.95-3.03 (m, 1H), 2.56-2.59 |
| | | (m, 1H), 2.28-2.39 (m, 1H), 2.07- |
| | | 2.25 (m, 5H), 1.76-1.92 (m, 1H), |
| | | 1.55-1.71 (m, 2H). |
| | | LCMS[M + H]+ = 378.2 |
| 89 | B | ¹H NMR (400 MHz, DMSO-d₆): δ |
| | | 8.05-8.20 (m,1H), 7.73 (s, 1H), |
| | | 7.07 (s, 1H), 6.75-6.84 (m, 2H), |
| | | 5.29-5.45 (m, 1H), 4.09 (s, 3H), |
| | | 2.82-2.91 (m, 1H), 2.38-2.45 (m, |
| | | 2H), 2.13-2.24 (m, 4H), 1.98-2.08 |
| | | (m, 1H), 1.73-1.86 (m, 1H), 1.54- |
| | | 1.69 (m, 2H). |
| | | LCMS[M + H]+ = 361.2 |
| 90 | A | ¹H NMR (, 400 MHz, DMSO-d₆): δ |
| | | 817-8.25 (m, 1H), 7.80 (s, 1H), |
| | | 7.19 (s, 1H), 6.78-6.85 (m, 2H), |
| | | 5.38-5.52 (m, 1H), 2.82-3.00 (m, |
| | | 1H), 2.30-2.41 (m, 2H), 2.11-2.25 |
| | | (m, 4H), 2.02-2.10 (m, 1H), 1.77- |
| | | 1.93 (m, 1H), 1.53-1.67 (m, 2H). |
| | | LCMS[M + H]+ = 361.2 |
| 91 | | ¹H NMR (400 MHz, DMSO-d₆): δ |
| | | 12.98 (brs, 1H), 8.46 (s, 1H), 7.89- |
| | | 7.96 (m, 1H), 7.47 (s, 1H), 6.64- |
| | | 7.02 (m, 2H), 5.41-5.52 (m, 1H), |
| | | 2.91-3.04 (m, 1H), 2.54-2.62 (m, |
| | | 1H), 2.30-2.43 (m, 1H), 2.03-2.27 |
| | | (m, 5H), 1.75-1.87 (m, 1H) 1.56- |
| | | 1.70 (m, 2H). |
| | | LCMS[M + H]+ = 344.1 |
| 92 | | ¹H NMR (400 MHz, CDCl₃): δ: |
| | | 9.07 (d, J = 8.4 Hz, 2H), 7.83 (s, |
| | | 1H), 7.76 (d, J = 8.0 Hz, 2H), 6.21 |
| | | (brd, J = 7.6 Hz, 1H), 4.87-4.96 |
| | | (m, 1H), 3.80-3.85 (m, 1H), 3.45- |
| | | 3.50 (m, 1H), 3.23-3.26 (m, 4H), |
| | | 3.17 (s, 3H), 3.05-3.13 (m, 1H), |
| | | 2.05-2.17 (m, 2H), 1.91-1.98 (m, |
| | | 1H), 1.65-1.73 (m, 1H) |
| | | LCMS[M + H]+ = 391.3 |
| 93 | | ¹H NMR (400 MHz, DMSO-d₆): δ |
| | | 8.40 (s, 1H), 8.17 (d, J = 7.6 Hz, |
| | | 2H), 7.89 (d, J = 8.0 Hz, 2H), 7.35 |
| | | (s, 1H), 4.77-4.90 (m, 1H), 3.27 (s, |
| | | 3H), 2.84-2.93 (m, 1H), 2.70-2.80 |
| | | (m, 1H), 2.08-2.25 (m, 4H), 1.57- |
| | | 1.85 (m, 5H). |
| | | LCMS[M + H]+ = 391.0 |
| 94 | C | ¹H NMR (400 MHz, DMSO-d₆): δ |
| | | 8.40-8.53 (m, 2H), 8.32 (s, 1H), |
| | | 7.87 (d, J = 8.0 Hz, 2H), 5.45- |
| | | 5.57 (m, 1H), 3.38 (s, 3H), 2.90- |
| | | 2.94 (m, 1H), 2.75-2.79 (m, 1H), |
| | | 2.25 (s, 3H), 2.19-2.24 (m, 1H), |
| | | 1.77-1.97 (m, 3H), 1.65-1.73 (m, 2H). |
| | | LCMS[M + H]+ = 391.3 |
| 95 | | ¹H NMR (400 MHz, DMSO-d₆) δ |
| | | 8.25 (d, J = 5.6 Hz, 1H), 8.03 (d, J = |
| | | 8.0 Hz, 2H), 7.92 (d, J = 8.0 Hz, |
| | | 2H), 7.63 (d, J = 5.2 Hz, 1H), 4.62- |
| | | 4.71 (m, 1H), 3.28 (s, 3H), 2.89- |
| | | 2.97 (m, 1H), 2.71-2.79 (m, 1H), |
| | | 2.18-2.24 (m, 4H), 1.62-1.85 (m, 5H). |
| | | LCMS[M + H]+ = 407.0 |
| 96 | C | ¹H NMR (400 MHz, DMSO-d₆): δ |
| | | 8.17 (dd, J = 9.6, 6.8 Hz, 1H), 7.75 |
| | | (d, J = 3.2 Hz, 1H), 7.16 (d, J = 3.2 |
| | | Hz, 1H), 6.71-6.82 (m, 2H), 4.28- |
| | | 4.39 (m, 1H), 3.16 (s, 3H), 2.83- |
| | | 2.87 (m, 1H), 2.69-2.75 (m, 1H), |
| | | 2.17 (s, 3H), 2.03-2.12 (m, 1H), |
| | | 1.56-1.89 (m, 5H). |
| | | LCMS[M + H]+ = 356.0 |
| 97 | B | ¹H NMR (400 MHz, DMSO-d₆): δ |
| | | 8.19 (s, 0.71H), 7.83 (d, J = 8.0 Hz, |
| | | 2H), 7.72 (d, J = 8.0 Hz, 2H), 7.24 |
| | | (s, 1H), 5.68-5.86 (m, 1H), 4.30- |
| | | 4.41 (m, 1H), 4.08 (s, 3H), 2.94- |
| | | 2.98 (m, 1H), 2.55-2.60 (m, 1H), |
| | | 2.24 (s, 3H), 2.11-2.20 (m, 2H), |
| | | 1.88-1.94 (m, 1H), 1.80 (s, 3H), |
| | | 1.70-1.77 (m, 1H), 1.51-1.62 (m, 2H). |
| | | LCMS[M + H]+ = 404.3 |
| 98 | | ¹H NMR (400 MHz, DMSO-d₆): δ |
| | | 10.49-11.51 (m, 1H), 8.14 (d, J = |
| | | 5.2 Hz, 1H), 7.48-7.57 (m, 1H), |
| | | 7.37 (d, J = 5.6 Hz, 1H), 6.75-6.87 |
| | | (m, 2H), 4.53-4.65 (m, 1H), 3.24 |
| | | (s, 3H), 2.88-2.98 (m, 1H), 2.71- |
| | | 2.78 (m, 1H), 2.16-2.28 (m, 4H), |
| | | 1.59-1.86 (m, 5H). |
| | | LCMS[M + H]+ = 373.0 |
| 99 | B | ¹H NMR (400 MHz, DMSO-d₆): δ |
| | | 8.22 (d, J = 8.0 Hz, 1H), 7.69 (d, J = |
| | | 2.4 Hz, 1H), 7.27-7.41 (m, 2H), |
| | | 7.06 (d, J = 2.8 Hz, 1H), 6.23 (brd, |
| | | J = 7.6 Hz, 1H), 4.31-4.40 (m, 1H), |
| | | 4.21 (s, 3H), 2.72-2.88 (m, 2H), |
| | | 2.52-2.61 (m, 1H), 2.32-2.46 (m, |
| | | 2H), 1.63-1.85 (m, 3H), 1.48-1.57 |
| | | (m, 1H), 0.98 (t, J = 5.6 Hz, 6H). |
| | | LCMS[M + H]+ = 391.1 |
| 100 | B | ¹H NMR (400 MHz, DMSO-d₆): δ |
| | | 8.23 (d, J = 7.2 Hz, 1H), 7.70 (s, |
| | | 1H), 7.28-7.44 (m, 2H), 7.07 (s, |
| | | 1H), 6.15 (brd, J = 4.4 Hz, 1H), |
| | | 4.32-4.36 (m, 1H), 4.20 (s, 3H), |
| | | 2.84-2.89 (m, 1H), 2.64-2.67 (m, |
| | | 1H), 2.12-2.31 (m, 5H), 1.86-1.95 |
| | | (m, 1H), 1.70-1.81 (m, 1H), 1.52- |
| | | 1.63 (m, 2H). |
| | | LCMS[M + H]+ = 363.0 |
| 101 | B | ¹H NMR (400 MHz, DMSO-d₆): δ |
| | | 8.22 (s, 0.70H), 8.11 (d, J = 8.0 Hz, |
| | | 2H), 7.85 (d, J = 8.4 Hz, 2H), 6.53 |
| | | (s, 1H), 5.88 (brs, 1H), 4.35-4.45 |
| | | (m, 1H), 3.98 (s, 3H), 2.99-3.06 |
| | | (m, 1H), 2.61-2.69 (m, 1H), 2.43 |
| | | (s, 3H), 2.29 (s, 3H), 2.13-2.27 (m, |
| | | 2H), 1.90-1.98 (m, 1H), 1.74-1.81 |
| | | (m, 1H), 1.52-1.66 (m, 2H). |
| | | LCMS[M + H]+ = 404.3 |
| 102 | D | ¹H NMR (400 MHz, DMSO-d₆): δ |
| | | 8.25 (d, J = 8.0 Hz, 1H), 8.20 (s, |
| | | 0.2H), 7.84 (d, J = 2.8 Hz, 1H), |
| | | 7.37-7.46 (m, 2H), 7.13 (d, J = 2.8 |
| | | Hz, 1H), 4.14 (s, 3H), 3.55-3.64 |
| | | (m, 1H), 2.93 (s, 3H), 2.78-2.83 |
| | | (m, 1H), 2.65-2.69 (m, 1H), 2.08- |
| | | 2.15 (m, 4H), 1.80-1.91 (m, 2H), |
| | | 1.65-1.72 (m, 1H), 1.49-1.61 (m, 2H). |
| | | LCMS[M + H]+ = 377.1 |
| 103 | B | ¹H NMR (400 MHz, DMSO-d₆): δ |
| | | 8.22 (d, J = 7.2 Hz, 1H), 7.70 (s, |
| | | 1H), 7.28-7.42 (m, 2H), 7.07 (s, |
| | | 1H), 6.19 (brd, J = 7.6 Hz, 1H), |
| | | 4.30-4.39 (m, 1H), 4.20 (s, 3H), |
| | | 2.81-2.99 (m, 2H), 2.18-2.35 (m, |
| | | 3H), 1.45-1.95 (m, 5H), 1.02 (t, J = |
| | | 6.8 Hz, 3H). |
| | | LCMS[M + H]+ = 377.1 |
| 104 | B | ¹H NMR (400 MHz, DMSO-d₆): |
| | | δ 10.25-11.10 (m, 1H), 8.36 (s, |
| | | 1H), 7.54 (d, J = 8.0 Hz, 1H), 7.44 |
| | | (d, J = 7.6 Hz, 1H), 7.31-7.41 (m, |
| | | 2H), 4.26-4.40 (m, 1H), 3.67 (s, |
| | | 3H), 2.96-3.08 (m, 1H), 2.53-2.54 |
| | | (m, 1H), 2.20 (s, 3H), 1.85-2.04 |
| | | (m, 3H), 1.70-1.80 (m, 1H), 1.52- |
| | | 1.65 (m, 1H), 1.30-1.46 (m, 1H). |
| | | LCMS[M + H]+ = |
| 105 | D | ¹H NMR (400 MHz, CDCl₃): δ 8.81 |
| | | (d, J = 8.0 Hz, 2H), 8.04 (s, 1H), |
| | | 7.79 (d, J = 8.4 Hz, 2H), |
| | | 4.17 (s, 3H), 3.57-3.68 (m, 1H), |
| | | 3.09 (s, 3H), 2.95-3.02 (m, 1H), |
| | | 2.79-2.86 (m, 1H), 2.26-2.37 (m, |
| | | 4H), 1.93-2.02 (m, 2H), 1.80-1.86 |
| | | (m, 1H), 1.65-1.72 (m, 2H). |
| | | LCMS[M + H]+ = 405.3 |
| 106 | D | ¹H NMR (400 MHz, CDCl₃): δ 7.85 |
| | | (s, 1H), 7.77 (d, J = 8.4 Hz, |
| | | 2H), 7.72 (d, J = 8.4 Hz, 2H), 5.55- |
| | | 5.65 (m, 1H), |
| | | 3.52 (s, 3H), 3.38 (s, 3H), 3.05- |
| | | 3.01 (m, 1H), 2.86-2.92 (m, |
| | | 1H), 3.34 (s, 3H), 2.60-2.29 (m, |
| | | 2H), 1.90-1.99 (m, 2H), 1.83-1.86 |
| | | (m, 1H), 1.66-1.76 (m, 1H). |
| | | LCMS[M + H]+ = 405.3 |
| 107 | | ¹H NMR (400 MHz, DMSO-d₆): δ |
| | | 8.45 (s, 1H), 8.19 (s, 0.5H), 7.92- |
| | | 8.01 (m, 1H), 7.52 (s, 1H), 6.76- |
| | | 6.86 (m, 2H), 4.72-4.84 (m, 1H), |
| | | 3.25 (s, 3H), 2.88-2.93 (m, 1H), |
| | | 2.72-2.78 (m, 1H), 2.13-2.26 (m, |
| | | 4H), 1.59-1.91 (m, 5H). |
| | | LCMS[M + H]+ = 357.0 |
| 108 | B | ¹H NMR (400 MHz, DMSO-d₆): δ |
| | | 8.39 (s, 1H), 8.22 (d, J = 8.0 Hz, |
| | | 2H), 7.88 (d, J = 8.0 Hz, 2H), 6.37 |
| | | (brd, J = 7.6 Hz, 1H), 4.33-4.48 |
| | | (m, 4H), 2.93-3.15 (m, 1H), 2.71- |
| | | 2.79 (m, 1H), 2.16-2.41 (m, 4H), |
| | | 1.90-1.96 (m, 1H), 1.73-1.83 (m, |
| | | 1H), 1.53-1.70 (m, 2H), 0.77-1.05 |
| | | (m, 1H), 0.37-0.55 (m, 2H), 0.05- |
| | | 0.15 (m, 2H). |
| | | LCMS[M + H]+ = 431.3 |
| 109 | B | ¹H NMR (400 MHz, DMSO-d₆): δ |
| | | 8.38 (s, 1H), 8.19-8.24 (m, 2H), |
| | | 7.84-7.92 (m, 2H), 6.24-6.44 (m, |
| | | 1H), 4.52-4.59 (m, 4H), 3.00-3.18 |
| | | (m, 1H), 2.52-2.73 (m, 1H), 2.41- |
| | | 2.45 (m, 2H), 1.91-1.98 (m, 1H), |
| | | 1.68-1.73 (m, 3H), 1.45-1.57 (m, |
| | | 1H), 0.22-0.52 (m, 4H). |
| | | LCMS[M + H]+ = 417.1 |
| 110 | B | ¹H NMR (400 MHz, DMSO-d₆): |
| | | δ 7.90 (s, 1H), 7.77 (s, 1H), 7.68 |
| | | (d, J = 8.0 Hz, 1 H), 7.61 (d, J = 8.0 |
| | | Hz, 1H), 6.25-6.35 (m, 1H), 4.30- |
| | | 4.53 (m, 4H), 2.91-3.12 (m, 1H), |
| | | 2.48-2.49 (m, 1H), 2.06-2.35 (m, |
| | | 8H), 1.90-2.00 (m, 1H), 1.73-1.85 |
| | | (m, 1H), 1.52-1.59 (m, 2H), 1.46- |
| | | 1.70 (m, 1H). |
| | | LCMS[M + H]+ = 405.0 |
| 111 | B | ¹H NMR (400 MHz, DMSO-d₆): |
| | | δ 8.26 (s, 1H), 7.98 (d, J = 8.0 Hz, |
| | | 1H), 7.90 (s, 1H), 7.75 (d, J = 8.0 |
| | | Hz, 1H), 6.35 (brd, J = 7.6 Hz, |
| | | 1H), 4.33-4.42 (m, 4H), 2.90-2.98 |
| | | (m, 1H), 2.52-2.57 (m, 1H), 2.22 |
| | | (s, 3H), 2.03-2.15 (m, 2H), 1.88- |
| | | 1.99 (m, 1H), 1.72-1.80 (m, 1H), |
| | | 1.52-1.62 (m, 2H). |
| | | LCMS[M + H]+ = 382 |
| 112 | B | ¹H NMR (400 MHz, DMSO-d₆): |
| | | δ 10.25-11.10 (m, 1H), 8.36 (s, |
| | | 1H), 7.54 (d, J = 8.0 Hz, 1H), 7.44 |
| | | (d, J = 7.6 Hz, 1H), 7.31-7.41 (m, |
| | | 2H), 4.26-4.40 (m, 1H), 3.67 (s, |
| | | 3H), 2.96-3.08 (m, 1H), 2.53-2.54 |
| | | (m, 1H), 2.20 (s, 3H), 1.85-2.04 |
| | | (m, 3H), 1.70-1.80 (m, 1H), 1.52- |
| | | 1.65 (m, 1H), 1.30-1.46 (m, 1H). |
| | | LCMS[M + H]+ = 362.5 |
| 113 | B | LCMS[M + H]+ = 375.2 (calc) |
| 114 | B | ¹H NMR (400 MHz, DMSO-d₆): δ |
| | | 14.92 (brs, 1H), 8.70 (s, 1H), 8.24 |
| | | (d, J = 8.0 Hz, 1H), 7.42 (s, 1H), |
| | | 7.36 (d, J = 8.0 Hz, 1H), 6.57 (brd, |
| | | J = 7.6 Hz, 1H), 4.35-4.41 (m, 4H), |
| | | 2.87-2.95 (m, 1H), 2.53-2.60 (m, |
| | | 1H), 2.23 (s, 3H), 2.06-2.20 (m, |
| | | 2H), 1.88-1.96 (m, 1H), 1.73-1.81 |
| | | (m, 1H), 1.55-1.64 (m, 2H). |
| | | LCMS[M + H]+ = 364.20 |
| 115 | B | ¹H NMR (400 MHz, DMSO-d₆): |
| | | δ 10.19 (brs, 1H), 7.68 (s, 1H), |
| | | 7.28 (s, 1H), 7.14 (s, 1H), 6.18 |
| | | (brd, J = 7.6 Hz, 1H), 4.35-4.41 |
| | | (m, 1H), 4.33 (s, 3H), 2.92-3.01 |
| | | (m, 1H), 2.55-2.63 (m, 1H), 2.22 |
| | | (s, 3H), 2.04-2.17 (m, 2H), 2.02 (s, |
| | | 3H), 1.89-1.98 (m, 1H), 1.71-1.83 |
| | | (m, 1H), 1.48-1.66 (m, 2H). |
| | | LCMS[M + H]+ = 378.1 |
| 116 | B | LCMS[M + H]+ = 420.2 (calc) |
| 117 | B | ¹H NMR (400 MHz, DMSO-d₆) δ |
| | | 8.30 (s, 1 H), 8.17 (d, J = 8.0 Hz, |
| | | 1H), 8.07 (s, 1H), 7.94 (d, J = 8.0 |
| | | Hz, 1H), 7.33 (t, J = 55.2 Hz, 1H), |
| | | 6.42 (brd, J = 7.6 Hz, 1H), 4.40- |
| | | 4.48 (m, 1H), 4.37 (s, 3H), 2.91- |
| | | 2.96 (m, 1H), 2.52-2.56 (m, 1H), |
| | | 2.22 (s, 3H), 2.01-2.18 (m, 2H), |
| | | 1.89-1.98 (m, 1H), 1.71-1.83 (m, |
| | | 1H), 1.50-1.64 (m, 2H). |
| | | LCMS[M + H]+ = 398.2 |
| 118 | B | ¹H NMR (400 MHz, DMSO-d₆): δ |
| | | 8.04 (s, 1H), 7.84 (s, 1H), 7.73- |
| | | 7.80 (m, 2H), 7.27 (t, J = 55.2 Hz, |
| | | 1H), 6.33 (brd, J = 7.6 Hz, 1H), |
| | | 4.31-4.44 (m, 4H), 2.91-3.01 (m, |
| | | 1H), 2.54-2.63 (m, 1H), 2.22 (s, |
| | | 3H), 2.04-2.14 (m, 2H), 1.89-1.98 |
| | | (m, 1H), 1.71-1.83 (m, 1H), 1.50- |
| | | 1.54 (m, 2H). |
| | | LCMS[M + H]+ = 407.1 |
| 119 | B | ¹H NMR (400 MHz, DMSO-d₆): |
| | | δ 8.04-8.15 (m, 3H), 7.97 (d, J = |
| | | 8.0 Hz, 1 H), 7.36 (t, J = 55.2 Hz, |
| | | 1H), 6.40 (brd, J = 7.6 Hz, |
| | | 1H), 4.33-4.45 (m, 4H), 2.85-3.05 |
| | | (m, 1H), 2.51-2.64 (m, 1H), 2.22 |
| | | (s, 3H), 2.00-2.17 (m, 2H), 1.88- |
| | | 1.98 (m, 1H), 1.71-1.82 (m, 1H), |
| | | 1.47-1.66 (m, 2H). |
| | | LCMS[M + H]+ = 441.1 |
| 120 | B | ¹H NMR (400 MHz, DMSO-d₆): δ |
| | | 8.27 (s, 1H), 8.14 (d, J = 8.0 Hz, |
| | | 1H), 7.85 (d, J = 8.0 Hz, 1H), 7.52 |
| | | (d, J = 2.8 Hz, 1H), 7.29 (t, J = 54.8 |
| | | Hz, 1H), 6.34 (d, J = 3.2 Hz, 1H), |
| | | 5.94 (brd, J = 7.6 Hz, 1H), 4.34- |
| | | 4.42 (m, 1H), 4.16 (s, 3H), 2.57- |
| | | 2.59 (m, 1H), 2.50-2.51 (m, 1H), |
| | | 2.21 (s, 3H), 2.06-2.17 (m, 2H), |
| | | 1.87-1.94 (m, 1H), 1.70-1.78 (m, |
| | | 1H), 1.52-1.62 (m, 2H). |
| | | LCMS[M + H]+ = 397.3 |
| 121 | B | ¹H NMR (400 MHz, DMSO-d₆): δ |
| | | 7.81 (d, J = 2.0 Hz, 1H), 7.73-7.77 |
| | | (m, 1H), 7.65-7.68 (m, 1H), 7.49 |
| | | (d, J = 2.8 Hz, 1H), 7.20 (t, J = 55.2 |
| | | Hz, 1H), 6.30 (d, J = 2.8 Hz, 1H), |
| | | 5.86 (brd, J = 7.6 Hz, 1H), 4.34- |
| | | 4.40 (m, 1H), 4.15 (s, 3H), 2.88- |
| | | 2.96 (m, 1H), 2.52-2.58 (m, 1H), |
| | | 2.21 (s, 3H), 2.05-2.16 (m, 2H), |
| | | 1.87-1.95 (m, 1H), 1.69-1.77 (m, |
| | | 1H), 1.51-1.61 (m, 2H). |
| | | LCMS[M + H]+ = 406.3 |
| 122 | B | ¹H NMR (400 MHz, DMSO-d₆): δ |
| | | 8.20 (s, 1H), 8.05-8.07 (m, 2H), |
| | | 7.90 (d, J = 8.0 Hz, 1H), 7.52 (d, J = |
| | | 2.8 Hz, 1H), 7.31 (t, J = 55.2 Hz, |
| | | 1H), 6.36 (d, J = 2.8 Hz, 1H), 5.95- |
| | | 6.10 (m, 1H), 4.40-4.51 (m, 1H), |
| | | 4.17 (s, 3H), 3.00-3.02 (m, 1H), |
| | | 2.64-2.66 (m, 1H), 2.22-2.29 (m, |
| | | 5H), 1.91-1.95 (m, 1H), 1.77-1.80 |
| | | (m, 1H), 1.58-1.63 (m, 2H). |
| | | LCMS[M + H]+ = 440.0 |
| 123 | B | ¹H NMR (400 MHz, DMSO-d6): δ |
| | | 7.74 (s, 1H), 7.66 (d, J = 8.0 Hz, |
| | | 1H), 7.55 (d, J = 8.0 Hz, 1H), 7.44 |
| | | (d, J = 2.8 Hz, 1H), 6.13 (d, J = 2.8 |
| | | Hz, 1H), 5.79 (brd, J = 7.6 Hz, 1H), |
| | | 4.31-4.40 (m, 1 H), 4.15 (s, 3H), |
| | | 2.89-2.99 (m, 1H), 2.51-2.53 (m, |
| | | 1H), 2.28 (s, 3H), 2.23 (s, 3H), |
| | | 2.05-2.18 (m, 2H), 2.89-2.97 (m, |
| | | 1H), 1.72-1.78 (m, 1H), 1.50-1.62 |
| | | (m, 2H). |
| | | LCMS[M + H]+ = 404.4 |
| 124 | B | ¹H NMR (400 MHz, DMSO-d₆): δ |
| | | 8.21 (s, 1H), 7.95 (dd, J = 8.8, 0.8 |
| | | Hz, 1H), 7.68 (d, J = 8.0 Hz, 1H), |
| | | 7.44 (d, J = 2.8 Hz, 1H), 6.12 (d, J = |
| | | 2.8 Hz, 1H), 5.87 (brd, J = 7.6 |
| | | Hz, 1H), 4.34-4.39 (m, 1H), 4.14 |
| | | (s, 3H), 2.90-2.95 (m, 1H), 2.54- |
| | | 2.57 (m, 1H), 2.22 (s, 3H), 2.09- |
| | | 2.17 (m, 2H), 1.88-1.92 (m, 1H), |
| | | 1.72-1.77 (m, 1H), 1.51-1.59 (m, 2H). |
| | | LCMS[M + H]+ = 381.3 |
| 125 | B | ¹H NMR (400 MHz, DMSO-d₆): δ |
| | | 8.21 (s, 1.4H), 7.85 (s, 1H), 7.76 |
| | | (d, J = 9.2 Hz, 1H), 7.52 (d, J = 8.0 |
| | | Hz, 1H), 7.46 (d, J = 2.8 Hz, 1H), |
| | | 6.12 (d, J = 2.8 Hz, 1H), 5.88-5.90 |
| | | (m, 1H), 4.35-4.45 (m, 1H), 4.17 |
| | | (s, 3H), 3.06-3.08 (m, 1H), 2.63- |
| | | 2.70 (m, 1H), 2.28-2.33 (m, 5H), |
| | | 2.24 (s, 3H), 1.93-1.95 (m, 1H), |
| | | 1.78-1.81 (m, 1H), 1.56-1.67 (m, 2H). |
| | | LCMS[M + H]+ = 361.0 |
| 126 | B | ¹H NMR (400 MHz, DMSO-d₆): δ: |
| | | 8.21 (s, 1H), 7.66 (s, 2H), 7.41 (d, |
| | | J = 2.8 Hz, 1H), 5.90 (d, J = 3.2 Hz, |
| | | 1H), 5.73-5.87 (m, 1H), 4.35-4.45 |
| | | (m, 1H), 4.14 (s, 3H), 2.95-3.05 |
| | | (m, 1H), 2.63-2.67 (m, 1H), 2.30 |
| | | (s, 3H), 2.16-2.27 (m, 2H), 1.94- |
| | | 1.96 (m, 7H), 1.76-1.81 (m, 1H), |
| | | 1.53-1.63 (m, 2H). |
| | | LCMS[M + H]+ = 375.1 |
| 127 | B | ¹H NMR (400 MHz, DMSO-d₆): δ |
| | | 9.98 (brs, 1H), 7.34 (d, J = 3.2 Hz, |
| | | 1H), 7.24 (s, 1H), 7.11 (s, 1H), |
| | | 5.93 (d, J = 2.8 Hz, 1H), 5.71 (brd, |
| | | J = 7.6 Hz, 1H), 4.31-4.37 (m, 1H), |
| | | 4.12 (s, 3H), 2.90-2.98 (m, 1H), |
| | | 2.60-2.62 (m, 1H), 2.22 (s, 3H), |
| | | 2.06-2.14 (m, 2H), 1.96 (s, 3H), |
| | | 1.88-1.93 (m, 1H), 1.71-1.77 (m, |
| | | 1H), 1.52-1.62 (m, 2H). |
| | | LCMS[M + H]+ = 377.1 |
| 128 | B | LCMS[M + H]+ = 419.2 (calc) |
| 129 | B | ¹H NMR (400 MHz, DMSO-d₆): δ |
| | | 9.08 (s, 1H), 8.63 (d, J = 8.8 Hz, |
| | | 1H), 8.29 (dd, J = 8.4, 2.4 Hz, 1H), |
| | | 7.52 (d, J = 2.8 Hz, 1H), 7.28 |
| | | (d, J = 2.8 Hz, 1H), 6.01 (brd, J = |
| | | 7.6 Hz, 1H), 4.40-4.46 (m, 1H), |
| | | 4.16 (s, 3H), 2.90-2.96 (m, 1H), |
| | | 2.52-2.54 (m, 1H), 2.22 (s, 3H), |
| | | 2.10-2.18 (m, 2H), 1.89-1.94 (m, |
| | | 1H), 1.72-1.77 (m, 1H), 1.54-1.61 |
| | | (m, 2H). |
| | | LCMS[M + H]+ = 391.3 |
| 130 | B | LCMS[M + H]+ = 404.2 (calc) |
| 131 | B | ¹HNMR (400 MHz, DMSO-d₆): δ |
| | | 9.07 (d, J = 1.2 Hz, 1H), 8.62 (s, |
| | | 1H), 7.46 (d, J = 2.8 Hz, 1H), 6.31 |
| | | (d, J = 2.8 Hz, 1H), 5.94 (brd, J = |
| | | 8.0 Hz, 1H), 4.35-4.46 (m, 1H), |
| | | 4.15 (s, 3H), 2.88-2.97 (m, 1H), |
| | | 2.54-2.57 (m, 1H), 2.22 (s, 3H), |
| | | 2.05-2.19 (m, 2H), 1.87-1.95 (m, |
| | | 1H), 1.71-1.82 (m, 1H), 1.51-1.64 |
| | | (m, 2H). |
| | | LCMS[M + H]+ = 425.0 |
| 132 | B | ¹H NMR (400 MHz, DMSO-d₆): δ |
| | | 8.96 (s, 1H), 8.32 (s, 1H), 7.44 |
| | | (d, J = 2.8 Hz, 1H), 6.48 (d, J = 2.8 |
| | | Hz, 1H), 5.92 (brd, J = 7.6 Hz, 1H), |
| | | 4.34-4.46 (m, 1H), 4.14 (s, 3H), |
| | | 2.86-2.96 (m, 1H), 2.52-2.57 (m, |
| | | 1H), 2.48 (s, 3H), 2.21 (s, 3H), |
| | | 2.07-2.18 (m, 2H), 1.86-1.95 (m, |
| | | 1H), 1.71-1.78 (m, 1H), 1.51-1.62 |
| | | (m, 2H). |
| | | LCMS[M + H]+ = 362.3 |
| 133 | B | LCMS[M + H]+ = 381.2 (calc) |
| 134 | B | LCMS[M + H]+ = 406.2 (calc) |
| 135 | B | ¹H NMR (400 MHz, DMSO-d₆): δ |
| | | 8.57 (d, J = 1.6 Hz, 1H), 8.17 (s, |
| | | 0.5H), 7.83 (d, J = 2.0 Hz, 1H), |
| | | 7.66 (d, J = 3.2 Hz, 1H), 7.49 (d, J = |
| | | 2.8 Hz, 1H), 6.32 (brd, J = 7.6 |
| | | Hz, 1H), 4.36-4.42 (m, 1H), 4.21 |
| | | (s, 3H), 2.94-3.00 (m, 1H), 2.59- |
| | | 2.66 (m, 1H), 2.32-2.37 (m, 1H), |
| | | 2.30 (s, 3H), 2.20-2.27 (m, 1H), |
| | | 1.87-1.94 (m, 1H), 1.76-1.82 (m, |
| | | 1H), 1.59-1.65 (m, 2H). |
| | | LCMS[M + H]+ = 364.1 |
| 136 | B | ¹H NMR (400 MHz, DMSO-d₆): δ |
| | | 9.14 (s, 1H), 8.71 (s, 1H), 8.65 (d, |
| | | J = 8.4 Hz, 1H), 8.32-8.38 (m, 1H), |
| | | 6.50 (brd, J = 7.6 Hz, 1H), 4.42- |
| | | 4.53 (m, 1H), 4.37 (s, 3H), 2.89- |
| | | 3.04 (m, 1H), 2.55-2.63 (m, 1H), |
| | | 2.23 (s, 4H), 2.03-2.12 (m, 1H), |
| | | 1.89-1.99 (m, 1H), 1.74-1.82 (m, |
| | | 1H), 1.49-1.67 (m, 2H). |
| | | LCMS[M + H]+ = 392.2 |
| 137 | B | ¹H NMR (400 MHz, DMSO-d₆): δ |
| | | 8.96 (s, 1H), 8.20-8.29 (m, 2H), |
| | | 6.38 (d, J = 7.6 Hz, 1H), 4.39-4.50 |
| | | (m, 1H), 4.35 (s, 3H), 2.88-3.00 |
| | | (m, 1H), 2.66 (s, 3H), 2.54-2.62 |
| | | (m, 1H), 2.02-2.26 (m, 5H), 1.88- |
| | | 1.97 (m, 1H), 1.71-1.82 (m, 1H), |
| | | 1.51-1.63 (m, 2H). |
| | | LCMS[M + H]+ = 406.1 |
| 138 | B | ¹H NMR (400 MHz, DMSO-d₆): δ |
| | | 9.11 (s, 1H), 8.66 (s, 1H), 8.09 (s, |
| | | 1H), 6.45 (brd, J = 7.6 Hz, 1H), |
| | | 4.42-4.49 (m, 1H), 4.36 (s, 3H), |
| | | 2.91-2.98 (m, 1H), 2.55-2.60 (m, |
| | | 1H), 2.23 (s, 3H), 2.14-2.20 (m, |
| | | 1H), 2.05-2.12 (m, 1H), 1.91-1.98 |
| | | (m, 1H), 1.73-1.80 (m, 1H), 1.55- |
| | | 1.64 (m, 2H). |
| | | LCMS[M + H]+ = 426.1 |
| 139 | B | ¹H NMR (400 MHz, DMSO-d₆): δ |
| | | 9.01 (d, J = 1.6 Hz, 1H), 8.35 (d, J = |
| | | 1.2 Hz, 1H), 8.24 (s, 1H), 6.42 |
| | | (brd, J = 7.6 Hz, 1H), 4.41-4.48 |
| | | (m, 1H), 4.35 (s, 3H), 2.87-2.96 |
| | | (m, 1H), 2.63 (s, 3H), 2.55-2.59 |
| | | (m, 1H), 2.22 (s, 3H), 2.04-2.18 |
| | | (m, 2H), 1.89-1.97 (m, 1H), 1.73- |
| | | 1.80 (m, 1H), 1.54-1.63 (m, 2H). |
| | | LCMS[M + H]+ = 363.2 |
| 140 | B | ¹H NMR (400 MHz, DMSO-d₆): δ |
| | | 9.15 (d, J = 1.6 Hz, 1H), 8.79 (d, J = |
| | | 1.6 Hz, 1H), 8.10 (s, 1H), 6.48 |
| | | (brd, J = 8.0 Hz, 1H), 4.45-4.48 |
| | | (m, 1H), 4.37 (s, 3H), 2.93-2.94 |
| | | (m, 1H), 2.33-2.44 (m, 1H), 2.24 |
| | | (s, 3H), 2.07-2.18 (m, 2H), 1.94- |
| | | 1.97 (m, 1H), 1.75-1.81 (m, 1H), |
| | | 1.58-1.76 (m, 2H). |
| | | LCMS[M + H]+ = 383.1 |
| 141 | B | LCMS[M + H]+ = 407.2 (calc) |
| 142 | B | LCMS[M + H]+ = 364.2 (calc) |
| 143 | B | ¹H NMR (400 MHz, DMSO-d₆): δ |
| | | 9.51-10.42 (m, 1H), 8.22 (s, 1H), |
| | | 7.34 (d, J = 2.8 Hz, 1H), 7.12 (s, |
| | | 1H), 7.08 (s, 1H), 5.95 (d, J = 2.8 |
| | | Hz, 1H), 5.68-5.78 (m, 1H), 4.34- |
| | | 4.40 (m, 1H), 4.13 (s, 3H), 2.97- |
| | | 3.05 (m, 1H), 2.53-2.55 (m, 1H), |
| | | 2.26 (s, 3H), 2.12-2.21 (m, 2H), |
| | | 2.00 (s, 3H), 1.91-1.97 (m, 1H), |
| | | 1.73-1.79 (m, 1H), 1.51-1.63 (m, 2H). |
| | | LCMS[M + H]+ = 420.3 |
| 144 | E | ¹H NMR (400 MHz, DMSO-d₆): δ |
| | | 11.31 (brs, 1H), 9.02 (s, 1H), 7.91 |
| | | (d, J = 7.2 Hz, 1H), 7.75 (brd, J = |
| | | 8.0 Hz, 1H), 7.43 (d, J = 8.0 Hz, |
| | | 1H), 7.38 (s, 1H), 4.30-4.45 (m, |
| | | 1H), 3.01-3.10 (m, 1H), 2.64-2.73 |
| | | (m, 1H), 2.21 (s, 3H), 1.86-2.06 |
| | | (m, 3H), 1.69-1.82 (m, 1H), 1.47- |
| | | 1.69 (m, 1H), 1.28-1.47 (m, 1H). |
| | | LCMS[M + H]+ = 367.0 |
| 145 | B | ¹H NMR (400 MHz, DMSO-d₆): δ |
| | | 8.22 (d, J = 8.0 Hz, 1H), 7.72 (d, J = |
| | | 2.8 Hz, 1H), 7.32-7.43 (m, 2H), |
| | | 7.07 (s, 1H), 6.25 (brd, J = 8.8 Hz, |
| | | 1H), 4.56-4.65 (m, 1H), 4.18 (s, |
| | | 3H), 2.83-3.02 (m, 2H), 2.28-2.43 |
| | | (m, 6H, overlapped with DMSO |
| | | peak), 2.08-2.24 (m, 1H). |
| | | LCMS[M + H]+ = 399.10 |
| 146 | B | ¹H NMR (400 MHz, DMSO-d6): δ |
| | | 8.21 (d, J = 8.4 Hz, 1H), 7.69 (d, J = |
| | | 3.2 Hz, 1H), 7.30-7.37 (m, 2H), |
| | | 7.06 (d, J = 3.2 Hz, 1H), 6.11 (brd, |
| | | J = 7.6 Hz, 1H), 4.25-4.30 (m, 1H), |
| | | 4.17 (s, 3H), 2.96-3.00 (m, 1H), |
| | | 2.61-2.72 (m, 1H), 2.51-2.56 (m, |
| | | 1H), 2.32-2.49 (m, 1H), 1.79-1.89 |
| | | (m, 1H), 1.62-1.73 (m, 3H), 1.47- |
| | | 1.52 (m, 1H), 0.30-0.48 (m, 4H). |
| | | LCMS[M + H]+ = 389.2 |
| 147 | B | ¹H NMR (400 MHz, DMSO-d₆): δ |
| | | 8.23 (d, J = 2.0 Hz, 1H), 8.21 (s, |
| | | 0.5H), 7.70 (d, J = 3.2 Hz, 1H), |
| | | 7.20-7.40 (m, 2H), 7.07 (d, J = 3.2 |
| | | Hz, 1H), 6.26 (brd, J = 7.6 Hz, 1H), |
| | | 4.32-4.41 (m, 1H), 4.21 (s, 3H), |
| | | 3.50-3.55 (m, 2H), 2.80-2.89 (m, |
| | | 1H), 2.52-2.60 (m, 1H), 2.25-2.48 |
| | | (m, 4H), 1.63-1.86 (m, 3H), 1.50- |
| | | 1.61 (m, 1H). |
| | | LCMS[M + H]+ = 393.0 |
| 148 | B | ¹H NMR (400 MHz, DMSO-d₆): δ |
| | | 15.74 (brs, 1H), 8.23 (d, J = 8.4 |
| | | Hz, 1H), 7.70 (d, J = 3.2 Hz, 1H), |
| | | 7.37 (d, J = 1.6 Hz, 1H), 7.33 (dd, J = |
| | | 8.0, 1.6 Hz, 1H), 7.08 (d, J = 3.6 |
| | | Hz, 1H), 6.23 (brd, J = 8.0 Hz, 1H), |
| | | 4.49-4.63 (m, 2H), 4.34-4.39 (m, |
| | | 1H), 4.20 (s, 3H), 2.90-2.96 (m, |
| | | 1H), 2.62-2.76 (m, 3H), 2.3-2.46 |
| | | (m, 2H), 1.59-1.86 (m, 4H). |
| | | LCMS[M + H]+ = 395.3 |
| 149 | B | ¹H NMR (400 MHz, DMSO-d₆): δ |
| | | 8.22 (d, J = 8.0 Hz, 1H), 7.71 (d, J = |
| | | 2.4 Hz, 1H), 7.30-7.44 (m, 2H), |
| | | 7.07 (d, J = 1.6 Hz, 1H), 6.27 (brd, |
| | | J = 8.0 Hz, 1H), 4.48-4.67 (m, 3H), |
| | | 4.17 (s, 3H), 2.61-3.10 (m, 6H), |
| | | 2.19-2.43 (m, 2H). |
| | | LCMS[M + H]+ = 431.10 |
| 150 | E | ¹H NMR (400 MHz, DMSO-d₆): δ |
| | | 11.30-11.33 (m, 1H), 9.03 (s, 1H), |
| | | 7.92 (d, J = 7.2 Hz, 1H), 7.76 (d, J = |
| | | 8.0 Hz, 1H), 7.44 (brd, J = 8.0 |
| | | Hz, 1H), 7.40 (s, 1H), 4.47-4.63 |
| | | (m, 2H), 4.32-4.42 (m, 1H), 3.32- |
| | | 3.40 (m, 1H), 2.80-2.90 (m, 1H), |
| | | 2.65-2.75 (m, 2H), 1.95-2.10 (m, |
| | | 3H), 1.70-1.85 (m, 1H), 1.55-1.68 |
| | | (m, 1H), 1.35-1.50 (m, 1H). |
| | | LCMS[M + H]+ = 399.0 |
| 151 | E | ¹H NMR (400 MHz, DMSO-d₆): δ |
| | | 11.16-11.53 (m, 1H), 9.03 (s, 1H), |
| | | 7.89 (d, J = 7.6 Hz, 1H), 7.76 (d, J = |
| | | 8.0 Hz, 1H), 7.44 (d, J = 8.0 Hz, |
| | | 1H), 7.39 (s, 1H), 4.34-4.45 (m, |
| | | 2H), 3.50-3.57 (m, 2H), 2.73-3.17 |
| | | (m, 2H), 2.43-2.48 (m, 2H), |
| | | overlapped with DMSO-d₆ peak), |
| | | 1.98-2.07 (m, 3H), 1.71-1.78 (m, |
| | | 1H), 1.53-1.64 (m, 1H), 1.37-1.47 |
| | | (m, 1H). |
| | | LCMS[M + H]+ = 397.0 |
| 152 | B | ¹H NMR (400 MHz, DMSO-d₆): δ |
| | | 15.81 (brs, 0.8H), 8.23 (d, J = 8.0 |
| | | Hz, 1H), 7.71 (d, J = 3.2 Hz, 1H), |
| | | 7.37 (d, J = 1.6 Hz, 1H), 7.30-7.35 |
| | | (m, 1H), 7.08 (d, J = 3.2 Hz, 1H), |
| | | 6.31 (brd, J = 7.6 Hz, 1H), 4.36- |
| | | 4.43 (m, 1H), 4.24 (s, 3H), 4.06- |
| | | 4.15 (m, 1H), 2.79-2.87 (m, 1H), |
| | | 2.65-2.71 (m, 1H), 2.54-2.62 (m, |
| | | 2H), 2.24 (s, 2H), 1.68-1.81 (m, |
| | | 3H), 1.49-1.57 (m, 1H), 1.10 (d, J = |
| | | 2.8 Hz, 6H). |
| | | LCMS[M + H]+ = 421.2 |
| 153 | B | ¹H NMR (400 MHz, DMSO-d₆): δ |
| | | 8.23 (d, J = 8.4 Hz, 1H), 7.70 (d, J = |
| | | 3.2 Hz, 1H), 7.25-7.41 (m, 2H), |
| | | 7.07 (d, J = 3.2 Hz, 1H), 6.24 (brd, |
| | | J = 7.2 Hz, 1H), 4.28-4.39 (m, 1H), |
| | | 4.21 (s, 3H), 3.42-3.48 (m, 2H), |
| | | 3.23 (s, 3H), 2.79-2.81 (m, 1H), |
| | | 2.51-2.67 (m, 3H), 2.30-2.41 (m, |
| | | 2H), 1.50-1.83 (m, 4H). |
| | | LCMS[M + H]+ = 407.1 |
| 154 | E | ¹H NMR (400 MHz, DMSO-d₆): δ |
| | | 9.00 (brs, 1H), 7.82 (d, J = 7.2 Hz, |
| | | 1H), 7.69 (d, J = 7.6 Hz, 1H), 7.22- |
| | | 7.35 (m, 2H), 4.30-4.40 (m, 1H), |
| | | 3.50-3.57 (m, 2H, overlapped |
| | | with water peak), 3.23 (s, 3H), |
| | | 3.08-3.16 (m, 1H), 2.80-2.87 (m, |
| | | 1H), 2.52-2.56 (m, 2H, |
| | | overlapped with DMSO-d₆ peak), |
| | | 1.91-2.08 (m, 3H), 1.70-1.80 (m, |
| | | 1H), 1.52-1.63 (m, 1H), 1.3-1.39 |
| | | (m, 1H). |
| | | LCMS[M + H]+ = 411.2 |
| 155 | B | ¹H NMR (400 MHz, DMSO-d₆): δ |
| | | 8.23 (d, J = 8.0 Hz, 1H), 7.70 (d, J = |
| | | 3.2 Hz, 1H), 7.29-7.39 (m, 2H), |
| | | 7.07 (d, J = 3.2 Hz, 1H), 6.23 (brd, |
| | | J = 8.0 Hz, 1H), 4.29-4.41 (m, 1H), |
| | | 4.22 (s, 3H), 2.74-2.84 (m, 1H), |
| | | 2.26-2.49 (m, 7H), 2.13 (s, 6H), |
| | | 1.65-1.85 (m, 3H), 1.49-1.59 (m, 1H). |
| | | LCMS[M + H]+ = 406.1 |
| 156 | B | ¹H NMR (400 MHz, DMSO-d₆): δ |
| | | 8.23 (d, J = 8.0 Hz, 1H), 7.70 (d, J = |
| | | 2.8 Hz, 1H), 7.37 (d, J = 2.0 Hz, |
| | | 1H), 7.30-7.35 (m, 1H), 7.07 (d, J = |
| | | 2.8 Hz, 1H), 6.21 (brd, J = 7.6 |
| | | Hz, 1H), 4.29-4.41 (m, 1H), 4.22 |
| | | (s, 3H), 2.79-2.87 (m, 1H), 2.55- |
| | | 2.60 (m, 2H), 2.37-2.48 (m, 4H), |
| | | 2.31-2.36 (m, 1H), 2.28 (s, 3H), |
| | | 1.65-1.85 (m, 3H), 1.51-1.59 (m, 1H). |
| | | LCMS[M + H]+ = 420.2 |
| 157 | B | ¹H NMR (400 MHz, DMSO-d₆): δ |
| | | 10.43 (brs, 1H), 7.60 (s, 1H), 7.40 |
| | | (d, J = 8.0 Hz, 1H), 7.33 (dd, J = |
| | | 8.8, 1.2 Hz, 1H), 7.23 (d, J = 1.2 |
| | | Hz, 1H), 5.88 (brd, J = 7.6 Hz, 1H), |
| | | 4.27-4.34 (m, 1H), 4.10 (s, 3H), |
| | | 2.87-2.96 (m, 1H), 2.52-2.56 (m, |
| | | 1H, overlapped with DMSO-d₆ |
| | | signal peak), 2.21 (s, 3H), 2.06- |
| | | 2.15 (m, 2H), 1.87-1.93 (m, 1H), |
| | | 1.71-1.77 (m, 1H), 1.51-1.60 (m, 2H). |
| | | LCMS[M + H]+ = 397.0 |
| 158 | B | ¹H NMR (400 MHz, DMSO-d₆): δ |
| | | 8.24 (d, J = 8.0 Hz, 1H), 7.71 (d, J = |
| | | 2.8 Hz, 1H), 7.37 (s, 1H), 7.33 |
| | | (d, J = 8.4 Hz, 1H), 7.08 (d, J = 2.8 |
| | | Hz, 1H), 6.17 (brd, J = 8.0 Hz, 1H), |
| | | 4.15-4.30 (m, 4H), 3.12-3.16 (m, |
| | | 1H), 2.76-2.81 (m, 1H), 2.59-2.65 |
| | | (m, 2H), 1.96-2.03 (m, 1H), 1.65- |
| | | 1.73 (m, 2H), 1.45-1.52 (m, 1H). |
| | | LCMS[M + H]+ = 349.0 |
| 159 | B | ¹H NMR (400 MHz, DMSO-d₆): δ |
| | | 8.14 (d, J = 8.0 Hz, 2H), 7.86 (d, J = |
| | | 8.4 Hz, 2H), 7.56 (d, J = 3.2 Hz, |
| | | 1H), 6.69 (d, J = 2.8 Hz, 1H), 5.95 |
| | | (brd, J = 7.2 Hz, 1H), 4.31-4.45 |
| | | (m, 1H), 4.17 (s, 3H), 3.86-3.95 |
| | | (m, 1H), 3.47-3.54 (m, 1H, |
| | | overlapped with water peak), |
| | | 2.90 (s, 3H), 2.77-2.88 (m, 2H), |
| | | 2.01-2.13 (m, 1H), 1.82-1.96 (m, |
| | | 1H), 1.57-1.77 (m, 2H). |
| | | LCMS[M + H]+ = 454.3 |
| 160 | B | ¹H NMR (400 MHz, DMSO-d₆): δ |
| | | 15.73 (brs, 1H), 8.23 (d, J = 8.4 |
| | | Hz, 1H), 7.71 (d, J = 2.8 Hz, 1H), |
| | | 7.37 (d, J = 1.6 Hz, 1H), 7.30-7.35 |
| | | (m, 1H), 7.07 (d, J = 3.2 Hz, 1H), |
| | | 6.23 (brd, J = 7.6 Hz, 1H), 4.52- |
| | | 4.57 (m, 2H), 4.43-4.49 (m, 2H), |
| | | 4.33-4.41 (m, 1H), 4.23 (s, 3H), |
| | | 3.43-3.50 (m, 1H), 2.70-2.81 (m, |
| | | 1H), 2.38-2.47 (m, 1H), 2.06-2.23 |
| | | (m, 2H), 1.86-1.96 (m, 1H), 1.74- |
| | | 1.82 (m, 1H), 1.54-1.70 (m, 2H). |
| | | LCMS[M + H]+ = 405.1 |
| 161 | B | ¹H NMR (400 MHz, DMSO-d₆): δ |
| | | 8.24 (d, J = 8.0 Hz, 1H), 7.71 (d, J = |
| | | 2.8 Hz, 1H), 7.38 (d, J = 2.0 Hz, |
| | | 1H), 7.34 (dd, J = 8.0, 2.0 Hz, 1H), |
| | | 7.17-7.22 (m, 2H), 7.08 (d, J = 3.2 |
| | | Hz, 1H), 7.01 (d, J = 8.0 Hz, 2H), |
| | | 6.75 (t, J = 7.2 Hz, 1H), 6.23 (brd, |
| | | J = 7.2 Hz, 1H), 4.36-4.45 (m, 1H), |
| | | 4.18 (s, 3H), 3.85-3.91 (m, 1H), |
| | | 3.54-3.60 (m, 1H), 2.89-3.03 (m, |
| | | 2H), 2.06-2.16 (m, 1H), 1.67-1.91 |
| | | (m, 3H). |
| | | LCMS[M + H]+ = 425.1 |
| 162 | B | ¹H NMR (400 MHz, DMSO-d₆): δ |
| | | 8.25 (d, J = 8.4 Hz, 1H), 8.11 (d, J = |
| | | 6.4 Hz, 2H), 7.70 (d, J = 3.2 Hz, |
| | | 1H), 7.31-7.43 (m, 2H), 7.09 (d, J = |
| | | 3.2 Hz, 1H), 6.87 (d, J = 6.4 Hz, |
| | | 2H), 6.24 (brd, J = 6.8 Hz, 1H), |
| | | 4.22-4.37 (m, 1H), 4.05-4.18 (m, |
| | | 4H), 3.76-3.89 (m, 1H), 3.04-3.27 |
| | | (m, 2H), 2.10-2.22 (m, 1H), 1.77- |
| | | 1.92 (m, 2H), 1.56-1.71 (m, 1H). |
| | | LCMS[M + H]+ = 426.1 |
| 163 | B | ¹HNMR (400 MHz, DMSO-d₆): δ |
| | | 8.37 (d, J = 2.4 Hz, 1H), 8.23 (d, J = |
| | | 8.4 Hz, 1H), 7.96 (d, J = 4.4 Hz, |
| | | 1H), 7.71 (d, J = 2.8 Hz, 1H), 7.32- |
| | | 7.42 (m, 3H), 7.16-7.22 (m, 1H), |
| | | 7.08 (d, J = 2.8 Hz, 1H), 6.25 (brd, |
| | | J = 7.2 Hz, 1H), 4.34-4.45 (m, 1H), |
| | | 4.18 (s, 3H), 3.91-4.00 (m, 1H), |
| | | 3.64-3.72 (m, 1H), 2.93-3.07 (m, |
| | | 2H), 2.08-2.16 (m, 1H), 1.67-1.92 |
| | | (m, 3H). |
| | | LCMS[M + H]+ = 426.1 |
| 164 | B | ¹H NMR (400 MHz, DMSO-d₆): δ |
| | | 8.23 (d, J = 8.4 Hz, 1H), 7.71 (d, J = |
| | | 3.2 hz, 1H), 7.37 (d, J = 1.6 Hz, |
| | | 1H), 7.31-7.35 (m, 1H), 7.07 (d, J = |
| | | 3.2 Hz, 1H), 6.20 (brd, J = 7.6 |
| | | Hz, 1H), 4.31-4.40 (m, 1H), 4.21 |
| | | (s, 3H), 3.06-3.14 (m, 1H), 2.79- |
| | | 2.91 (m, 1H), 2.62-2.70 (m, 2H), |
| | | 2.28-2.39 (m, 4H), 1.74-1.86 (m, |
| | | 2H), 1.63-1.69 (m, 1H), 1.53-1.59 |
| | | (m, 1H). |
| | | LCMS[M + H]+ = 392.1 |
| 165 | B | ¹H NMR (400 MHz, DMSO-d₆): δ |
| | | 8.15 (d, J = 8.0 Hz, 1H), 7.38 (d, J = |
| | | 1.6 Hz, 1H), 7.32 (dd, J = 8.0, |
| | | 2.0 Hz, 1H), 7.28 (s, 1H), 6.26 |
| | | (brd, J = 7.2 Hz, 1H), 4.28-4.36 |
| | | (m, 1H), 4.10 (s, 3H), 2.87-2.95 |
| | | (m, 1H), 2.55-2.59 (m, 1H), |
| | | overlapped with DMSO-d₆ peak), |
| | | 2.22 (s, 3H), 2.07-2.18 (m, 2H), |
| | | 1.87-1.94 (m, 1H), 1.72-1.79 (m, |
| | | 1H), 1.52-1.62 (m, 2H). |
| | | LCMS[M + H]+ = 397.1 |
| 166 | B | ¹H NMR (400 MHz, DMSO-d₆): δ |
| | | 15.22 (brs, 1H), 8.80 (s, 1H), 8.59 |
| | | (s, 1H), 7.75 (s, 1H), 6.65 (brd, J = |
| | | 7.6 Hz, 1H), 4.32-4.44 (m, 4H), |
| | | 2.84-2.98 (m, 1H), 2.53-2.62 (m, |
| | | 1H), 2.00-2.27 (m, 5H), 1.86-1.98 |
| | | (m, 1H), 1.71-1.84 (m, 1H), 1.51- |
| | | 1.60 (m, 2H). |
| | | LCMS[M + H]+ = 408.1 |
| 167 | B | ¹H NMR (400 MHz, DMSO-d₆): δ |
| | | 8.21 (s, 0.6H), 8.13 (s, 1H), 7.87 |
| | | (s, 1H), 7.48 (s, 1H), 6.19 (brd, J = |
| | | 7.2 Hz, 1H), 4.27-4.41 (m, 4H), |
| | | 3.76 (s, 3H), 2.93-3.04 (m, 1H), |
| | | 2.53-2.63 (m, 1H), 2.40 (s, 3H), |
| | | 2.23 (s, 3H), 2.03-2.17 (m, 2H), |
| | | 1.88-2.01 (m, 1H), 1.70-1.81 (m, |
| | | 1H), 1.45-1.62 (m, 2H). |
| | | LCMS[M + H]+ = 368.1 |
| 168 | B | ¹HNMR (400 MHz, DMSO-d₆): δ |
| | | 8.55 (s, 1H), 7.67 (d, J = 8.4 Hz, |
| | | 2H), 7.54 (s, 1H), 6.18-6.29 (m, |
| | | 1H), 4.28-4.42 (m, 1H), 4.21 (s, |
| | | 3H), 2.83-2.93 (m, 1H), 2.53-2.55 |
| | | (m, 1H, overlapped with DMSO- |
| | | d₆ peak), 2.09-2.27 (m, 5H), 1.86- |
| | | 1.94 (m, 1H), 1.72-1.81 (m, 1H), |
| | | 1.53-1.65 (m, 2H). |
| | | LCMS[M + H]+ = 407.1 |
| 169 | B | LCMS[M + H]+ = 365.1 |
| 170 | B | LCMS[M + H]+ = 379.1 |

### Example 7

### NLRP3 Inflammasome Activation and Cell Viability Assessment in THP1 Macrophages

THP1—Human acute monocytic leukemia cells were cultured (ATCC, cat #TIB-202) in Gibco RPMI-1640 medium (ThermoFisher cat #72400054) supplemented with 10% Heat Inactivated FBS at density between 3-8×10\hat{ }5 viable cells/mi. The cells were then subcultured when the cell concentration reached 8×10⁵ cells/mL (every 2-3 days).

To determine the compounds' IC₅₀, 1.75×10⁴ cells/well were plated in CELLSTAR 384 well plates (Greiner cat #781091) in 50 ul/well DMEM (ThermoFisher, cat #10393021), 10% FBS, 1× GlutaMax (ThermoFisher, cat #35050038)+20 nM PMA (Sigma, cat #P1585) and only the inner 224 wells of a 384 well plate were used. The parameter wells were filled with 50 ul PBS and incubated at 37° C., 5% CO₂ for 48 hrs.

After 48 hrs incubation, the PMA containing media was removed, and changed for 40 ul/well of fresh DMEM, 10% FBS, 1× GlutaMax and incubated at 37° C., 5% CO₂ for 24 hrs.

The following day the cells were primed with LPS (E. coli) (Sigma, cat #L3129) at ng/ml in DMEM, 10% FBS, 1× GlutaMax for 3 hrs at 37° C., 5% CO₂.

Following the LPS priming step, the cells were treated with compounds at 10 uM top final concentration, 1:4 dilution, 8 times: (10 uM, 2.5 uM, 0.625 uM, 0.156 uM, 0.039 uM, 0.0097 uM, 0.0024 uM, 0.0006 uM). DMSO was used as a vehicle control, and MCC950 (InvivoGen, cat #inh-mcc) was used at 1 uM as a positive control, and incubated for 1 hrs at 37° C., 5% CO₂.

Following the 1 hr compound incubation, the NLRP3 inflammasome activation step was conducted by treating cells with Nigericin (InvivoGen, cat #tlrl-nig) at 6.7 uM final concentration for 3 hrs at 37° C., 5% CO₂.

Thereafter, 30 ul samples of cells' supernatants were collected for cytokine analysis which was conducted on Hu IL-1β AlphaLISA (Perkin Elmer, cat #AL220C) and Hu IL-6 AlphaLISA (Perkin Elmer, cat #AL220C). The cells' viability was assessed by performing CellTiter-Glo Luminescent Assay (Promega, cat #G7572) according to the manufacturer's protocol.

Cell viability assessment for select NLRP3 inflammasome modulators is displayed in Tables 3 and 4.

For Table 3—The activity ranges are as follows:
“++++++” denotes IL-1β activity of 10 nM;
“+++++” denotes IL-1β activity of >10 nM and ≤100 nM;
“++++” denotes IL-1β activity of >100 nM and ≤500 nM;
“+++” denotes IL-1β activity of >500 nM and ≤1,000 nM;
“++” denotes IL-1β activity of >1,000 nM and <10,000 nM;
“+” denotes IL-1β activity of ≥10,000 nM; and
“*” denotes not yet tested.

TABLE 3

| | IL-1β |
|---|---|
| | Activity |
| Cpd No. | (nM) |
| 1 | ++++++ |
| 2 | ++++ |
| 3 | +++ |
| 4 | ++++++ |
| 5 | +++++ |
| 6 | ++++++ |
| 7 | ++++ |
| 8 | ++++ |
| 9 | + |
| 10 | +++++ |
| 11 | ++ |
| 12 | ++++ |
| 13 | + |
| 14 | ++++++ |
| 15 | +++ |
| 16 | * |
| 17 | +++++ |
| 18 | ++ |
| 19 | ++++ |
| 20 | +++ |
| 21 | ++++++ |
| 22 | * |
| 23 | ++ |
| 24 | ++++ |
| 25 | ++++ |
| 26 | +++++ |
| 27 | ++ |
| 28 | * |
| 29 | +++ |
| 30 | ++ |
| 31 | +++++ |
| 32 | ++++++ |
| 33 | * |
| 34 | * |
| 35 | * |
| 36 | * |
| 37 | * |
| 38 | * |
| 39 | ++++ |
| 40 | * |
| 41 | * |
| 42 | * |
| 43 | * |
| 44 | * |
| 45 | * |
| 46 | * |
| 47 | * |
| 48 | * |
| 49 | * |
| 50 | * |
| 51 | * |
| 52 | * |
| 53 | +++++ |
| 54 | ++++ |
| 55 | * |
| 56 | * |
| 57 | * |
| 58 | ++++ |
| 59 | + |
| 60 | * |
| 61 | +++++ |
| 62 | +++++ |
| 63 | +++++ |
| 64 | +++++ |
| 65 | ++++++ |
| 66 | + |
| 67 | +++++ |
| 68 | + |
| 69 | +++++ |
| 70 | + |
| 71 | ++ |
| 72 | ++ |
| 73 | ++++ |
| 74 | + |
| 75 | ++ |
| 76 | ++++++ |
| 77 | +++ |
| 78 | ++++++ |
| 79 | ++++ |
| 80 | ++++++ |
| 81 | ++++ |
| 82 | ++ |
| 83 | +++++ |
| 84 | + |
| 85 | ++++ |
| 86 | ++++ |
| 87 | ++++ |
| 88 | ++ |
| 89 | ++++ |
| 90 | ++++ |
| 91 | ++++ |
| 92 | + |
| 93 | ++ |
| 94 | + |
| 95 | +++ |
| 96 | +++++ |
| 97 | ++++ |
| 98 | ++++ |
| 99 | ++++++ |
| 100 | +++++ |
| 101 | +++++ |
| 102 | +++ |
| 103 | ++++++ |
| 104 | +++++ |
| 105 | + |
| 106 | + |
| 107 | ++++ |
| 108 | +++++ |
| 109 | +++++ |
| 110 | +++++ |
| 111 | ++++ |
| 112 | +++++ |
| 113 | * |
| 114 | +++++ |
| 115 | ++++++ |
| 116 | * |
| 117 | ++++ |
| 118 | +++++ |
| 119 | +++++ |
| 120 | ++++ |
| 121 | +++++ |
| 122 | ++++++ |
| 123 | ++++++ |
| 124 | ++++ |
| 125 | +++++ |
| 126 | + |
| 127 | ++++++ |
| 128 | * |
| 129 | ++++ |
| 130 | * |
| 131 | +++++ |
| 132 | ++ |
| 133 | * |
| 134 | * |
| 135 | + |
| 136 | ++++ |
| 137 | +++++ |
| 138 | * |
| 139 | + |
| 140 | ++ |
| 141 | * |
| 142 | * |
| 143 | ++++++ |
| 144 | ++++++ |
| 145 | + |
| 146 | +++++ |
| 147 | ++++++ |
| 148 | +++++ |
| 149 | + |
| 150 | +++++ |
| 151 | ++++++ |
| 152 | +++++ |
| 153 | +++++ |
| 154 | +++++ |
| 155 | ++++ |
| 156 | +++++ |
| 157 | ++++ |
| 158 | +++++ |
| 159 | ++ |
| 160 | ++ |
| 161 | + |
| 162 | ++ |
| 163 | + |
| 164 | ++++ |
| 165 | ++++++ |
| 166 | +++++ |
| 167 | ++ |
| 168 | ++ |
| 169 | * |
| 170 | * |

For Table 4—The activity ranges are as follows:
“++++” denotes IL-6 activity of ≤1 μM;
“+++” denotes IL-6 activity of >1 μM and ≤5 μM;
“++” denotes IL-6 activity of >5 μM and <10 μM;
“+” denotes IL-6 activity of ≥10 μM; and
   “*” denotes not yet tested.

TABLE 4

| | IL-6 |
|---|---|
| | Activity |
| Cpd No. | (μM) |
| 1 | +++ |
| 2 | + |
| 3 | + |
| 4 | ++ |
| 5 | +++ |
| 6 | ++ |
| 7 | + |
| 8 | + |
| 9 | + |
| 10 | +++ |
| 11 | + |
| 12 | + |
| 13 | + |
| 14 | * |
| 15 | * |
| 16 | * |
| 17 | + |
| 18 | +++ |
| 19 | ++ |
| 20 | + |
| 21 | + |
| 22 | * |
| 23 | ++ |
| 24 | ++ |
| 25 | + |
| 26 | ++ |
| 27 | + |
| 28 | * |
| 29 | + |
| 30 | + |
| 31 | + |
| 32 | + |
| 33 | * |
| 34 | * |
| 35 | * |
| 36 | * |
| 37 | * |
| 38 | * |
| 39 | ++ |
| 40 | * |
| 41 | * |
| 42 | * |
| 43 | * |
| 44 | * |
| 45 | * |
| 46 | * |
| 47 | * |
| 48 | * |
| 49 | * |
| 50 | * |
| 51 | * |
| 52 | * |
| 53 | + |
| 54 | +++ |
| 55 | * |
| 56 | * |
| 57 | * |
| 58 | + |
| 59 | + |
| 60 | * |
| 61 | + |
| 62 | +++ |
| 63 | ++ |
| 64 | ++ |
| 65 | ++ |
| 66 | + |
| 67 | + |
| 68 | ++ |
| 69 | * |
| 70 | * |
| 71 | + |
| 72 | + |
| 73 | + |
| 74 | + |
| 75 | + |
| 76 | ++++ |
| 77 | ++++ |
| 78 | +++ |
| 79 | + |
| 80 | + |
| 81 | +++ |
| 82 | +++ |
| 83 | +++ |
| 84 | + |
| 85 | ++ |
| 86 | + |
| 87 | + |
| 88 | + |
| 89 | + |
| 90 | + |
| 91 | + |
| 92 | + |
| 93 | + |
| 94 | + |
| 95 | + |
| 96 | + |
| 97 | + |
| 98 | + |
| 99 | ++ |
| 100 | ++ |
| 101 | ++ |
| 102 | + |
| 103 | ++ |
| 104 | + |
| 105 | + |
| 106 | + |
| 107 | + |
| 108 | + |
| 109 | + |
| 110 | + |
| 111 | + |
| 112 | + |
| 113 | * |
| 114 | + |
| 115 | + |
| 116 | * |
| 117 | + |
| 118 | + |
| 119 | + |
| 120 | * |
| 121 | + |
| 122 | * |
| 123 | +++ |
| 124 | + |
| 125 | * |
| 126 | + |
| 127 | ++ |
| 128 | * |
| 129 | + |
| 130 | * |
| 131 | + |
| 132 | + |
| 133 | * |
| 134 | * |
| 135 | * |
| 136 | +++ |
| 137 | + |
| 138 | * |
| 139 | + |
| 140 | * |
| 141 | * |
| 142 | * |
| 143 | ++++ |
| 144 | + |
| 145 | + |
| 146 | + |
| 147 | + |
| 148 | + |
| 149 | + |
| 150 | * |
| 151 | + |
| 152 | + |
| 153 | + |
| 154 | + |
| 155 | + |
| 156 | + |
| 157 | + |
| 158 | ++ |
| 159 | + |
| 160 | + |
| 161 | + |
| 162 | + |
| 163 | + |
| 164 | + |
| 165 | + |
| 166 | + |
| 167 | + |
| 168 | + |
| 169 | * |
| 170 | * |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/357,916, filed on Jul. 1, 2022, are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having structure (A): 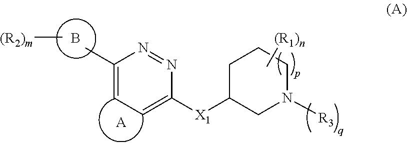
or a pharmaceutically acceptable salt, isomer, hydrate, solvate or isotope thereof, wherein:
ring A forms a fused 5-membered carbocyclic or heterocyclic ring optionally substituted by one or more R₄;
ring B is piperidinyl or an aryl ring;
X₁ is CRₐR_{b}, NR₄, or O;
Rₐ and R_{b} are each independently H or alkyl;
R₁ is halo;
R₂ is halo, OH, CN, alkyl, haloalkyl, alkoxy, haloalkoxy, or cycloalkyl;
R₃ is H, cycloalkyl, or alkyl optionally substituted with one or more R_{3′};
R_{3′} is OH, halo, alkoxy, aminyl, alkylsulfonyl, cycloalkyl, aryl, or heterocyclyl;
each R₄ is independently H, halo, or alkyl, or when attached to a carbon atom, two R₄ may join together to form oxo;
m is 0-5
n is 0-4;
p is 0-2; and
q is 1 or 2.

2. The compound of claim 1, having structure (Ia): 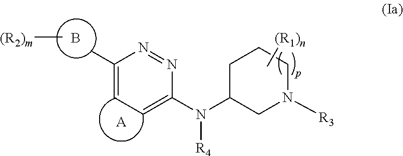
or a pharmaceutically acceptable salt, isomer, hydrate, solvate or isotope thereof.

3. The compound of claim 1, having structure (Ib): 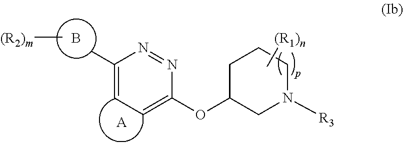
or a pharmaceutically acceptable salt, isomer, hydrate, solvate or isotope thereof.

4. The compound of claim 1, having structure (Ic): 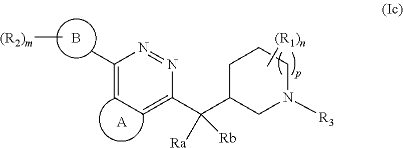
or a pharmaceutically acceptable salt, isomer, hydrate, solvate or isotope thereof.

5. The compound of any one of claims 1-4, wherein 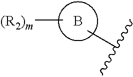
has one of the following structures:

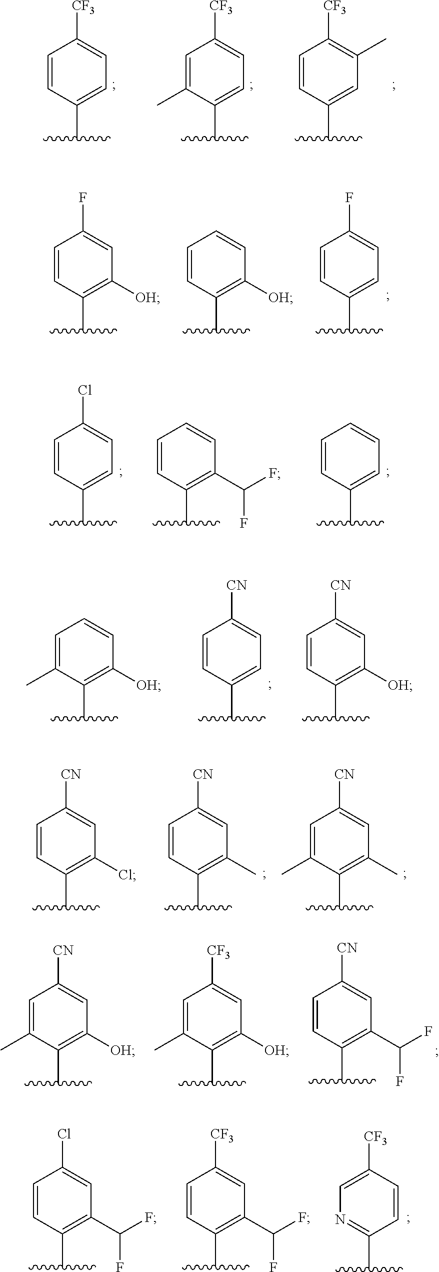

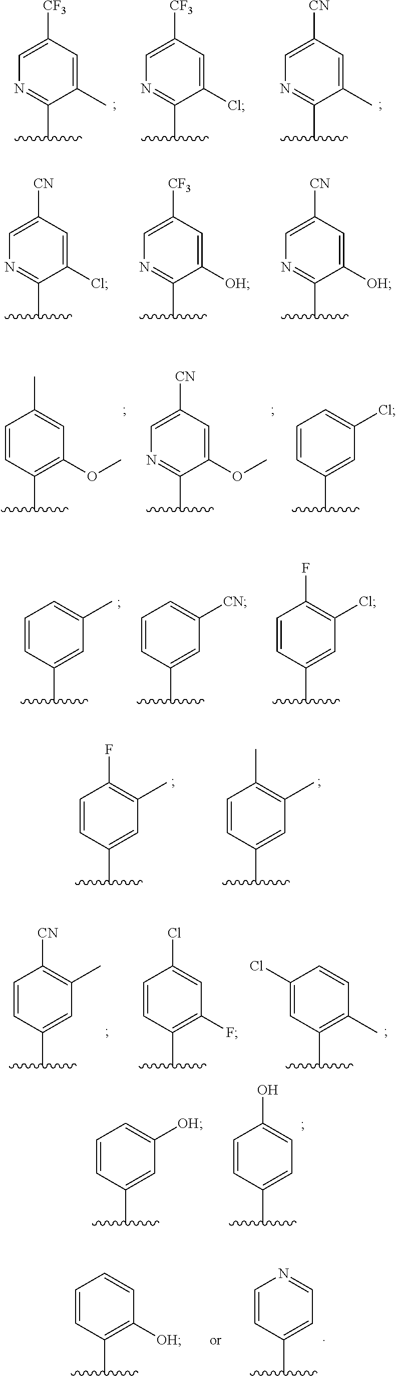

6. The compound of any one of claims 1-4, wherein B is piperidinyl.

7. The compound of claim 1, having structure (I′): 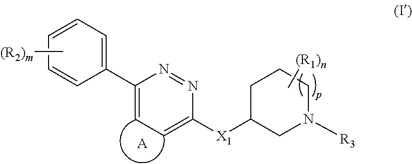
or a pharmaceutically acceptable salt, isomer, hydrate, solvate or isotope thereof, wherein:
ring A forms a fused 5-membered carbocyclic or heterocyclic ring optionally substituted by one or more R₄;
X₁ is CRₐR_{b}, NR₄, or O;
Rₐ and R_{b} are each independently H or alkyl;
R₁ is halo;
R₂ is halo, OH, CN, alkyl, haloalkyl, alkoxy, haloalkoxy, or cycloalkyl;
R₃ is H, cycloalkyl, or alkyl optionally substituted with one or more R_{3′};
R_{3′} is OH, halo, or cycloalkyl;
each R₄ is independently H or alkyl, or when attached to a carbon atom, two R₄ may join together to form oxo;
m is 0-4;
n is 0-4; and
p is 0-2.

8. The compound of any one of claims 1-2 or 7, having structure (I′a): 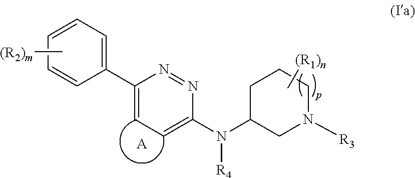
or a pharmaceutically acceptable salt, isomer, hydrate, solvate or isotope thereof.

9. The compound of any one of claims 1, 3, or 7, having structure (I′b): 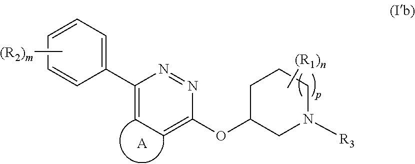
or a pharmaceutically acceptable salt, isomer, hydrate, solvate or isotope thereof

10. The compound of any one of claims 1, 4, or 7, having structure (I′c): 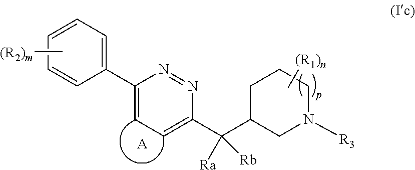
or a pharmaceutically acceptable salt, isomer, hydrate, solvate or isotope thereof

11. The compound of claim 1, having structure (I″): 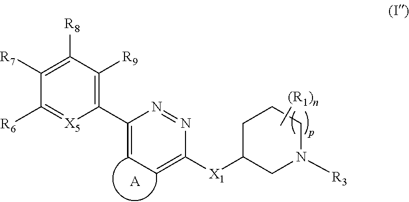
or a pharmaceutically acceptable salt, isomer, hydrate, solvate or isotope thereof, wherein:
ring A forms a fused 5-membered carbocyclic or heterocyclic ring optionally substituted by one or more R₄;
X₁ is CRₐR_{b}, NR₄, or O;
X₅ is CR₅ or N;
Rₐ and R_{b} are each independently H or alkyl;
R₁ is halo;
R₃ is H, cycloalkyl, or alkyl optionally substituted with one or more R_{3′};
R_{3′} is OH, halo, or cycloalkyl;
each R₄ is independently H or alkyl, or when attached to a carbon atom, two R₄ may join together to form oxo;
R₅ is H, halo, OH, CN, alkyl, haloalkyl, alkoxy, haloalkoxy, or cycloalkyl;
R₆ is H, halo, OH, CN, alkyl, haloalkyl, alkoxy, haloalkoxy, or cycloalkyl;
R₇ is H, halo, OH, CN, alkyl, haloalkyl, alkoxy, haloalkoxy, or cycloalkyl;
R₈ is H, halo, OH, CN, alkyl, haloalkyl, alkoxy, haloalkoxy, or cycloalkyl;
R₉ is H, halo, OH, CN, alkyl, haloalkyl, alkoxy, haloalkoxy, or cycloalkyl;
n is 0-4; and
p is 0-2.

12. The compound of any one of claims 1 or 11, having structure (I″): 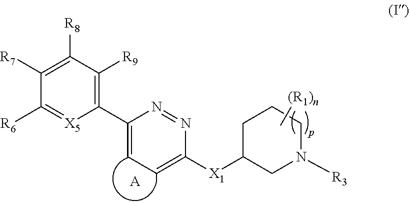
or a pharmaceutically acceptable salt, isomer, hydrate, solvate or isotope thereof, wherein:
ring A forms a fused 5-membered heterocyclic ring comprising nitrogen and optionally substituted by one or more R₄; and
R₇ is CN.

13. The compound of claim 1 or 11-12, having structure (I″a): 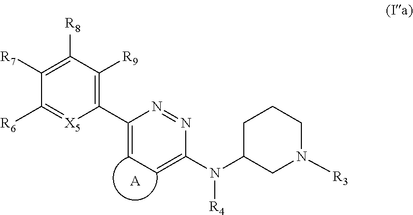
or a pharmaceutically acceptable salt, isomer, hydrate, solvate or isotope thereof, wherein:
ring A forms a fused 5-membered heterocyclic ring and optionally substituted by one or more R₄;
X₅ is CR₅ or N;
R₃ is H, cycloalkyl, or alkyl optionally substituted with one or more R_{3′};
R_{3′} is OH, halo, or cycloalkyl;
each R₄ is independently H or alkyl, or when attached to a carbon atom, two R₄ may join together to form oxo;
R₅ is H, halo, OH, CN, alkyl, haloalkyl, alkoxy, haloalkoxy, or cycloalkyl;
R₆ is H, halo, OH, CN, alkyl, haloalkyl, alkoxy, haloalkoxy, or cycloalkyl;
R₇ is H, halo, OH, CN, alkyl, haloalkyl, alkoxy, haloalkoxy, or cycloalkyl;
R₈ is H, halo, OH, CN, alkyl, haloalkyl, alkoxy, haloalkoxy, or cycloalkyl; and
R₉ is H, halo, OH, CN, alkyl, haloalkyl, alkoxy, haloalkoxy, or cycloalkyl.

14. The compound of claim 13, wherein:
ring A forms a fused 5-membered heterocyclic ring comprising nitrogen and optionally substituted by one or more R₄;
X₅ is CR₅; and
R₇ is CN.

15. The compound of claim 13, wherein:
X₅ is N; and
R₇ is CN or haloalkyl.

16. The compound of any one of claims 1-15, wherein ring A forms a fused 5-membered carbocyclic ring.

17. The compound of any one of claims 1-15, wherein ring A forms a fused 5-membered heterocyclic ring.

18. The compound any one of claims 1-15, wherein ring A forms a fused 5-membered ring having one of the following structures:

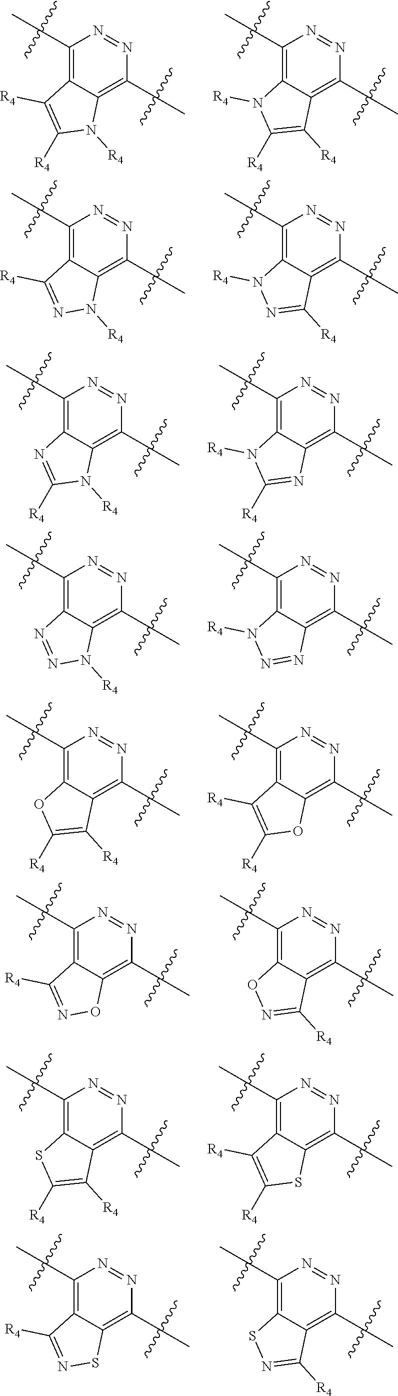

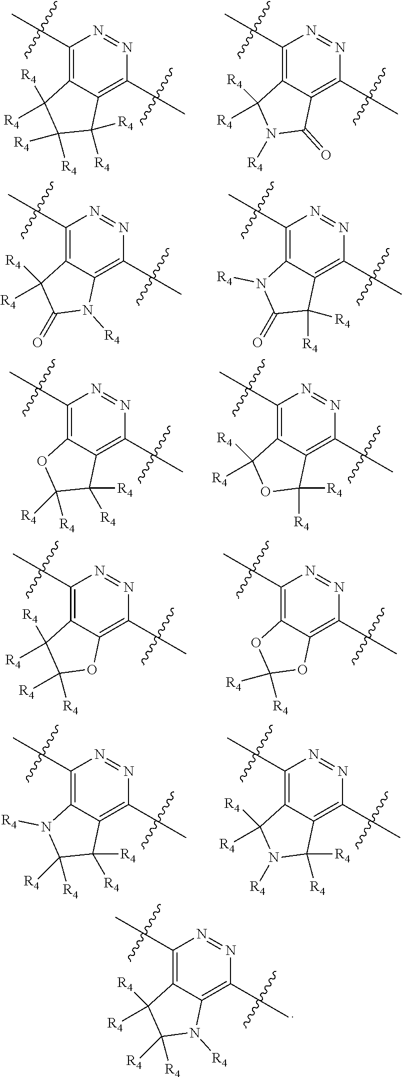

19. The compound of claim 18, wherein ring A forms a fused 5-membered ring having one of the following structures:

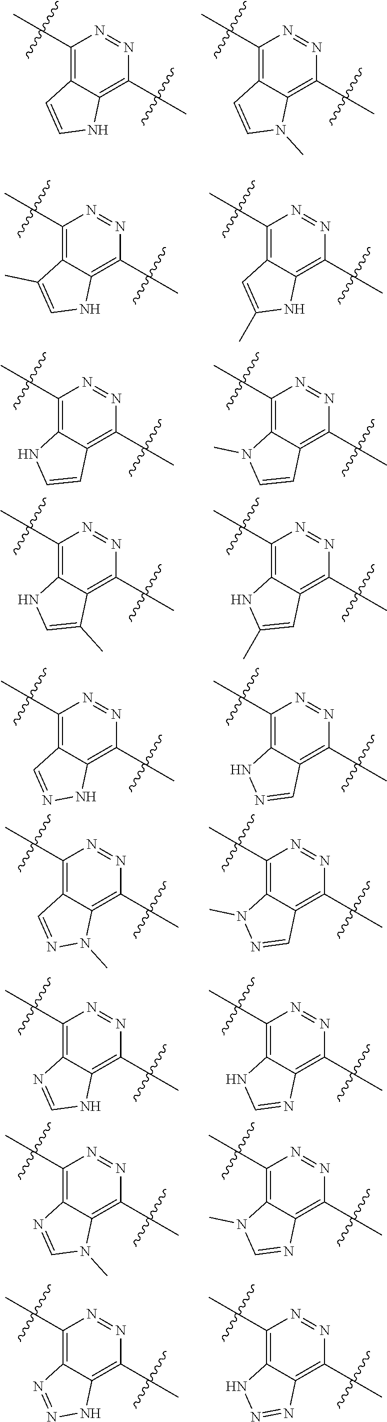

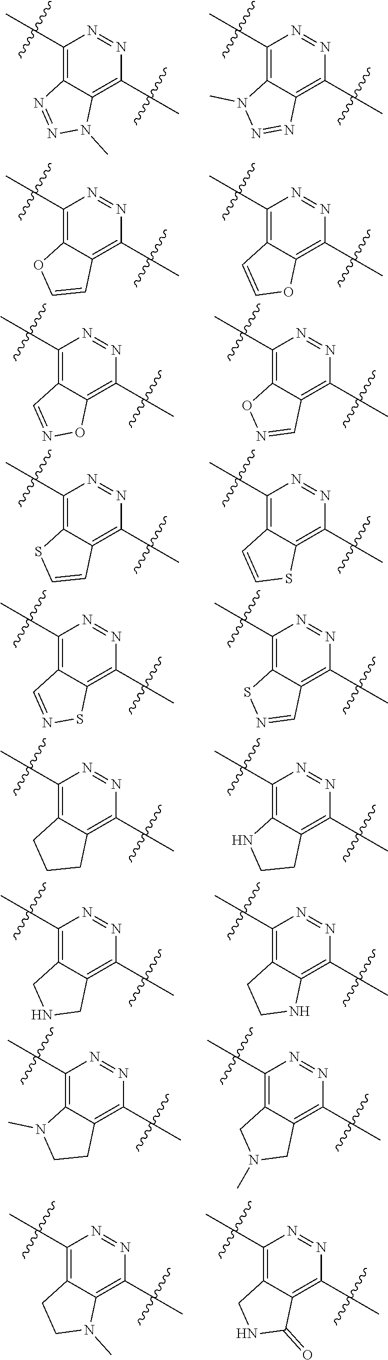

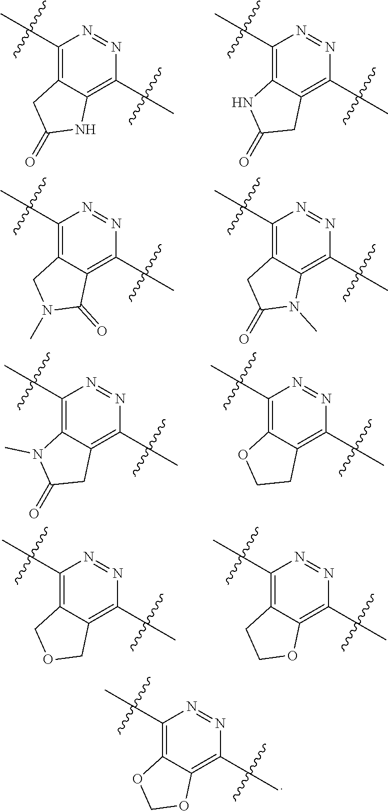

20. The compound of claim 1 or 7, having structure (I′): 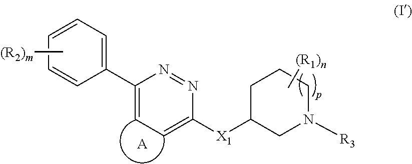
or a pharmaceutically acceptable salt, isomer, hydrate, solvate or isotope thereof, wherein:
ring A forms a fused 5-membered non-aromatic carbocyclic ring optionally substituted by one or more R₄.

21. The compound of claim 20, wherein ring A forms a fused 5-membered carbocyclic ring having the following structure:

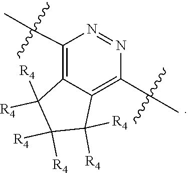

22. The compound of claim 21, wherein ring A forms a fused 5-membered carbocyclic ring having the following structure:

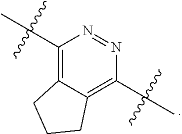 [SMILES: *c1nnc(*)c2c1CCC2]

23. The compound of claim 1 or 7, having structure (I′): 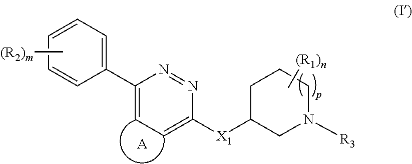
or a pharmaceutically acceptable salt, isomer, hydrate, solvate or isotope thereof, wherein:
ring A forms a fused 5-membered heterocyclic ring comprising 1-3 nitrogen atoms optionally substituted by one or more R₄.

24. The compound of any one of claims 1-17, or 23, wherein ring A forms a fused 5-membered heterocyclic ring having one of the following structures:

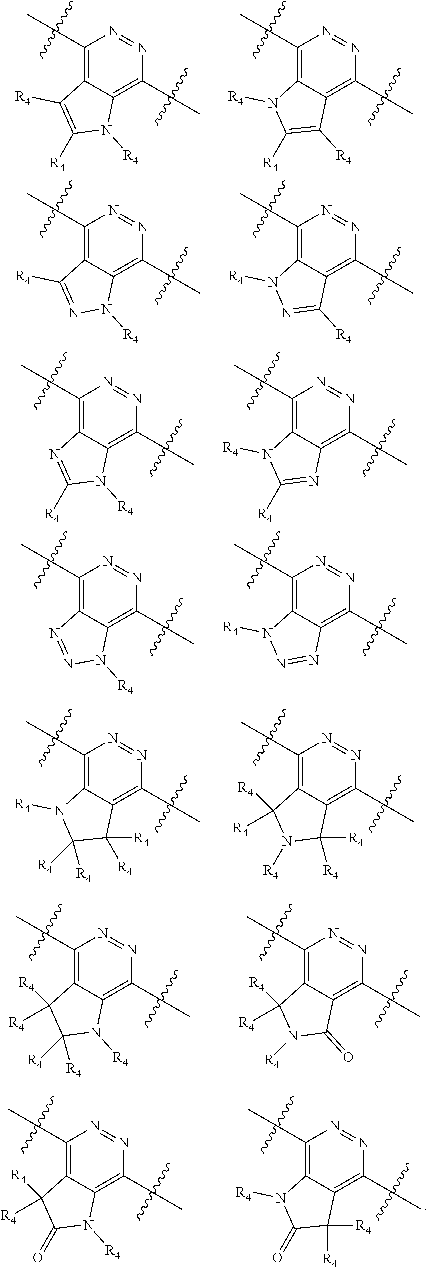

25. The compound of claim 24, wherein ring A forms a fused 5-membered heterocyclic ring having one of the following structures:

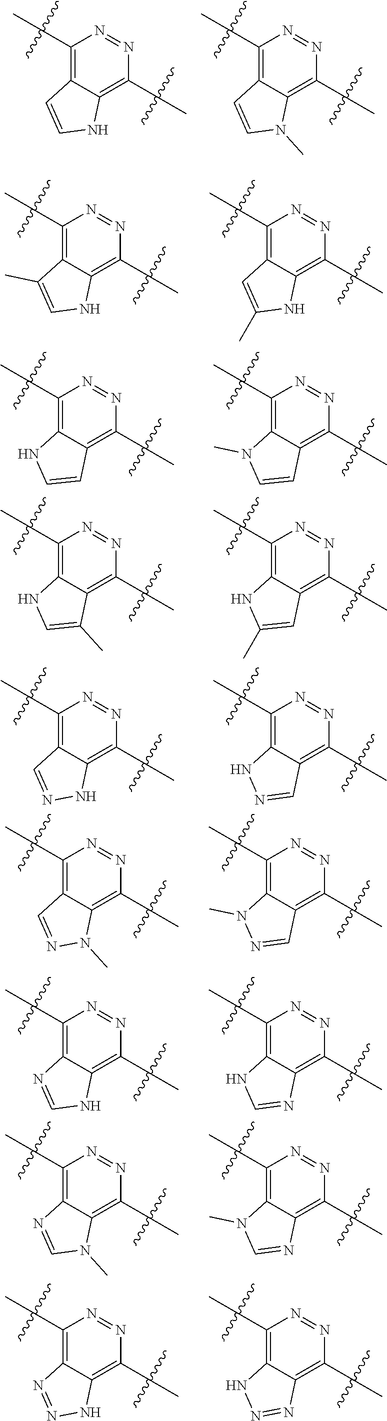

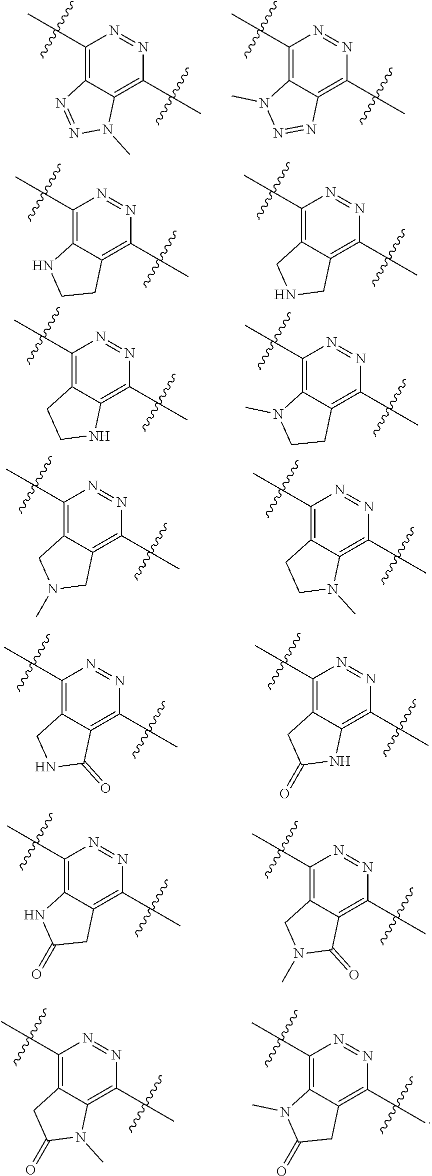

26. The compound of claim 24, wherein ring A forms a fused 5-membered heterocyclic ring having one of the following structures:

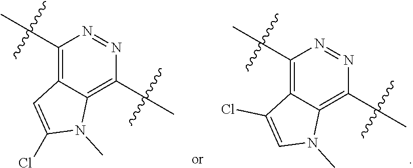 [SMILES: *c1nnc(*)c2c1cc(Cl)n2C ; *c1nnc(*)c2c1c(Cl)cn2C]

27. The compound of claim 1 or 7, having structure (I′): 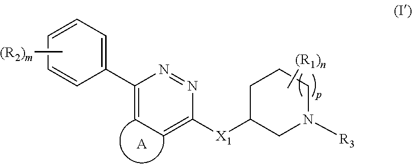
or a pharmaceutically acceptable salt, isomer, hydrate, solvate or isotope thereof, wherein:
ring A forms a fused 5-membered heterocyclic ring comprising at least one oxygen atom optionally substituted by one or more R₄.

28. The compound of any one of claims 1-17, or 27, wherein ring A forms a fused 5-membered heterocyclic ring having one of the following structures:

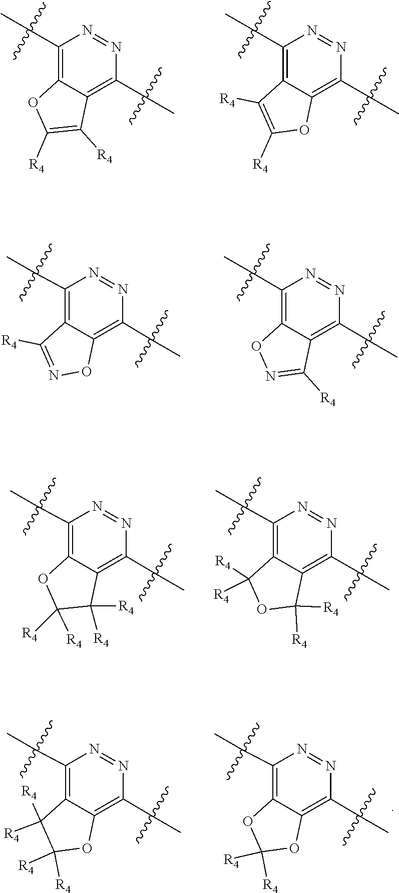

29. The compound of claim 28, wherein ring A forms a fused 5-membered heterocyclic ring having one of the following structures:

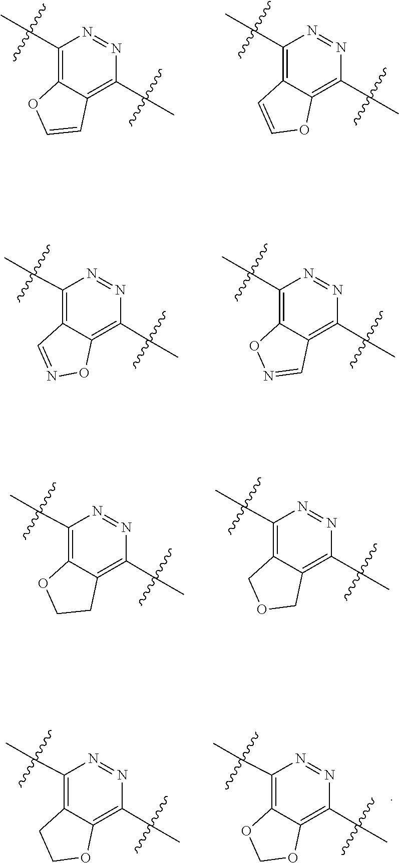

30. The compound of claim 1 or 7, having structure (I): 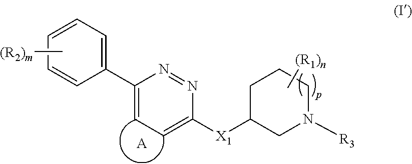
or a pharmaceutically acceptable salt, isomer, hydrate, solvate or isotope thereof, wherein:
ring A forms a fused 5-membered heterocyclic ring comprising at least one sulfur atom optionally substituted by one or more R₄.

31. The compound of any one of claims 1-17, or 30, wherein ring A forms a fused 5-membered heterocyclic ring having one of the following structures:

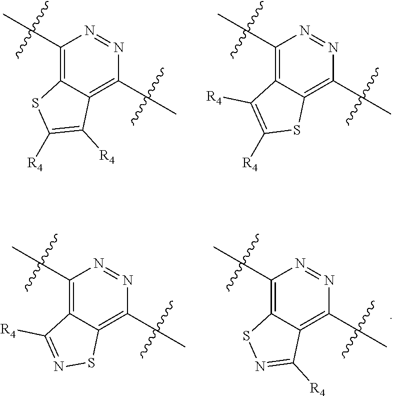

32. The compound of claim 31, wherein ring A forms a fused 5-membered heterocyclic ring having one of the following structures:

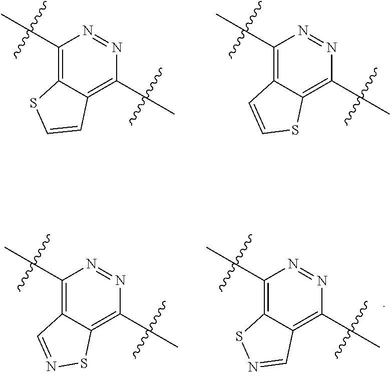 [SMILES: *c1nnc(*)c2sccc12 ; *c1nnc(*)c2sccc12 ; *c1nnc(*)c2sncc12 ; *c1nnc(*)c2sncc12]

33. The compound of claim 1 or 7, having structure (II): 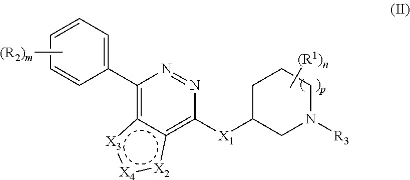
or a pharmaceutically acceptable salt, isomer, hydrate, solvate or isotope thereof, wherein: [symbol]
indicates an aromatic or non-aromatic ring system; and
X₂, X₃, and X₄ are each independently ═N—, —NR₄—, —O—, or —S—, —C(R₄)₂—, or ═CR₄—.

34. The compound of any one of claims 1, 7, or 33, having structure (II): 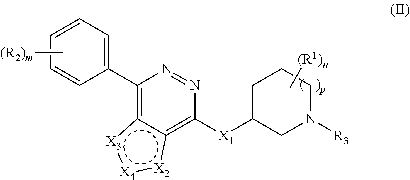
or a pharmaceutically acceptable salt, isomer, hydrate, solvate or isotope thereof, wherein: [symbol]
indicates an non-aromatic ring system; and
X₂, X₃, and X₄ are each independently —C(R₄)₂—.

35. The compound of any one of claims 1, 7, or 33, having structure (II): 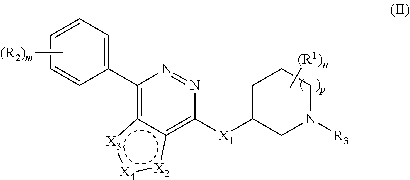
or a pharmaceutically acceptable salt, isomer, hydrate, solvate or isotope thereof, wherein: [symbol]
indicates an non-aromatic ring system; and
X₂, X₃, and X₄ are each independently —O— or —C(R₄)₂—.

36. The compound of any one of claims 1, 7, or 33, having structure (II): 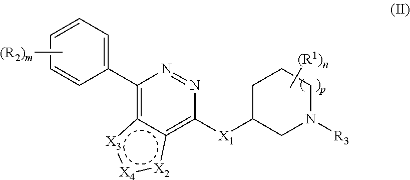
or a pharmaceutically acceptable salt, isomer, hydrate, solvate or isotope thereof, wherein: [symbol]
indicates an non-aromatic ring system; and
X₂, X₃, and X₄ are each independently —NR₄— or —C(R₄)₂—.

37. The compound of any one of claims 1, 7, or 33, having structure (II): 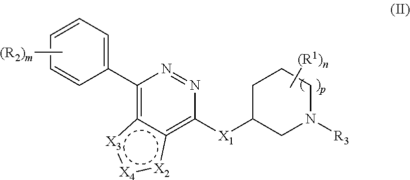
or a pharmaceutically acceptable salt, isomer, hydrate, solvate or isotope thereof, wherein: [symbol]
indicates an aromatic ring system; and
X₂, X₃, and X₄ are each independently ═N—, —NR₄— or ═CR₄—, wherein at least one of X₂ or X₃ is —NR₄—.

38. The compound of any one of claims 1, 7, or 33, having structure (II): 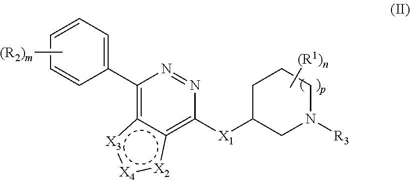
or a pharmaceutically acceptable salt, isomer, hydrate, solvate or isotope thereof, wherein: [symbol]
indicates an aromatic ring system; and
X₂, X₃, and X₄ are each independently —O—, ═CR₄—, or ═N—, wherein at least one of X₂ or X₃ is —O—.

39. The compound of any one of claims 1, 7, or 33, having structure (II): 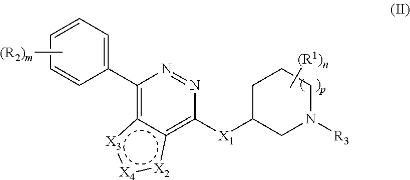
or a pharmaceutically acceptable salt, isomer, hydrate, solvate or isotope thereof, wherein: [symbol]
indicates an aromatic ring system; and
X₂, X₃, and X₄ are each independently —S—, ═CR₄—, or ═N—, wherein at least one of X₂ or X₃ is —S—.

40. The compound of any one of claims 1, 7, or 33, having structure (Ila): 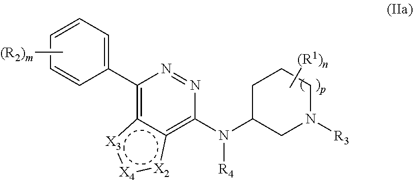
or a pharmaceutically acceptable salt, isomer, hydrate, solvate or isotope thereof.

41. The compound of any one of claims 1, 7, or 33, having structure (IIb): 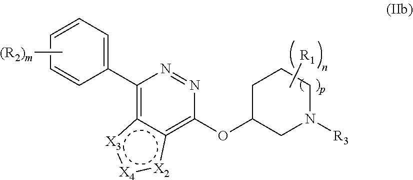
or a pharmaceutically acceptable salt, isomer, hydrate, solvate or isotope thereof.

42. The compound of any one of claims 1, 7, or 33, having structure (IIc): 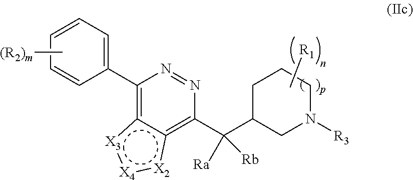
or a pharmaceutically acceptable salt, isomer, hydrate, solvate or isotope thereof.

43. The compound of any one of claims 1, 7, or 36, having-structure (IVa-i): 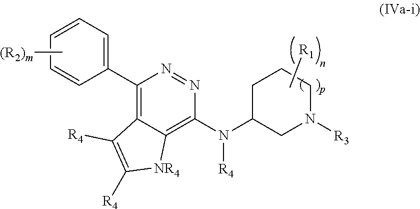
or a pharmaceutically acceptable salt, isomer, hydrate, solvate or isotope thereof, wherein:
R₁ is halo;
R₂ is halo, CN, alkyl, haloalkyl, alkoxy, haloalkoxy, or cycloalkyl;
R₃ is alkylsulfonyl, cycloalkyl, aryl, heterocyclyl, or alkyl optionally substituted with one or more R_{3′};
R_{3′} is OH, halo, alkoxy, aminyl, or cycloalkyl;
each R₄ is independently H, halo, or alkyl when attached to a carbon atom and H or alkyl when attached to a nitrogen atom;
m is 0-4;
n is 0-4; and
p is 0-2.

44. The compound of any one of claims 1, 7, or 36, having structure (Va-i): 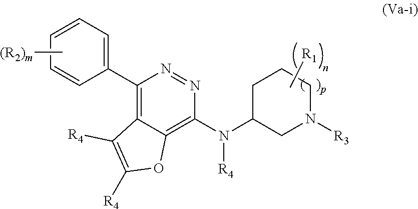
or a pharmaceutically acceptable salt, isomer, hydrate, solvate or isotope thereof, wherein:
R₁ is halo;
R₂ is halo, CN, alkyl, haloalkyl, or cycloalkyl;
R₃ is alkylsulfonyl, cycloalkyl, aryl, heterocyclyl, or alkyl optionally substituted with one or more R_{3′};
R_{3′} is OH, halo, alkoxy, aminyl, or cycloalkyl;
each R₄ is independently H, halo, or alkyl when attached to a carbon atom and H or alkyl when attached to a nitrogen atom;
m is 0-4;
n is 0-4; and
p is 0-2.

45. The compound of any one of claims 1, 7, or 36, having structure (VIa-i): 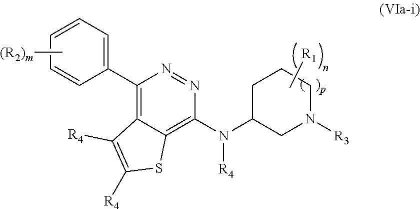
or a pharmaceutically acceptable salt, isomer, hydrate, solvate or isotope thereof, wherein:
R₁ is halo;
R₂ is halo, CN, alkyl, haloalkyl, alkoxy, haloalkoxy, or cycloalkyl;
R₃ is alkylsulfonyl, cycloalkyl, aryl, heterocyclyl, or alkyl optionally substituted with one or more R_{3′};
R_{3′} is OH, halo, alkoxy, aminyl, or cycloalkyl;
each R₄ is independently H, halo, or alkyl when attached to a carbon atom and H or alkyl when attached to a nitrogen atom;
m is 0-4;
n is 0-4; and
p is 0-2,
wherein R₃ is not ethyl.

46. The compound of any one of claims 1, 7, 11-12, or 16-39, wherein X₁ is CRₐR_{b}.

47. The compound of claim 46, wherein Rₐ and R_{b} are both H.

48. The compound of claim 46, wherein one of Rₐ and R_{b} is H and the other is alkyl.

49. The compound of claim 46, wherein Rₐ and R_{b} are both alkyl.

50. The compound of claim 48 or 49, wherein the alkyl is methyl.

51. The compound of any one of claims 1, 7, 11-12, or 16-39, wherein X₁ is NR₄.

52. The compound of claim 51, wherein R₄ is H.

53. The compound of claim 51, wherein R₄ is alkyl.

54. The compound of claim 53, wherein R₄ is methyl.

55. The compound of any one of claims 1, 7, 11-12, or 16-39, wherein X₁ is O.

56. The compound of any one of claims 1-55, wherein R₁ is F.

57. The compound of any one of claims 1-55, wherein R₁ is Cl.

58. The compound of any one of claims 1-55, wherein R₁ is Br.

59. The compound of any one of claims 1-55, wherein R₁ is I.

60. The compound of any one of claims 1-59, wherein R₂ is halo.

61. The compound of claim 60, wherein R₂ is F.

62. The compound of claim 60, wherein R₂ is Cl.

63. The compound of any one of claims 1-59, wherein R₂ is OH.

64. The compound of any one of claims 1-59, wherein R₂ is CN.

65. The compound of any one of claims 1-59, wherein R₂ is alkyl.

66. The compound of claim 65, wherein R₂ is methyl.

67. The compound of claim 65, wherein R₂ is ethyl.

68. The compound of any one of claims 1-59, wherein R₂ is haloalkyl.

69. The compound of claim 68, wherein R2 is CF₃.

70. The compound of claim 68, wherein R2 is CHF₂.

71. The compound of any one of claims 1-59, wherein R₂ is alkoxy.

72. The compound of claim 71, wherein R₂ is OCH₃.

73. The compound of any one of claims 1-59, wherein R₂ is haloalkoxy.

74. The compound of claim 73, wherein R₂ is OCF₃.

75. The compound of any one of claims 1-59, wherein R₂ is cycloalkyl.

76. The compound of claim 75, wherein R₂ is cyclopropyl.

77. The compound of any one of claims 1-76, wherein R₃ is H.

78. The compound of any one of claims 1-76, wherein R₃ is not H.

79. The compound of any one of claims 1-76, wherein R₃ is C₁-C₆ alkyl.

80. The compound of claim 79, wherein R₃ is methyl.

81. The compound of claim 79, wherein R₃ is ethyl.

82. The compound of claim 79, wherein R₃ is isopropyl.

83. The compound of any one of claims 1-76, wherein R₃ is alkyl substituted with one or more F.

84. The compound of claim 83, wherein R₃ is —CH₂CH₂F.

85. The compound of claim 83, wherein R₃ is —CH₂CHF₂.

86. The compound of claim 83, wherein R₃ is —CH₂CF₃.

87. The compound of any one of claims 1-76, wherein R₃ is alkyl substituted with one or more cycloalkyl.

88. The compound of claim 87, wherein R₃ is

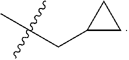 [SMILES: *CC1CC1]

89. The compound of any one of claims 1-76, wherein R₃ is alkyl substituted with one or more OH.

90. The compound of claim 89, wherein R₃ is

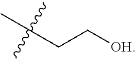 [SMILES: *CCO]

91. The compound of claim 89, wherein R₃ is

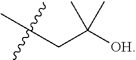 [SMILES: *CC(C)(C)O]

92. The compound of any one of claims 1-76, wherein R₃ is alkyl substituted with one or more alkoxy.

93. The compound of claim 92, wherein R₃ is

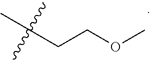 [SMILES: *CCOC]

94. The compound of any one of claims 1-76, wherein R₃ is alkyl substituted with one or more aminyl.

95. The compound of claim 94, wherein R₃ is)

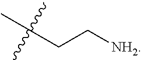 [SMILES: *CCN]

96. The compound of claim 94, wherein R₃ is

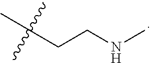 [SMILES: *CCNC]

97. The compound of claim 94, wherein R₃ is.

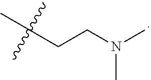 [SMILES: *CCN(C)C]

98. The compound of any one of claims 1-76, wherein R₃ is alkylsulfonyl.

99. The compound of claim 98, wherein R₃ is

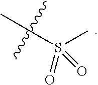 [SMILES: *S(C)(=O)=O]

100. The compound of any one of claims 1-76, wherein R₃ is cycloalkyl.

101. The compound of claim 100, wherein R₃ is cyclopropyl.

102. The compound of any one of claims 1-76, wherein R₃ is aryl.

103. The compound of claim 102, wherein R₃ is phenyl.

104. The compound of any one of claims 1-76, wherein R₃ is heterocyclyl.

105. The compound of claim 104, wherein R₃ is

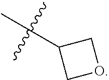 [SMILES: *C1COC1]

106. The compound of any one of claims 1-76, wherein R₃ is heteroaryl.

107. The compound of claim 106, wherein R₃ is pyridinyl.

108. The compound of any one of claims 1-107, wherein 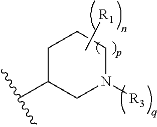
has the following structure:

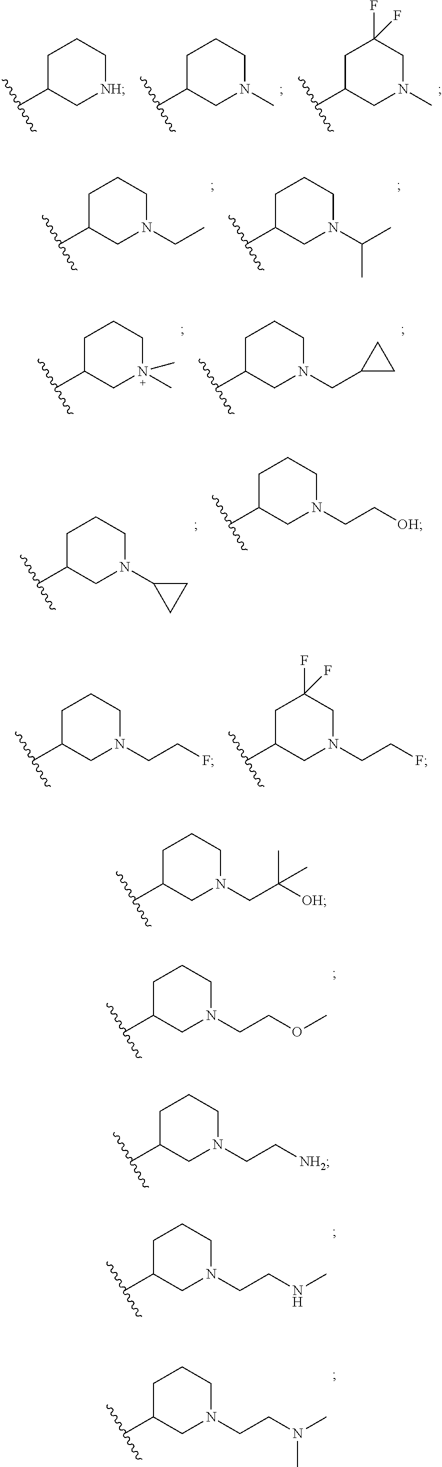

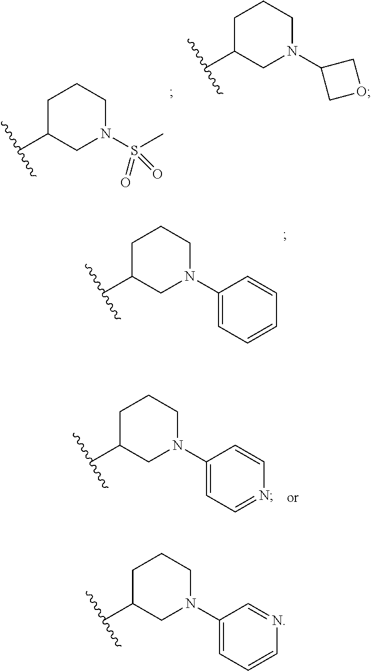 [SMILES: *C1CCCN(S(C)(=O)=O)C1 ; *C1CCCN(C2COC2)C1 ; *C1CCCN(c2ccccc2)C1 ; *C1CCCN(c2ccncc2)C1 ; *C1CCCN(c2cccnc2)C1]

109. The compound of any one of claims 1-108, wherein an R₄ is H.

110. The compound of any one of claims 1-108, wherein an R₄ is alkyl.

111. The compound of claim 110, wherein an R₄ is methyl.

112. The compound of any one of claims 1-108, wherein each R₄ is H or methyl.

113. The compound of any one of claims 1-108, wherein when attached to a carbon atom, two R₄ join together to form oxo.

114. The compound of any one of claims 11-19, 24-26, 28-29, 31-32, 46-59, or 77-113, wherein R₅ is H or alkyl.

115. The compound of any one of claims 11-19, 24-26, 28-29, 31-32, 46-59, or 77-113, wherein R₆ is H.

116. The compound of any one of claims 11-19, 24-26, 28-29, 31-32, 46-59, 77-113, or 115, wherein R₇ is H, halo, OH, CN, alkyl, or haloalkyl.

117. The compound of any one of claims 11-19, 24-26, 28-29, 31-32, 46-59, 77-113, or 115-116, wherein R₈ is H, alkyl, halo, or OH.

118. The compound of any one of claims 11-19, 24-26, 28-29, 31-32, 46-59, 77-113, or 115-117, wherein R₉ is H, OH, halo, haloalkyl, alkyl, or alkoxy.

119. The compound of any one of claims 1-4, 6-10, or 16-113, wherein m is 0.

120. The compound of any one of claims 1-4, 6-10, or 16-113, wherein m is 1.

121. The compound of any one of claims 1-4, 6-10, or 16-113, wherein m is 2.

122. The compound of any one of claims 1-4, 6-10, or 16-113, wherein m is 3.

123. The compound of any one of claims 1-4, 6-10, or 16-113, wherein m is 4.

124. The compound of any one of claims 1-4, 6-10, or 16-113, wherein m is 5.

125. The compound of any one of claims 1-124, wherein n is 0.

126. The compound of any one of claims 1-124, wherein n is 1.

127. The compound of any one of claims 1-124, wherein n is 2.

128. The compound of any one of claims 1-124, wherein n is 3.

129. The compound of any one of claims 1-124, wherein n is 4.

130. The compound of any one of claims 1-129, wherein p is 0.

131. The compound of any one of claims 1-129, wherein p is 1.

132. The compound of any one of claims 1-129, wherein p is 2.

133. The compound of claim 1, wherein q is 1.

134. The compound of claim 1, wherein q is 2.

135. The compound of any one of claims 1-12 or 16-113, wherein p is 1, n is 0, and m is 0.

136. The compound of any one of claims 1-12 or 16-113, wherein p is 1, n is 0, and m is 1.

137. The compound of any one of claims 1-12 or 16-113, wherein p is 1, n is 0, and m is 2.

138. The compound of any one of claims 1-12 or 16-113, wherein p is 1, n is 2, and m is 1.

139. The compound of claim 1, or a pharmaceutically acceptable salt, isomer, hydrate, solvate or isotope thereof, wherein the compound has a structure as found in Table 1.

140. The compound of claim 1, or a pharmaceutically acceptable salt, isomer, hydrate, solvate or isotope thereof, wherein the compound has the following structure:

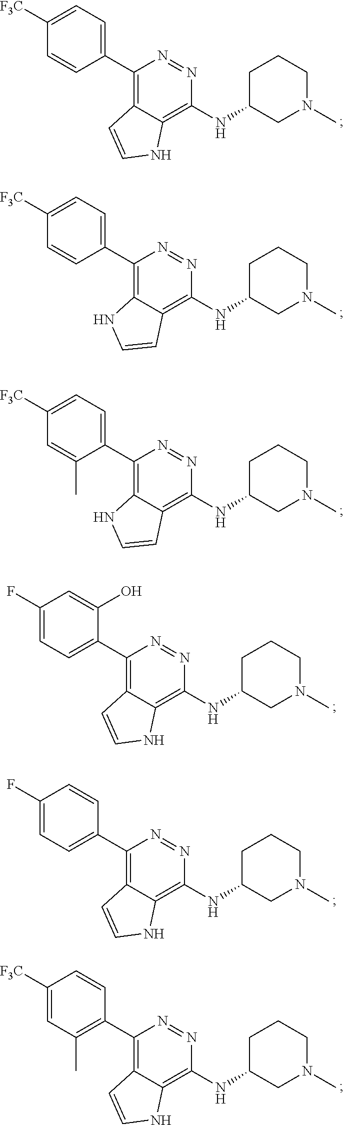

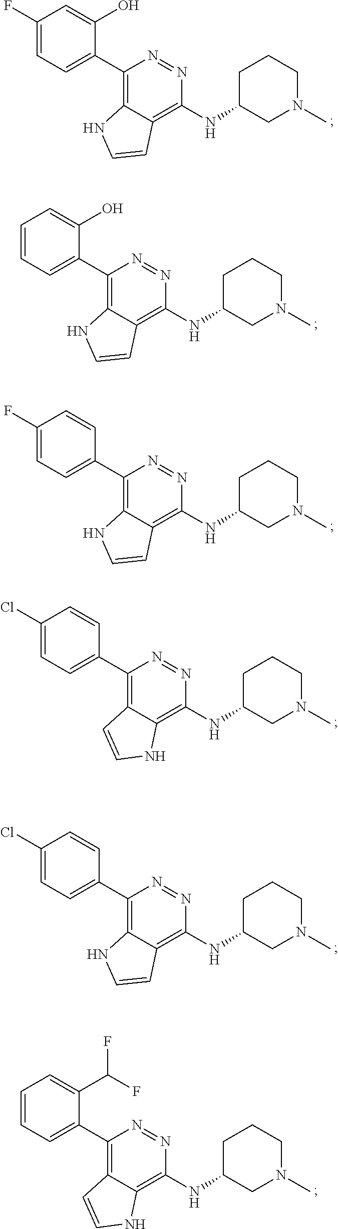 [SMILES: CN1CCC[C@@H](Nc2nnc(-c3ccc(F)cc3O)c3[nH]ccc23)C1 ; CN1CCC[C@@H](Nc2nnc(-c3ccccc3O)c3[nH]ccc23)C1 ; CN1CCC[C@@H](Nc2nnc(-c3ccc(F)cc3)c3[nH]ccc23)C1 ; CN1CCC[C@@H](Nc2nnc(-c3ccc(Cl)cc3)c3cc[nH]c23)C1 ; CN1CCC[C@@H](Nc2nnc(-c3ccc(Cl)cc3)c3[nH]ccc23)C1 ; CN1CCC[C@@H](Nc2nnc(-c3ccccc3C(F)F)c3cc[nH]c23)C1]

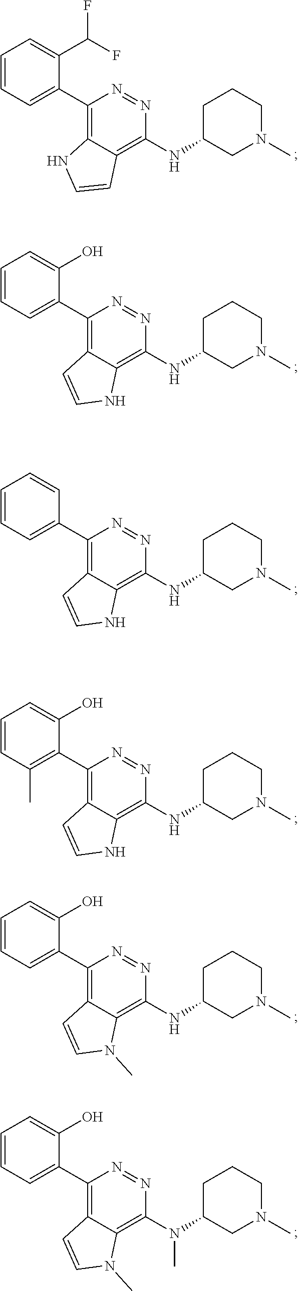 [SMILES: CN1CCC[C@@H](Nc2nnc(-c3ccccc3C(F)F)c3[nH]ccc23)C1 ; CN1CCC[C@@H](Nc2nnc(-c3ccccc3O)c3cc[nH]c23)C1 ; CN1CCC[C@@H](Nc2nnc(-c3ccccc3)c3cc[nH]c23)C1 ; Cc1cccc(O)c1-c1nnc(N[C@@H]2CCCN(C)C2)c2[nH]ccc12 ; CN1CCC[C@@H](Nc2nnc(-c3ccccc3O)c3ccn(C)c23)C1 ; CN1CCC[C@@H](N(C)c2nnc(-c3ccccc3O)c3ccn(C)c23)C1]

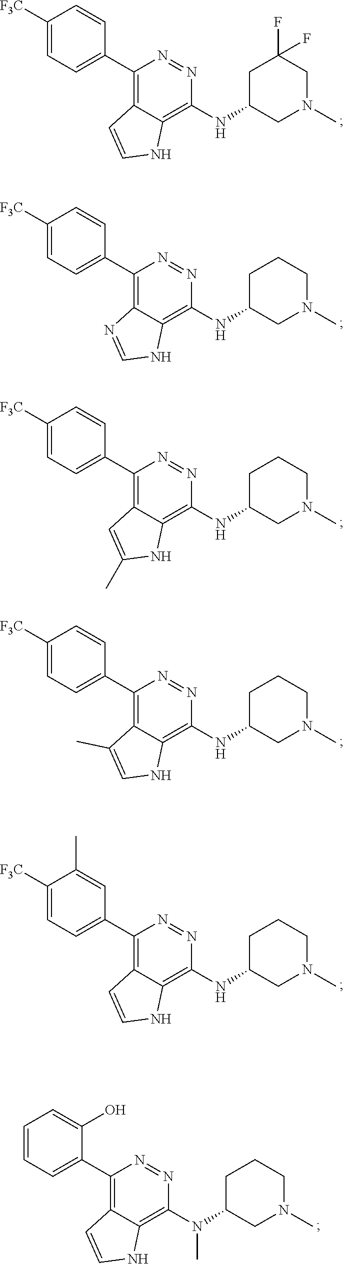 [SMILES: CN1C[C@H](Nc2nnc(-c3ccc(C(F)(F)F)cc3)c3cc[nH]c23)CC(F)(F)C1 ; CN1CCC[C@@H](Nc2nnc(-c3ccc(C(F)(F)F)cc3)c3nc[nH]c23)C1 ; Cc1cc2c(-c3ccc(C(F)(F)F)cc3)nnc(N[C@@H]3CCCN(C)C3)c2[nH]1 ; Cc1c[nH]c2c(N[C@@H]3CCCN(C)C3)nnc(-c3ccc(C(F)(F)F)cc3)c12 ; Cc1cc(-c2nnc(N[C@@H]3CCCN(C)C3)c3[nH]ccc23)ccc1C(F)(F)F ; CN1CCC[C@@H](N(C)c2nnc(-c3ccccc3O)c3cc[nH]c23)C1]

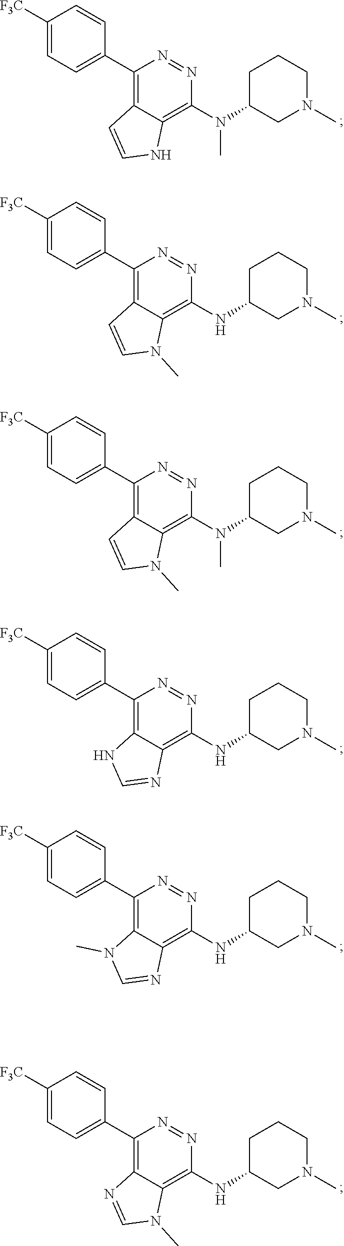 [SMILES: CN1CCC[C@@H](N(C)c2nnc(-c3ccc(C(F)(F)F)cc3)c3cc[nH]c23)C1 ; CN1CCC[C@@H](Nc2nnc(-c3ccc(C(F)(F)F)cc3)c3ccn(C)c23)C1 ; CN1CCC[C@@H](N(C)c2nnc(-c3ccc(C(F)(F)F)cc3)c3ccn(C)c23)C1 ; CN1CCC[C@@H](Nc2nnc(-c3ccc(C(F)(F)F)cc3)c3[nH]cnc23)C1 ; CN1CCC[C@@H](Nc2nnc(-c3ccc(C(F)(F)F)cc3)c3c2ncn3C)C1 ; CN1CCC[C@@H](Nc2nnc(-c3ccc(C(F)(F)F)cc3)c3ncn(C)c23)C1]

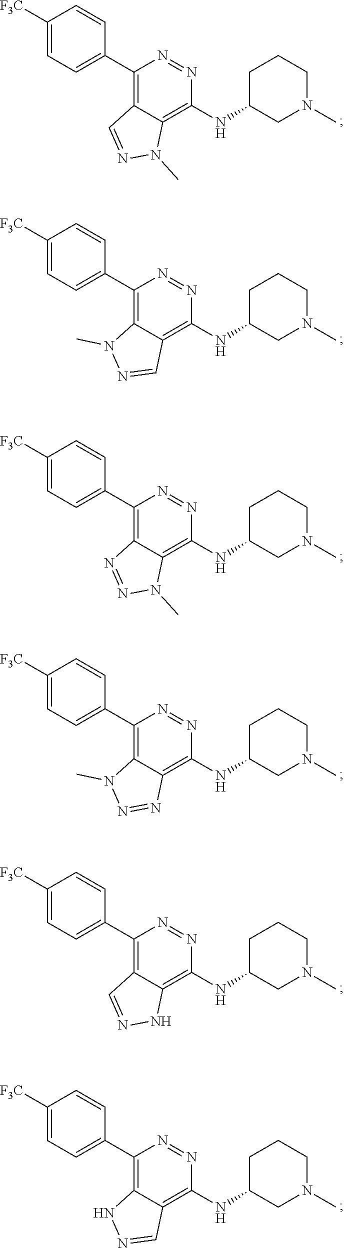 [SMILES: CN1CCC[C@@H](Nc2nnc(-c3ccc(C(F)(F)F)cc3)c3cnn(C)c23)C1 ; CN1CCC[C@@H](Nc2nnc(-c3ccc(C(F)(F)F)cc3)c3c2cnn3C)C1 ; CN1CCC[C@@H](Nc2nnc(-c3ccc(C(F)(F)F)cc3)c3nnn(C)c23)C1 ; CN1CCC[C@@H](Nc2nnc(-c3ccc(C(F)(F)F)cc3)c3c2nnn3C)C1 ; CN1CCC[C@@H](Nc2nnc(-c3ccc(C(F)(F)F)cc3)c3cn[nH]c23)C1 ; CN1CCC[C@@H](Nc2nnc(-c3ccc(C(F)(F)F)cc3)c3[nH]ncc23)C1]

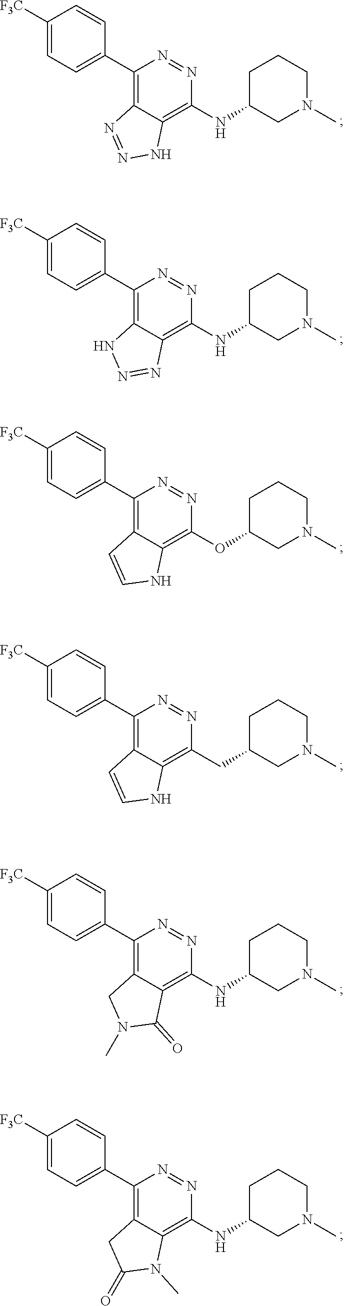 [SMILES: CN1CCC[C@@H](Nc2nnc(-c3ccc(C(F)(F)F)cc3)c3nn[nH]c23)C1 ; CN1CCC[C@@H](Nc2nnc(-c3ccc(C(F)(F)F)cc3)c3[nH]nnc23)C1 ; CN1CCC[C@@H](Oc2nnc(-c3ccc(C(F)(F)F)cc3)c3cc[nH]c23)C1 ; CN1CCC[C@@H](Cc2nnc(-c3ccc(C(F)(F)F)cc3)c3cc[nH]c23)C1 ; CN1CCC[C@@H](Nc2nnc(-c3ccc(C(F)(F)F)cc3)c3c2C(=O)N(C)C3)C1 ; CN1CCC[C@@H](Nc2nnc(-c3ccc(C(F)(F)F)cc3)c3c2N(C)C(=O)C3)C1]

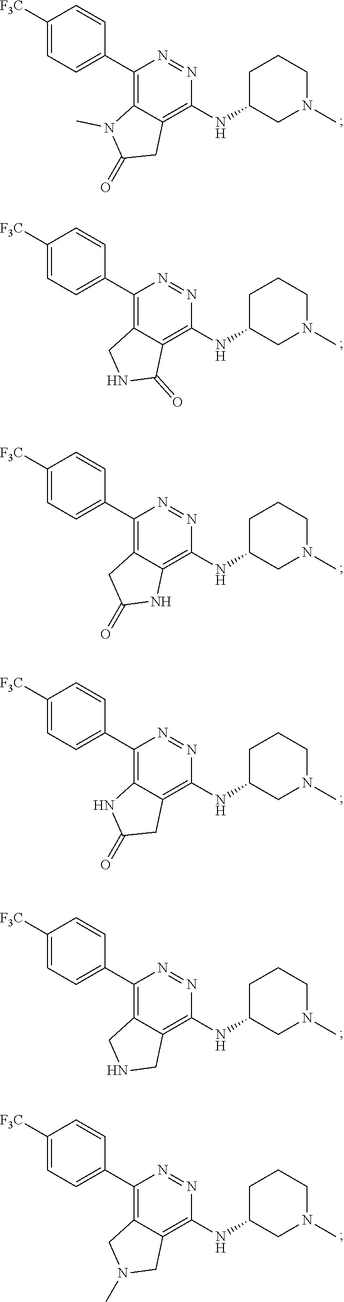 [SMILES: CN1CCC[C@@H](Nc2nnc(-c3ccc(C(F)(F)F)cc3)c3c2CC(=O)N3C)C1 ; CN1CCC[C@@H](Nc2nnc(-c3ccc(C(F)(F)F)cc3)c3c2C(=O)NC3)C1 ; CN1CCC[C@@H](Nc2nnc(-c3ccc(C(F)(F)F)cc3)c3c2NC(=O)C3)C1 ; CN1CCC[C@@H](Nc2nnc(-c3ccc(C(F)(F)F)cc3)c3c2CC(=O)N3)C1 ; CN1CCC[C@@H](Nc2nnc(-c3ccc(C(F)(F)F)cc3)c3c2CNC3)C1 ; CN1Cc2c(N[C@@H]3CCCN(C)C3)nnc(-c3ccc(C(F)(F)F)cc3)c2C1]

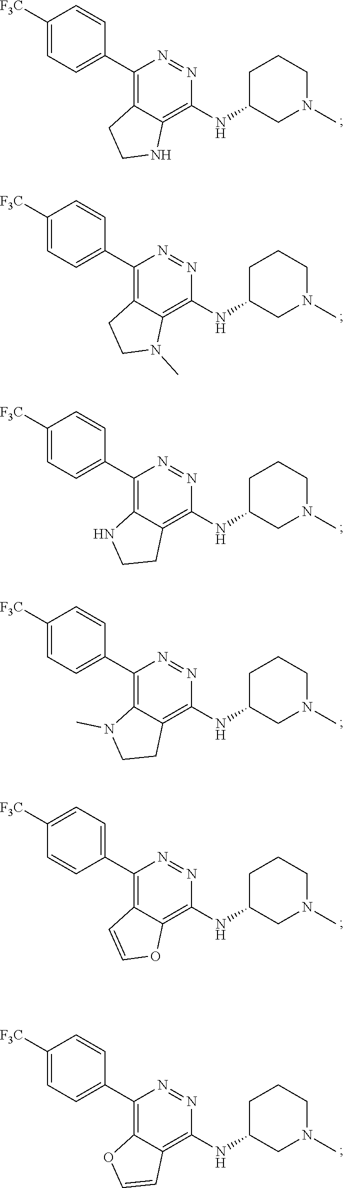 [SMILES: CN1CCC[C@@H](Nc2nnc(-c3ccc(C(F)(F)F)cc3)c3c2NCC3)C1 ; CN1CCC[C@@H](Nc2nnc(-c3ccc(C(F)(F)F)cc3)c3c2N(C)CC3)C1 ; CN1CCC[C@@H](Nc2nnc(-c3ccc(C(F)(F)F)cc3)c3c2CCN3)C1 ; CN1CCC[C@@H](Nc2nnc(-c3ccc(C(F)(F)F)cc3)c3c2CCN3C)C1 ; CN1CCC[C@@H](Nc2nnc(-c3ccc(C(F)(F)F)cc3)c3ccoc23)C1 ; CN1CCC[C@@H](Nc2nnc(-c3ccc(C(F)(F)F)cc3)c3occc23)C1]

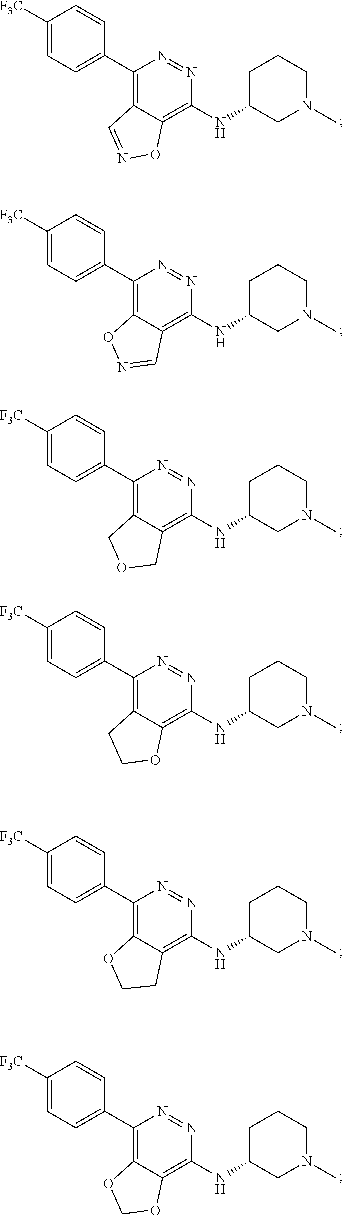 [SMILES: CN1CCC[C@@H](Nc2nnc(-c3ccc(C(F)(F)F)cc3)c3cnoc23)C1 ; CN1CCC[C@@H](Nc2nnc(-c3ccc(C(F)(F)F)cc3)c3oncc23)C1 ; CN1CCC[C@@H](Nc2nnc(-c3ccc(C(F)(F)F)cc3)c3c2COC3)C1 ; CN1CCC[C@@H](Nc2nnc(-c3ccc(C(F)(F)F)cc3)c3c2OCC3)C1 ; CN1CCC[C@@H](Nc2nnc(-c3ccc(C(F)(F)F)cc3)c3c2CCO3)C1 ; CN1CCC[C@@H](Nc2nnc(-c3ccc(C(F)(F)F)cc3)c3c2OCO3)C1]

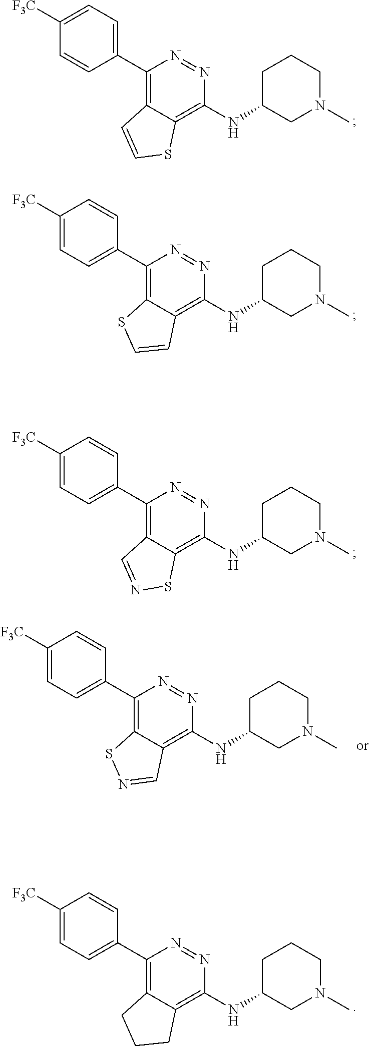 [SMILES: CN1CCC[C@@H](Nc2nnc(-c3ccc(C(F)(F)F)cc3)c3ccsc23)C1 ; CN1CCC[C@@H](Nc2nnc(-c3ccc(C(F)(F)F)cc3)c3sccc23)C1 ; CN1CCC[C@@H](Nc2nnc(-c3ccc(C(F)(F)F)cc3)c3cnsc23)C1 ; CN1CCC[C@@H](Nc2nnc(-c3ccc(C(F)(F)F)cc3)c3sncc23)C1 ; CN1CCC[C@@H](Nc2nnc(-c3ccc(C(F)(F)F)cc3)c3c2CCC3)C1]

141. The compound of claim 1, or a pharmaceutically acceptable salt, isomer, hydrate, solvate or isotope thereof, wherein the compound has the following structure:

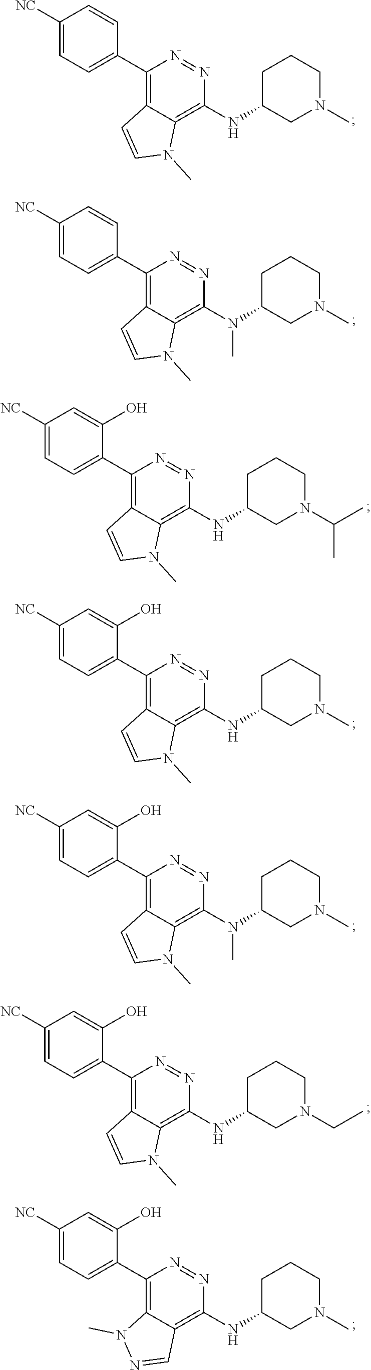

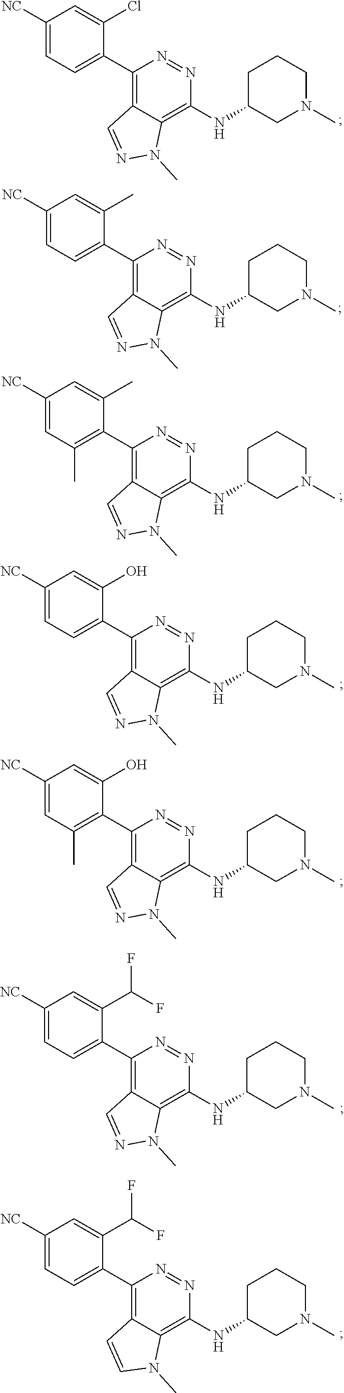

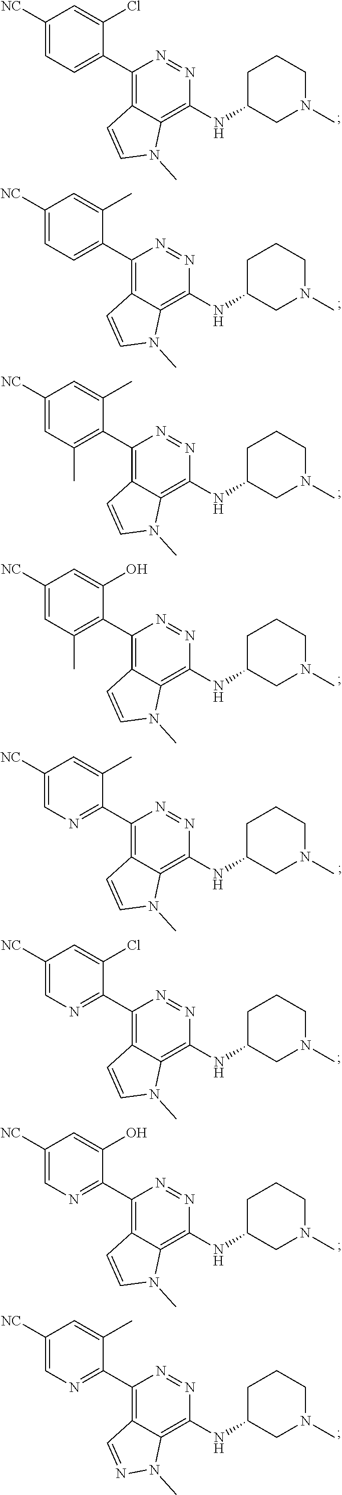

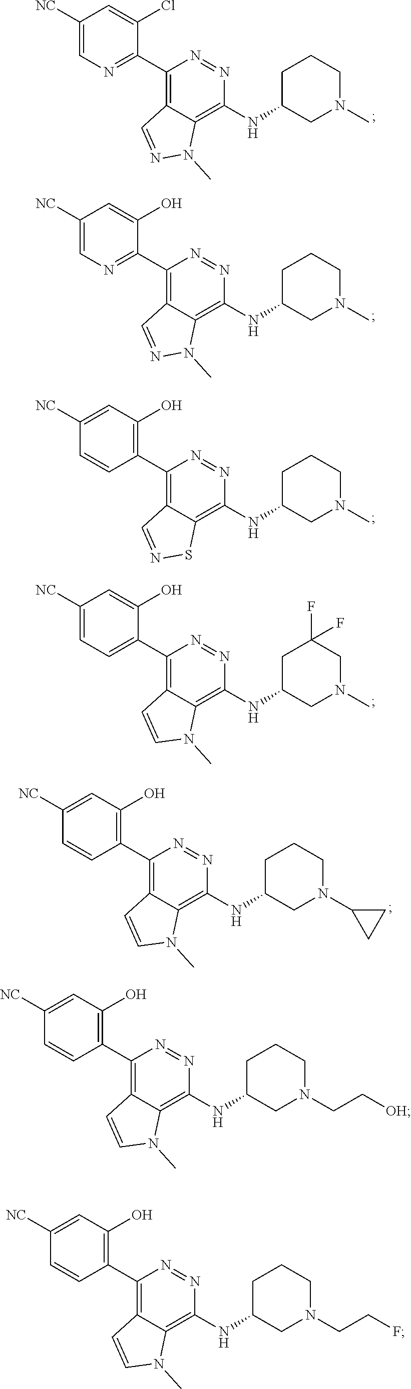

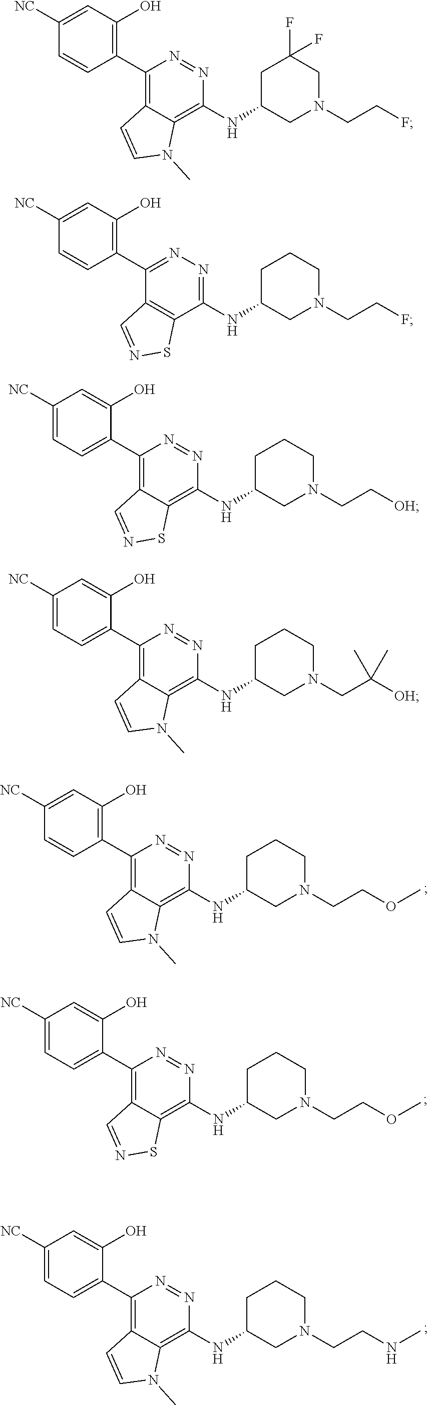

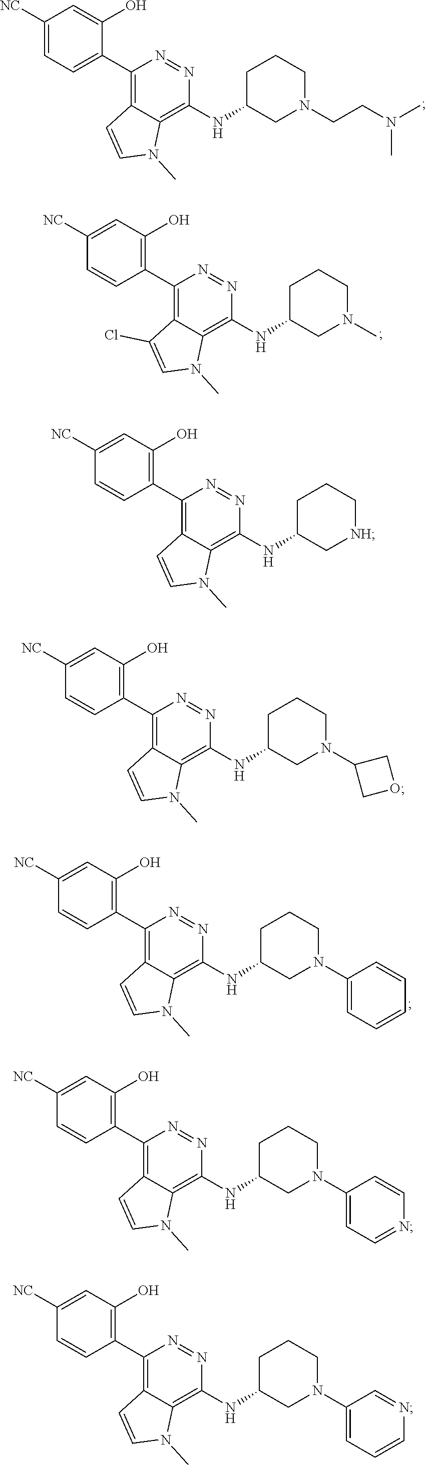

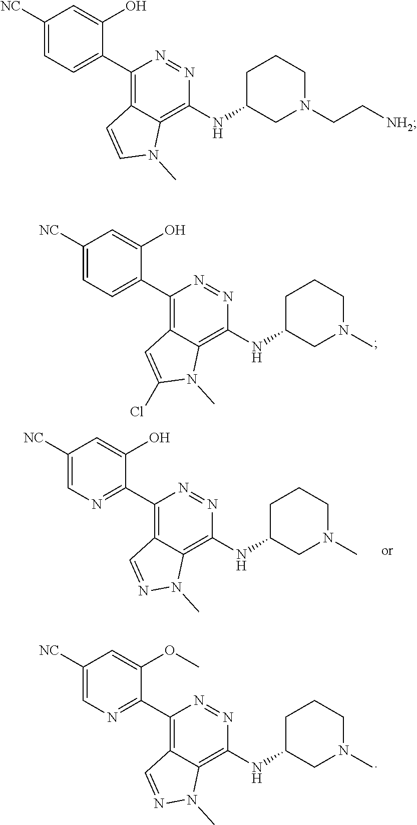

142. A pharmaceutical composition comprising the compound of any one of claims 1-141, or a pharmaceutically acceptable salt, isomer, hydrate, solvate or isotope thereof, and at least one pharmaceutically acceptable excipient.

143. A method of modulating NLRP3 inflammasome activity by contacting NLRP3 inflammasome with an effective amount of the compound of any one of claims 1-141 or the pharmaceutical composition of claim 142.

144. A method of treating NLRP3 inflammasome dependent condition by administering to a subject in need thereof an effective amount of the pharmaceutical composition of claim 142.

145. The method of claim 144, wherein the NLRP3 inflammasome dependent condition is neuroinflammation-related disorders or neurodegenerative diseases.
